(19) United States
(12) Patent Application Publication
(10) Pub. No.: US 2010/0331305 A1
(43) Pub. Date: Dec. 30, 2010

(54) OXO-HETEROCYCLE FUSED PYRIMIDINE COMPOUNDS, COMPOSITIONS AND METHODS OF USE

(75) Inventors: Philippe Bergeron, San Francisco, CA (US); Frederick Cohen, San Francisco, CA (US); Anthony Estrada, San Carlos, CA (US); Michael F.T. Koehler, Palo Alto, CA (US); Cuong Ly, Daly City, CA (US); Joseph P. Lyssikatos, Piedmont, CA (US); Zhonghua Pei, South San Francisco, CA (US); Wendy Lee, San Ramon, CA (US); Xianrui Zhao, San Mateo, CA (US)

Correspondence Address: GENENTECH, INC., 1 DNA WAY, SOUTH SAN FRANCISCO, CA, 94080, US

(73) Assignee: Genentech, Inc., South San Francisco, CA (US)

(21) Appl. No.: 12/821,998

(22) Filed: Jun. 23, 2010

Related U.S. Application Data
(60) Provisional application No. 61/252,284, filed on Oct. 16, 2009.
(60) Provisional application No. 61/220,011, filed on Jun. 24, 2009.

(51) Int. Cl. A61K 31/5377; C07D 491/052; A61P 35/00; A61P 35/04; A61K 31/519; C07D 491/10; C07D 498/08; A61K 31/5386

(52) U.S. Cl. 514/210.21; 544/117; 514/234.2; 544/278; 514/260.1; 544/70; 544/105; 514/230.5

(57) ABSTRACT

Disclosed are compounds of Formula I, including steroisomers, geometric isomers, tautomers, solvates, metabolites and pharmaceutically acceptable salts thereof, that are useful in modulating PIKK related kinase signaling, e.g., mTOR, and for the treatment of diseases (e.g., cancer) that are mediated at least in part by the dysregulation of the PIKK signaling pathway (e.g., mTOR).

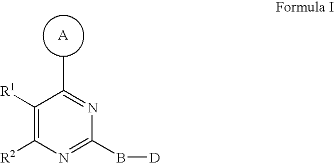

### CROSS-REFERENCE TO RELATED APPLICATIONS

This application claims priority to U.S. provisional application Nos. 61/252,284 filed on Oct. 16, 2009 and 61/220,011, filed on Jun. 24, 2009, each of which is incorporated herein by reference for all purposes.

### BACKGROUND OF INVENTION

The mammalian target of rapamycin (mTOR) is a 289 kDa serine/threonine kinase that is considered a member of the phosphoinositide-3-kinase-like kinase (PIKK) family, because it contains a carboxyl terminal kinase domain that has significant sequence homology to the catalytic domain of phosphoinositide 3-kinase (PI3K) lipid kinases. In addition to the catalytic domain at the C-terminus, mTOR kinase also contains a FKBP12-Rapamycin binding (FRB) domain, a putative repressor domain near the C-terminus and up to 20 tandemly-repeated HEAT motifs at the N-terminus as well as a FRAP-ATM-TRRAP (FAT) and FAT C-terminus domain. See, Huang and Houghton, Current Opinion in Pharmacology, 2003, 3, 371-377.) In the literature, mTOR kinase is also referred to as FRAP (FKBP12 and rapamycin associated protein), RAFT1 (rapamycin and FKBP12 target 1), RAPT1 (rapamycin target 1)).

mTOR kinase can be activated by growth factors through the PI3K-Akt pathway or by cellular stresses, such as deprivation of nutrients or hypoxia. The activation of mTOR kinase is thought to play a central role in regulating cell growth and cell survival via a wide range of cellular functions including translation, transcription, mRNA turnover, protein stability, actin cytoskeleton reorganization and autophagy. For a detailed review of mTOR cell signaling biology and potential therapeutic effects of modulating the mTOR signaling interactions, see Sabatini, D. M. and Guertin, D. A. (2005) An Expanding Role for mTOR in Cancer TRENDS in Molecular Medicine, 11, 353-361; Chiang, G. C. and Abraham, R. T. (2007) Targeting the mTOR signaling network in cancer TRENDS 13, 433-442; Jacinto and Hall (2005) Tor signaling in bugs, brain and brawn Nature Reviews Molecular and Cell Biology, 4, 117-126; and Sabatini, D. M. and Guertin, D. A. (2007) Defining the Role of mTOR in Cancer Cancer Cell, 12, 9-22.

Researchers studying mTOR kinase biology have discovered a pathological connection between the dysregulation of mTOR cell signaling and a number of diseases including immunological disorders, cancer, metabolic diseases, cardiovascular diseases and neurological disorders.

For example, there is evidence to show that PI3K-AKT signaling pathway, which lies upstream of mTOR kinase, is frequently overactivated in cancer cells, which subsequently results in the hyperactivation of downstream targets like mTOR kinase. More specifically, the components of the PI3K-AKT pathway that are mutated in different human tumors include, activation mutations of growth factor receptors and the amplification and overexpression of PI3K and AKT. In addition, there is evidence which shows that many tumor types, including glioblastoma, hepatocellular carcinoma, lung carcinoma, melanoma, endometrial carcinomas, and prostate cancer, contain loss-of-function mutations of negative regulators of the PI3K-AKT pathways, such as phosphatases and tensin homolog deleted on chromosome 10 (PTEN) and tuberous sclerosis complex (TSC1/TSC2), which also results in hyperactive signaling of mTOR kinase. The above suggests that inhibitors of mTOR kinase can be effective therapeutics for the treatment of diseases caused, at least in part, by the hyperactivity of the mTOR kinase signalling.

mTOR kinase exists as two physically and functionally distinct signaling complexes (i.e., mTORC1 and mTORC2). mTORC1, also known as the “mTOR-Raptor complex” or the “rapamycin-sensitive complex” because it binds to and is inhibited by the small molecule inhibitor rapamycin. mTORC1 is defined by the presence of the proteins mTOR, Raptor and mLST8. Rapamycin, itself, is a macrolide and was discovered as the first small molecule inhibitor of mTOR kinase. To be biologically active, rapamycin forms a ternary complex with mTOR and FKBP12, which is a cytosolic binding protein collectively called immunophilin. Rapamycin acts to induce the dimerization of mTOR and FKBP12. The formation of rapamycin-FKBP12 complex results in a gain-of-function, because the complex binds directly to mTOR and inhibits the function of mTOR.

A second, more recently discovered mTORC complex, mTORC2, is characterized by the presence of the proteins mTOR, Rictor, Protor-1, mLST8 and mSIN1. mTORC2 is also referred to as the “mTOR-Rictor complex” or the “rapamycin-insensitive” complex because it does not bind to rapamycin.

Both mTOR complexes play important roles in intracellular signaling pathways that affect a cell's growth, and proliferation, and survival. For example, the downstream target proteins of mTORC1 include Ribosomal S6 kinases (e.g., S6K1, S6K2) and eukaryotic initiation factor 4E binding protein (4E-BP1), which are key regulators of protein translation in cells. Also, mTORC2 is responsible for the phosphorylation of AKT (S473); and studies have shown that uncontrolled cell proliferation due to hyperactivation of AKT to be a hallmark of several cancer types.

Currently, several rapamycin analogues are in clinical development for cancer (e.g., Wyeth's CCI-779, Novartis' RAD001 and Ariad Pharmaceuticals' AP23573). Interestingly, the clinical data shows that the rapamycin analogs appear to be effective for certain cancer types, such as mantle-cell lymphoma, endometrial cancer, and renal cell carcinoma.

The discovery of a second mTOR protein complex (mTORC2) that is not inhibited by rapamycin or its analogs suggest that inhibition of mTOR by rapamycin is incomplete and that a direct mTOR kinase inhibitor which can inhibit both mTORC1 and mTORC2 at the catalytic ATP binding site can be more efficacious and have broader anti-tumor activity than rapamycin and its analogs.

Recently, small molecule mTOR inhibitors have been disclosed, including in U.S. patent application Ser. Nos. 11/599,663 and 11/657,156 to OSI Pharmaceuticals Inc.; in International Applications WO/2008/023161 and WO/2006/090169 to Kudos Pharmacuticals; and in International Applications WO/2008/032060, WO/2008/032086, WO/2008032033, WO/2008/032028, WO/2008/032036, WO/2008/032089, WO/2008/032072, WO/2008/031091 to AstraZeneca.

U.S. Provisional Application 61/085,309 discloses a class of N-heterocyclic fused pyrimidine compounds with mTOR activity.

In view of the increased knowledge of the role of mTOR signaling in diseases (e.g., cancer), it is desirable to have small molecule inhibitors of mTOR (including mTORC1 and mTORC2) that can be used to treat diseases wherein aberrant mTOR activity is observed, such as, for example, in cancer. In addition, it can be desirable to have small molecule inhibitors of related enzymes (e.g., PI3K, AKT) that functions upstream or downstream of the mTOR signaling pathway.

### SUMMARY OF INVENTION

In one aspect, the present invention provides for a compound of Formula I

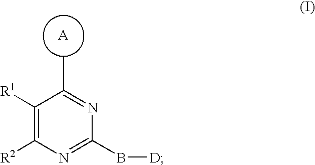

or a pharmaceutically acceptable salt thereof, wherein in Formula I, A is a 5- to 8-membered heterocyclic ring having from 1 to 3 heteroatoms independently selected from N, O and S as ring vertices, and having from 0 to 2 double bonds; wherein the A ring is further substituted with from 0 to 5 R^{A} substituents selected from the group consisting of —C(O)OR^{a}, —C(O)NR^{a}R^{b}, —NR^{a}R^{b}, —OC(O)R^{c}—OR^{a}, —SR^{a}, —S(O)₂R^{c}, —S(O)R^{c}, —R^{c}, —(CH₂)₁₋₄—NR^{a}R^{b}, —(CH₂)₁₋₄—NR^{a}C(O)R^{c}, —(CH₂)₁₋₄—OR^{a}, —(CH₂)₁₋₄—SR^{a}, —(CH₂)₁₋₄—S(O)₂R^{c}, —(CH₂)₁₋₄—S(O)R^{c}, halogen, —NO₂, —CN and —N₃, wherein R^{a} and R^{b} are each independently selected from hydrogen, C₁₋₆ alkyl, C₁₋₆ haloalkyl, C₁₋₆ heteroalkyl, C₂₋₆ alkenyl, C₂₋₆ alkynyl, C₃₋₆ cycloalkyl, phenyl and —(CH₂)₁₋₄(phenyl), and optionally R^{a} and R^{b}, together with the nitrogen atom to which each is attached, are combined to form a 3- to 7-membered heterocyclic ring comprising 1 to 2 heteroatoms selected from N, O and S; R^{c} is selected from C₁₋₆ alkyl, C₁₋₆ haloalkyl, C₁₋₆ heteroalkyl, C₂₋₆ alkenyl, C₂₋₆ alkynyl, C₃₋₆ cycloalkyl, phenyl and —(CH₂)₁₋₄ (phenyl); and any two substituents attached to the same atom in the 5- to 8-membered heterocyclic ring are optionally combined to form a 3- to 5-membered carbocyclic or 3 to 5-membered heterocyclic ring. R¹ and R² are combined with the atoms to which they are attached to form a 5- to 8-membered monocyclic or bridged bicyclic heterocyclic ring comprising —O— as one of the ring vertices; wherein the 5- to 8-membered monocyclic or bridged-bicyclic heterocyclic ring formed by combining R¹ and R² further optionally comprises one additional heteroatom selected from the group consisting of N, O and S, and is substituted with from 0 to 5 R^{R} substituents selected from the group consisting of halogen, —NR^{j}R^{k}, —SR^{j}, —OR^{j}, —C(O)OR^{j}, —C(O)NR^{j}R^{k}, —NHC(O)R^{j}, —OC(O)R^{j}, —R^{m}, —CN, ═O, ═S, ═N—CN, —(CH₂)₁₋₄—CN, —(CH₂)₁₋₄—OR^{j}, —(CH₂)₁₋₄—NR^{j}R^{k}, —C₁₋₄ alkylene-OR^{j}, —C₁₋₄ alkenylene-R^{m}, —C₂₋₄ alkenylene-R^{m} and —C₂₋₄ alkynylene-R^{m}, —C₁₋₄ alkylene-C₁₋₉ heteroaryl, C₂₋₄ alkenylene-C₁₋₉ heteroaryl, C₂₋₄ alkynylene-C₁₋₉ heteroaryl, —C₁₋₄ alkylene-C₆₋₄₀ aryl, C₂₋₄ alkenylene-C₆₋₁₀ aryl and C₂₋₄ alkynylene-C₆₋₁₀ aryl, wherein R^{j} and R^{k} are each independently selected from hydrogen, C₁₋₆ alkyl, C₁₋₆ haloalkyl, C₁₋₆ heteroalkyl, C₂₋₆ alkenyl, C₂₋₆ alkynyl, C₃₋₇ cycloalkyl, C₂₋₆ heterocycloalkyl, phenyl, pyridyl and —(CH₂)₁₋₄-(Ph), and R^{j} and R^{k}, when attached to the same nitrogen atom, are optionally combined to form a 3- to 6-membered heterocyclic ring comprising 1 to 2 heteroatoms selected from N, O and S; and R^{m} is selected from C₁₋₆ alkyl, C₁₋₆ haloalkyl, C₁₋₆ heteroalkyl, C₂₋₆ alkenyl, C₂₋₆ alkynyl, C₃₋₇ cycloalkyl, C₂₋₆ heterocycloalkyl and —(CH₂)₁₋₄—(Ph), and wherein a C₃₋₇ cycloalkyl, C₂₋₆ heterocycloalkyl, C₁₋₉ heteroaryl or C₆₋₁₀ aryl portion of a R^{R} substituent is substituted with from 0 to 3 substituents selected from the group consisting of F, Cl, Br, I, —NH(C₁₋₄ alkyl), —N(diC₁₋₄ alkyl), O(C₁₋₄ alkyl), C₁₋₆ alkyl, C₁₋₆ heteroalkyl, —C(O)O(C₁₋₄ alkyl), —C(O)NH(C₁₋₄alkyl), —C(O)N(diC₁₋₄ alkyl), —NO₂, —CN; wherein when R¹ and R² are combined to form a monocyclic 5- to 8-membered heterocyclic ring then any two R^{R} substitutents attached to the same atom or adjacent carbon atoms in said 5- to 8-membered heterocyclic ring are optionally combined to form a 3- to 7-membered cycloalkyl ring or a 3- to 7-membered heterocycloalkyl ring comprising 1 to 2 heteroatoms selected from N, O and S as ring vertices. B is a member selected from the group consisting of phenylene and 5- to 6-membered heteroarylene, and is substituted with from 0 to 4 R^{B} substituents selected from halogen, —CN, —N₃, —NO₂, —C(O)ORⁿ, —C(O)NRⁿR^{o}, —NRⁿC(O)R^{o}, —NRⁿC(O)NRⁿR^{o}, —ORⁿ, —NRⁿR^{o}, —(CH₂)₁₋₄—C(O)ORⁿ, —(CH₂)₁₋₄—C(O)NRⁿR^{o}, —(CH₂)₁₋₄—ORⁿ, —(CH₂)₁₋₄—NRⁿR^{o}, —(CH₂)₁₋₄—SR^{p} and R^{p}; wherein Rⁿ and R^{o} are independently selected from hydrogen and C₁₋₆ alkyl, C₁₋₆ haloalkyl, C₁₋₆ heteroalkyl, C₂₋₆ alkenyl, C₂₋₆ alkynyl, C₃₋₇ cycloalkyl, C₂₋₆ heterocycloalkyl, phenyl and —(CH₂)₁₋₄-(phenyl) or when attached to the same nitrogen atom, Rⁿ and R^{o} are optionally are combined to form a 3- to 6-membered heterocyclic ring comprising 1 to 2 heteroatoms selected from N, O and S; R^{p} is C₁₋₆ alkyl, C₁₋₆ haloalkyl, C₁₋₆ heteroalkyl, C₂₋₆ alkenyl, C₂₋₆ alkynyl, C₃₋₇ cycloalkyl, C₂₋₆ heterocycloalkyl, phenyl and —(CH₂)₁₋₄-(phenyl), wherein any two substituents, not including the D group, located on adjacent atoms of B are optionally combined to form a 5- to 6-membered carbocyclic, heterocyclic, aryl or heteroaryl ring. Finally, D is a member selected from the group consisting of —NR³C(O)NR⁴R⁵, —NR⁴R⁵, —C(O)NR⁴R⁵, —OC(O)OR⁴, —OC(O)NR⁴R⁵, —NR³C(═N—CN)NR⁴R⁵, —NR³C(═N—OR⁴)NR⁴R⁵, —NR³C(═N—NR⁴)NR⁴R⁵, —NR³C(O)R⁴, —NR³C(O)OR⁴, —NR³S(O)₂NR⁴R⁵, —NR³S(O)₂R⁴, —NR³C(═S)NR⁴R⁵ and —S(O)₂R⁴R⁵, wherein R³ is selected from the group consisting of hydrogen, C₁₋₆ alkyl, C₁₋₆ haloalkyl and C₂₋₆ alkenyl; R⁴ and R⁵ are each independently selected from the group consisting of hydrogen, C₁₋₆ alkyl, C₁₋₆ haloalkyl, C₁₋₆ alkylamino-C(═O)—, C₂₋₆ alkenyl, C₂₋₆ alkynyl, C₃₋₁₀ cycloalkyl, C₂₋₉ heterocycloalkyl, C₆₋₁₀ aryl and C₁₋₉ heteroaryl, and R⁴ and R⁵, when attached to the same nitrogen atom, are optionally combined to form a 5- to 7-membered heterocyclic or 5- to 6-membered heteroaryl ring comprising 1 to 3 heteroatoms selected from N, O and S; and wherein R³, R⁴ and R⁵ are further substituted with from 0 to 3 R^{D} substituents independently selected from the group consisting of halogen, —NO₂, —CN, —NR^{q}R^{r}, —OR^{q}, —SR^{q}, —C(O)OR^{q}, —C(O)NR^{q}R^{r}, —NR^{q}C(O)R^{r}, —NR^{q}C(O)OR^{s}, —(CH₂)₁₋₄—NR^{q}R^{r}, —(CH₂)₁₋₄—OR^{q}, —(CH₂)₁₋₄—SR^{q}, —(CH₂)₁₋₄—C(O)OR^{q}, —(CH₂)₁₋₄—C(O)NR^{q}R^{r}, —(CH₂)₁₋₄—NR^{q}C(O)R^{r}, —(CH₂)₁₋₄—NR^{q}C(O)OR^{r}, —(CH₂)₁₋₄—CN, —(CH₂)₁₋₄—NO₂, —S(O)R^{r}, —S(O)₂R^{r}, —(CH₂)₁₋₄R^{s}, ═O, and —R^{s}; wherein R^{q} and R^{r} is selected from hydrogen, C₁₋₆ alkyl, C₁₋₆ haloalkyl, C₂₋₆ alkenyl, C₂₋₆ alkynyl, C₁₋₆ heteroalkyl, C₃₋₇ cycloalkyl, C₂₋₆ heterocycloalkyl, C₆₋₁₀ aryl, C₁₋₉ heteroaryl; and R^{s}, at each occurrence, is independently selected from C₁₋₆ alkyl, C₁₋₆ haloalkyl, C₃₋₇ cycloalkyl, C₂₋₆ heterocycloalkyl, C₆₋₁₀ aryl and C₁₋₉ heteroaryl; and wherein the D group and a substituent located on an adjacent atom of the B ring are optionally combined to form a 5- to 6-membered heterocyclic or heteroaryl ring, optionally substituted with 1 to 2 R^{D} substituents.

The present invention also provides for a compound of Formula I

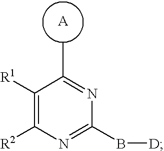

or a pharmaceutically acceptable salt thereof, wherein in Formula I, A is a 5- to 8-membered heterocyclic ring having from 1 to 3 heteroatoms independently selected from N, O and S as ring vertices, and having from 0 to 2 double bonds; wherein the A ring is further substituted with from 0 to 5 R^{A} substituents selected from the group consisting of C(O)OR^{a}, —C(O)NR^{a}R^{b}, —NR^{a}R^{b}, —OC(O)R^{c}, —OR^{a}, —SR^{a}, —S(O)₂R^{c}, —S(O)R^{c}, —R^{c}, —(CH₂)₁₋₄—NR^{a}R^{b}, —(CH₂)₁₋₄—NR^{a}C(O)R^{c}, —(CH₂)₁₋₄—OR^{a}, —(CH₂)₁₋₄—SR^{a}, —(CH₂)₁₋₄—S(O)₂R^{c}, —(CH₂)₁₋₄—S(O)R^{c}, halogen, —NO₂, —CN and —N₃, wherein R^{a} and R^{b} are each independently selected from hydrogen, C₁₋₆ alkyl, C₁₋₆ haloalkyl, C₁₋₆ heteroalkyl, C₂₋₆ alkenyl, C₂₋₆ alkynyl, C₃₋₆ cycloalkyl, phenyl and —(CH₂)₁₋₄(phenyl), and optionally R^{a} and R^{b}, together with the nitrogen atom to which each is attached, are combined to form a 3- to 7-membered heterocyclic ring comprising 1 to 2 heteroatoms selected from N, O and S; R^{c} is selected from C₁₋₆ alkyl, C₁₋₆ haloalkyl, C₁₋₆ heteroalkyl, C₂₋₆ alkenyl, C₂₋₆ alkynyl, C₃₋₆ cycloalkyl, phenyl and —(CH₂)₁₋₄ (phenyl); and any two substituents attached to the same atom in the 5- to 8-membered heterocyclic ring are optionally combined to form a 3- to 5-membered carbocyclic or a 3 to 5-membered heterocyclic ring. R¹ and R² are combined with the atoms to which they are attached to form a 5- to 8-membered monocyclic or bridged bicyclic heterocyclic ring comprising —O— as one of the ring vertices; wherein the 5- to 8-membered monocyclic or bridged-bicyclic heterocyclic ring formed by combining R¹ and R² further optionally comprises one additional heteroatom selected from the group consisting of N, O and S, and is substituted with from 0 to 5 R^{R} substituents selected from the group consisting of halogen, —NR^{j}R^{k}, —C(O)OR^{j}, —C(O)NR^{j}R^{k}, —NHC(O)R^{j}, —OC(O)R^{j}, —R^{m}, —CN, ═O, ═S, ═N—CN, —(CH₂)₁₋₄—CN, —(CH₂)₁₋₄—OR^{j}, —(CH₂)₁₋₄—NR^{j}R^{k}, —C₁₋₄ alkylene-R^{m}, —C₂₋₄ alkenylene-R^{m}, —C₂₋₄ alkynylene-R^{m}, —C₁₋₄ alkylene-C₁₋₉ heteroaryl, C₂₋₄ alkenylene-C₁₋₉ heteroaryl, C₂₋₄ alkynylene-C₁₋₉ heteroaryl, C₁₋₄ alkylene-C₆₋₄₀ aryl, C₂₋₄ alkynylene-C₆₋₄₀ aryl and C₂₋₄ alkynylene-C₆₋₄₀ aryl, wherein R^{j} and R^{k} are each independently selected from hydrogen, C₁₋₆ alkyl, C₁₋₆ haloalkyl, C₁₋₆ heteroalkyl, C₂₋₆ alkenyl, C₂₋₆ alkynyl, C₃₋₇ cycloalkyl, C₂₋₆ heterocycloalkyl, phenyl, and —(CH₂)₁₋₄-(Ph), and R^{j} and R^{k}, when attached to the same nitrogen atom, are optionally combined to form a 3- to 6-membered heterocyclic ring comprising 1 to 2 heteroatoms selected from N, O and S; and R^{m} is selected from C₁₋₆ alkyl, C₁₋₆ haloalkyl, C₁₋₆ heteroalkyl, C₂₋₆ alkenyl, C₂₋₆ alkynyl, C₃₋₇ cycloalkyl, C₂₋₆ heterocycloalkyl and —(CH₂)₁₋₄—(Ph), and wherein a C₃₋₇ cycloalkyl, C₂₋₆ heterocycloalkyl, C₁₋₉ heteroaryl or C₆₋₁₀ aryl portion of a R^{R} substituent is substituted with from 0 to 3 substituents selected from the group consisting of F, Cl, Br, I, —NH(C₁₋₄ alkyl), —N(diC₁₋₄ alkyl), 0(C₁₋₄ alkyl), C₁₋₆ alkyl, C₁₋₆ heteroalkyl, —C(O)O(C₁₋₄ alkyl), —C(O)NH(C₁₋₄alkyl), —C(O)N(diC₁₋₄ alkyl), —NO₂, —CN; wherein when R¹ and R² are combined to form a monocyclic 5- to 8-membered heterocyclic ring then any two R^{R} substitutents attached to the same atom or adjacent carbon atoms in said 5- to 8-membered heterocyclic ring are optionally combined to form a 3- to 7-membered cycloalkyl ring or a 3- to 7-membered heterocycloalkyl ring comprising 1 to 2 heteroatoms selected from N, O and S as ring vertices. B is a member selected from the group consisting of phenylene and 5- to 6-membered heteroarylene, and is substituted with from 0 to 4 R^{B} substituents selected from halogen, —CN, —N₃, —NO₂, —C(O)ORⁿ, —C(O)NRⁿR^{o}, —NRⁿC(O)R^{o}, —NRⁿC(O)NRⁿR^{o}, —NRⁿR^{o}, —(CH₂)₁₋₄—C(O)ORⁿ, —(CH₂)₁₋₄—C(O)NRⁿR^{o}, —(CH₂)₁₋₄—ORⁿ, —(CH₂)₁₋₄—NRⁿR^{o}, —(CH₂)₁₋₄—SR^{p} and R^{p}; wherein Rⁿ and R^{o} are independently selected from hydrogen and C₁₋₆ alkyl, C₁₋₆ haloalkyl, C₁₋₆ heteroalkyl, C₂₋₆ alkenyl, C₂₋₆ alkynyl, C₃₋₇ cycloalkyl, C₂₋₆ heterocycloalkyl, phenyl and —(CH₂)₁₋₄-(phenyl) or when attached to the same nitrogen atom, Rⁿ and R^{o} are optionally are combined to form a 3- to 6-membered heterocyclic ring comprising 1 to 2 heteroatoms selected from N, O and S; R^{p} is C₁₋₆ alkyl, C₁₋₆ haloalkyl, C₁₋₆ heteroalkyl, C₂₋₆ alkenyl, C₂₋₆ alkynyl, C₃₋₇ cycloalkyl, C₂₋₆ heterocycloalkyl, phenyl and —(CH₂)₁₋₄-(phenyl), wherein any two substituents, not including the D group, located on adjacent atoms of B are optionally combined to form a 5- to 6-membered carbocyclic, heterocyclic, aryl or heteroaryl ring. D is a member selected from the group consisting of —NR³C(O)NR⁴R⁵, —NR⁴R⁵, —C(O)NR⁴R⁵, —OC(O)OR⁴, —OC(O)NR⁴R⁵, —NR³C(═N—CN)NR⁴R⁵, —NR³C(═N—OR⁴)NR⁴R⁵, —NR³C(═N—NR⁴)NR⁴R⁵, —NR³C(O)R⁴, —NR³C(O)OR⁴, —NR³S(O)₂NR⁴R⁵, —NR³S(O)₂R⁴, —NR³C(═S)NR⁴R⁵ and —S(O)₂R⁴R⁵, wherein R³ is selected from the group consisting of hydrogen, C₁₋₆ alkyl, C₁₋₆ haloalkyl and C₂₋₆ alkenyl; R⁴ and R⁵ are each independently selected from the group consisting of hydrogen, C₁₋₆ alkyl, C₁₋₆ haloalkyl, C₁₋₆ alkylamino-C(═O)—, C₂₋₆ alkenyl, C₂₋₆ alkynyl, C₃₋₁₀ cycloalkyl, C₂₋₉ heterocycloalkyl, C₆₋₁₀ aryl and C₁₋₉ heteroaryl, and R⁴ and R⁵, when attached to the same nitrogen atom, are optionally combined to form a 5- to 7-membered heterocyclic or 5- to 6-membered heteroaryl ring comprising 1 to 3 heteroatoms selected from N, O and S; and wherein R³, R⁴ and R⁵ are further substituted with from 0 to 3 R^{D} substituents independently selected from the group consisting of halogen, —NO₂, —CN, —NR^{q}R^{r}, —OR^{q}, —SR^{q}, —C(O)OR^{q}, —C(O)NR^{q}R^{r}, —NR^{q}C(O)R^{r}, —NR^{q}C(O)OR^{s}, —(CH₂)₁₋₄—NR^{q}R^{r}, —(CH₂)₁₋₄—OR^{q}, —(CH₂)₁₋₄—SR^{q}, —(CH₂)₁₋₄—C(O)OR^{q}, —(CH₂)₁₋₄—C(O)NR^{q}R^{r}, —(CH₂)₁₋₄—NR^{q}C(O)R^{r}, —(CH₂)₁₋₄—NR^{q}C(O)OR^{r}, —(CH₂)₁₋₄—CN, —(CH₂)₁₋₄—NO₂, —S(O)R^{r}, —S(O)₂R^{r}, —(CH₂)₁₋₄R^{s}, ═O, and —R^{s}; wherein R^{q} and R^{r} is selected from hydrogen, C₁₋₆ alkyl, C₁₋₆ haloalkyl, C₂₋₆ alkenyl, C₂₋₆ alkynyl, C₁₋₆ heteroalkyl, C₃₋₇ cycloalkyl, C₂₋₆ heterocycloalkyl, C₆₋₁₀ aryl, C₁₋₉ heteroaryl; and R^{s}, at each occurrence, is independently selected from C₁₋₆ alkyl, C₁₋₆ haloalkyl, C₃₋₇ cycloalkyl, C₂₋₆ heterocycloalkyl, C₆₋₁₀ aryl and C₁₋₉ heteroaryl; and wherein the D group and a substituent located on an adjacent atom of the B ring are optionally combined to form a 5- to 6-membered heterocyclic or heteroaryl ring.

In another aspect, the present invention provides for pharmaceutical compositions comprising a compound of Formula I (or embodiments thereof), and therapeutic methods of using such compounds (or embodiments thereof) or pharmaceutical compositions of compounds of Formula I (or embodiements thereof) for inhibiting mTOR activity in a mammal (e.g., a human) and treating diseases (such as, for example, cancer) that are associated with dysregulated mTOR activity.

In another aspect, the present invention provides for the use of a compound of Formula I (or embodiments thereof) for the treatment of diseases (such as, for example, cancer) that are associated with dysregulated mTOR activity.

Additional aspects of the invention are described in detail herein.

### DESCRIPTION OF THE DRAWINGS

FIG. 1, FIG. 2 and FIG. 3 illustrate certain embodiments of D groups in compounds of Formula I.

### DETAILED DESCRIPTION OF THE INVENTION

### I. Definitions

As used herein, the term “alkyl”, by itself or as part of another substituent, means, unless otherwise stated, a straight or branched chain hydrocarbon radical, having the number of carbon atoms designated (i.e., C₁₋₈ means one to eight carbons). Examples of alkyl groups include methyl, ethyl, n-propyl, iso-propyl, n-butyl, t-butyl, iso-butyl, sec-butyl, n-pentyl, n-hexyl, n-heptyl, n-octyl, and the like. The term “alkenyl” refers to an unsaturated alkyl radical having one or more double bonds. Similarly, the term “alkynyl” refers to an unsaturated alkyl radical having one or more triple bonds. Examples of such unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butyryl, and the higher homologs and isomers. The term “cycloalkyl,” “carbocyclic,” or “carbocycle” refers to hydrocarbon rings having the indicated number of ring atoms (e.g., C₃₋₆ cycloalkyl) and being fully saturated or having no more than one double bond between ring vertices. As used herein, “cycloalkyl,” “carbocyclic,” or “carbocycle” is also meant to refer to bicyclic, polycyclic and spirocyclic hydrocarbon rings such as, for example, bicyclo[2.2.1]heptane, pinane, bicyclo[2.2.2]octane, adamantane, norborene, spirocyclic C₅₋₁₂ alkane, etc. As used herein, the terms, “alkenyl,” “alkynyl,” “cycloalkyl,”, “carbocycle,” and “carbocyclic,” are meant to include mono and polyhalogenated variants thereof.

The term “heteroalkyl,” by itself or in combination with another term, means, unless otherwise stated, a stable straight or branched chain hydrocarbon radical, consisting of the stated number of carbon atoms and from one to three heteroatoms selected from the group consisting of O, N, Si and S, and wherein the nitrogen and sulfur atoms can optionally be oxidized and the nitrogen heteroatom can optionally be quaternized. The heteroatom(s) O, N and S can be placed at any interior position of the heteroalkyl group. The heteroatom Si can be placed at any position of the heteroalkyl group, including the position at which the alkyl group is attached to the remainder of the molecule. A “heteroalkyl” can contain up to three units of unsaturation (e.g., double bond, triple bond, a combination of both), and also include mono- and poly-halogenated variants, or combinations thereof. Examples include —CH₂—CH₂—O—CH₃, —CH₂—CH₂—O—CF₃, —CH₂—CH₂—NH—CH₃, —CH₂—CH₂—N(CH₃)—CH₃, —CH₂—S—CH₂—CH₃, —S(O)—CH₃, —CH₂—CH₂—S(O)₂—CH₃, —CH═CH—O—CH₃, —Si(CH₃)₃, —CH₂—CH═N—OCH₃, and —CH═CH=N(CH₃)—CH₃. Up to two heteroatoms can be consecutive, such as, for example, —CH₂—NH—OCH₃ and —CH₂—O—Si(CH₃)₃.

The term “heterocycloalkyl,” “heterocyclic,” or “heterocycle” refers to a cycloalkane group that contain from one to five heteroatoms selected from N, O, and S, wherein the nitrogen and sulfur atoms are optionally oxidized, and the nitrogen atom(s) are optionally quaternized. Unless otherwise stated, a “heterocycloalkyl,” “heterocyclic,” or “heterocycle” ring can be a monocyclic, a bicyclic, spirocyclic or a polycylic ring system. Non limiting examples of “heterocycloalkyl,” “heterocyclic,” or “heterocycle” rings include pyrrolidine, piperidine, imidazolidine, pyrazolidine, butyrolactam, valerolactam, imidazolidinone, hydantoin, dioxolane, phthalimide, piperidine, pyrimidine-2,4(1H,3H)-dione, 1,4-dioxane, morpholine, thiomorpholine, thiomorpholine-5-oxide, thiomorpholine-S,S-oxide, piperazine, pyran, pyridone, 3-pyrroline, thiopyran, pyrone, tetrahydrofuran, tetrhydrothiophene, quinuclidine, tropane and the like. A “heterocycloalkyl,” “heterocyclic,” or “heterocycle” group can be attached to the remainder of the molecule through one or more ring carbons or heteroatoms. A “heterocycloalkyl,” “heterocyclic,” or “heterocycle” can include mono- and poly-halogenated variants thereof.

The term “alkylene” by itself or as part of another substituent means a divalent radical derived from an alkane, as exemplified by —CH₂CH₂CH₂CH₂—. Typically, an alkyl (or alkylene) group will have from 1 to 24 carbon atoms, with those groups having 10 or fewer carbon atoms being preferred in the present invention. “Haloalkylene” refers to mono and poly halogenated variants of alkylene. “Alkenylene” and “alkynylene” refer to the unsaturated forms of “alkylene” having double or triple bonds, respectively and are also meant to include mono and poly-halogenated variants.

The term “heteroalkylene” by itself or as part of another substituent means a divalent radical, saturated or unsaturated or polyunsaturated, derived from heteroalkyl, as exemplified by —CH₂—CH₂—S—CH₂CH₂— and —CH₂—S—CH₂—CH₂—NH—CH₂—, —O—CH₂—CH═CH—, —CH₂—CH═C(H)CH₂—O—CH₂— and —S—CH₂—C≡C—. For heteroalkylene groups, heteroatoms can also occupy either or both of the chain termini (e.g., alkyleneoxy, alkylenedioxy, alkyleneamino, alkylenediamino, and the like).

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively. Additionally, for dialkylamino groups, the alkyl portions can be the same or different and can also be combined to form a 3-7 membered ring with the nitrogen atom to which each is attached. Accordingly, a group represented as —NR^{a}R^{b} is meant to include piperidinyl, pyrrolidinyl, morpholinyl, azetidinyl and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “C₁₋₄ haloalkyl” is mean to include trifluoromethyl, 2,2,2-trifluoroethyl, 4-chlorobutyl, 3-bromopropyl, difluoromethyl, and the like.

The term “aryl” means, unless otherwise stated, a polyunsaturated, typically aromatic, hydrocarbon group, which can be a single ring or multiple rings (up to three rings) which are fused together. The term “heteroaryl” refers to aryl groups (or rings) that contain from one to five heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl groups include phenyl and naphthyl, while non-limiting examples of heteroaryl groups include pyridyl, pyridazinyl, pyrazinyl, pyrimindinyl, triazinyl, quinolinyl, quinoxalinyl, quinazolinyl, cinnolinyl, phthalaziniyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiaxolyl, benzofuranyl, benzothienyl, indolyl, quinolyl, isoquinolyl, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furyl, thienyl and the like. Optional substituents for each of the above noted aryl and heteroaryl ring systems can be selected from the group of acceptable substituents described further below.

As used herein, the term “arylene” generically refers to any aryl that is a divalent radical. For a more specific example, “phenylene” refers to a divalent phenyl ring radical. The terms “1,2-arylene,” “1,3-arylene” or “1,4-arylene” refer to geometrical isomers of a particular arylene wherein, two groups attached to an aryl as depicted in a formula are situated in an ortho, meta or para geometrical relationship about the aryl, respectively.

As used herein, the term “heteroarylene” generically refers to any heteroaryl is a divalent radical. For a more specific example, “pyridylene” refers to a divalent pyridyl ring radical. For example, the terms “2,5-pyridylene” refers to a divalent pyridyl ring radical wherein the two groups shown attached to the pyridylene group as depicted in a formula are attached in at the 2- and 5-position of the pyridine ring as shown below:

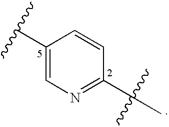

The above terms (e.g., “alkyl,” “aryl” and “heteroaryl”), in some embodiments, will include both substituted and unsubstituted forms of the indicated radical. Preferred substituents for each type of radical are provided below.

Substituents for the alkyl radicals (including those groups often referred to as alkylene, alkenyl, alkynyl, heteroalkyl and cycloalkyl) can be a variety of groups including, but not limited to, -halogen, —OR′, —NR′R″, —SR′, —SiR′R″R′″, —OC(O)R′, —C(O)R′, —CO₂R′, —CONR′R″, —OC(O)NR′R″, —NR″C(O)R′, —NR″C(O)NR′R″, —NR″C(O)₂R′, —NHC(NH₂)═NH, —NRC(NH₂)═NH, —NHC(NH₂)═NR′, —NR′″C(NR′R″)═N—CN, —NR′″C(NR′R″)═NOR′, —NHC(NH₂)═NR′, —S(O)R′, —S(O)₂R′, —S(O)₂NR′R″, —NR′S(O)₂R″, —NR″S(O)₂NR′R″, —CN, —NO₂, —(CH₂)₁₋₄—OR′, —(CH₂)₁₋₄—NR′R″, —(CH₂)₁₋₄—SR′, —(CH₂)₁₋₄—SiR′R″R′″, —(CH₂)₁₋₄—OC(O)R′, —(CH₂)₁₋₄—C(O)R′, —(CH₂)₁₋₄—CO₂R′, —(CH₂)₁₋₄CONR′R″, in a number ranging from zero to (2 m′+1), where m′ is the total number of carbon atoms in such radical. R′, R″ and R′″ each independently refer to groups including, for example, hydrogen, unsubstituted C₁₋₆ alkyl, unsubstituted heteroalkyl, unsubstituted aryl, aryl substituted with 1-3 halogens, unsubstituted C₁₋₆ alkyl, C₁₋₆ alkoxy or C₁₋₆ thioalkoxy groups, or unsubstituted aryl-C₁₋₄ alkyl groups, unsubstituted heteroaryl, substituted heteroaryl, among others. When R′ and R″ are attached to the same nitrogen atom, they can be combined with the nitrogen atom to form a 3-, 4-, 5-, 6-, or 7-membered ring. For example, —NR′R″ is meant to include 1-pyrrolidinyl and 4-morpholinyl. Other substitutents for alkyl radicals, including heteroalkyl, alkylene, include for example, ═O, ═NR′, ═N—OR′, ═N—CN, ═NH, wherein R′ include substituents as described above. When a substituent for the alkyl radicals (including those groups often referred to as alkylene, alkenyl, alkynyl, heteroalkyl and cycloalkyl) contains an alkylene linker (e.g., —(CH₂)₁₋₄—NR′R″), the alkylene linker includes halo variants as well. For example, the linker “—(CH₂)₁₋₄—” when used as part of a substituent is meant to include difluoromethylene, 1,2-difluoroethylene, etc.

Similarly, substituents for the aryl and heteroaryl groups are varied and are generally selected from the group including, but not limited to, -halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′C(O)NR″R′″, —NHC(NH₂)═NH, —NR′C(NH₂)═NH, —NHC(NH₂)═NR′, —S(O)R′, —S(O)₂R′, —S(O)₂NR′R″, —NR′S(O)₂R″, —N₃, perfluoro-C₁₋₄ alkoxy, and perfluoro-C₁₋₄ alkyl, —(CH₂)₁₋₄—OR′, —(CH₂)₁₋₄—NR′R″, —(CH₂)₁₋₄—SR′, —(CH₂)₁₋₄—SiR'R″R′″, —(CH₂)₁₋₄—OC(O)R′, —(CH₂)₁₋₄—C(O)R′, —(CH₂)₁₋₄—CO₂R′, —(CH₂)₁₋₄CONR′R″, in a number ranging from zero to the total number of open valences on the aromatic ring system; and where R′, R″ and R′″ are independently selected from hydrogen, C₁₋₆ alkyl, C₃₋₆ cycloalkyl, C₂₋₆ alkenyl, C₂₋₆ alkynyl, unsubstituted aryl and heteroaryl, (unsubstituted aryl)-C₁₋₄ alkyl, and unsubstituted aryloxy-C₁₋₄ alkyl. Other suitable substituents include each of the above aryl substituents attached to a ring atom by an alkylene tether of from 1-4 carbon atoms. When a substituent for the aryl or heteroaryl group contains an alkylene linker (e.g., —(CH₂)₁₋₄—NR′R″), the alkylene linker includes halo variants as well. For example, the linker “—(CH₂)₁₋₄—” when used as part of a substituent is meant to include difluoromethylene, 1,2-difluoroethylene, etc.

As used herein, the term “heteroatom” is meant to include oxygen (O), nitrogen (N), sulfur (S) and silicon (Si).

As used herein, the term “chiral” refers to molecules which have the property of non-superimposability of the mirror image partner, while the term “achiral” refers to molecules which are superimposable on their mirror image partner.

As used herein, the term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers can separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention can contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these stereoisomers are identical except that they are mirror images of one another. A specific stereoisomer can also be referred to as an enantiomer, and a mixture of such isomers is often called an enantiomeric mixture. A 50:50 mixture of enantiomers is referred to as a racemic mixture or a racemate, which can occur where there has been no stereoselection or stereospecificity in a chemical reaction or process. The terms “racemic mixture” and “racemate” refer to an equimolar mixture of two enantiomeric species, devoid of optical activity.

As used herein, the term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

As used herein, the term “solvate” refers to an association or complex of one or more solvent molecules and a compound of the invention. Examples of solvents that form solvates include, but are not limited to, water, isopropanol, ethanol, methanol, DMSO, ethyl acetate, acetic acid, and ethanolamine. The term “hydrate” refers to the complex where the solvent molecule is water.

As used herein, the term “protecting group” refers to a substituent that is commonly employed to block or protect a particular functional group on a compound. For example, an “amino-protecting group” is a substituent attached to an amino group that blocks or protects the amino functionality in the compound. Suitable amino-protecting groups include acetyl, trifluoroacetyl, t-butoxycarbonyl (BOC), benzyloxycarbonyl (CBZ) and 9-fluorenylmethylenoxycarbonyl (Fmoc). Similarly, a “hydroxy-protecting group” refers to a substituent of a hydroxy group that blocks or protects the hydroxy functionality. Suitable protecting groups include acetyl and silyl. A “carboxy-protecting group” refers to a substituent of the carboxy group that blocks or protects the carboxy functionality. Common carboxy-protecting groups include phenylsulfonylethyl, cyanoethyl, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see P. G. M. Wuts and T. W. Greene, Greene's Protective Groups in Organic Synthesis 4^{th} edition, Wiley-Interscience, New York, 2006.

As used herein, the term “mammal” includes, but is not limited to, humans, mice, rats, guinea pigs, monkeys, dogs, cats, horses, cows, pigs, and sheep

As used herein, the term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds can be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. As used herein the term “prodrug” refers to those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Prodrugs of the invention include compounds wherein an amino acid residue, or a polypeptide chain of two or more (e.g., two, three or four) amino acid residues, is covalently joined through an amide or ester bond to a free amino, hydroxy or carboxylic acid group of a compound of the present invention. The amino acid residues include but are not limited to the 20 naturally occurring amino acids commonly designated by three letter symbols and also includes phosphoserine, phosphothreonine, phosphotyrosine, 4-hydroxyproline, hydroxylysine, demosine, isodemosine, gamma-carboxyglutamate, hippuric acid, octahydroindole-2-carboxylic acid, statine, 1,2,3,4-tetrahydroisoquinoline-3-carboxylic acid, penicillamine, ornithine, 3-methylhistidine, norvaline, beta-alanine, gamma-aminobutyric acid, citrulline, homocysteine, homoserine, methyl-alanine, para-benzoylphenylalanine, phenylglycine, propargylglycine, sarcosine, methionine sulfone and tert-butylglycine.

Additional types of prodrugs are also encompassed. For instance, a free carboxyl group of a compound of the invention can be derivatized as an amide or alkyl ester. As another example, compounds of this invention comprising free hydroxy groups can be derivatized as prodrugs by converting the hydroxy group into a group such as, but not limited to, a phosphate ester, hemisuccinate, dimethylaminoacetate, or phosphoryloxymethyloxycarbonyl group, as outlined in Fleisher, D. et al., (1996) Improved oral drug delivery: solubility limitations overcome by the use of prodrugs Advanced Drug Delivery Reviews, 19:115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers, wherein the acyl group can be an alkyl ester optionally substituted with groups including, but not limited to, ether, amine and carboxylic acid functionalities, or where the acyl group is an amino acid ester as described above, are also encompassed. Prodrugs of this type are described in J. Med. Chem., (1996), 39:10. More specific examples include replacement of the hydrogen atom of the alcohol group with a group such as (C₁₋₆)alkanoyloxymethyl, 1-((C₁₋₆)alkanoyloxy)ethyl, 1-methyl-1-((C₁₋₆)alkanoyloxy)ethyl, (C₁₋₆)alkoxycarbonyloxymethyl, N—(C₁₋₆)alkoxycarbonylaminomethyl, succinoyl, (C₁₋₆)alkanoyl, alpha-amino(C₁₋₄)alkanoyl, arylacyl and alpha-aminoacyl, or alpha-aminoacyl-alpha-aminoacyl, where each alpha-aminoacyl group is independently selected from the naturally occurring L-amino acids, P(O)(OH)₂, —P(O)(O(C₁₋₆)alkyl)₂ or glycosyl (the radical resulting from the removal of a hydroxyl group of the hemiacetal form of a carbohydrate).

For additional examples of prodrug derivatives, see, for example, a) Design of Prodrugs, edited by H. Bundgaard, (Elsevier, 1985) and Methods in Enzymology, Vol. 42, p. 309-396, edited by K. Widder, et al. (Academic Press, 1985); b) A Textbook of Drug Design and Development, edited by Krogsgaard-Larsen and H. Bundgaard, Chapter 5 “Design and Application of Prodrugs,” by H. Bundgaard p. 113-191 (1991); c) H. Bundgaard, Advanced Drug Delivery Reviews, 8:1-38 (1992); d) H. Bundgaard, et al., Journal of Pharmaceutical Sciences, 77:285 (1988); and e) N. Kakeya, et al., Chem. Pharm. Bull., 32:692 (1984), each of which is specifically incorporated herein by reference.

Additionally, the present invention provides for metabolites of compounds of the invention. As used herein, a “metabolite” refers to a product produced through metabolism in the body of a specified compound or salt thereof. Such products can result for example from the oxidation, reduction, hydrolysis, amidation, deamidation, esterification, deesterification, enzymatic cleavage, and the like, of the administered compound.

Metabolite products typically are identified by preparing a radiolabelled (e.g., ¹⁴C or ³H) isotope of a compound of the invention, administering it parenterally in a detectable dose (e.g., greater than about 0.5 mg/kg) to an animal such as rat, mouse, guinea pig, monkey, or to man, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g., by MS, LC/MS or NMR analysis. In general, analysis of metabolites is done in the same way as conventional drug metabolism studies well known to those skilled in the art. The metabolite products, so long as they are not otherwise found in vivo, are useful in diagnostic assays for therapeutic dosing of the compounds of the invention.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention can exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers, regioisomers and individual isomers (e.g., separate enantiomers) are all intended to be encompassed within the scope of the present invention.

The compounds of the present invention can also contain unnatural proportions of atomic isotopes at one or more of the atoms that constitute such compounds. For example, the present invention also embraces isotopically-labeled compounds of the present invention which are identical to those recited herein, but for the fact that one or more atoms are replaced by an atom having an atomic mass or mass number different from the atomic mass or mass number usually found in nature. All isotopes of any particular atom or element as specified are contemplated within the scope of the compounds of the invention, and their uses. Exemplary isotopes that can be incorporated into compounds of the invention include isotopes of hydrogen, carbon, nitrogen, oxygen, phosphorus, sulfur, fluorine, chlorine and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Certain isotopically-labeled compounds of the present invention (e.g., those labeled with ³H and ¹⁴C) are useful in compound and/or substrate tissue distribution assays. Tritiated (³H) and carbon-14 (¹⁴C) isotopes are useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C and ¹⁸F are useful for positron emission tomography (PET) studies to examine substrate receptor occupancy. Isotopically labeled compounds of the present invention can generally be prepared by following procedures analogous to those disclosed in the Schemes and/or in the Examples herein below, by substituting an isotopically labeled reagent for a non-isotopically labeled reagent.

The terms “treat” and “treatment” refer to both therapeutic treatment and prophylactic or preventative measures, wherein the object is to prevent or slow down (lessen) an undesired physiological change or disorder, such as the development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The phrase “therapeutically effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the drug can reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug can prevent growth and/or kill existing cancer cells, it can be cytostatic and/or cytotoxic. For cancer therapy, efficacy can be measured, for example, by assessing the time to disease progression (TTP) and/or determining the response rate (RR).

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

As used herein, the term “adjunct” relates to the use of active compounds in conjunction with known therapeutic means. Such means include cytotoxic regimes of drugs and/or ionising radiation as used in the treatment of different cancer types. Examples of chemotherapeutic agents that can be combined with compounds of the invention include Erlotinib (TARCEVA®, Genentech/OSI Pharm.), Bortezomib (VELCADE®, Millennium Pharm.), Fulvestrant (FASLODEX®, AstraZeneca), Sutent (SU11248, Pfizer), Letrozole (FEMARA®, Novartis), Imatinib mesylate (GLEEVECO, Novartis), PTK787/ZK 222584 (Novartis), Oxaliplatin (Eloxatin®, Sanofi), 5-FU (5-fluorouracil), Leucovorin, Rapamycin (Sirolimus, RAPAMUNE®, Wyeth), Lapatinib (TYKERB®, GSK572016, Glaxo Smith Kline), Lonafarnib (SCH 66336), Sorafenib (BAY43-9006, Bayer Labs), and Gefitinib (IRESSA®, AstraZeneca), AG1478, AG1571 (SU 5271; Sugen), alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (Angew Chem. Intl. Ed. Engl. (1994) 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, N.J.), ABRAXANE™ (Cremophor-free), albumin-engineered nanoparticle formulations of paclitaxel (American Pharmaceutical Partners, Schaumberg, Ill.), and TAXOTERE® (doxetaxel; Rhone-Poulenc Rorer, Antony, France); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Also included in the definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors, for example a PI3K inhibitor, a MEK inhibitor, etc; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN® rIL-2; a topoisomerase 1 inhibitor such as LURTOTECAN®; ABARELIX® rmRH; (ix) anti-angiogenic agents such as bevacizumab (AVASTIN®, Genentech); and (x) pharmaceutically acceptable salts, acids and derivatives of any of the above.

II.A Compounds

In a first embodiment, the present invention provides for a compound of Formula I

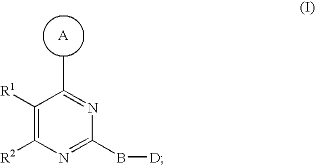

or a pharmaceutically acceptable salt thereof, wherein in Formula I, A is a 5- to 8-membered heterocyclic ring having from 1 to 3 heteroatoms independently selected from N, O and S as ring vertices, and having from 0 to 2 double bonds; wherein the A ring is further substituted with from 0 to 5 R^{A} substituents selected from the group consisting of —C(O)OR^{a}, —C(O)NR^{a}R^{b}, —NR^{a}R^{b}, —OC(O)R^{c}—OR^{a}, —SR^{a}, —S(O)₂R^{c}, —S(O)R^{c}, —R^{c}, —(CH₂)₁₋₄—NR^{a}R^{b}, —(CH₂)₁₋₄—NR^{a}C(O)R^{c}, —(CH₂)₁₋₄—OR^{a}, —(CH₂)₁₋₄—SR^{a}, —(CH₂)₁₋₄—S(O)₂R^{c}, —(CH₂)₁₋₄—S(O)R^{c}, halogen, —NO₂, —CN and —N₃, wherein R^{a} and R^{b} are each independently selected from hydrogen, C₁₋₆ alkyl, C₁₋₆ haloalkyl, C₁₋₆ heteroalkyl, C₂₋₆ alkenyl, C₂₋₆ alkynyl, C₃₋₆ cycloalkyl, phenyl and —(CH₂)₁₋₄(phenyl), and optionally R^{a} and R^{b}, together with the nitrogen atom to which each is attached, are combined to form a 3- to 7-membered heterocyclic ring comprising 1 to 2 heteroatoms selected from N, O and S; R^{c} is selected from C₁₋₆ alkyl, C₁₋₆ haloalkyl, C₁₋₆ heteroalkyl, C₂₋₆ alkenyl, C₂₋₆ alkynyl, C₃₋₆ cycloalkyl, phenyl and —(CH₂)₁₋₄ (phenyl); and any two substituents attached to the same atom in the 5- to 8-membered heterocyclic ring are optionally combined to form a 3- to 5-membered carbocyclic or 3 to 5-membered heterocyclic ring. R¹ and R² are combined with the atoms to which they are attached to form a 5- to 8-membered monocyclic or bridged bicyclic heterocyclic ring comprising —O— as one of the ring vertices; wherein the 5- to 8-membered monocyclic or bridged-bicyclic heterocyclic ring formed by combining R¹ and R² further optionally comprises one additional heteroatom selected from the group consisting of N, O and S, and is substituted with from 0 to 5 R^{R} substituents selected from the group consisting of halogen, —NR^{j}R^{k}, —C(O)OR^{j}, —C(O)NR^{j}R^{k}, —NHC(O)R^{j}, —OC(O)R^{j}, —R^{m}, —CN, ═O, ═S, ═N—CN, —(CH₂)₁₋₄—CN, —(CH₂)₁₋₄—OR^{j}, —(CH₂)₁₋₄—NR^{j}R^{k}, —C₁₋₄ alkylene-OR^{j}, —C₁₋₄ alkylene-R^{m}, —C₂₋₄ alkenylene-R^{m}, —C₂₋₄ alkynylene-R^{m}, —C₁₋₄ alkylene-C₁₋₉ heteroaryl, C₂₋₄ alkenylene-C₁₋₉ heteroaryl, C₂₋₄ alkynylene-C₁₋₉ heteroaryl, —C₁₋₄ alkylene-C₆₋₁₀ aryl, C₂₋₄ alkenylene-C₆₋₁₀ aryl and C₂₋₄ alkynylene-C₆₋₁₀ aryl, wherein R^{j} and R^{k} are each independently selected from hydrogen, C₁₋₆ alkyl, C₁₋₆ haloalkyl, C₁₋₆ heteroalkyl, C₂₋₆ alkenyl, C₂₋₆ alkynyl, C₃₋₇ cycloalkyl, C₂₋₆ heterocycloalkyl, phenyl, pyridyl and —(CH₂)₁₋₄-(Ph), and R^{j} and R^{k}, when attached to the same nitrogen atom, are optionally combined to form a 3- to 6-membered heterocyclic ring comprising 1 to 2 heteroatoms selected from N, O and S; and R^{m} is selected from C₁₋₆ alkyl, C₁₋₆ haloalkyl, C₁₋₆ heteroalkyl, C₂₋₆ alkenyl, C₂₋₆ alkynyl, C₃₋₇ cycloalkyl, C₂₋₆ heterocycloalkyl and —(CH₂)₁₋₄—(Ph) and wherein a C₃₋₇ cycloalkyl, C₂₋₆ heterocycloalkyl, C₁₋₉ heteroaryl or C₆₋₁₀ aryl portion of a R^{R} substituent is substituted with from 0 to 3 substituents selected from the group consisting of F, Cl, Br, I, —NH(C₁₋₄ alkyl), —N(diC₁₋₄ alkyl), 0(C₁₋₄ alkyl), C₁₋₆ alkyl, C₁₋₆ heteroalkyl, —C(O)O(C₁₋₄alkyl), —C(O)NH(C₁₋₄alkyl), —C(O)N(diC₁₋₄ alkyl), —NO₂, —CN; wherein when R¹ and R² are combined to form a monocyclic 5- to 8-membered heterocyclic ring then any two R^{R} substitutents attached to the same atom or adjacent carbon atoms in said 5- to 8-membered heterocyclic ring are optionally combined to form a 3- to 7-membered cycloalkyl ring or a 3- to 7-membered heterocycloalkyl ring comprising 1 to 2 heteroatoms selected from N, O and S as ring vertices. B is a member selected from the group consisting of phenylene and 5- to 6-membered heteroarylene, and is substituted with from 0 to 4 R^{B} substituents selected from halogen, —CN, —N₃, —NO₂, —C(O)ORⁿ, —C(O)NRⁿR^{o}, —NRⁿC(O)R^{o}, —NRⁿC(O)NRⁿR^{o}, —ORⁿ, —NRⁿR^{o}, —(CH₂)₁₋₄—C(O)ORⁿ, —(CH₂)₁₋₄—C(O)NRⁿR^{o}, —(CH₂)₁₋₄—ORⁿ, —(CH₂)₁₋₄—NRⁿR^{o}, —(CH₂)₁₋₄—SR^{p} and R^{p}; wherein Rⁿ and R^{o} are independently selected from hydrogen and C₁₋₆ alkyl, C₁₋₆ haloalkyl, C₁₋₆ heteroalkyl, C₂₋₆ alkenyl, C₂₋₆ alkynyl, C₃₋₇ cycloalkyl, C₂₋₆ heterocycloalkyl, phenyl and —(CH₂)₁₋₄-(phenyl) or when attached to the same nitrogen atom, Rⁿ and R^{o} are optionally are combined to form a 3- to 6-membered heterocyclic ring comprising 1 to 2 heteroatoms selected from N, O and S; R^{p} is C₁₋₆ alkyl, C₁₋₆ haloalkyl, C₁₋₆ heteroalkyl, C₂₋₆ alkenyl, C₂₋₆ alkynyl, C₃₋₇ cycloalkyl, C₂₋₆ heterocycloalkyl, phenyl and —(CH₂)₁₋₄-(phenyl), wherein any two substituents, not including the D group, located on adjacent atoms of B are optionally combined to form a 5- to 6-membered carbocyclic, heterocyclic, aryl or heteroaryl ring. Finally, D is a member selected from the group consisting of —NR³C(O)NR⁴R⁵, —NR⁴R⁵, —C(O)NR⁴R⁵, —OC(O)OR⁴, —OC(O)NR⁴R⁵, —NR³C(═N—CN)NR⁴R⁵, —NR³C(═N—OR⁴)NR⁴R⁵, —NR³C(═N—NR⁴)NR⁴R⁵, —NR³C(O)R⁴, —NR³C(O)OR⁴, —NR³S(O)₂NR⁴R⁵, —NR³S(O)₂R⁴, —NR³C(═S)NR⁴R⁵ and —S(O)₂R⁴R⁵, wherein R³ is selected from the group consisting of hydrogen, C₁₋₆ alkyl, C₁₋₆ haloalkyl and C₂₋₆ alkenyl; R⁴ and R⁵ are each independently selected from the group consisting of hydrogen, C₁₋₆ alkyl, C₁₋₆ haloalkyl, C₁₋₆ alkylamino-C(═O)—, C₂₋₆ alkenyl, C₂₋₆ alkynyl, C₃₋₁₀ cycloalkyl, C₂₋₉ heterocycloalkyl, C₆₋₁₀ aryl and C₁₋₉ heteroaryl, and R⁴ and R⁵, when attached to the same nitrogen atom, are optionally combined to form a 5- to 7-membered heterocyclic or 5- to 6-membered heteroaryl ring comprising 1 to 3 heteroatoms selected from N, O and S; and wherein R³, R⁴ and R⁵ are further substituted with from 0 to 3 R^{D} substituents independently selected from the group consisting of halogen, —NO₂, —CN, —NR^{q}R^{r}, —OR^{q}, —SR^{q}, —C(O)OR^{q}, —C(O)NR^{q}R^{r}, —NR^{q}C(O)R^{r}, —NR^{q}C(O)OR^{s}, —(CH₂)₁₋₄—NR^{q}R^{r}, —(CH₂)₁₋₄—OR^{q}, —(CH₂)₁₋₄—SR^{q}, —(CH₂)₁₋₄—C(O)OR^{q}, —(CH₂)₁₋₄—C(O)NR^{q}R^{r}, —(CH₂)₁₋₄—NR^{q}C(O)R^{r}, —(CH₂)₁₋₄—NR^{q}C(O)OR^{r}, —(CH₂)₁₋₄—CN, —(CH₂)₁₋₄—NO₂, —S(O)R^{r}, —S(O)₂R^{r}, —(CH₂)₁₋₄R^{s}, ═O, and —R^{s}; wherein R^{q} and R^{r} is selected from hydrogen, C₁₋₆ alkyl, C₁₋₆ haloalkyl, C₂₋₆ alkenyl, C₂₋₆ alkynyl, C₁₋₆ heteroalkyl, C₃₋₇ cycloalkyl, C₂₋₆ heterocycloalkyl, C₆₋₁₀ aryl, C₁₋₉ heteroaryl; and R^{s}, at each occurrence, is independently selected from C₁₋₆ alkyl, C₁₋₆ haloalkyl, C₃₋₇ cycloalkyl, C₂₋₆ heterocycloalkyl, C₆₋₁₀ aryl and C₁₋₉ heteroaryl; and wherein the D group and a substituent located on an adjacent atom of the B ring are optionally combined to form a 5- to 6-membered heterocyclic or heteroaryl ring, optionally substituted with 1 to 2 R^{D} substituents.

In a second embodiment, the present invention provides for a compound of Formula I

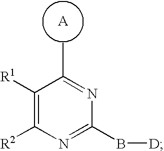

or a pharmaceutically acceptable salt thereof, wherein in Formula I, A is a 5- to 8-membered heterocyclic ring having from 1 to 3 heteroatoms independently selected from N, O and S as ring vertices, and having from 0 to 2 double bonds; wherein the A ring is further substituted with from 0 to 5 R^{A} substituents selected from the group consisting of C(O)OR^{a}, —C(O)NR^{a}R^{b}, —NR^{a}R^{b}, —OC(O)R^{c}, —OR^{a}, —SR^{a}, —S(O)₂R^{c}, —S(O)R^{c}, —R^{c}, —(CH₂)₁₋₄—NR^{a}R^{b}, —(CH₂)₁₋₄—NR^{a}C(O)R^{c}, —(CH₂)₁₋₄—OR^{a}, —(CH₂)₁₋₄—SR^{a}, —(CH₂)₁₋₄—S(O)₂R^{c}, —(CH₂)₁₋₄—S(O)R^{c}, halogen, —NO₂, —CN and —N₃, wherein R^{a} and R^{b} are each independently selected from hydrogen, C₁₋₆ alkyl, C₁₋₆ haloalkyl, C₁₋₆ heteroalkyl, C₂₋₆ alkenyl, C₂₋₆ alkynyl, C₃₋₆ cycloalkyl, phenyl and —(CH₂)₁₋₄(phenyl), and optionally R^{a} and R^{b}, together with the nitrogen atom to which each is attached, are combined to form a 3- to 7-membered heterocyclic ring comprising 1 to 2 heteroatoms selected from N, O and S; R^{c} is selected from C₁₋₆ alkyl, C₁₋₆ haloalkyl, C₁₋₆ heteroalkyl, C₂₋₆ alkenyl, C₂₋₆ alkynyl, C₃₋₆ cycloalkyl, phenyl and —(CH₂)₁₋₄ (phenyl); and any two substituents attached to the same atom in the 5- to 8-membered heterocyclic ring are optionally combined to form a 3- to 5-membered carbocyclic or a 3 to 5-membered heterocyclic ring. R¹ and R² are combined with the atoms to which they are attached to form a 5- to 8-membered monocyclic or bridged bicyclic heterocyclic ring comprising —O— as one of the ring vertices; wherein the 5- to 8-membered monocyclic or bridged-bicyclic heterocyclic ring formed by combining R¹ and R² further optionally comprises one additional heteroatom selected from the group consisting of N, O and S, and is substituted with from 0 to 5 R^{R} substituents selected from the group consisting of halogen, —NR^{j}R^{k}, —SR^{j}, —OR^{j}, —C(O)OR^{j}, —C(O)NR^{j}R^{k}, —NHC(O)R^{j}, —OC(O)R^{j}, —R^{m}, —CN, ═O, ═S, ═N—CN, —(CH₂)₁₋₄—CN, —(CH₂)₁₋₄—OR^{j}, —(CH₂)₁₋₄—NR^{j}R^{k}, —C₁₋₄ alkylene-R^{m}, —C₂₋₄ alkenylene-R^{m}, —C₂₋₄ alkynylene-R^{m}, —C₁₋₄ alkylene-C₁₋₉ heteroaryl, C₂₋₄ alkenylene-C₁₋₉ heteroaryl, C₂₋₄ alkynylene-C₁₋₉ heteroaryl, C₁₋₄ alkylene-C₆₋₁₀ aryl, C₂₋₄ alkynylene-C₆₋₁₀ aryl and C₂₋₄ alkynylene-C₆₋₁₀ aryl, wherein and R^{k} are each independently selected from hydrogen, C₁₋₆ alkyl, C₁₋₆ haloalkyl, C₁₋₆ heteroalkyl, C₂₋₆ alkenyl, C₂₋₆ alkynyl, C₃₋₇ cycloalkyl, C₂₋₆ heterocycloalkyl, phenyl, and —(CH₂)₁₋₄-(Ph), and R^{j} and R^{k}, when attached to the same nitrogen atom, are optionally combined to form a 3-5 to 6-membered heterocyclic ring comprising 1 to 2 heteroatoms selected from N, O and S; and R^{m} is selected from C₁₋₆ alkyl, C₁₋₆ haloalkyl, C₁₋₆ heteroalkyl, C₂₋₆ alkenyl, C₂₋₆ alkynyl, C₃₋₇ cycloalkyl, C₂₋₆ heterocycloalkyl and —(CH₂)₁₋₄-(Ph), and wherein a C₃₋₇ cycloalkyl, C₂₋₆ heterocycloalkyl, C₁₋₉ heteroaryl or C₆₋₁₀ aryl portion of a R^{R} substituent is substituted with from 0 to 3 substituents selected from the group consisting of F, Cl, Br, I, —NH(C₁₋₄ alkyl), —N(diC₁₋₄ alkyl), O(C₁₋₄ alkyl), C₁₋₆ alkyl, C₁₋₆ heteroalkyl, —C(O)O(C₁₋₄ alkyl), —C(O)NH(C₁₋₄alkyl), —C(O)N(diC₁₋₄ alkyl), —NO₂, —CN; wherein when R¹ and R² are combined to form a monocyclic 5- to 8-membered heterocyclic ring then any two R^{R} substitutents attached to the same atom or adjacent carbon atoms in said 5- to 8-membered heterocyclic ring are optionally combined to form a 3- to 7-membered cycloalkyl ring or a 3- to 7-membered heterocycloalkyl ring comprising 1 to 2 heteroatoms selected from N, O and S as ring vertices. B is a member selected from the group consisting of phenylene and 5- to 6-membered heteroarylene, and is substituted with from 0 to 4 R^{B} substituents selected from halogen, —CN, —N₃, —NO₂, —C(O)ORⁿ, —C(O)NRⁿR^{o}, —NRⁿC(O)R^{o}, —NRIV(O)NRⁿR^{o}, —ORⁿ, —NRⁿR^{o}, —(CH₂)₁₋₄—C(O)ORⁿ, —(CH₂)₁₋₄—C(O)NRⁿR^{o}, —(CH₂)₁₋₄—ORⁿ, —(CH₂)₁₋₄—NRⁿR^{o}, —(CH₂)₁₋₄—SRP and R^{p}; wherein Rⁿ and R^{o} are independently selected from hydrogen and C₁₋₆ alkyl, C₁₋₆ haloalkyl, C₁₋₆ heteroalkyl, C₂₋₆ alkenyl, C₂₋₆ alkynyl, C₃₋₇ cycloalkyl, C₂₋₆ heterocycloalkyl, phenyl and —(CH₂)₁₋₄-(phenyl) or when attached to the same nitrogen atom, Rⁿ and R^{o} are optionally are combined to form a 3- to 6-membered heterocyclic ring comprising 1 to 2 heteroatoms selected from N, O and S; R^{p} is C₁₋₆ alkyl, C₁₋₆ haloalkyl, C₁₋₆ heteroalkyl, C₂₋₆ alkenyl, C₂₋₆ alkynyl, C₃₋₇ cycloalkyl, C₂₋₆ heterocycloalkyl, phenyl and —(CH₂)₁₋₄-(phenyl), wherein any two substituents, not including the D group, located on adjacent atoms of B are optionally combined to form a 5- to 6-membered carbocyclic, heterocyclic, aryl or heteroaryl ring. D is a member selected from the group consisting of —NR³C(O)NR⁴R⁵, —NR⁴R⁵, —C(O)NR⁴R⁵, —OC(O)OR⁴, —OC(O)NR⁴R⁵, —NR³C(═N—CN)NR⁴R⁵, —NR³C(═N—OR⁴)NR⁴R⁵, —NR³C(═N—NR⁴)NR⁴R⁵, —NR³C(O)R⁴, —NR³C(O)OR⁴, —NR³S(O)₂NR⁴R⁵, —NR³S(O)₂R⁴, —NR³C(═S)NR⁴R⁵ and —S(O)₂R⁴R⁵, wherein R³ is selected from the group consisting of hydrogen, C₁₋₆ alkyl, C₁₋₆ haloalkyl and C₂₋₆ alkenyl; R⁴ and R⁵ are each independently selected from the group consisting of hydrogen, C₁₋₆ alkyl, C₁₋₆ haloalkyl, C₁₋₆ alkylamino-C(═O)—, C₂₋₆ alkenyl, C₂₋₆ alkynyl, C₃₋₁₀ cycloalkyl, C₂₋₉ heterocycloalkyl, C₆₋₁₀ aryl and C₁₋₉ heteroaryl, and R⁴ and R⁵, when attached to the same nitrogen atom, are optionally combined to form a 5- to 7-membered heterocyclic or 5- to 6-membered heteroaryl ring comprising 1 to 3 heteroatoms selected from N, O and S; and wherein R³, R⁴ and R⁵ are further substituted with from 0 to 3 R^{D} substituents independently selected from the group consisting of halogen, —NO₂, —CN, —NR^{q}R^{r}, —OR^{q}, —SR^{q}, —C(O)OR^{q}, —C(O)NR^{q}R^{r}, —NR^{q}C(O)R^{r}, —NR^{q}C(O)OR^{s}, —(CH₂)₁₋₄—NR^{q}R^{r}, —(CH₂)₁₋₄—OR^{q}, —(CH₂)₁₋₄—SR^{q}, —(CH₂)₁₋₄—C(O)OR^{q}, —(CH₂)₁₋₄—C(O)NR^{q}R^{r}, —(CH₂)₁₋₄—NR^{q}C(O)R^{r}, —(CH₂)₁₋₄—NR^{q}C(O)OR^{r}, —(CH₂)₁₋₄—CN, —(CH₂)₁₋₄—NO₂, —S(O)R^{r}, —S(O)₂R^{r}, —(CH₂)₁₋₄R^{s}, ═O, and —R^{s}; wherein R^{q} and R^{r} is selected from hydrogen, C₁₋₆ alkyl, C₁₋₆ haloalkyl, C₂₋₆ alkenyl, C₂₋₆ alkynyl, C₁₋₆ heteroalkyl, C₃₋₇ cycloalkyl, C₂₋₆ heterocycloalkyl, C₆₋₁₀ aryl, C₁₋₉ heteroaryl; and R^{s}, at each occurrence, is independently selected from C₁₋₆ alkyl, C₁₋₆ haloalkyl, C₃₋₇ cycloalkyl, C₂₋₆ heterocycloalkyl, C₆₋₁₀ aryl and C₁₋₉ heteroaryl; and wherein the D group and a substituent located on an adjacent atom of the B ring are optionally combined to form a 5- to 6-membered heterocyclic or heteroaryl ring.

In a third embodiment of compounds of Formula I, and within certain aspects fo the first or second embodiment, R¹ and R² are combined to form a 5- to 8-membered heterocyclic ring comprising —O— as the only heteroatom in the 5- to 8-membered heterocyclic ring.

In a fourth embodiment of compounds of Formula I, and within certain aspects of the first or second embodiment, in Formula I the A ring comprises from 0 to 1 double bond.

In a fifth embodiment of compounds of Formula I, and within certain aspects of the first, second, third or fourth embodiment, A is a 5- to 8-membered monocyclic or bicyclic-bridged heterocyclic ring and is further substituted with from 0 to 3 R^{A} substituents selected from the group consisting of —C(O)OR^{a}, —C(O)NR^{a}R^{b}, —NR^{a}R^{b}, —OC(O)R^{c}—OR^{a}, —SR^{a}, —S(O)₂R^{c}, —S(O)R^{c}, —R^{c}, —(CH₂)₁₋₄—NR^{a}R^{b}, —(CH₂)₁₋₄—OR^{a}, halogen, —NO₂, —CN and —N₃, wherein R^{a} and R^{b} are each independently selected from hydrogen, C₁₋₆ alkyl, C₁₋₆ haloalkyl, C₁₋₆ heteroalkyl and C₃₋₆ cycloalkyl, and optionally R^{a} and R^{b}, together with the nitrogen atom to which each is attached, are combined to form a 3- to 6-membered ring; R^{c} is selected from C₁₋₆ alkyl, C₁₋₆ haloalkyl, C₁₋₆ heteroalkyl, C₂₋₆ alkenyl, C₂₋₆ alkynyl, C₃₋₆ cycloalkyl, phenyl and —(CH₂)₁₋₄ (phenyl); and wherein any two substituents located on the same atom of the A ring are optionally combined to form a 3- to 5-membered cycloalkyl ring. B is selected from the group consisting of 1,4-phenylene, 2,5-pyridylene and 3,6-pyridylene and is substituted with from 0 to 2 substituents selected from halogen, —CN, —N₃, —NO₂, —C(O)ORⁿ, —C(O)NRⁿR^{o}, —NRⁿC(O)R^{o}, —NRⁿC(O)NRⁿR^{o}, —ORⁿ, —NRⁿR^{o} and R^{p}; wherein Rⁿ and R^{o} are independently selected from hydrogen and C₁₋₆ alkyl, C₁₋₆ haloalkyl, C₁₋₆ heteroalkyl, C₃₋₇ cycloalkyl and C₂₋₆ heterocycloalkyl, or when attached to the same nitrogen atom, Rⁿ and R^{o} are optionally are combined to form a 3- to 6-membered ring; R^{p} is C₁₋₆ alkyl, C₁₋₆ haloalkyl, C₃₋₇ cycloalkyl and C₂₋₆ heterocycloalkyl. D is a member selected from the group consisting of —NR³C(O)NR⁴R⁵, —NR⁴R⁵, —C(O)NR⁴R⁵, —OC(O)NR⁴R⁵, —NR³C(═N—CN)NR⁴R⁵, —NR³C(O)R⁴, —NR³C(O)OR⁴, —NR³S(O)₂NR⁴R⁵, —NR³S(O)₂R⁴, —NR³C(═S)NR⁴R⁵ and —S(O)₂R⁴R⁵ wherein R³ is selected from the group consisting of hydrogen, C₁₋₆ alkyl, C₁₋₆ haloalkyl and C₂₋₆ alkenyl; R⁴ and R⁵ are each independently selected from the group consisting of hydrogen, C₁₋₆ alkyl, C₁₋₆ haloalkyl, C₂₋₆ alkenyl, C₂₋₆ alkynyl, C₃₋₇ cycloalkyl, C₂₋₆ heterocycloalkyl, C₆₋₁₀ aryl and C₁₋₉ heteroaryl, and R⁴ and R⁵, when attached to the same nitrogen atom, are optionally combined to form a 5- to 7-membered heterocyclic or 5- to 6-membered heteroaryl ring; and wherein R³, R⁴ and R⁵ are further substituted with from 0 to 3 R^{D} substituents independently selected from the group consisting of halogen, —NO₂, —CN, —NR^{q}R^{r}, —OR^{q}, —SR^{q}, —C(O)OR^{q}, —C(O)NR^{q}R^{r}, —NR^{q}C(O)R^{r}, —NR^{q}C(O)OR^{s}, —(CH₂)₁₋₄—NR^{q}R^{r}, —(CH₂)₁₋₄—OR^{q}, —(CH₂)₁₋₄—SR^{q}, —(CH₂)₁₋₄—C(O)OR^{q}, —(CH₂)₁₋₄—C(O)NR^{q}R^{r}, —(CH₂)₁₋₄—NR^{q}C(O)R^{r}, —(CH₂)₁₋₄—NR^{q}C(O)OR^{r}, —(CH₂)₁₋₄—CN, —(CH₂)₁₋₄—NO₂, —S(O)R^{r}, —S(O)₂R^{r}, ═O, and —R^{s}; wherein R^{q} and R^{r} is each independently selected from hydrogen, C₁₋₆ alkyl, C₁₋₆ haloalkyl, C₂₋₆ alkenyl, C₂₋₆ alkynyl, C₁₋₆ heteroalkyl, C₃₋₇ cycloalkyl, C₂₋₆ heterocycloalkyl, C₆₋₁₀ aryl, C₁₋₉ heteroaryl; and R^{s}, at each occurrence, is independently selected from C₁₋₄ alkyl, C₁₋₄ haloalkyl, C₃₋₇ cycloalkyl, C₂₋₆ heterocycloalkyl, C₆ aryl and C₁₋₅ heteroaryl; and wherein the D group and a substituent located on an adjacent atom of the B ring are optionally combined to form a 5- to 6-membered heterocyclic or heteroaryl ring.

In a sixth embodiment of compounds of Formula I, and within certain aspects of the first, second, third, fourth or fifth embodiment, the compound of the invention has the Formula II-A:

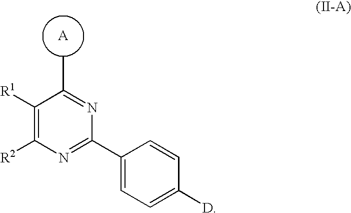

In seventh embodiment of compounds of Formula I, and within certain aspects of the first, second, third, fourth, fifth or sixth embodiment, A is a 5- to 7-membered monocyclic or bicyclic bridged heterocyclic ring and is further substituted with from 0 to 3 R^{A} substituents selected from the group consisting of —C(O)OR^{a}, —C(O)NR^{a}R^{b}, —NR^{a}R^{b}, —OR^{a}, —SR^{a}, —S(O)₂R^{c}, —S(O)R^{c}, —R^{c}, halogen, —NO₂, —CN and —N₃, wherein R^{a} and R^{b} are each independently selected from hydrogen, C₁₋₄ alkyl, C₁₋₄ haloalkyl, C₁₋₄ heteroalkyl and C₃₋₆ cycloalkyl, and optionally R^{a} and R^{b}, together with the nitrogen atom to which each is attached, are combined to form a 3- to 6-membered ring; R^{c} is selected from C₁₋₄ alkyl, C₁₋₄ haloalkyl, C₁₋₄ heteroalkyl, C₂₋₆ alkenyl, C₂₋₆ alkynyl and C₃₋₆ cycloalkyl.

In an eighth embodiment of compounds of Formula I, and within certain aspects of the seventh embodiment, the A ring is a ring selected from the group consisting of morpholin-4-yl, 3,4-dihydro-2H-pyran-4-yl, 3,6-dihydro-2H-pyran-4-yl, tetrahydro-2H-pyran-4-yl, 1,4-oxazepan-4-yl, 2-oxa-5-azabicyclo[2.2.1]heptan-5-yl, piperazin-1-yl and piperidin-1-yl, and is substituted with from 0 to 2 R^{A} substituents selected from the group consisting of —C(O)OR^{a}, —C(O)NR^{a}R^{b}, —NR^{a}R^{b}, —OR^{a}, —SR^{a}, —S(O)₂R^{c}, —S(O)R^{c}, —R^{c} halogen, —NO₂, —CN and —N₃, wherein R^{a} and R^{b} are each independently selected from hydrogen, C₁₋₄ alkyl, C₁₋₄ haloalkyl, C₁₋₄ heteroalkyl, C₂₋₆ alkenyl and C₃₋₆ cycloalkyl, wherein optionally R^{a} and R^{b}, together with the nitrogen atom to which each is attached, are combined to form a 3- to 6-membered heterocyclic ring, and R^{c} is selected from C₁₋₄ alkyl, C₁₋₄ haloalkyl, C₁₋₄ heteroalkyl, C₂₋₆ alkenyl, C₃₋₆ cycloalkyl.

In a ninth embodiment of compounds of Formula I, and within a certain aspect of the eighth embodiment, the A ring is selected from the group consisting of morpholin-4-yl, 3-methyl-morpholin-4-yl, 3-ethyl-morpholin-4-yl, 3,4-dihydro-2H-pyran-4-yl, 3,6-dihydro-2H-pyran-4-yl, tetrahydro-2H-pyran-4-yl, 1,4-oxazepan-4-yl, 2-oxa-5-azabicyclo[2.2.1]heptan-5-y1 and 4-methoxypiperidin-1-yl.

In a tenth embodiment of compounds of Formula I, and within certain aspects of the first, second, third, fifth, sixth, seventh, eighth or ninth embodiment, R¹ and R² are combined to form a 5- to 7-membered monocyclic heterocyclic ring, wherein the 5- to 7-membered ring is substituted with from 0 to 5 R^{R} substituents selected from the group consisting of halogen, —R^{m}, —C₁₋₄ alkylene-R^{m}, —C₂₋₄ alkenylene-R^{m}, —C₂₋₄ alkynylene-R^{m}, wherein R^{m} is selected from C₁₋₆ alkyl, C₁₋₆ haloalkyl, C₁₋₆ heteroalkyl, C₂₋₆ alkenyl, C₂₋₆ alkynyl, C₃₋₇ cycloalkyl, C₂₋₆ heterocycloalkyl and —(CH₂)₁₋₄-(Ph), and wherein halogen is selected from F, Cl and Br, wherein any two substituents attached to the same atom or to adjacent atoms in said 5- to 7-membered heterocyclic ring are optionally combined to form a 3- to 6-membered cycloalkyl or 3- to 6-membered heterocycloalkyl ring having 1 to 2 heteroatoms selected from N, O and S as ring vertices.

In the eleventh embodiment of compounds of Formula I, and within certain aspects of the tenth embodiment, R^{m} is selected from C₁₋₆ alkyl and C₁₋₆ heteroalkyl, and any two R^{m} groups located on the same or adjacent atoms is optionally combined to from a 3- to 6-membered cycloalkyl ring or a 3- to 6-membered heterocycloalkyl ring having 1 to 2 heteroatoms selected from N, O and S as ring vertices.

In a twelfth embodiment of compounds of Formula I, and within certain aspects of the first, second, third, fourth, fifth, sixth, seventh, eighth, ninth or tenth embodiment, in a compound of Formula I or Fomula II-A, the 5- to 7-membered heterocyclic ring formed by combining R¹ and R² comprises a carbon atom, which is substituted with two R^{R} substituents independently selected from F, Cl, Br and R^{m}, as a ring vertex.

In thirteenth embodiment of compounds of Formula I, and within certain aspects of the first, second, third, fifth, sixth, seventh, eighth or ninth embodiment, in a compound of Formula I or Formula II-A, the ring formed by combining R¹ and R², as fused to the pyrimidine ring of Formula I, has a structure selected from the group consisting of ii-A, ii-B, ii-C, ii-D, ii-E, ii-F, ii-G, ii-H, ii-J, ii-K, ii-L, ii-M, ii-N, ii-O, ii-P, ii-Q, ii-R, ii-S, ii-T, ii-U, ii-V, ii-W, ii-X, ii-Y, ii-Z, ii-AA, ii-BB and ii-CC shown below:

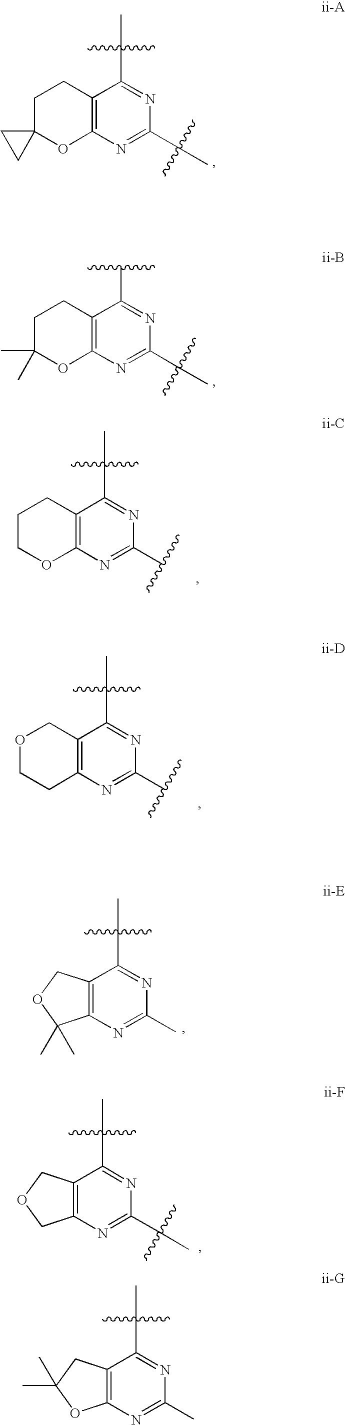

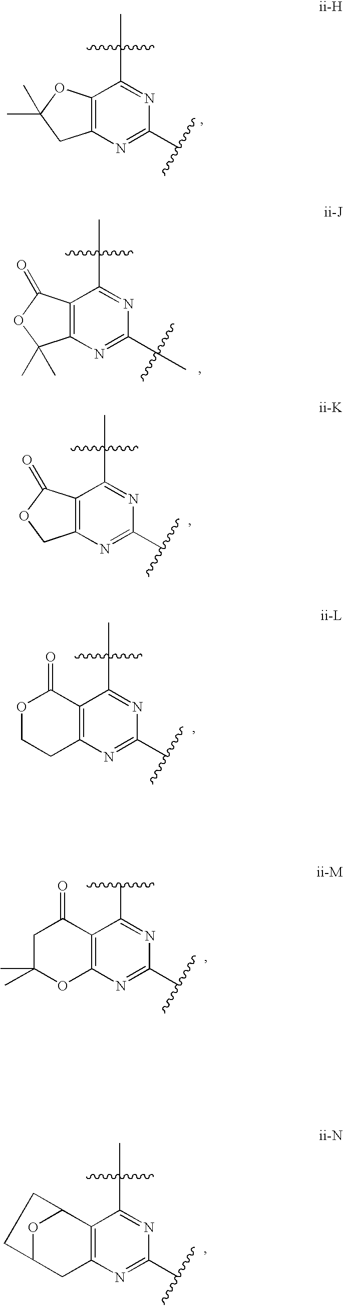

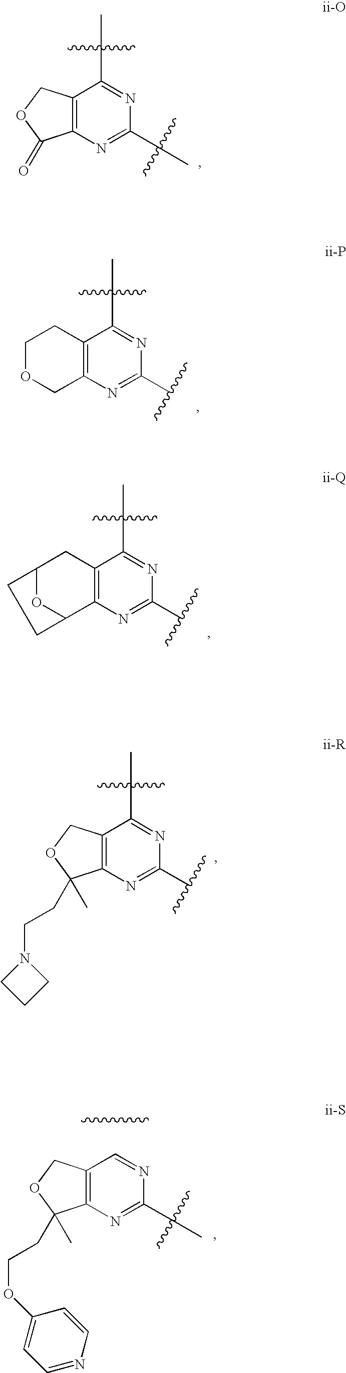

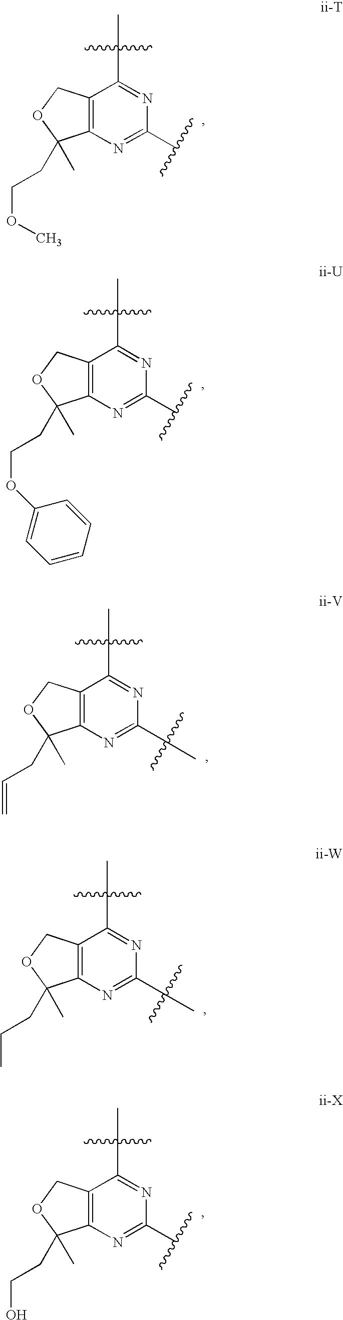

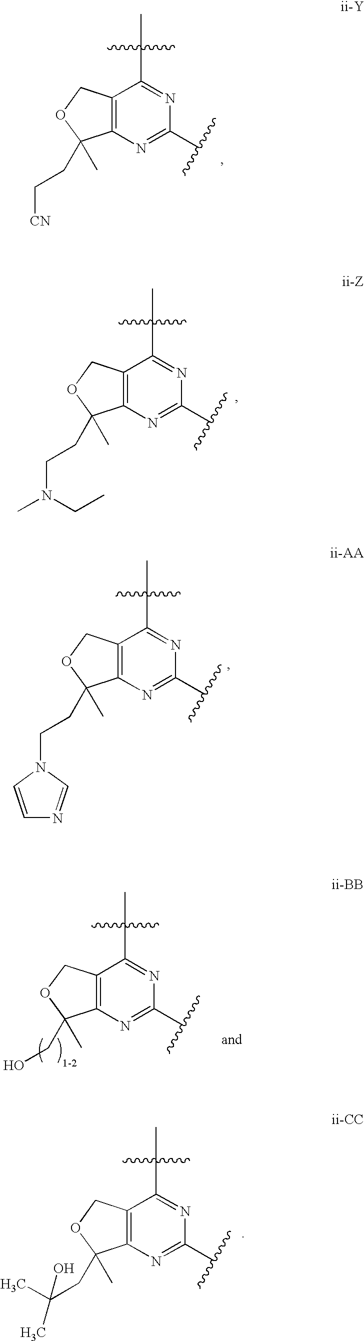

In fourteenth embodiment of compounds of Formula I, and within certain aspects of the first, second, third, fourth, fifth, sixth, seventh, eighth, ninth, tenth or thirteenth embodiment, D is selected from the group consisting of —NR³C(O)NR⁴R⁵, —NR⁴R⁵, —C(O)NR⁴R⁵, —NR³C(═N—CN)NR⁴R⁵, —NR³C(O)R⁴, —NR³C(O)OR⁴, —NR³S(O)R⁴, —NR³C(═S)NR⁴R⁵ and —S(O)₂NR⁴R⁵.

In a fifteenth embodiment of compounds of Formula I, and within certain aspects of the fourteenth embodiment, D is selected from —NR³C(O)NR⁴R⁵ and —NR⁴R⁵, wherein R³ is hydrogen; R⁴ and R⁵ are each independently selected from the group consisting of hydrogen, C₁₋₆ alkyl, C₁₋₆ haloalkyl, C₃₋₇ cycloalkyl, C₂₋₆ heterocycloalkyl, C₆₋₁₀ aryl and C₁₋₉ heteroaryl, wherein R⁴ and R⁵ are each independently optionally substituted; and R⁴ and R⁵, when attached to the same nitrogen atom, are optionally combined to form a 5- to 7-membered heterocyclic or 5- to 10-membered heteroaryl ring comprising 1 to 3 heteroatoms selected from N, O and S as ring vertices.

In sixteenth embodiment of compounds of Formula I, and within certain aspects of the fifteenth embodiment, D is —NR⁴R⁵, wherein R⁴ is hydrogen or C₁₋₃ alkyl, and R⁵ is selected from phenyl, C₁₋₅ heteroaryl, and C₂₋₆ heterocycloalkyl, wherein R⁵ is substituted with from 0 to 3 R^{D} substituents.

In a seventeenth embodiment of compounds of Formula I, and within certain aspects of the sixteenth embodiment, R⁵ is selected from the group consisting of:

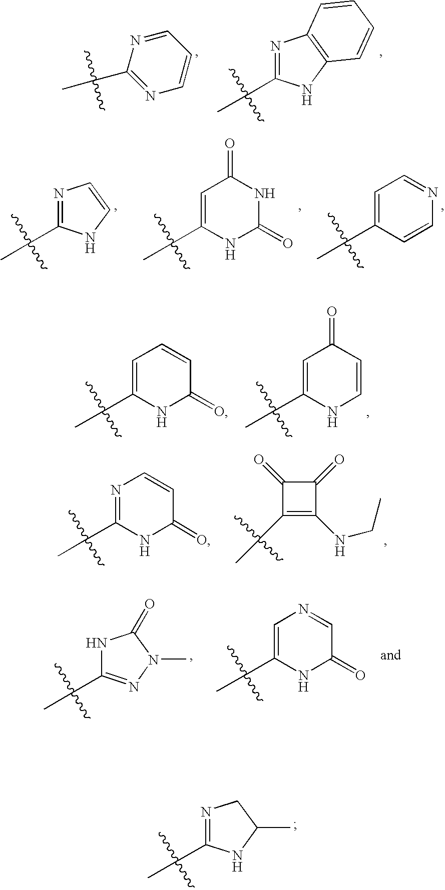

wherein from 0 to 3 hydrogen atoms attached to a carbon or nitrogen atom of R⁵ is optionally independently replaced with a R^{D} substitutent selected from the group consisting of halogen, F, Cl, Br, halogen, —NO₂, —CN, —NR^{q}R^{r}, —OR^{q}, —(CH₂)₁₋₄R^{s}, ═O, and —R^{s}; wherein R^{q} and R^{r} is selected from hydrogen, C₁₋₆ alkyl, C₁₋₆ haloalkyl, C₂₋₆ alkenyl, C₂₋₆ alkynyl, C₁₋₆ heteroalkyl; and R^{s}, at each occurrence, is independently selected from C₁₋₆ alkyl, C₁₋₆ haloalkyl, C₃₋₇ cycloalkyl and C₂₋₆ heterocycloalkyl.

In an eighteenth embodiment of compounds of Formula I, and within certain aspects of the fifteenth embodiment, D is —NR³C(O)NR⁴R⁵, wherein R³ is hydrogen; R⁴ and R⁵ are each independently selected from the group consisting of hydrogen, C₁₋₆ alkyl, C₁₋₆ heteroalkyl, C₃₋₇ cycloalkyl and C₂₋₆ heterocycloalkyl, wherein R⁴ and R⁵ at each occurrence are each independently optionally substituted.

In a nineteenth embodiment of compounds of Formula I, and within certain aspects of the eighteenth embodiment, R³ is hydrogen, R⁴ is hydrogen or C₁₋₃ alkyl, R⁵ is selected from the group consisting of methyl, ethyl, propyl, isopropyl, butyl, tert-butyl, isobutyl, cyclopropylmethyl, pentyl, hexyl, oxazolyl, isoxazolyl, pyrazolyl, pyrrolyl, furanyl, thiophenyl, tetrahydrofuranyl, tetrahydropyranyl, oxetanyl, oxadiazolyl, phenyl, pyridinyl, cyclobutyl, cyclopropyl, cyclopentyl, cyclohexyl, wherein the R⁵ group is substituted with from 0 to 3 substituents selected from the group consisting of halogen, F, Cl, Br, R^{m}, —NO₂, —CN, —NR^{q}R^{r}, —OR^{q}, —C(O)₂NR^{q}R^{r}, —NR^{q}C(O)R^{r}, —S(O)₂R^{r}, —SR^{q} and phenyl.

In a twentieth embodiment of compounds of Formula I, and within certain aspects of the nineteenth embodiment, R⁵ is selected from the group consisting of:

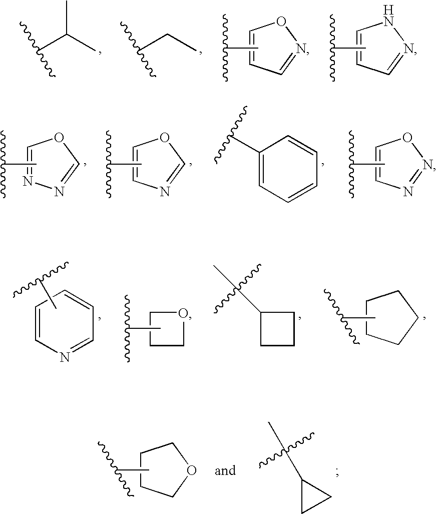

wherein from 0 to 3 hydrogen atoms attached to a carbon or nitrogen atom of R⁵ is optionally independently replaced with a R^{D} substitutent selected from the group consisting of halogen, C₁₋₃ haloalkyl, C₁₋₃ alkyl, —NR^{q}R^{r}, —OR^{q}, —S(O)₂R^{r}, halogen, F, Cl, and Br.

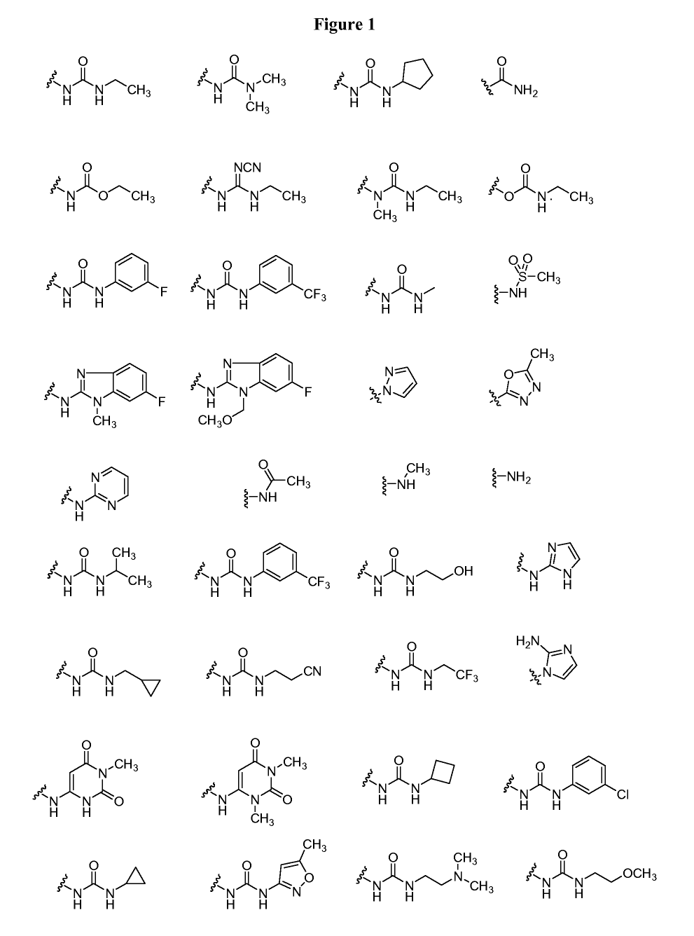

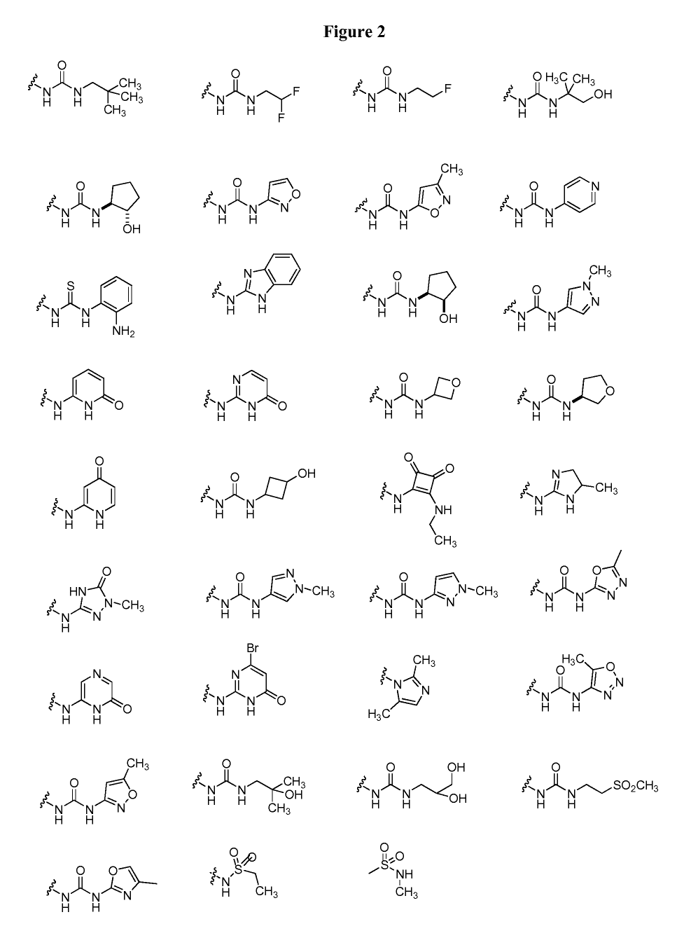

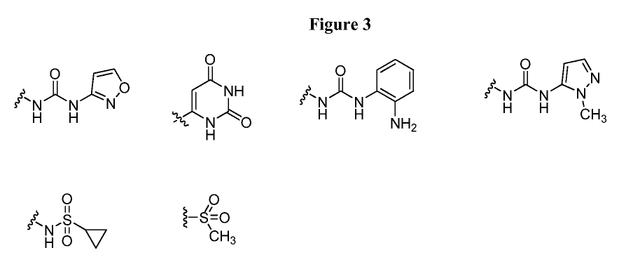

In a twenty-first embodiment of compounds of Formula I, D is selected from the group set forth in FIG. 1, FIG. 2 or FIG. 3.

In a twenty-second embodiment of compounds of Formula I, D is selected from the group consisting:

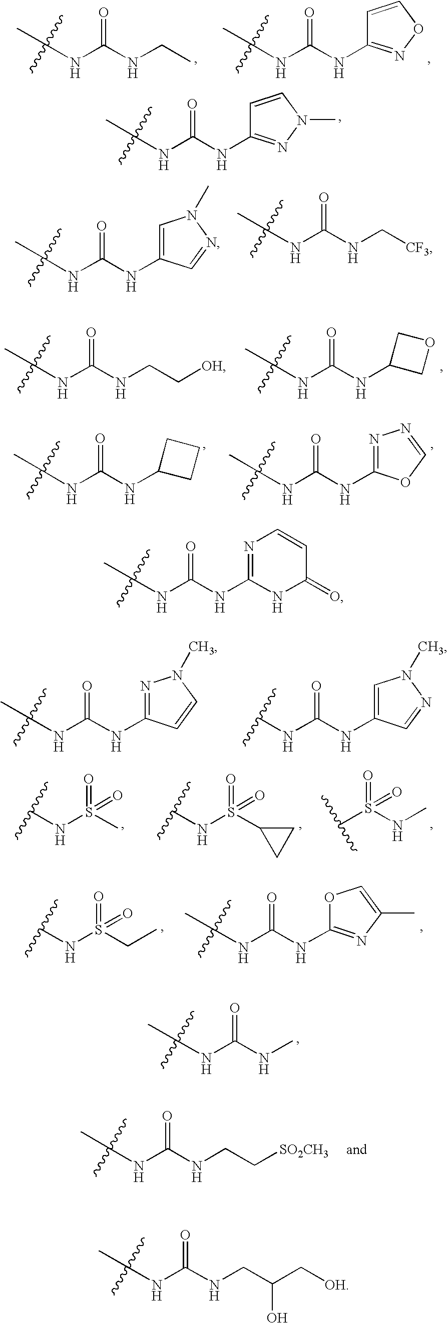

In a twenty-third embodiment of compounds of Formula I, -B-D in Formula I is selected from the group consisting of:

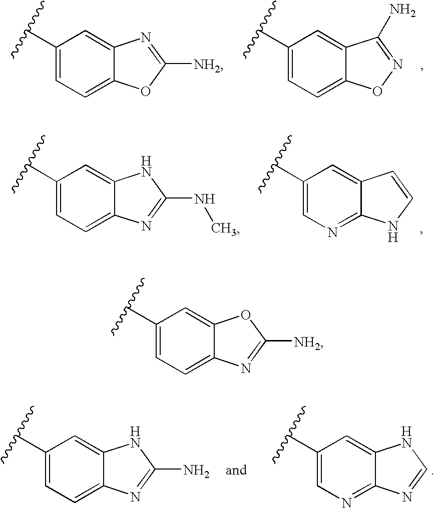

In a twenty-fourth embodiment of compounds of Formula I, the compound is selected from the group of discrete compounds in Table 1.

TABLE 1
1-ethyl-3-(4-(4-morpholino-6,7-dihydro-5H-pyrano[2,3-d]pyrimidin-2-yl)phenyl)urea;
(S)-1-ethyl-3-(4-(4-(3-methylmorpholino)-6,7-dihydro-5H-pyrano[2,3-d]pyrimidin-2-
yl)phenyl)urea;
(S)-1-ethyl-3-(4-(4-(3-methylmorpholino)-7,8-dihydro-5H-pyrano[4,3-d]pyrimidin-2-
yl)phenyl)urea;
1-ethyl-3-(4-(4-morpholino-7,8-dihydro-5H-pyrano[4,3-d]pyrimidin-2-yl)phenyl)urea;
(S)-1-ethyl-3-(4-(4-(3-ethylmorpholino)-7,8-dihydro-5H-pyrano[4,3-d]pyrimidin-2-
yl)phenyl)urea;
(S)-1-(isoxazol-3-yl)-3-(4-(4-(3-methylmorpholino)-7,8-dihydro-5H-pyrano[4,3-d]pyrimidin-
2-yl)phenyl)urea;
(S)-1-(1-methyl-1H-pyrazol-3-yl)-3-(4-(4-(3-methylmorpholino)-7,8-dihydro-5H-pyrano[4,3-
d]pyrimidin-2-yl)phenyl)urea;
(S)-1-(1-methyl-1H-pyrazol-4-yl)-3-(4-(4-(3-methylmorpholino)-7,8-dihydro-5H-pyrano[4,3-
d]pyrimidin-2-yl)phenyl)urea;
(S)-1-(4-(4-(3-methylmorpholino)-7,8-dihydro-5H-pyrano[4,3-d]pyrimidin-2-yl)phenyl)-3-
(2,2,2-trifluoroethyl)urea;
(S)-1-(2-hydroxyethyl)-3-(4-(4-(3-methylmorpholino)-7,8-dihydro-5H-pyrano[4,3-
d]pyrimidin-2-yl)phenyl)urea;
(S)-1-(4-(4-(3-methylmorpholino)-7,8-dihydro-5H-pyrano[4,3-d]pyrimidin-2-yl)phenyl)-3-
(oxetan-3-yl)urea;
(S)-1-cyclobutyl-3-(4-(4-(3-methylmorpholino)-7,8-dihydro-5H-pyrano[4,3-d]pyrimidin-2-
yl)phenyl)urea;
(S)-1-(5-methyl-1,3,4-oxadiazol-2-yl)-3-(4-(4-(3-methylmorpholino)-7,8-dihydro-5H-
pyrano[4,3-d]pyrimidin-2-yl)phenyl)urea;
(S)-1-ethyl-3-(4-(4-(3-ethylmorpholino)-6,7-dihydro-5H-pyrano[2,3-d]pyrimidin-2-
yl)phenyl)urea;
(S)-2-(4-(4-(3-methylmorpholino)-7,8-dihydro-5H-pyrano[4,3-d]pyrimidin-2-
yl)phenylamino)pyrimidin-4(3H)-one;
(S)-6-(4-(4-(3-methylmorpholino)-7,8-dihydro-5H-pyrano[4,3-d]pyrimidin-2-
yl)phenylamino)pyridin-2(1H)-one;
(S)-1-(1-methyl-1H-pyrazol-3-yl)-3-(4-(4-(3-methylmorpholino)-6,7-dihydro-5H-pyrano[2,3-
d]pyrimidin-2-yl)phenyl)urea;
(S)-1-(1-methyl-1H-pyrazol-4-yl)-3-(4-(4-(3-methylmorpholino)-6,7-dihydro-5H-pyrano[2,3-
d]pyrimidin-2-yl)phenyl)urea;
(S)-1-(4-(4-(3-methylmorpholino)-6,7-dihydro-5H-pyrano[2,3-d]pyrimidin-2-yl)phenyl)-3-
(oxetan-3-yl)urea;
(S)-1-(2-hydroxyethyl)-3-(4-(4-(3-methylmorpholino)-6,7-dihydro-5H-pyrano[2,3-
d]pyrimidin-2-yl)phenyl)urea;
(S)-1-(4-(7,7-dimethyl-4-(3-methylmorpholino)-5,7-dihydrofuro[3,4-d]pyrimidin-2-
yl)phenyl)-3-ethylurea;
1-(4-(4-((1S,4S)-2-oxa-5-azabicyclo[2.2.1]heptan-5-yl)-6,7-dihydro-5H-pyrano[2,3-
d]pyrimidin-2-yl)phenyl)-3-ethylurea;
(S)-2-(4-(4-(3-methylmorpholino)-6,7-dihydro-5H-pyrano[2,3-d]pyrimidin-2-
yl)phenylamino)pyrimidin-4(3H)-one;
(S)-6-(4-(4-(3-methylmorpholino)-6,7-dihydro-5H-pyrano[2,3-d]pyrimidin-2-
yl)phenylamino)pyridin-2(1H)-one;
(S)-4-(3-methylmorpholino)-2-(4-(methylsulfonyl)phenyl)-7,8-dihydro-5H-pyrano[4,3-
d]pyrimidine;
(S)—N-methyl-4-(4-(3-methylmorpholino)-7,8-dihydro-5H-pyrano[4,3-d]pyrimidin-2-
yl)benzenesulfonamide;
(S)—N-(4-(4-(3-methylmorpholino)-7,8-dihydro-5H-pyrano[4,3-d]pyrimidin-2-
yl)phenyl)methanesulfonamide;
(S)—N-(4-(4-(3-methylmorpholino)-7,8-dihydro-5H-pyrano[4,3-d]pyrimidin-2-
yl)phenyl)cyclopropanesulfonamide;
(S)-6-(4-(7,7-dimethyl-4-(3-methylmorpholino)-5,7-dihydrofuro[3,4-d]pyrimidin-2-
yl)phenylamino)pyridin-2(1H)-one;
1-ethyl-1-((ethylamino)carbonyl)-3-(4-(4-morpholino-6,8-dihydro-5H-pyrano[3,4-
d]pyrimidin-2-yl)phenyl)urea;
(S)—N-(4-(4-(3-methylmorpholino)-7,8-dihydro-5H-pyrano[4,3-d]pyrimidin-2-
yl)phenyl)ethanesulfonamide;
(S)-1-ethyl-3-(4-(4-(3-methylmorpholino)-6,8-dihydro-5H-pyrano[3,4-d]pyrimidin-2-
yl)phenyl)urea;
(S)-1-ethyl-1-((ethylamino)carbonyl)-3-(4-(4-(3-methylmorpholino)-6,8-dihydro-5H-
pyrano[3,4-d]pyrimidin-2-yl)phenyl)urea;
1-ethyl-3-(4-(4-morpholino-7,8-dihydro-6H-pyrano[3,2-d]pyrimidin-2-yl)phenyl)urea;
(S)-2-(4-(4-(3-ethylmorpholino)-7,8-dihydro-5H-pyrano[4,3-d]pyrimidin-2-
yl)phenylamino)pyrimidin-4(3H)-one;
(S)-6-(4-(4-(3-ethylmorpholino)-7,8-dihydro-5H-pyrano[4,3-d]pyrimidin-2-
yl)phenylamino)pyridin-2(1H)-one;
(S)-1-(4-(4-(3-ethylmorpholino)-7,8-dihydro-5H-pyrano[4,3-d]pyrimidin-2-yl)phenyl)-3-
(oxetan-3-yl)urea;
1-ethyl-3-(4-(4′-morpholino-5′,6′-dihydrospiro[cyclopropane-1,7′-pyrano[2,3-d]pyrimidine]-
2′-yl)phenyl)urea;
2-(4-(4′-morpholino-5′,6′-dihydrospiro[cyclopropane-1,7′-pyrano[2,3-d]pyrimidine]-2′-
yl)phenylamino)pyrimidin-4(3H)-one;
1-(4-(4′-morpholino-5′,6′-dihydrospiro[cyclopropane-1,7′-pyrano[2,3-d]pyrimidine]-2′-
yl)phenyl)-3-(oxetan-3-yl)urea;
1-(1-methyl-1H-pyrazol-3-yl)-3-(4-(4′-morpholino-5′,6′-dihydrospiro[cyclopropane-1,7′-
pyrano[2,3-d]pyrimidine]-2′-yl)phenyl)urea;
1-(1-methyl-1H-pyrazol-4-yl)-3-(4-(4′-morpholino-5′,6′-dihydrospiro[cyclopropane-1,7′-
pyrano[2,3-d]pyrimidine]-2′-yl)phenyl)urea;
1-(4-(4′-(4-methoxypiperidin-1-yl)-5′,6′-dihydrospiro[cyclopropane-1,7′-pyrano[2,3-
d]pyrimidine]-2′-yl)phenyl)-3-(oxetan-3-yl)urea;
1-(4-(4′-(4-methoxypiperidin-1-yl)-5′,6′-dihydrospiro[cyclopropane-1,7′-pyrano[2,3-
d]pyrimidine]-2′-yl)phenyl)-3-(1-methyl-1H-pyrazol-3-yl)urea;
2-(4-(4′-(4-methoxypiperidin-1-yl)-5′,6′-dihydrospiro[cyclopropane-1,7′-pyrano[2,3-
d]pyrimidine]-2′-yl)phenylamino)pyrimidin-4(3H)-one;
(S)-1-ethyl-3-(4-(4′-(3-methylmorpholino)-5′,6′-dihydrospiro[cyclopropane-1,7′-pyrano[2,3-
d]pyrimidine]-2′-yl)phenyl)urea;
(S)-1-(1-methyl-1H-pyrazol-4-yl)-3-(4-(4′-(3-methylmorpholino)-5′,6′-
dihydrospiro[cyclopropane-1,7′-pyrano[2,3-d]pyrimidine]-2′-yl)phenyl)urea;
(S)-1-(4-(4′-(3-methylmorpholino)-5′,6′-dihydrospiro[cyclopropane-1,7′-pyrano[2,3-
d]pyrimidine]-2′-yl)phenyl)-3-(oxetan-3-yl)urea;
(S)-1-(1-methyl-1H-pyrazol-3-yl)-3-(4-(4′-(3-methylmorpholino)-5′,6′-
dihydrospiro[cyclopropane-1,7′-pyrano[2,3-d]pyrimidine]-2′-yl)phenyl)urea;
(S)-1-(4-(7,7-dimethyl-4-(3-methylmorpholino)-5,7-dihydrofuro[3,4-d]pyrimidin-2-
yl)phenyl)-3-(1-methyl-1H-pyrazol-4-yl)urea;
(S)-1-(4-(4′-(3-methylmorpholino)-5′,6′-dihydrospiro[cyclopropane-1,7′-pyrano[2,3-
d]pyrimidine]-2′-yl)phenyl)-3-(4-methyloxazol-2-yl)urea;
(S)-6-(4-(4′-(3-methylmorpholino)-5′,6′-dihydrospiro[cyclopropane-1,7′-pyrano[2,3-
d]pyrimidine]-2′-yl)phenylamino)pyridin-2(1H)-one;
(S)-2-(4-(4′-(3-methylmorpholino)-5′,6′-dihydrospiro[cyclopropane-1,7′-pyrano[2,3-
d]pyrimidine]-2′-yl)phenylamino)pyrimidin-4(3H)-one;
(S)-1-methyl-3-(4-(4′-(3-methylmorpholino)-5′,6′-dihydrospiro[cyclopropane-1,7′-
pyrano[2,3-d]pyrimidine]-2′-yl)phenyl)urea;
(S)-1-(4-(4′-(3-methylmorpholino)-5′,6′-dihydrospiro[cyclopropane-1,7′-pyrano[2,3-
d]pyrimidine]-2′-yl)phenyl)-3-(2-(methylsulfonyl)ethyl)urea;
(S)-1-methyl-3-(4-(4-(3-methylmorpholino)-7,8-dihydro-5H-pyrano[4,3-d]pyrimidin-2-
yl)phenyl)urea;
(S)-1-(4-(4-(3-methylmorpholino)-7,8-dihydro-5H-pyrano[4,3-d]pyrimidin-2-yl)phenyl)-3-
(2-(methylsulfonyl)ethyl)urea;
(S)-1-(4-(7,7-dimethyl-4-(3-methylmorpholino)-5,7-dihydrofuro[3,4-d]pyrimidin-2-
yl)phenyl)-3-(oxetan-3-yl)urea;
(S)-1-(4-(7,7-dimethyl-4-(3-methylmorpholino)-5,7-dihydrofuro[3,4-d]pyrimidin-2-
yl)phenyl)-3-(2-hydroxyethyl)urea;
(S)-1-(2-cyanoethyl)-3-(4-(7,7-dimethyl-4-(3-methylmorpholino)-5,7-dihydrofuro[3,4-
d]pyrimidin-2-yl)phenyl)urea;
1-(4-(4-((1R,5S)-3-oxa-8-azabicyclo[3.2.1]octan-8-yl)-6,7-dihydro-5H-pyrano[2,3-
d]pyrimidin-2-yl)phenyl)-3-ethylurea;
1-((R)-2,3-dihydroxypropyl)-3-(4-(7,7-dimethyl-4-((S)-3-methylmorpholino)-5,7-
dihydrofuro[3,4-d]pyrimidin-2-yl)phenyl)urea;
(S)-1-(2-hydroxyethyl)-3-(4-(4′-(3-methylmorpholino)-5′,6′-dihydrospiro[cyclopropane-1,7′-
pyrano[2,3-d]pyrimidine]-2′-yl)phenyl)urea;
(S)-1-(2-cyanoethyl)-3-(4-(4′-(3-methylmorpholino)-5′,6′-dihydrospiro[cyclopropane-1,7′-
pyrano[2,3-d]pyrimidine]-2′-yl)phenyl)urea;
1-(4-(7,7-dimethyl-4-morpholino-5-oxo-5,7-dihydrofuro[3,4-d]pyrimidin-2-yl)phenyl)-3-
ethylurea;
1-((S)-2,3-dihydroxypropyl)-3-(4-(4′-((S)-3-methylmorpholino)-5′,6′-
dihydrospiro[cyclopropane-1,7′-pyrano[2,3-d]pyrimidine]-2′-yl)phenyl)urea;
(S)-1-methoxy-3-(4-(4′-(3-methylmorpholino)-5′,6′-dihydrospiro[cyclopropane-1,7′-
pyrano[2,3-d]pyrimidine]-2′-yl)phenyl)urea;
1-((R)-2,3-dihydroxypropyl)-3-(4-(4′-((S)-3-methylmorpholino)-5′,6′-
dihydrospiro[cyclopropane-1,7′-pyrano[2,3-d]pyrimidine]-2′-yl)phenyl)urea;
1-(4-(7-(benzyloxymethyl)-4-((S)-3-methylmorpholino)-7,8-dihydro-5H-pyrano[4,3-
d]pyrimidin-2-yl)phenyl)-3-ethylurea;
1-Ethyl-3-{4-[(1R,9S)-3-((S)-3-methyl-morpholin-4-yl)-12-oxa-4,6-diaza-tricyclo[7.2.1.0-
2,7]dodeca-2(7),3,5-trien-5-yl]-phenyl}-urea;
1-Ethyl-3-{4-[(1S,9R)-3-((S)-3-methyl-morpholin-4-yl)-12-oxa-4,6-diaza-tricyclo[7.2.1.0-
2,7]dodeca-2(7),3,5-trien-5-yl]-phenyl}-urea;
1-(4-(4-((1R,5S)-3-oxa-8-azabicyclo[3.2.1]octan-8-yl)-6,7-dihydro-5H-pyrano[2,3-
d]pyrimidin-2-yl)phenyl)-3-(oxetan-3-yl)urea;
1-ethyl-3-(4-(7-(2-hydroxyethyl)-7-methyl-4-((S)-3-methylmorpholino)-5,7-dihydrofuro[3,4-
d]pyrimidin-2-yl)phenyl)urea;
2-(4-(7-(hydroxymethyl)-4-((S)-3-methylmorpholino)-7,8-dihydro-5H-pyrano[4,3-
d]pyrimidin-2-yl)phenylamino)pyrimidin-4(3H)-one;
1-ethyl-3-(4-((R)-7-(2-hydroxyethyl)-7-methyl-4-((S)-3-methylmorpholino)-5,7-
dihydrofuro[3,4-d]pyrimidin-2-yl)phenyl)urea;
1-ethyl-3-(4-((S)-7-(2-hydroxyethyl)-7-methyl-4-((S)-3-methylmorpholino)-5,7-
dihydrofuro[3,4-d]pyrimidin-2-yl)phenyl)urea;
1-{4-[(1R,9S)-3-((S)-3-Methyl-morpholin-4-yl)-12-oxa-4,6-diaza-tricyclo[7.2.1.0-
2,7]dodeca-2(7),3,5-trien-5-yl]-phenyl}-3-oxetan-3-yl-urea;
1-{4-[(1S,9R)-3-((S)-3-Methyl-morpholin-4-yl)-12-oxa-4,6-diaza-tricyclo[7.2.1.0-
2,7]dodeca-2(7),3,5-trien-5-yl]-phenyl}-3-oxetan-3-yl-urea;
1-(4-(4′-((1R,5S)-3-oxa-8-azabicyclo[3.2.1]octan-8-yl)-5′,6′-dihydrospiro[cyclopropane-1,7′-
pyrano[2,3-d]pyrimidine]-2′-yl)phenyl)-3-(oxetan-3-yl)urea;
1-(4-(4′-((1R,5S)-3-oxa-8-azabicyclo[3.2.1]octan-8-yl)-5′,6′-dihydrospiro[cyclopropane-1,7′-
pyrano[2,3-d]pyrimidine]-2′-yl)phenyl)-3-(2-hydroxyethyl)urea;
(S)-1-(1-(hydroxymethyl)cyclopropyl)-3-(4-(4′-(3-methylmorpholino)-5′,6′-
dihydrospiro[cyclopropane-1,7′-pyrano[2,3-d]pyrimidine]-2′-yl)phenyl)urea;
1-ethyl-3-(4-(7-(hydroxymethyl)-4-((S)-3-methylmorpholino)-7,8-dihydro-5H-pyrano[4,3-
d]pyrimidin-2-yl)phenyl)urea;
(S)-1-(4-(7,7-dimethyl-4-(3-methylmorpholino)-7,8-dihydro-5H-pyrano[4,3-d]pyrimidin-2-
yl)phenyl)-3-ethylurea;
1-(4-((R)-7-allyl-7-methyl-4-((S)-3-methylmorpholino)-5,7-dihydrofuro[3,4-d]pyrimidin-2-
yl)phenyl)-3-ethylurea;
1-(4-((S)-7-allyl-7-methyl-4-((S)-3-methylmorpholino)-5,7-dihydrofuro[3,4-d]pyrimidin-2-
yl)phenyl)-3-ethylurea;
1-(4-(7-(cyclopropylmethyl)-7-methyl-4-((S)-3-methylmorpholino)-5,7-dihydrofuro[3,4-
d]pyrimidin-2-yl)phenyl)-3-ethylurea;
3-ethyl-1-(4-((S)-7-(2-hydroxyethyl)-7-methyl-4-((S)-3-methylmorpholino)-5,7-
dihydrofuro[3,4-d]pyrimidin-2-yl)phenyl)-1-methylurea;
3-ethyl-1-(4-((R)-7-(2-hydroxyethyl)-7-methyl-4-((S)-3-methylmorpholino)-5,7-
dihydrofuro[3,4-d]pyrimidin-2-yl)phenyl)-1-methylurea;
1-ethyl-3-(4-(4-morpholino-7-(pyridin-2-yl)-7,8-dihydro-5H-pyrano[4,3-d]pyrimidin-2-
yl)phenyl)urea;
1-ethyl-3-(4-(7-methyl-4-((S)-3-methylmorpholino)-7-propyl-5,7-dihydrofuro[3,4-
d]pyrimidin-2-yl)phenyl)urea;
1-ethyl-3-(4-((S)-7-(2-methoxyethyl)-7-methyl-4-((S)-3-methylmorpholino)-5,7-
dihydrofuro[3,4-d]pyrimidin-2-yl)phenyl)urea;
1-ethyl-3-(4-((R)-7-(2-methoxyethyl)-7-methyl-4-((S)-3-methylmorpholino)-5,7-
dihydrofuro[3,4-d]pyrimidin-2-yl)phenyl)urea;
1-ethyl-3-(4-((S)-7-(3-hydroxypropyl)-7-methyl-4-((S)-3-methylmorpholino)-5,7-
dihydrofuro[3,4-d]pyrimidin-2-yl)phenyl)urea;
1-ethyl-3-(4-((R)-7-(3-hydroxypropyl)-7-methyl-4-((S)-3-methylmorpholino)-5,7-
dihydrofuro[3,4-d]pyrimidin-2-yl)phenyl)urea;
1-ethyl-3-(4-((7S)-7-(2-hydroxypropyl)-7-methyl-4-((S)-3-methylmorpholino)-5,7-
dihydrofuro[3,4-d]pyrimidin-2-yl)phenyl)urea;
1-ethyl-3-(4-((7R)-7-(2-hydroxypropyl)-7-methyl-4-((S)-3-methylmorpholino)-5,7-
dihydrofuro[3,4-d]pyrimidin-2-yl)phenyl)urea;
1-ethyl-3-(4-((S)-7-methyl-4-((S)-3-methylmorpholino)-7-(2-morpholinoethyl)-5,7-
dihydrofuro[3,4-d]pyrimidin-2-yl)phenyl)urea;
1-ethyl-3-(4-((R)-7-methyl-4-((S)-3-methylmorpholino)-7-(2-morpholinoethyl)-5,7-
dihydrofuro[3,4-d]pyrimidin-2-yl)phenyl)urea;
1-ethyl-3-(4-((S)-7-methyl-7-(2-(2-methyl-1H-imidazol-1-yl)ethyl)-4-((S)-3-
methylmorpholino)-5,7-dihydrofuro[3,4-d]pyrimidin-2-yl)phenyl)urea;
1-ethyl-3-(4-((R)-7-methyl-7-(2-(2-methyl-1H-imidazol-1-yl)ethyl)-4-((S)-3-
methylmorpholino)-5,7-dihydrofuro[3,4-d]pyrimidin-2-yl)phenyl)urea;
1-(4-((R)-7-(2-(azetidin-1-yl)ethyl)-7-methyl-4-((S)-3-methylmorpholino)-5,7-
dihydrofuro[3,4-d]pyrimidin-2-yl)phenyl)-3-ethylurea;
1-(4-((S)-7-(2-(azetidin-1-yl)ethyl)-7-methyl-4-((S)-3-methylmorpholino)-5,7-
dihydrofuro[3,4-d]pyrimidin-2-yl)phenyl)-3-ethylurea;
5-(4-((1R,5S)-3-oxa-8-azabicyclo[3.2.1]octan-8-yl)-6,7-dihydro-5H-pyrano[2,3-d]pyrimidin-
2-yl)pyrimidin-2-amine;
5-(4-((1R,5S)-3-oxa-8-azabicyclo[3.2.1]octan-8-yl)-6,7-dihydro-5H-pyrano[2,3-d]pyrimidin-
2-yl)pyridin-2-amine;
5-(4-((1R,5S)-8-oxa-3-azabicyclo[3.2.1]octan-3-yl)-6,7-dihydro-5H-pyrano[2,3-d]pyrimidin-
2-yl)pyrimidin-2-amine;
5-(4-((1R,5S)-8-oxa-3-azabicyclo[3.2.1]octan-3-yl)-6,7-dihydro-5H-pyrano[2,3-d]pyrimidin-
2-yl)pyridin-2-amine;
(S)-1-ethyl-3-(4-(4-(3-methylmorpholino)-7-oxo-5,7-dihydrofuro[3,4-d]pyrimidin-2-
yl)phenyl)urea;
1-ethyl-3-(4-((S)-7-methyl-4-((S)-3-methylmorpholino)-7-(2-(pyridin-4-yloxy)ethyl)-5,7-
dihydrofuro[3,4-d]pyrimidin-2-yl)phenyl)urea;
1-ethyl-3-(4-((R)-7-methyl-4-((S)-3-methylmorpholino)-7-(2-(pyridin-4-yloxy)ethyl)-5,7-
dihydrofuro[3,4-d]pyrimidin-2-yl)phenyl)urea;
5-((S)-7-(2-methoxyethyl)-7-methyl-4-((S)-3-methylmorpholino)-5,7-dihydrofuro[3,4-
d]pyrimidin-2-yl)pyrimidin-2-amine;
5-((R)-7-(2-methoxyethyl)-7-methyl-4-((S)-3-methylmorpholino)-5,7-dihydrofuro[3,4-
d]pyrimidin-2-yl)pyrimidin-2-amine;
1-ethyl-3-(4-(7-methyl-4-(3-methylmorpholino)-7-(2-phenoxyethyl)-5,7-dihydrofuro[3,4-
d]pyrimidin-2-yl)phenyl)urea;
1-ethyl-3-(4-(7-methyl-4-(3-methylmorpholino)-7-(2-phenoxyethyl)-5,7-dihydrofuro[3,4-
d]pyrimidin-2-yl)phenyl)urea;
(S)-1-(4-(7-allyl-7-methyl-4-morpholino-5,7-dihydrofuro[3,4-d]pyrimidin-2-yl)phenyl)-3-
ethylurea;
(R)-1-(4-(7-allyl-7-methyl-4-morpholino-5,7-dihydrofuro[3,4-d]pyrimidin-2-yl)phenyl)-3-
ethylurea;
5-(7,7-dimethyl-4-morpholino-5,7-dihydrofuro[3,4-d]pyrimidin-2-yl)pyridin-2-amine;
(R)-1-ethyl-3-(4-(7-methyl-4-morpholino-7-propyl-5,7-dihydrofuro[3,4-d]pyrimidin-2-
yl)phenyl)urea;
(S)-1-ethyl-3-(4-(7-methyl-4-morpholino-7-propyl-5,7-dihydrofuro[3,4-d]pyrimidin-2-
yl)phenyl)urea;
5-((S)-7-(2-methoxyethyl)-7-methyl-4-((S)-3-methylmorpholino)-5,7-dihydrofuro[3,4-
d]pyrimidin-2-yl)benzo[d]oxazol-2-amine;
5-((R)-7-(2-methoxyethyl)-7-methyl-4-((S)-3-methylmorpholino)-5,7-dihydrofuro[3,4-
d]pyrimidin-2-yl)benzo[d]oxazol-2-amine;
5-((S)-7-(2-methoxyethyl)-7-methyl-4-((S)-3-methylmorpholino)-5,7-dihydrofuro[3,4-
d]pyrimidin-2-yl)pyridin-2-amine;
5-((R)-7-(2-methoxyethyl)-7-methyl-4-((S)-3-methylmorpholino)-5,7-dihydrofuro[3,4-
d]pyrimidin-2-yl)pyridin-2-amine;
6-((S)-7-(2-methoxyethyl)-7-methyl-4-((S)-3-methylmorpholino)-5,7-dihydrofuro[3,4-
d]pyrimidin-2-yl)benzo[d]oxazol-2-amine;
6-((R)-7-(2-methoxyethyl)-7-methyl-4-((S)-3-methylmorpholino)-5,7-dihydrofuro[3,4-
d]pyrimidin-2-yl)benzo[d]oxazol-2-amine;
(S)-1-ethyl-3-(4-(7-(2-hydroxyethyl)-7-methyl-4-morpholino-5,7-dihydrofuro[3,4-
d]pyrimidin-2-yl)phenyl)urea;
(R)-1-ethyl-3-(4-(7-(2-hydroxyethyl)-7-methyl-4-morpholino-5,7-dihydrofuro[3,4-
d]pyrimidin-2-yl)phenyl)urea;
1-ethyl-3-(4-((R)-7-(2-(ethyl(methyl)amino)ethyl)-7-methyl-4-((S)-3-methylmorpholino)-
5,7-dihydrofuro[3,4-d]pyrimidin-2-yl)phenyl)urea;
1-ethyl-3-(4-((S)-7-(2-(ethyl(methyl)amino)ethyl)-7-methyl-4-((S)-3-methylmorpholino)-5,7-
dihydrofuro[3,4-d]pyrimidin-2-yl)phenyl)urea;
1-(4-((R)-7-(2-cyanoethyl)-7-methyl-4-((S)-3-methylmorpholino)-5,7-dihydrofuro[3,4-
d]pyrimidin-2-yl)phenyl)-3-ethylurea;
1-(4-((S)-7-(2-cyanoethyl)-7-methyl-4-((S)-3-methylmorpholino)-5,7-dihydrofuro[3,4-
d]pyrimidin-2-yl)phenyl)-3-ethylurea;
(S)-5-(7,7-dimethyl-4-(3-methylmorpholino)-5,7-dihydrofuro[3,4-d]pyrimidin-2-
yl)pyrimidin-2-amine;
1-(4-((R)-7-(2-(1H-imidazol-1-yl)ethyl)-7-methyl-4-((S)-3-methylmorpholino)-5,7-
dihydrofuro[3,4-d]pyrimidin-2-yl)phenyl)-3-ethylurea;
1-(4-((S)-7-(2-(1H-imidazol-1-yl)ethyl)-7-methyl-4-((S)-3-methylmorpholino)-5,7-
dihydrofuro[3,4-d]pyrimidin-2-yl)phenyl)-3-ethylurea;
5-((S)-7-methyl-4-((S)-3-methylmorpholino)-7-(2-phenoxyethyl)-5,7-dihydrofuro[3,4-
d]pyrimidin-2-yl)pyrimidin-2-amine;
5-((R)-7-methyl-4-((S)-3-methylmorpholino)-7-(2-phenoxyethyl)-5,7-dihydrofuro[3,4-
d]pyrimidin-2-yl)pyrimidin-2-amine;
6-((S)-7-(2-methoxyethyl)-7-methyl-4-((S)-3-methylmorpholino)-5,7-dihydrofuro[3,4-
d]pyrimidin-2-yl)benzo[d]isoxazol-3-amine;
6-((R)-7-(2-methoxyethyl)-7-methyl-4-((S)-3-methylmorpholino)-5,7-dihydrofuro[3,4-
d]pyrimidin-2-yl)benzo[d]isoxazol-3-amine;
5-(7,7-dimethyl-4-morpholino-5,7-dihydrofuro[3,4-d]pyrimidin-2-yl)benzo[d]oxazol-2-
amine;
6-(7,7-dimethyl-4-morpholino-5,7-dihydrofuro[3,4-d]pyrimidin-2-yl)benzo[d]oxazol-2-
amine;
6-(4-((1R,5S)-8-oxa-3-azabicyclo[3.2.1]octan-3-yl)-6,7-dihydro-5H-pyrano[2,3-d]pyrimidin-
2-yl)-1H-benzo[d]imidazol-2-amine;
6-(7,7-dimethyl-4-morpholino-5,7-dihydrofuro[3,4-d]pyrimidin-2-yl)benzo[d]isoxazol-3-
amine;
5-(7,7-dimethyl-4-morpholino-5,7-dihydrofuro[3,4-d]pyrimidin-2-yl)benzo[d]isoxazol-3-
amine;
6-(7,7-dimethyl-4-morpholino-5,7-dihydrofuro[3,4-d]pyrimidin-2-yl)-1H-benzo[d]imidazol-
2-amine;
5-((S)-7-(2-methoxyethyl)-7-methyl-4-((S)-3-methylmorpholino)-5,7-dihydrofuro[3,4-
d]pyrimidin-2-yl)benzo[d]isoxazol-3-amine;
5-((R)-7-(2-methoxyethyl)-7-methyl-4-((S)-3-methylmorpholino)-5,7-dihydrofuro[3,4-
d]pyrimidin-2-yl)benzo[d]isoxazol-3-amine;
1-ethyl-3-(4-((S)-7-(hydroxymethyl)-7-methyl-4-((S)-3-methylmorpholino)-5,7-
dihydrofuro[3,4-d]pyrimidin-2-yl)phenyl)urea;
1-ethyl-3-(4-((R)-7-(hydroxymethyl)-7-methyl-4-((S)-3-methylmorpholino)-5,7-
dihydrofuro[3,4-d]pyrimidin-2-yl)phenyl)urea;
1-(4-((R)-7-allyl-4-((1R,5S)-8-oxa-3-azabicyclo[3.2.1]octan-3-yl)-7-methyl-5,7-
dihydrofuro[3,4-d]pyrimidin-2-yl)phenyl)-3-ethylurea;
1-(4-((S)-7-allyl-4-((1R,5S)-8-oxa-3-azabicyclo[3.2.1]octan-3-yl)-7-methyl-5,7-
dihydrofuro[3,4-d]pyrimidin-2-yl)phenyl)-3-ethylurea;
1-(4-((S)-4-((1R,5S)-8-oxa-3-azabicyclo[3.2.1]octan-3-yl)-7-methyl-7-propyl-5,7-
dihydrofuro[3,4-d]pyrimidin-2-yl)phenyl)-3-ethylurea;
1-(4-((R)-4-((1R,5S)-8-oxa-3-azabicyclo[3.2.1]octan-3-yl)-7-methyl-7-propyl-5,7-
dihydrofuro[3,4-d]pyrimidin-2-yl)phenyl)-3-ethylurea a;
(S)-6-(7,7-dimethyl-4-(3-methylmorpholino)-5,7-dihydrofuro[3,4-d]pyrimidin-2-
yl)benzo[d]isoxazol-3-amine;
(S)-5-(7,7-dimethyl-4-(3-methylmorpholino)-5,7-dihydrofuro[3,4-d]pyrimidin-2-
yl)benzo[d]isoxazol-3-amine;
(S)-6-(7,7-dimethyl-4-(3-methylmorpholino)-5,7-dihydrofuro[3,4-d]pyrimidin-2-yl)-1H-
benzo[d]imidazol-2-amine;
1-(4-((S)-4-((1R,5S)-8-oxa-3-azabicyclo[3.2.1]octan-3-yl)-7-(2-hydroxyethyl)-7-methyl-5,7-
dihydrofuro[3,4-d]pyrimidin-2-yl)phenyl)-3-ethylurea;
1-(4-((R)-4-((1R,5S)-8-oxa-3-azabicyclo[3.2.1]octan-3-yl)-7-(2-hydroxyethyl)-7-methyl-5,7-
dihydrofuro[3,4-d]pyrimidin-2-yl)phenyl)-3-ethylurea;
5-(4-((1R,4R)-2-oxa-5-azabicyclo[2.2.1]heptan-5-yl)-7,7-dimethyl-5,7-dihydrofuro[3,4-
d]pyrimidin-2-yl)pyrimidin-2-amine;
5-(4-((1R,5S)-3-oxa-8-azabicyclo[3.2.1]octan-8-yl)-7,7-dimethyl-5,7-dihydrofuro[3,4-
d]pyrimidin-2-yl)pyrimidin-2-amine;
5-(4-((1R,5S)-8-oxa-3-azabicyclo[3.2.1]octan-3-yl)-7,7-dimethyl-5,7-dihydrofuro[3,4-
d]pyrimidin-2-yl)pyrimidin-2-amine;
(S)-5-(7,7-dimethyl-4-(3-methylmorpholino)-5,7-dihydrofuro[3,4-d]pyrimidin-2-
yl)benzo[d]oxazol-2-amine;
6-(7-(2-methoxyethyl)-7-methyl-4-((S)-3-methylmorpholino)-5,7-dihydrofuro[3,4-
d]pyrimidin-2-yl)-1H-benzo[d]imidazol-2-amine;
6-((R)-7-(2-methoxyethyl)-7-methyl-4-((S)-3-methylmorpholino)-5,7-dihydrofuro[3,4-
d]pyrimidin-2-yl)-1H-benzo[d]imidazol-2-amine;
(S)-6-(4-(3-methylmorpholino)-6,7-dihydro-5H-pyrano[2,3-d]pyrimidin-2-yl)-1H-
benzo[d]imidazol-2-amine;
(S)-5-(4-(3-ethylmorpholino)-6,7-dihydro-5H-pyrano[2,3-d]pyrimidin-2-yl)pyrimidin-2-
amine;
(S)-5-(4-(3-ethylmorpholino)-6,7-dihydro-5H-pyrano[2,3-d]pyrimidin-2-yl)pyridin-2-amine;
(S)-5-(7,7-dimethyl-4-(3-methylmorpholino)-5,7-dihydrofuro[3,4-d]pyrimidin-2-yl)-N-
methyl-1H-benzo[d]imidazol-2-amine;
2-((S)-2-(2-amino-1H-benzo[d]imidazol-5-yl)-7-methyl-4-((S)-3-methylmorpholino)-5,7-
dihydrofuro[3,4-d]pyrimidin-7-yl)ethanol;
1-ethyl-3-(4-((S)-7-(2-hydroxy-2-methylpropyl)-7-methyl-4-((S)-3-methylmorpholino)-5,7-
dihydrofuro[3,4-d]pyrimidin-2-yl)phenyl)urea;
1-ethyl-3-(4-((R)-7-(2-hydroxy-2-methylpropyl)-7-methyl-4-((S)-3-methylmorpholino)-5,7-
dihydrofuro[3,4-d]pyrimidin-2-yl)phenyl)urea;
2-((R)-2-(2-amino-1H-benzo[d]imidazol-5-yl)-7-methyl-4-((S)-3-methylmorpholino)-5,7-
dihydrofuro[3,4-d]pyrimidin-7-yl)ethanol;
(S)-1-ethyl-3-(4-(7-(2-hydroxy-2-methylpropyl)-7-methyl-4-morpholino-5,7-
dihydrofuro[3,4-d]pyrimidin-2-yl)phenyl)urea;
(R)-1-ethyl-3-(4-(7-(2-hydroxy-2-methylpropyl)-7-methyl-4-morpholino-5,7-
dihydrofuro[3,4-d]pyrimidin-2-yl)phenyl)urea;
(S)-1-ethyl-3-(4-(4-(3-methylmorpholino)-5,7-dihydrofuro[3,4-d]pyrimidin-2-yl)phenyl)urea;
(R)-1-ethyl-3-(4-(7-(hydroxymethyl)-7-methyl-4-morpholino-5,7-dihydrofuro[3,4-
d]pyrimidin-2-yl)phenyl)urea;
(S)-1-ethyl-3-(4-(7-(hydroxymethyl)-7-methyl-4-morpholino-5,7-dihydrofuro[3,4-
d]pyrimidin-2-yl)phenyl)urea;
1-ethyl-3-(4-(4-morpholino-5,7-dihydrofuro[3,4-d]pyrimidin-2-yl)phenyl)urea;
1-(4-(4-((1R,5S)-3-oxa-8-azabicyclo[3.2.1]octan-8-yl)-5,7-dihydrofuro[3,4-d]pyrimidin-2-
yl)phenyl)-3-ethylurea; and
1-(4-(4-(8-oxa-3-azabicyclo[3.2.1]octan-3-yl)-5,7-dihydrofuro[3,4-d]pyrimidin-2-yl)phenyl)-
3-ethylurea.

It is understood that the embodiments described hereinabove are for illustrative purposes only and that the different combinations of embodiments are suggested to persons skilled in the art and are to be included within the purview of this application and scope of the appended claims.

Also falling within the scope of this invention are the in vivo metabolic products of Formula I described herein or any subgenus (e.g., Formula II-A) or species thereof. The invention includes metabolites of compounds of Formula I, including compounds produced by a process comprising contacting a compound of this invention with a mammal for a period of time sufficient to yield a metabolic product thereof.

Also falling with in the scope of the invention, are pharmaceutically acceptable prodrugs of compounds of Formula I or radiolabelled compounds of Formula I described herein or any subgenus (e.g., Formula II-A) or species thereof.

II.B Synthesis of Compounds

As shown in the Examples section below, there are a variety of synthetic routes by which a skilled artisan can prepare compounds of the present invention and intermediates used to prepare such compounds. The following schemes illustrate some general methods for the preparation of compounds of the invention along with key intermediates. When present in the Schemes described below, P represents a protecting group; X is a leaving group, such as a halogen, tosylate, etc; (H)Ar is an aryl or heteroaryl group that is optionally substituted with non-interferring substitutents; the subscript n, at each occurrence, is independently an integer from 0 to 2. Other non-interferring substitutents are noted as —R, —R′, —R″ and —R′″ groups. In R—NH—R′, the —R and —R′ are combined to form a heterocyclic ring comprising an oxygen atom. The symbols A¹ and A² each independently represents —CH₂—, —CHR—, —CRR—, —C(═O)—, etc.

Scheme 1 illustrates a general synthetic method of oxo-ring fused pyrimidines that are useful in the synthesis of compounds of the invention of Formula I. For example, compound 1e and related analogs can be elaborated as described in Scheme 4 (below) to form compounds of the invention. In more detail, a tetrahydropyranone 1a can be treated with 2 equivalents of methylthiocyanide to produce a pyranyl fused pyrimidine compound 1b, which upon oxidation, e.g., using a peroxide reagent, to produce disulfone 1c. Treatment of 1c under basic hydrolysis conditions followed by treatment of the resultant producing using halogenating conditions such as, for example, P(O)Cl₃ or PBr₃, can produce the dihalogonated product, 1e, wherein X═Cl, or Br, among others.

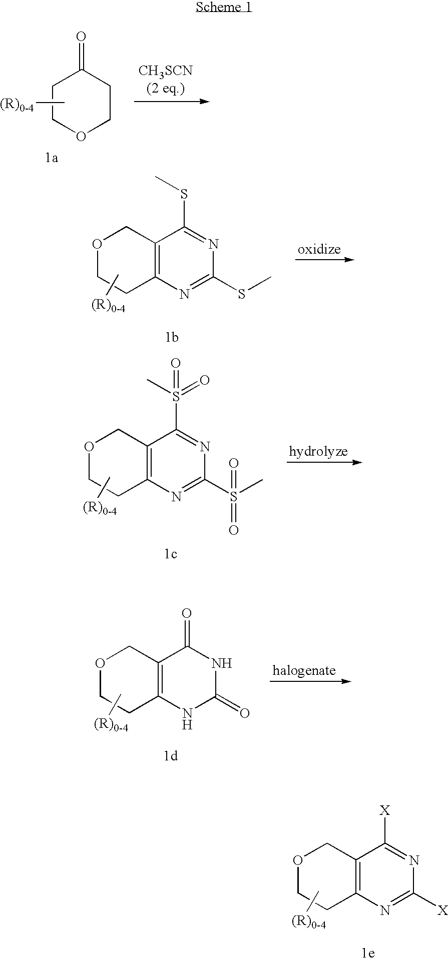

It is understood that modifications of starting materials shown in Scheme 1 can be done with no additional or only routine experimentation to form other compounds of the invention. For example, the synthetic route shown in Scheme 1 can be performed using related compounds (e.g., 5-, 6-, 7- and 8-membered oxo-containing heterocyclic rings other than 1a, such as for example optionally substituted, dihydro-2H-pyran-3(4H)-one, tetrahydro-2H-pyran-2-one, dihydrofuran-2(3H)-one, oxepan-4-one, among others. Also, as discussed above, the intermediate compound 1e can be further transformed into compounds of Formula I, using methods as described in Scheme 4 below.

Scheme 2 illustrates another general method for the synthesis of oxo-ring fused pyrimidine compounds of Formula I beginning with a ketoester starting material such as compound 2a. Condensation of ketoester 2a with an aryl or heteroaryl amidine 2b in the presence of a base (e.g., sodium ethoxide), followed by chlorination of the resultant pyrimidinone product (using for example P(O)Cl₃ or oxalyl chloride) can provide chloro compound 2c. Amidines, such as as 2b can be prepared as described by Ishida, J. et al. Bioorg. Med. Chem. Lett. 15 (2005) 4221-4225. Displacement of the chloro group in 2c with an amino group will provide oxo-ring fused pyrimidine compound 2d.

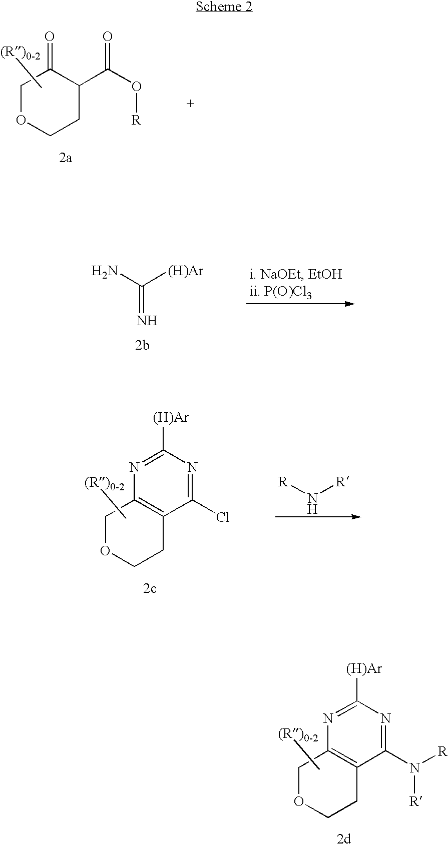

It is understood that the synthetic procedure outlined in Scheme 2 is not only applicable to the synthesis of oxo fused pyrimidine compounds using ketoester 2a as starting material, but is also applicable to other ketoesters starting materials including, without limitation, methyl 2-oxo-1,4-oxathiane-3-carboxylate, methyl 2-oxomorpholine-3-carboxylate, methyl 3-oxo-1,4-oxathiane-2-carboxylate, methyl 2-oxotetrahydro-2H-pyran-3-carboxylate, methyl 3-oxotetrahydro-2H-pyran-4-carboxylate and methyl 3-oxotetrahydro-2H-pyran-2-carboxylate, among others.

Bicyclic (and also monocyclic) oxo fused pyrimidines useful for the preparation of compounds of Formula I can be prepared as illustrated below in Scheme 3. For example, an optionally substituted 8-oxabicyclo[3.2.1]octan-2-one (3a) is treated with a benzylamine under acidic conditions to form the enamine derivative of 3a, which was then acylated with an activated ester of para-nitro-phenylcarboxylic acid to produce tertiary amide 3b. Lewis acid promoted cyclization of 3b in the presence of morpholinecarbonitrile can provide pyrimidine compound 3c. This intermediate can be further elaborated into compounds of the invention according to the synthetic scheme outlined in Scheme 5.

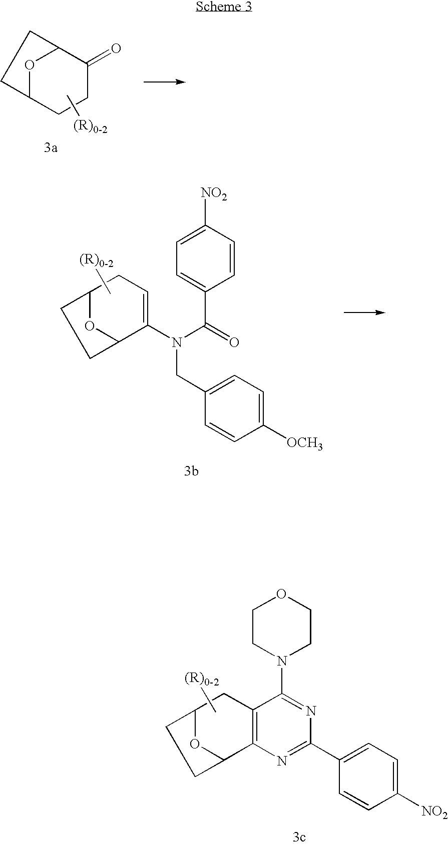

Scheme 4 illustrates the synthesis of compounds of the invention in which a halogenated oxo-ring fused pyrimidine 4a (e.g., 1e) is combined with an amine to provide amino compound 4b. Subsequent, Suzuki-cross coupling procedure can be used to affect the coupling of halo pyrimidine 4b to an aryl or heteroaryl (H)Ar boronate ester/boronic acid to produce 2-aryl substituted pyrimidine derivatives 4c. For a review of Suzuki coupling procedures see, Buchwald, S. J. et al. J. AM. CHEM SOC. 2005, 127, 4685-4696.

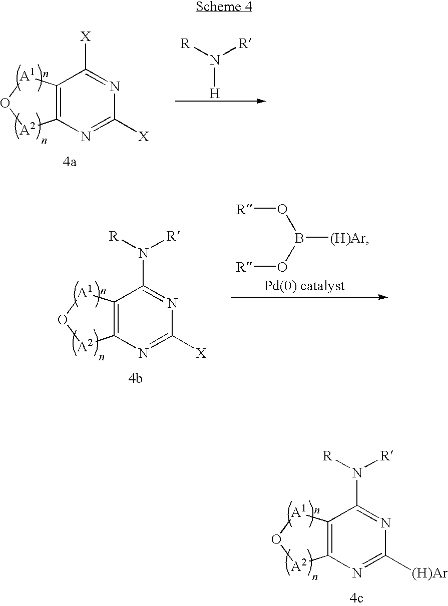

Scheme 5 illustrates several methods to derivatize the (H)Ar group located off the 2-position of the oxo ring-fused pyrimidine. As shown herein, when the (H)Ar group off of the 2-position of the pyrimidine ring is a para-nitro-phenyl group (see, compound 5a), then hydrogenation of a nitro group in 5a will provide a free primary amine derivative 5b. Compound 5b can then react with various electrophiles, e.g., sulfonyl chloride, isocyanates, acyl halides, respectively, to provide the corresponding, sulfonamide 5b1, urea 5b2, and amide 5b3.

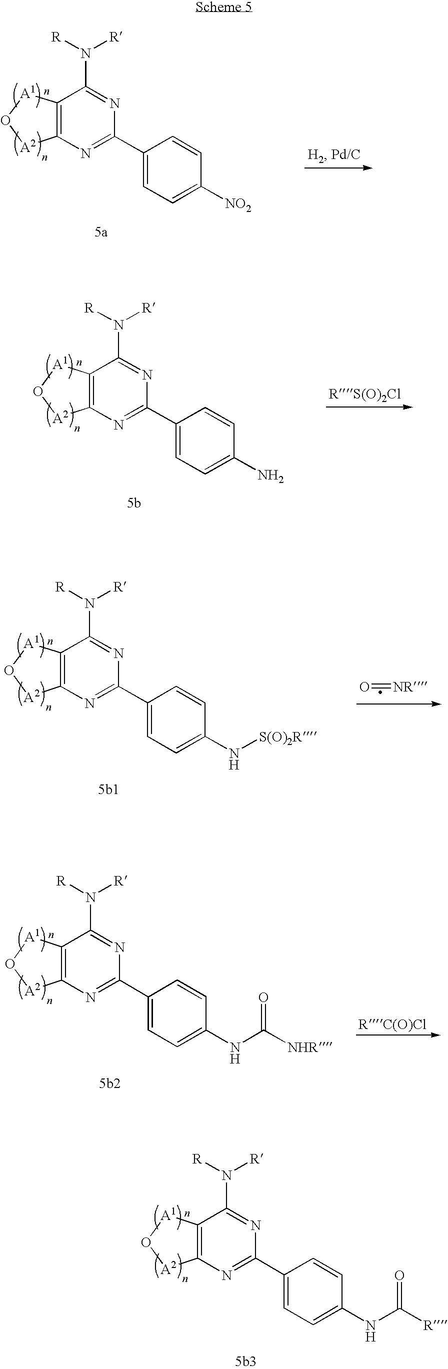

As illustrated in Scheme 6 below, oxo-ring fused pyrimidines such as 6a can be oxidized at a benzylic carbon under conditions described by Dohi, T. et al. J. Org. Chem., 2008, 73 (18) 7365-7368, using an mild oxidant such as iodosobenzene to provide the keto compound 6b.

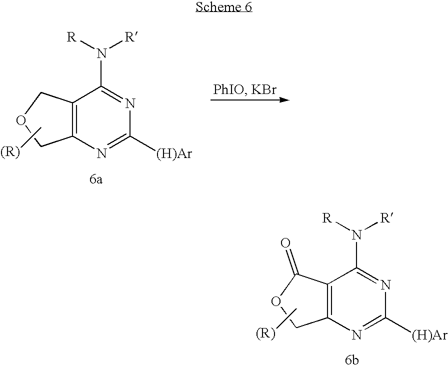

As illustrated below in Scheme 7, monothiomaleic anhydride fused pyrimidines 7b can be prepared as described in the Journal of Heterocyclic Chemistry, 14(4), 695-6; 1977

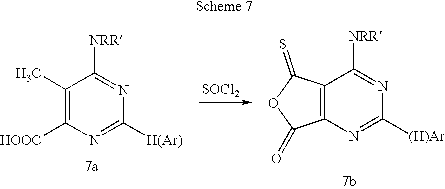

### III Pharmaceutical Compositions

In addition to one or more of the compounds provided above (or stereoisomers, geometric isomers, tautomers, solvates, metabolites or pharmaceutically acceptable salts, or prodrugs thereof), compositions for modulating mTOR activity in humans and animals will typically contain a pharmaceutically acceptable carrier, diluent or excipient.

The term “composition,” as used herein, is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

In order to use a compound of this invention for the therapeutic treatment (including prophylactic treatment) of mammals including humans, it is normally formulated in accordance with standard pharmaceutical practice as a pharmaceutical composition. According to this aspect of the invention there is provided a pharmaceutical composition comprising a compound of this invention in association with a pharmaceutically acceptable diluent, carrier or excipient.

A typical formulation is prepared by mixing a compound of the present invention and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water and the like. The particular carrier, diluent or excipient used will depend upon the means and purpose for which a compound of the present invention is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof. The formulations can also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present invention or pharmaceutical composition thereof) or aid in the manufacturing of the pharmaceutical product (i.e., medicament).

The formulations can be prepared using conventional dissolution and mixing procedures. For example, the bulk drug substance (i.e., compound of the present invention or stabilized form of the compound (e.g., complex with a cyclodextrin derivative or other known complexation agent) is dissolved in a suitable solvent in the presence of one or more of the excipients described above. A compound of the present invention is typically formulated into pharmaceutical dosage forms to provide an easily controllable dosage of the drug and to enable patient compliance with the prescribed regimen.

The pharmaceutical composition (or formulation) for application can be packaged in a variety of ways depending upon the method used for administering the drug. Generally, an article for distribution includes a container having deposited therein the pharmaceutical formulation in an appropriate form. Suitable containers are well known to those skilled in the art and include materials such as bottles (plastic and glass), sachets, ampoules, plastic bags, metal cylinders, and the like. The container can also include a tamper-proof assemblage to prevent indiscreet access to the contents of the package. In addition, the container has deposited thereon a label that describes the contents of the container. The label can also include appropriate warnings.

Pharmaceutical formulations of a compound of the present invention can be prepared for various routes and types of administration. For example, a compound of the invention (e.g., a compound of Formula I or II-A) having the desired degree of purity can optionally be mixed with pharmaceutically acceptable diluents, carriers, excipients or stabilizers (see, Remington: The Science and Practice of Pharmacy: Remington the Science and Practice of Pharmacy (2005) 21^{st} Edition, Lippincott Williams & Wilkins, Philidelphia, Pa.), in the form of a lyophilized formulation, milled powder, or an aqueous solution. Formulation can be conducted by mixing at ambient temperature at the appropriate pH, and at the desired degree of purity, with physiologically acceptable carriers, i.e., carriers that are non-toxic to recipients at the dosages and concentrations employed. The pH of the formulation depends mainly on the particular use and the concentration of compound, but can range from about 3 to about 8. Formulation in an acetate buffer at pH 5 is a suitable embodiment.

A compound of this invention (e.g., compound of Formula I or II-A) for use herein is preferably sterile. In particular, formulations to be used for in vivo administration must be sterile. Such sterilization is readily accomplished by filtration through sterile filtration membranes.

A compound of the invention ordinarily can be stored as a solid composition, a lyophilized formulation or as an aqueous solution.

A pharmaceutical composition of the invention will be formulated, dosed and administered in a fashion, i.e., amounts, concentrations, schedules, course, vehicles and route of administration, consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the compound to be administered will be governed by such considerations, and is the minimum amount necessary to prevent, ameliorate, or treat the coagulation factor mediated disorder. Such amount is preferably below the amount that is toxic to the host or renders the host significantly more susceptible to bleeding.

As a general proposition, the initial pharmaceutically effective amount of an inhibitor compound of the invention administered parenterally per dose will be in the range of about 0.01-100 mg/kg, namely about 0.1 to 20 mg/kg of patient body weight per day, with the typical initial range of compound used being 0.3 to 15 mg/kg/day.

Acceptable diluents, carriers, excipients and stabilizers are nontoxic to recipients at the dosages and concentrations employed, and include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). A active pharmaceutical ingredient of the invention (e.g., compound of Formula I or II-A) can also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington: The Science and Practice of Pharmacy: Remington the Science and Practice of Pharmacy (2005) 21^{st} Edition, Lippincott Williams & Wilkins, Philidelphia, Pa.

Sustained-release preparations of a compound of the invention (e.g., compound of Formula I or II-A) can be prepared. Suitable examples of sustained-release preparations include semipermeable matrices of solid hydrophobic polymers containing a compound of Formula I, which matrices are in the form of shaped articles, e.g., films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate) and poly-D-(−)-3-hydroxybutyric acid.

The formulations include those suitable for the administration routes detailed herein. The formulations can conveniently be presented in unit dosage form and can be prepared by any of the methods well known in the art of pharmacy. Techniques and formulations generally are found in Remington: The Science and Practice of Pharmacy: Remington the Science and Practice of Pharmacy (2005) 21^{st} Edition, Lippincott Williams & Wilkins, Philidelphia, P A. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of a compound of the invention (e.g., compound of Formula I or II-A) suitable for oral administration can be prepared as discrete units such as pills, capsules, cachets or tablets each containing a predetermined amount of a compound of the invention.

Compressed tablets can be prepared by compressing in a suitable machine the active ingredient in a free-flowing form such as a powder or granules, optionally mixed with a binder, lubricant, inert diluent, preservative, surface active or dispersing agent. Molded tablets can be made by molding in a suitable machine a mixture of the powdered active ingredient moistened with an inert liquid diluent. The tablets can optionally be coated or scored and optionally are formulated so as to provide slow or controlled release of the active ingredient therefrom.

Tablets, troches, lozenges, aqueous or oil suspensions, dispersible powders or granules, emulsions, hard or soft capsules, e.g., gelatin capsules, syrups or elixirs can be prepared for oral use. Formulations of a compound of the invention (e.g., compound of Formula I or II-A) intended for oral use can be prepared according to any method known to the art for the manufacture of pharmaceutical compositions and such compositions can contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients can be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets can be uncoated or can be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax can be employed.

For treatment of the eye or other external tissues, e.g., mouth and skin, the formulations are preferably applied as a topical ointment or cream containing the active ingredient(s) in an amount of, for example, 0.075 to 20% w/w. When formulated in an ointment, the active ingredient can be employed with either a paraffinic or a water-miscible ointment base. Alternatively, the active ingredients can be formulated in a cream with an oil-in-water cream base.

If desired, the aqueous phase of the cream base can include a polyhydric alcohol, i.e., an alcohol having two or more hydroxyl groups such as propylene glycol, butane 1,3-diol, mannitol, sorbitol, glycerol and polyethylene glycol (including PEG 400) and mixtures thereof. The topical formulations can desirably include a compound which enhances absorption or penetration of the active ingredient through the skin or other affected areas. Examples of such dermal penetration enhancers include dimethyl sulfoxide and related analogs.

The oily phase of the emulsions of this invention can be constituted from known ingredients in a known manner. While the phase can comprise merely an emulsifier, it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations. Emulsifiers and emulsion stabilizers suitable for use in the formulation of the invention include Tween® 60, Span® 80, cetostearyl alcohol, benzyl alcohol, myristyl alcohol, glyceryl mono-stearate and sodium lauryl sulfate.

Aqueous suspensions of a compound of the invention (e.g., compound of Formula I or II-A) contain the active materials in admixture with excipients suitable for the manufacture of aqueous suspensions. Such excipients include a suspending agent, such as sodium carboxymethylcellulose, croscarmellose, povidone, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension can also contain one or more preservatives such as ethyl or n-propyl p-hydroxybenzoate, one or more coloring agents, one or more flavoring agents and one or more sweetening agents, such as sucrose or saccharin.

A pharmaceutical composition of a compound of the invention (e.g., compound of Formula I or II-A) can be in the form of a sterile injectable preparation, such as a sterile injectable aqueous or oleaginous suspension. This suspension can be formulated according to the known art using those suitable dispersing or wetting agents and suspending agents which have been mentioned above. The sterile injectable preparation can also be a sterile injectable solution or suspension in a non-toxic parenterally acceptable diluent or solvent, such as a solution in 1,3-butanediol or prepared as a lyophilized powder. Among the acceptable vehicles and solvents that can be employed are water, Ringer's solution and isotonic sodium chloride solution. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables.

The amount of active ingredient that can be combined with the carrier material to produce a single dosage form will vary depending upon the host treated and the particular mode of administration. For example, a time-release formulation intended for oral administration to humans can contain approximately 1 to 1000 mg of active material compounded with an appropriate and convenient amount of carrier material which can vary from about 5 to about 95% of the total compositions (weight:weight). The pharmaceutical composition can be prepared to provide easily measurable amounts for administration. For example, an aqueous solution intended for intravenous infusion can contain from about 3 to 500 μg of the active ingredient per milliliter of solution in order that infusion of a suitable volume at a rate of about 30 mL/hr can occur.

Formulations suitable for parenteral administration include aqueous and non-aqueous sterile injection solutions which can contain anti-oxidants, buffers, bacteriostats and solutes which render the formulation isotonic with the blood of the intended recipient; and aqueous and non-aqueous sterile suspensions which can include suspending agents and thickening agents.

Formulations suitable for topical administration to the eye also include eye drops wherein the active ingredient is dissolved or suspended in a suitable carrier, especially an aqueous solvent for the active ingredient. The active ingredient is preferably present in such formulations in a concentration of about 0.5 to 20% w/w, for example about 0.5 to 10% w/w, for example about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration can be presented as a suppository with a suitable base comprising for example cocoa butter or a salicylate.

Formulations suitable for intrapulmonary or nasal administration have a particle size for example in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration can be prepared according to conventional methods and can be delivered with other therapeutic agents such as compounds heretofore used in the treatment or prophylaxis disorders as described below.

Formulations suitable for vaginal administration can be presented as pessaries, tampons, creams, gels, pastes, foams or spray formulations containing in addition to the active ingredient such carriers as are known in the art to be appropriate.

The formulations can be packaged in unit-dose or multi-dose containers, for example sealed ampoules and vials, and can be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

The invention further provides veterinary compositions comprising at least one active ingredient (e.g., compound of Formula I or II-A) as above defined together with a veterinary carrier therefore. Veterinary carriers are materials useful for the purpose of administering the composition and can be solid, liquid or gaseous materials which are otherwise inert or acceptable in the veterinary art and are compatible with the active ingredient. These veterinary compositions can be administered parenterally, orally or by any other desired route.

### IV Methods of Use

In another aspect, the present invention provides for a compound of the invention (e.g., compound of Formula I or II-A), or a stereoisomer, geometric isomer, tautomer, solvate, metabolite, or pharmaceutically acceptable salt, prodrug thereof that inhibits the activity of mTOR kinase. In one embodiment, a compound of the invention (e.g., compound of Formula I or II-A), or a stereoisomer, geometric isomer, tautomer, solvate, metabolite, or pharmaceutically acceptable salt, prodrug thereof inhibits the activity of mTORC1 and mTORC2. In another embodiment, a compound of the invention (e.g., compound of Formula I or II-A), or a stereoisomer, geometric isomer, tautomer, solvate, metabolite, or pharmaceutically acceptable salt, prodrug thereof, inhibits the activity of mTORC1. In another embodiment, a compound of the invention (e.g., compound of Formula I or II-A), or a stereoisomer, geometric isomer, tautomer, solvate, metabolite, or pharmaceutically acceptable salt, prodrug thereof, inhibits the activity of mTORC2. In certain embodiments, a compound of Formula I is 1×, 2×, 3×, 4×, 5×, 6×, 7×, 8×, 9×, 10×, 11×, 12×, 13×, 14×, 15×, 16×, 17×, 18×, 19×, 20×, 25×, 30×, 40×, 50×, 60×, 70×, 80×, 90×, 100×, 200×, 300×, 400×, 500×, 600×, 700×, 800×, 900×, 1000× more selective at inhibiting the actively of mTORC1 over mTORC2. In certain other embodiment, a compound of Formula I is 1×, 2×, 3×, 4×, 5×, 6×, 7×, 8×, 9×, 10×, 11×, 12×, 13×, 14×, 15×, 16×, 17×, 18×, 19×, 20×, 25×, 30×, 40×, 50×, 60×, 70×, 80×, 90×, 100×, 200×, 300×, 400×, 500×, 600×, 700×, 800×, 900×, 1000× more selective at inhibiting the actively of mTORC2 over mTORC1. In each of the above embodiment, in one particular aspect, a compound of the invention (e.g., compound of Formula I or II-A), or stereoisomer, geometric isomer, tautomer, solvate, metabolite, or pharmaceutically acceptable salt, or prodrug thereof, is formulated as a pharmaceutical composition.

The present invention further provides for a method of inhibiting the activity of mTOR kinase in a cell, comprising contacting said cell with an effective amount of an active compound of the invention (e.g., compound of Formula I or II-A), or a stereoisomer, geometric isomer, tautomer, solvate, metabolite, or pharmaceutically acceptable salt or prodrug thereof. The present invention further provides for a method of inhibiting cell proliferation comprising contacting the cell with a compound of Formula I or a subgenus thereof. Such methods can be practiced in vitro or in vivo.

A compound of the present invention, or stereoisomer, geometric isomer, tautomer, solvate, metabolite, or pharmaceutically acceptable salt, prodrug thereof, is useful for treating diseases, conditions and/or disorders including, but not limited to, those characterized by over expression of PIKK kinases, e.g. mTOR kinase. Accordingly, another aspect of this invention includes methods of treating diseases or conditions that can be treated by inhibiting mTOR kinase and use of a compound of Formula I (or an embodiment thereof) for the treatment of diseases or disorders caused by dysregulated mTOR activity. In one embodiment, the method comprises administering to a mammal in need thereof a therapeutically effective amount of a compound of the invention (e.g., compound of Formula I or II-A), or a stereoisomer, geometric isomer, tautomer, solvate, metabolite, or pharmaceutically acceptable salt or prodrug thereof. Within the above embodiment, in one particular aspect, a compound of the invention (e.g., compound of Formula I or II-A), or stereoisomer, geometric isomer, tautomer, solvate, metabolite, or pharmaceutically acceptable salt, prodrug thereof, is formulated as a pharmaceutical composition.

The compounds of the invention can be administered by any route appropriate to the condition to be treated. Suitable routes include oral, parenteral (including subcutaneous, intramuscular, intravenous, intraarterial, intradermal, intrathecal and epidural), transdermal, rectal, nasal, topical (including buccal and sublingual), vaginal, intraperitoneal, intrapulmonary and intranasal. For local immunosuppressive treatment, the compounds can be administered by intralesional administration, including perfusing or otherwise contacting the graft with the inhibitor before transplantation. It will be appreciated that the preferred route can vary with for example the condition of the recipient. Where the compound is administered orally, it can be formulated as a pill, capsule, tablet, etc. with a pharmaceutically acceptable carrier or excipient. Where the compound is administered parenterally, it can be formulated with a pharmaceutically acceptable parenteral vehicle and in a unit dosage injectable form, as detailed below.

A dose to treat mammal (e.g., human) can range from about 10 mg to about 1000 mg of a Formula I compound. A typical dose can be about 100 mg to about 300 mg of the compound. A dose can be administered once a day (QID), twice per day (BID), or more frequently, depending on the pharmacokinetic and pharmacodynamic properties, including absorption, distribution, metabolism, and excretion of the particular compound. In addition, toxicity factors can influence the dosage and administration regimen. When administered orally, the pill, capsule, or tablet can be ingested daily or less frequently for a specified period of time. The regimen can be repeated for a number of cycles of therapy.

Diseases and conditions treatable according to the methods of this invention include, but are not limited to, cancer, stroke, diabetes, hepatomegaly, cardiovascular disease, Alzheimer's disease, cystic fibrosis, viral disease, autoimmune diseases, atherosclerosis, restenosis, psoriasis, allergic disorders, inflammation, neurological disorders, a hormone-related disease, conditions associated with organ transplantation, immunodeficiency disorders, destructive bone disorders, proliferative disorders, infectious diseases, conditions associated with cell death, thrombin-induced platelet aggregation, chronic myelogenous leukemia (CML), liver disease, Peutz-Jegher syndrome, Tuberous Sclerosis, pathologic immune conditions involving T cell activation, and CNS disorders in a patient. In one embodiment, a human patient is treated with a compound of a compound of the invention (e.g., compound of Formula I or II-A) and a pharmaceutically acceptable carrier, adjuvant, or vehicle, wherein a compound of the invention is present in an amount to detectably inhibit mTOR kinase activity.

Cancers which can be treated according to the methods of this invention include, but are not limited to, breast, ovary, cervix, prostate, testis, genitourinary tract, esophagus, larynx, glioblastoma, neuroblastoma, stomach, skin, keratoacanthoma, lung, epidermoid carcinoma, large cell carcinoma, non-small cell lung carcinoma (NSCLC), small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, hairy cells, buccal cavity and pharynx (oral), lip, tongue, mouth, pharynx, small intestine, colon-rectum, large intestine, rectum, brain and central nervous system, Hodgkin's and leukemia. In cetain embodiment, compounds of the invention are useful for the treatment of cancer selected from the group consisting of breast, NSCLC, small cell carcinoma, liver carcinoma, lymphoid disorders, sarcoma, colon-rectum, rectum and leukemia.

Cardiovascular diseases which can be treated according to the methods of this invention include, but are not limited to, restenosis, cardiomegaly, atherosclerosis, myocardial infarction, and congestive heart failure.

Neurodegenerative disease which can be treated according to the methods of this invention include, but are not limited to, Alzheimer's disease, Parkinson's disease, amyotrophic lateral sclerosis, Huntington's disease, and cerebral ischemia, and neurodegenerative disease caused by traumatic injury, glutamate neurotoxicity and hypoxia.

Inflammatory diseases which can be treated according to the methods of this invention include, but are not limited to, rheumatoid arthritis, psoriasis, contact dermatitis, and delayed hypersensitivity reactions.

Another aspect of this invention provides a compound of the invention, or stereoisomer, geometric isomer, tautomer, solvate, metabolite, or pharmaceutically acceptable salt, or prodrug thereof, in the treatment of the diseases or conditions described herein in a mammal, for example, a human, suffering from such disease or condition. Also provided is the use of a compound of this invention, or stereoisomer, geometric isomer, tautomer, solvate, metabolite, or pharmaceutically acceptable salt, or prodrug thereof, in the preparation of a medicament for the treatment of the diseases and conditions described herein in a mammal, for example a human, suffering from such disorder.

In one embodiment, a compound of the invention (e.g., compound of Formula I or II-A), or stereoisomer, geometric isomer, tautomer, solvate, metabolite, or pharmaceutically acceptable salt, prodrug thereof, is used as an anticancer agent or as an adjunct agent for the treatment of cancer in a combination therapy. One of ordinary skill in the art is readily able to determine whether or not a candidate compound treats a cancerous condition for any particular cell type, either alone or in combination. Within certain aspects of this embodiment, compounds of the invention are used in adjunct with other therapies, including conventional surgery, radiotherapy and chemotherapy, for the treatment of cancer. Such chemotherapy can include, but are not limited to one or more of the chemotherapeutic agents described herein.

The combination therapy can be administered as a simultaneous or sequential regimen. When administered sequentially, the combination can be administered in two or more administrations. The combined administration includes coadministration, using separate formulations or a single pharmaceutical formulation, and consecutive administration in either order, wherein preferably there is a time period while both (or all) active agents simultaneously exert their biological activities.

Suitable dosages for any of the above coadministered agents are those presently used and can be lowered due to the combined action (synergy) of the newly identified agent and other chemotherapeutic agents or treatments.

The combination therapy can provide “synergy” and prove “synergistic”, i.e., the effect achieved when the active ingredients used together is greater than the sum of the effects that results from using the compounds separately. A synergistic effect can be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect can be attained when the compounds are administered or delivered sequentially, e.g., by different injections in separate syringes, separate pills or capsules, or in separate infusions. In general, during alternation therapy, an effective dosage of each active ingredient is administered sequentially, i.e., serially, whereas in combination therapy, effective dosages of two or more active ingredients are administered together.

### V EXAMPLES

These examples are not intended to limit the scope of the present invention, but rather to provide guidance to a skilled artisan to prepare and use the compounds, compositions, and methods of the present invention. While particular embodiments of the present invention are described, the skilled artisan will appreciate that various changes and modifications can be made without departing from the spirit and scope of the invention.

The chemical reactions in the Examples described can be readily adapted to prepare a number of other mTOR inhibitors of the invention, and alternative methods for preparing the compounds of this invention are deemed to be within the scope of this invention. For example, the synthesis of non-exemplified compounds according to the invention can be successfully performed by modifications apparent to those skilled in the art, e.g., by appropriately protecting interferring groups, by utilizing other suitable reagents known in the art other than those described, and/or by making routine modifications of reaction conditions. Alternatively, other reactions disclosed herein or known in the art will be recognized as having applicability for preparing other compounds of the invention. Accordingly, the following examples are provided to illustrate but not limit the invention.

In the Examples described below, unless otherwise indicated all temperatures are set forth in degrees Celsius. Commercially available reagents were purchased from suppliers such as Aldrich Chemical Company, Lancaster, TCI or Maybridge, and were used without further purification unless otherwise indicated. The reactions set forth below were done generally under a positive pressure of nitrogen or argon or with a drying tube (unless otherwise stated) in anhydrous solvents, and the reaction flasks were typically fitted with rubber septa for the introduction of substrates and reagents via syringe. Glassware was oven dried and/or heat dried. Column chromatography was conducted on a Biotage system (Manufacturer: Dyax Corporation) having a silica gel column or on a silica SEP PAK® cartridge (Waters); or alternatively column chromatography was carried out using on an ISCO chromatography system (Manufacturer: Teledyne ISCO) having a silica gel column. ¹H NMR spectra were recorded on a Varian instrument operating at 400 MHz. ¹H NMR spectra were obtained in deuterated CDCl₃, d₆-DMSO, CH₃OD or d₆-acetone solutions (reported in ppm), using chloroform as the reference standard (7.25 ppm). When peak multiplicities are reported, the following abbreviations are used: s (singlet), d (doublet), t (triplet), m (multiplet), br (broadened), dd (doublet of doublets), dt (doublet of triplets). Coupling constants, when given, are reported in Hertz (Hz). When possible, product formation in the reaction mixtures were monitored by LC/MS was performed either on an Agilent 1200 Series LC coupled to a 6140 quadrupole mass spectrometer using a Supelco Ascentis Express C18 column with a linear gradient of 5%-95% acetonitrile/water (with 0.1% trifluoroacetic acid in each mobile phase) within 1.4 minutes and held at 95% for 0.3 minute, or on a PE Sciex API 150 EX using a Phenomenex DNYC monolithic C18 column with a linear gradient of 5%-95% acetonitrile/water (with 0.1% trifluoroacetic acid in each mobile phase) within 5 minutes and held at 95% for 1 minute. All abbreviations used to described reagents, reaction conditions, or equipment used are consistent with the definitions set forth in the “List of standard abbreviations and acronyms” published yearly by the Journal of Organic Chemistry (an American Chemical Society journal). The chemical names of discrete compounds of the invention were obtained using the structure naming feature ChemBioDraw Version 11.0 or from Accelrys' Pipeline Pilot IUPAC compound naming program.

### Example 1

### Preparation of 1-ethyl-3-(4-(4-morpholino-7,8-dihydro-6H-pyrano[3,2-d]pyrimidin-2-yl)phenyl)urea (f)

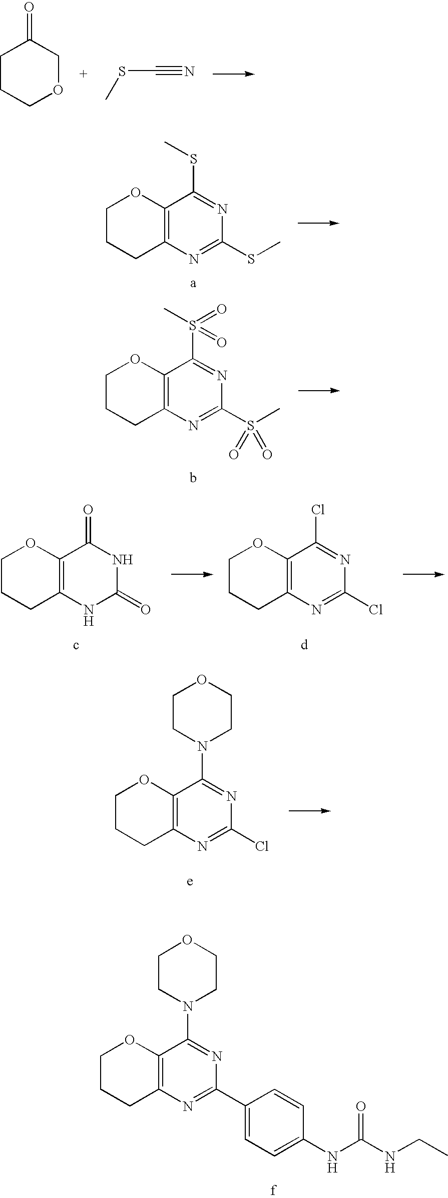

Step 1—Synthesis of a: To a mixture of dihydro-2H-pyran-3(4H)-one (9.2 mL, 99.8 mmol) and methylthiocyanide (32 mL, 401.0 mmol) in nitromethane (75 mL) at −40° C. was added trifluoromethane sulfonic anhydride (25 mL, 148.3 mmol). The mixture was stirred at −40° C. for 6 h then at room temperature overnight. The reaction was quenched by slow addition of saturated aqueous sodium bicarbonate. The layers were separated and the aqueous phase was extratec with 2×20 mL of dichloromethane. The combined organic phases were dried with MgSO₄, filtered and concentrated. The crude material was purified by flash column chromatography (100% Hex to 80% EtOAc/Hex) to give 2,4-bis(methylthio)-7,8-dihydro-6H-pyrano[3,2-d]pyrimidine (a) (1.7 g, 7%): LC-MS: m/z=229 (M+H): ¹H NMR (400 MHz, CDCl₃) δ 4.30-4.19 (m, 2H), 2.79 (t, J=6.6, 2H), 2.56 (s, 3H), 2.54 (s, 3H), 2.16-1.98 (m, 2H).

Step 2—Synthesis of b: To a solution of 2,4-bis(methylthio)-7,8-dihydro-6H-pyrano[3,2-d]pyrimidine (1.7 g, 7.3 mmol) in dichloromethane (30 mL) was added m-chloroperoxybenzoic acid (10.0 g, 44.6 mmol) at room temperature, over a period of 2 h. The mixture was stirred at room temperature overnight. The reaction was then cooled to 0° C. and quenched by slow addition of 10% aqueous Na₂S₂O₃. The phases were shaken and separated. The aqueous phase was extracted with 2×100 mL of dichloromethane. The combined organic phases were washed with 2×75 mL of saturated aqueous NaHCO₃ and concentrated to give 2,4-bis(methylsulfonyl)-7,8-dihydro-6H-pyrano[3,2-d]pyrimidine (b) (1.5 g, 69%): LC-MS: m/z=293 (M+H).

Step 3—Synthesis of c: 2,4-bis(methylsulfonyl)-7,8-dihydro-6H-pyrano[3,2-d]pyrimidine (1.5 g, 5.1 mmol) was suspended in 3.7 M sodium hydroxide (30 mL) and the mixture was stirred at 100° C. After 15 min, the suspension turned into a clear solution. Heating was continued for 4 h. Then, the mixture was cooled to 5° C. and acidified by addition of concentrated aqueous HCl. The solid that crashed was collected by filtration and washed with cold water to give 7,8-dihydro-1H-pyrano[3,2-d]pyrimidine-2,4(3H,6H)-dione (c) (900 mg, 100%): LC-MS: m/z=169 (M+H).

Step 4—Synthesis of d: 7,8-dihydro-1H-pyrano[3,2-d]pyrimidine-2,4(3H,6H)-dione (900 mg, 5.4 mmol) was suspended in phosphoryl chloride (10 mL, 107.6 mmol) and the reaction was stirred at 100° C. overnight. The mixture was then cooled down and neutralized by addition of saturated aqueous NaHCO₃. The phases were separated and the aqueous phase was extracted with 2×20 mL of dichloromethane. The combined organic phases were dried with MgSO₄, filtered and concentrated. The crude product was purified by flash column chromatography (100% Hex to 60% EtOAc/Hex) to give 2,4-dichloro-7,8-dihydro-6H-pyrano[3,2-d]pyrimidine (d) (100 mg, 9.1%): LC-MS: m/z=206 (M+H): ¹H NMR (500 MHz, CDCl₃) δ 4.44-4.31 (m, 2H), 2.96 (t, J=6.6, 2H), 2.23-2.00 (m, 2H).

Step 5—Synthesis of e: To a solution of 2,4-dichloro-7,8-dihydro-6H-pyrano[3,2-d]pyrimidine (100 mg, 0.5 mmol) and diisopropylethylamine (0.25 mL, 1.5 mmol) in dimethylformamide (2.0 mL) was added morpholine (51 μL, 0.6 mmol) and the mixture was stirred at 50° C. for 1 h. Then the mixture was cooled to room temperature, water was added and the aqueous phase was extracted with 2×25 mL of dichloromethane. The combined organic phases were dried with MgSO₄, filtered and concentrated. The crude material was purified by flash column chromatography to give 2-chloro-4-morpholino-7,8-dihydro-6H-pyrano[3,2-d]pyrimidine (e) (90 mg, 72%): LC-MS: m/z=256 (M+H).

Step 6—Synthesis off: A microwave vial was charged with 2-chloro-4-morpholino-7,8-dihydro-6H-pyrano[3,2-d]pyrimidine (90 mg, 0.35 mmol), 4-(3-ethylureido)phenylboronic acid, pinacol ester (123 mg, 0.42 mmol), tetrakis(triphenylphosphine)palladium (41 mg, 0.03 mmol), potassium acetate (34 mg, 0.34 mmol) and sodium carbonate (35 mg, 0.3 mmol) in acetonitrile (2 mL) and water (1 mL). The mixture was heated at 110° C. for 20 min in the microwave. The crude product was purified by reverse phase HPLC to give 1-ethyl-3-(4-(4-morpholino-7,8-dihydro-6H-pyrano[3,2-d]pyrimidin-2-yl)phenyl)urea (f) (8.5 mg, 6.5%): LC-MS: m/z=384 (M+H): ¹H NMR (400 MHz, DMSO) δ 8.65 (s, 1H), 8.08 (d, J=8.7, 2H), 7.46 (d, J=8.6, 2H), 6.16 (s, 1H), 4.31-4.07 (m, 2H), 3.72 (s, 8H), 3.20-2.98 (m, 2H), 2.88-2.68 (m, 2H), 2.04 (dd, J=13.8, 8.6, 2H), 1.05 (t, J=7.2, 3H).

### Example 2

### Preparation of (S)-1-ethyl-3-(4-(4-(3-ethylmorpholino)-6,7-dihydro-5H-pyrano[2,3-d]pyrimidin-2-yl)phenyl)urea (g)

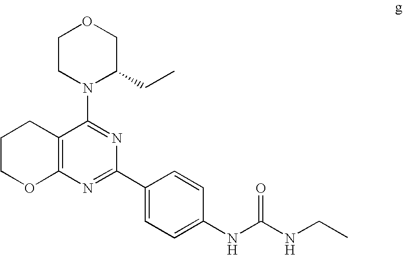

(S)-1-ethyl-3-(4-(4-(3-ethylmorpholino)-6,7-dihydro-5H-pyrano[2,3-d]pyrimidin-2-yl)phenyl)urea (g) was prepared in a similar manner as described for Example 1 with the exceptions that tetrahydro-2H-pyran-2-one was used in Step 1 instead of dihydro-2H-pyran-3(4H)-one and (S)-3-ethylmorpholine was used in Step 5 instead of morpholine. LC-MS: m/z=412 (M+H). ¹H NMR (500 MHz, DMSO) δ 8.71 (s, 1H), 8.10 (d, J=8.7, 2H), 7.45 (d, J=8.8, 2H), 6.24 (s, 1H), 4.34 (s, 1H), 4.24 (s, 1H), 3.85 (s, 2H), 3.77 (d, J=11.3, 1H), 3.67 (d, J=8.7, 1H), 3.57 (t, J=11.3, 2H), 3.41 (s, 1H), 3.18-3.05 (m, 2H), 2.64 (s, 2H), 1.93 (s, 1H), 1.77 (d, J=48.0, 3H), 1.05 (t, J=7.2, 3H), 0.84 (t, J=7.5, 3H).

### Example 3

### Preparation of 1-(4-(4-(2-oxa-5-azabicyclo[2.2.1]heptan-5-yl)-6,7-dihydro-5H-pyrano[2,3-d]pyrimidin-2-yl)phenyl)-3-ethylurea (h)

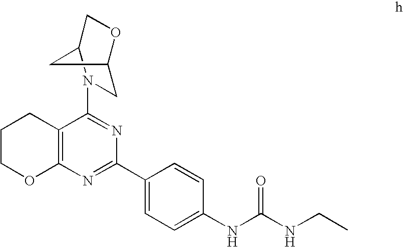

1-(4-(4-(2-oxa-5-azabicyclo[2.2.1]heptan-5-yl)-6,7-dihydro-5H-pyrano[2,3-d]pyrimidin-2-yl)phenyl)-3-ethylurea (h) was prepared in a similar manner as described for Example 1 with the exceptions that tetrahydro-2H-pyran-2-one was used in Step 1 instead of dihydro-2H-pyran-3(4H)-one and 2-oxa-5-azabicyclo[2.2.1]heptane was used in Step 5 instead of morpholine. LC-MS: m/z=396 (M+H). ¹H NMR (400 MHz, DMSO) δ 8.63 (s, 1H), 8.08 (d, J=8.8, 2H), 7.44 (d, J=8.8, 2H), 6.18 (t, J=5.5, 1H), 5.01 (s, 1H), 4.61 (s, 1H), 4.34 (d, J=11.0, 1H), 4.15 (t, J=9.4, 1H), 3.88 (dd, J=23.1, 7.3, 2H), 3.74 (d, J=9.6, 1H), 3.45 (d, J=9.7, 1H), 3.21-3.03 (m, 3H), 2.83-2.59 (m, 1H), 2.03-1.64 (m, 4H), 1.06 (t, J=7.2, 3H).

### Example 4

### Preparation of (S)-2-(4-(4-(3-ethylmorpholino)-7,8-dihydro-5H-pyrano[4,3-d]pyrimidin-2-yl)phenylamino)pyrimidin-4(3H)-one (i)

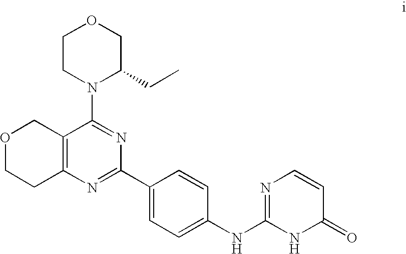

(S)-2-(4-(4-(3-ethylmorpholino)-7,8-dihydro-5H-pyrano[4,3-d]pyrimidin-2-yl)phenylamino)pyrimidin-4(3H)-one (i) was prepared in a similar manner as described for Example 1 with the exceptions that dihydro-2H-pyran-4(3H)-one was used in Step 1 instead of dihydro-2H-pyran-3(4H)-one, (S)-3-ethylmorpholine was used in Step 5 instead of morpholine, and 2-(4-(4,4,5,5-tetramethyl-1,3,2-dioxaborolan-2-yl)phenylamino)pyrimidin-4(3H)-one was used in Step 6 instead of 1-ethyl-3-(4-(4,4,5,5-tetramethyl-1,3,2-dioxaborolan-2-yl)phenyl)urea. LC-MS: m/z=435 (M+H). ¹H NMR (400 MHz, DMSO) δ 8.25 (d, J=8.6, 2H), 7.74 (s, 3H), 6.01-5.58 (m, 1H), 4.58 (q, J=14.3, 2H), 4.12-3.99 (m, 1H), 3.94 (d, J=7.6, 1H), 3.89-3.72 (m, 3H), 3.67 (d, J=8.7, 1H), 3.53 (d, J=11.1, 2H), 3.42 (d, J=11.2, 1H), 2.86 (d, J=8.6, 2H), 1.78 (dd, J=18.2, 7.3, 2H), 0.84 (t, J=7.4, 3H).

### Example 5

### Preparation of (S)-6-(4-(4-(3-ethylmorpholino)-7,8-dihydro-5H-pyrano[4,3-d]pyrimidin-2-yl)phenylamino)pyridin-2(1H)-one (j)

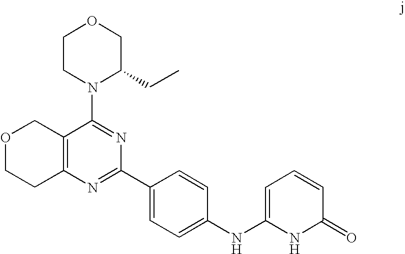

(S)-6-(4-(4-(3-ethylmorpholino)-7,8-dihydro-5H-pyrano[4,3-d]pyrimidin-2-yl)phenylamino)pyridin-2(1H)-one (j) was prepared in a similar manner as described for Example 1 with the exceptions that dihydro-2H-pyran-4(3H)-one was used in Step 1 instead of dihydro-2H-pyran-3(4H)-one, (S)-3-ethylmorpholine was used in Step 5 instead of morpholine, and 6-(4-(4,4,5,5-tetramethyl-1,3,2-dioxaborolan-2-yl)phenylamino)pyridin-2(1H)-one was used in Step 6 instead of 1-ethyl-3-(4-(4,4,5,5-tetramethyl-1,3,2-dioxaborolan-2-yl)phenyl)urea. LC-MS: m/z=434 (M+H). ¹H NMR (400 MHz, DMSO) δ 10.40-9.95 (m, 1H), 9.33-8.82 (m, 1H), 8.19 (d, J=8.7, 2H), 7.78 (s, 2H), 7.43 (s, 1H), 7.24-6.83 (m, 1H), 6.32 (s, 1H), 6.03 (s, 1H), 4.60 (d, J=7.3, 2H), 4.06 (d, J=5.1, 1H), 4.00-3.72 (m, 4H), 3.67 (d, J=8.9, 1H), 3.62-3.39 (m, 3H), 2.86 (s, 2H), 2.29 (s, 2H), 1.80 (d, J=7.6, 2H), 0.85 (t, J=7.4, 3H).

### Example 6

### Preparation of (S)-1-(4-(4-(3-ethylmorpholino)-7,8-dihydro-5H-pyrano[4,3-d]pyrimidin-2-yl)phenyl)-3-(oxetan-3-yl)urea (k)

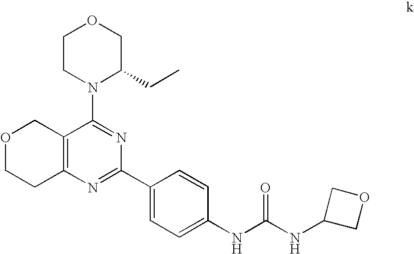

(S)-1-(4-(4-(3-ethylmorpholino)-7,8-dihydro-5H-pyrano[4,3-d]pyrimidin-2-yl)phenyl)-3-(oxetan-3-yl)urea (k) was prepared in a similar manner as described for Example 1 with the exceptions that dihydro-2H-pyran-4(3H)-one was used in Step 1 instead of dihydro-2H-pyran-3(4H)-one, (S)-3-ethylmorpholine was used in Step 5 instead of morpholine, and 1-(oxetan-3-yl)-3-(4-(4,4,5,5-tetramethyl-1,3,2-dioxaborolan-2-yl)phenyl)urea was used in Step 6 instead of 1-ethyl-3-(4-(4,4,5,5-tetramethyl-1,3,2-dioxaborolan-2-yl)phenyl)urea. LC-MS: m/z=440 (M+H). ¹H NMR (400 MHz, DMSO) δ 8.77 (s, 1H), 8.18 (d, J=8.7, 2H), 7.47 (d, J=8.7, 2H), 6.98 (d, J=6.5, 1H), 4.85-4.66 (m, 3H), 4.57 (q, J=14.2, 2H), 4.44 (t, J=5.8, 2H), 4.12-3.98 (m, 1H), 3.99-3.87 (m, 1H), 3.83 (d, J=11.0, 1H), 3.77 (d, J=10.4, 2H), 3.68 (t, J=10.5, 1H), 3.59-3.46 (m, 2H), 3.40 (t, J=11.5, 1H), 2.83 (dd, J=23.0, 12.6, 2H), 1.91-1.60 (m, 2H), 0.83 (t, J=7.4, 3H).

### Example 7

### Preparation of (S)-1-(4-(7,7-dimethyl-4-(3-methylmorpholino)-5,7-dihydrofuro[3,4-d]pyrimidin-2-yl)phenyl)-3-ethylurea (r)

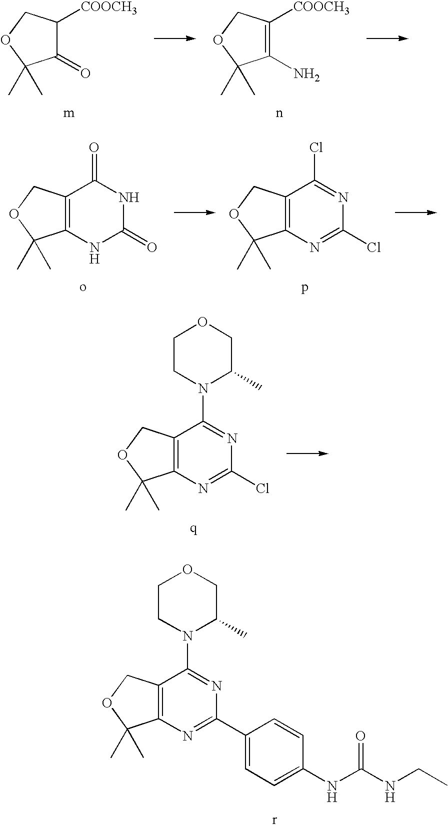

Step 1—Synthesis of n: A solution of methyl 5,5-dimethyl-4-oxotetrahydrofuran-3-carboxylate (m) [prepared according to Gianturco, Tetrahedron, 1964, 20, 1763-1772] (19.1 g, 111 mmol), ammonium acetate (89 g, 1150 mmol) and methanol (225 mL) was heated at reflux for 20 h. The solvent was removed under reduce pressure and the residue partitioned between saturated NaHCO₃ (500 mL) and ethyl acetate (150 mL). The phases were separated and the aq. phase was extracted with ethyl acetate (2×150 mL). The combined organic phases were washed with brine (1×50 mL), dried (Na₂SO₄), filtered, and concentrated onto Celite to afford a free-flowing powder. The residue was chromatographed: ISCO 330 g column, 5-30% ethyl acetate-heptane to afford 12.34 g (65%) of methyl 4-amino-5,5-dimethyl-2,5-dihydrofuran-3-carboxylate (n) as a colorless solid: ¹H NMR (400 MHz, CDCl₃) δ 5.34 (s, 2H), 4.66 (s, 2H), 3.71 (s, 3H), 1.45-1.21 (m, 6H); LC-MS: m/z=+172 (M+H)⁺.

Step 2—Synthesis of o: To a cool (0° C.) solution of methyl 4-amino-5,5-dimethyl-2,5-dihydrofuran-3-carboxylate (n)(12.34 g, 72.1 mmol), pyridine (23.3 mL, 288 mmol) and 1,2-dichloroethane (250 mL) was added phosgene (20% solution in toluene, 50 mL, 86.5 mmol) in one portion. The mixture was maintained at 0° C. for 3 h, then 28% NH₄OH (80 mL) was added in one portion and the mixture was stirred gently for 3 h, then heated at 50° C. for 16 h. Water (200 mL) was added and the phases separated. The organic phase was extracted with 1% NH₄OH (2×100 mL). The combined aq. phases were washed with dichloromethane (3×20 mL), and concentrated to approximately 150 mL, which caused the product to precipitate. The ppt. was collected on paper, rinsed with water, and dried under high vacuum to afford 8.27 g of 7,7-dimethyl-5,7-dihydrofuro[3,4-d]pyrimidine-2,4(1H,3H)-dione (o) as colorless crystals, further concentration of the mother liquor provided a second crop of product 1.07 g (71% combined yield): ¹H NMR (400 MHz, DMSO) δ 11.37 (s, 1H), 11.00 (m, 1H), 4.73 (s, 2H), 1.30 (s, 6H); LC-MS: m/z=+182 (M+H)⁺.

Step 3—Synthesis of p: A mixture of 7,7-dimethyl-5,7-dihydrofuro[3,4-d]pyrimidine-2,4(1H,3H)-dione (o) (2.55 g, 14.0 mmol), phosphoryl chloride (15 mL, 160 mmol) and 1,2-dichloroethane (80 mL) was heated at 80° C. for 20 h. The mixture was concentrated to a solid and partitioned between dichloromethane (250 mL) and saturated NaHCO₃ (500 mL). The phases were separated and the aq. phase was extracted with dichloromethane (3×50 mL). The combined org. phases were dried (Na₂SO₄), filtered and concentrated to afford 2.53 g (82%) of 2,4-dichloro-7,7-dimethyl-5,7-dihydrofuro[3,4-d]pyrimidine (p) as a colorless solid: ¹H NMR (400 MHz, CDCl₃) δ 5.02 (s, 2H), 1.51 (s, 6H); LC-MS: m/z=+219 (M+H)⁺.

Step 4—Synthesis of q: To a cool (0° C.) solution of 2,4-dichloro-7,7-dimethyl-5,7-dihydrofuro[3,4-d]pyrimidine (p) (2.53 g, 11.5 mmol), DIPEA (4.8 mL, 28 mmol) and DMF (15 mL) was added (3S)-3-methylmorpholine (1.42 g, 14 mmol), the solution was allowed to warm slowly over 15 h. The solution was poured into sat. NH₄Cl (100 mL) and extracted with ether (3×50 mL). The combined org. phases were washed with brine (1×25 mL), dried (MgSO₄), filtered, and concentrated to afford 3.18 g (95%) of (S)-2-chloro-7,7-dimethyl-4-(3-methylmorpholino)-5,7-dihydrofuro[3,4-d]pyrimidine (q) as a colorless solid: ¹H NMR (400 MHz, CDCl₃) δ 5.10 (d, J=11.3 Hz, 1H), 5.05 (d, J=11.3 Hz, 1H), 4.11 (s, 1H), 3.85-4.00 (m, 2H), 3.84-3.66 (m, 2H), 3.55 (ddd, J=11.9, 11.9, 2.8 Hz, 1H), 3.39 (ddd, J=13.0, 13.0, 3.2 Hz, 1H), 1.47 (s, 3H), 1.46 (s, 3H), 1.36 (d, J=6.8 Hz, 3H); LC-MS: m/z=+284 (M+H)⁺.

Step 5—Synthesis of r: A mixture of (S)-2-chloro-7,7-dimethyl-4-(3-methylmorpholino)-5,7-dihydrofuro[3,4-d]pyrimidine (q) (1.65 g, 5.66 mmol), [4-ethylureido)phenyl]boronic acid, picacol ester (2.87 g, 9.90 mmol), tetrakis(triphenylphosphine)palladium(0) (440 mg, 0.38 mmol), 1.0 M Na₂CO₃ (7.4 mL, 7.40 mmol), 1.0 M potassium acetate (7.4 mL, 7.40 mmol), and acetonitrile (15 mL) was heated at 110° C. in a microwave reactor for 30 min. The mixture was partitioned between saturated NH₄Cl (100 mL) and ethyl acetate (50 mL). The phases were separated and the aq. extracted with ethyl acetate (2×50 mL). The combined organic phases were dried (Na₂SO₄), filtered, adsorbed onto Celite and chromatographed ISCO 80 g column 0-75% ethyl acetate in heptane to afford 2.12 g of (S)-1-(4-(7,7-dimethyl-4-(3-methylmorpholino)-5,7-dihydrofuro[3,4-d]pyrimidin-2-yl)phenyl)-3-ethylurea (r) as a colorless solid with 90% purity. A portion of this material (0.50 g) was slurried in iPrOH (5 mL) at 50° C. for 1 h. The material was collected by filtration on paper, washing with iPrOH. Drying under vacuum afforded 325 mg of pure material: ¹H NMR (400 MHz, CDCl₃) δ 8.37 (d, J=8.6 Hz, 2H), 7.36 (d, J=8.6 Hz, 2H), 6.30 (s, 1H), 5.16 (d, J=11.3 Hz, 1H), 5.12 (d, J=11.3 Hz, 1H), 4.68 (s, 1H), 4.23 (s, 1H), 4.14-3.95 (m, 2H), 3.87-3.70 (m, 2H), 3.62 (ddd, J=12.0, 12.0, 2.8 Hz, 1H), 3.43 (ddd, J=12.9, 12.9, 3.6 Hz, 1H), 3.37-3.22 (m, 2H), 1.52 (s, 3H), 1.49 (s, 3H), 1.37 (d, J=6.8 Hz, 3H), 1.18 (t, J=7.3 Hz, 3H); LC-MS: m/z=+412 (M+H)⁺.

### Example 8

### Preparation of (S)-1-(4-(7,7-dimethyl-4-(3-methylmorpholino)-5,7-dihydrofuro[3,4-d]pyrimidin-2-yl)phenyl)-3-(oxetan-3-yl)urea (u)

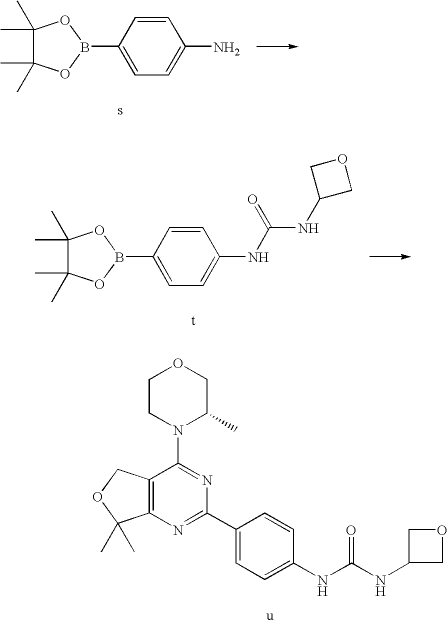

Step 1—Synthesis of t: A solution of 4-(4,4,5,5-tetramethyl-1,3,2-dioxaborolan-2-yl)aniline (s) (1.50 g, 6.85 mmol), pyridine (2.21 mL, 27.4 mmol) and dichloromethane (8 mL) was added dropwise to a cool (0° C.) solution of phosgene (20% in toluene, 4.32 mL, 8.22 mmol) and dichloromethane (15 mL). The solution was maintained at 0° C. for 1 h, then 3-oxetanamine.HCl (900 mg, 8.22 mmol), and DIPEA (8.0 mL, 6.7 mmol) were added and the mixture allowed to come to rt over 12 h. The mixture was partitioned between sat. NH₄Cl (75 mL) and ethyl acetate (50 mL). The phases were separated and the aq. extracted with ethyl acetate (2×20 mL). The combined organic phases were washed with brine (1×20 mL), dried (Na₂SO₄), filtered, adsorbed onto Celite and chromatographed ISCO 40 g column 0-75% ethyl acetate in dichloromethane to afford 1.23 g (56%) of 1-(oxetan-3-yl)-3-(4-(4,4,5,5-tetramethyl-1,3,2-dioxaborolan-2-yl)phenyl)urea (t) as a colorless solid: ¹H NMR (400 MHz, CDCl₃) δ 7.77 (d, J=8.3 Hz, 2H), 7.29 (d, J=8.2 Hz, 2H), 6.41 (s, 1H), 5.24 (s, 1H), 5.04-4.96 (m, 1H), 4.93 (t, J=7.0 Hz, 2H), 4.48 (t, J=6.3 Hz, 2H), 1.34 (s, 12H); LC-MS: m/z=+319 (M+H)⁺.

Step 2—Synthesis of u: A mixture of (S)-2-chloro-7,7-dimethyl-4-(3-methylmorpholino)-5,7-dihydrofuro[3,4-d]pyrimidine (q) (300 mg, 1.06 mmol), 1-(oxetan-3-yl)-3-(4-(4,4,5,5-tetramethyl-1,3,2-dioxaborolan-2-yl)phenyl)urea (470 mg, 1.48 mmol), tetrakis(triphenylphosphine)palladium(0) (97 mg, 0.084 mmol), 1.0 M Na₂CO₃ (1.3 mL, 1.3 mmol), 1.0 M potassium acetate (1.3 mL, 1.3 mmol), and acetonitrile (3 mL) was heated at 110° C. in a microwave reactor for 30 min. The mixture was partitioned between saturated NH₄Cl (50 mL) and ethyl acetate (25 mL). The phases were separated and the aq. extracted with ethyl acetate (2×10 mL). The combined organic phases were dried (Na₂SO₄), filtered, adsorbed onto Celite and chromatographed ISCO 12 g column 0-100% ethyl acetate in dichloromethane to afford 98 mg (21%) of (5)-1-(4-(7,7-dimethyl-4-(3-methylmorpholino)-5,7-dihydrofuro[3,4-d]pyrimidin-2-yl)phenyl)-3-(oxetan-3-yl)urea as a colorless solid: ¹H NMR (400 MHz, DMSO) δ 8.76 (s, 1H), 8.22 (d, J=8.7 Hz, 2H), 7.48 (d, J=8.7 Hz, 2H), 6.93 (d, J=6.6 Hz, 1H), 5.15 (d, J=8.0 Hz, 1H), 5.08 (d, J=8.0 Hz, 1H), 4.83-4.70 (m, 3H), 4.44 (t, J=5.9 Hz, 2H), 4.27 (s, 1H), 4.08-3.84 (m, 2H), 3.72 (d, J=11.4 Hz, 1H), 3.65 (dd, J=11.4, 2.4 Hz, 1H), 3.49 (ddd J=11.8, 11.8, 5.9 Hz, 1H), 3.39-3.31 (m, 1H), 1.39 (s, 6H), 1.26 (d, J=6.7 Hz, 3H); LC-MS: m/z=+440 (M+H)⁺.

### Example 9

### Preparation of (5)-1-(4-(7,7-dimethyl-4-(3-methylmorpholino)-5,7-dihydrofuro[3,4-d]pyrimidin-2-yl)phenyl)-3-(2-hydroxyethyl)urea (w)

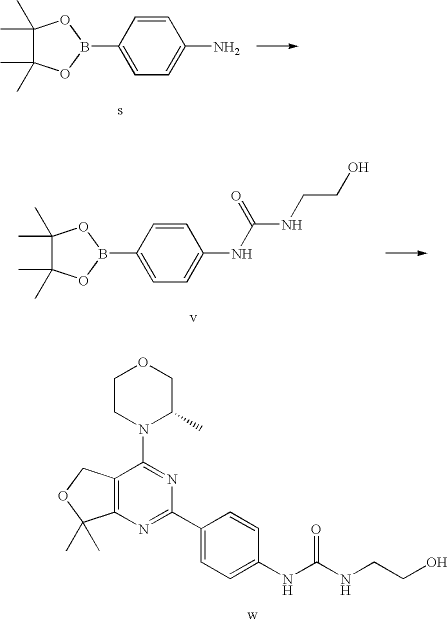

Step 1—Synthesis of 1-(2-hydroxyethyl)-3-(4-(4,4,5,5-tetramethyl-1,3,2-dioxaborolan-2-yl)phenyl)urea (v). The compound v was made by the procedure described in Example 8 Step 1, substituting 2-amino ethanol for 3-oxetanamine.HCl: LC-MS: m/z=+307 (M+H)⁺.

Step 2—Synthesis of (S)-1-(4-(7,7-dimethyl-4-(3-methylmorpholino)-5,7-dihydrofuro[3,4-d]pyrimidin-2-yl)phenyl)-3-(2-hydroxyethyl)urea (w). The compound w was made by the procedure described in Example 8 Step 2, substituting 1-(2-hydroxyethyl)-3-(4-(4,4,5,5-tetramethyl-1,3,2-dioxaborolan-2-yl)phenyl)urea for 1-(oxetan-3-yl)-3-(4-(4,4,5,5-tetramethyl-1,3,2-dioxaborolan-2-yl)phenyl)urea, and purification by reverse-phase HPLC: LC-MS: m/z=+428 (M+H)⁺.

### Example 10

### Preparation of (5)-1-(2-cyanoethyl)-3-(4-(7,7-dimethyl-4-(3-methylmorpholino)-5,7-dihydrofuro[3,4-d]pyrimidin-2-yl)phenyl)urea (z)

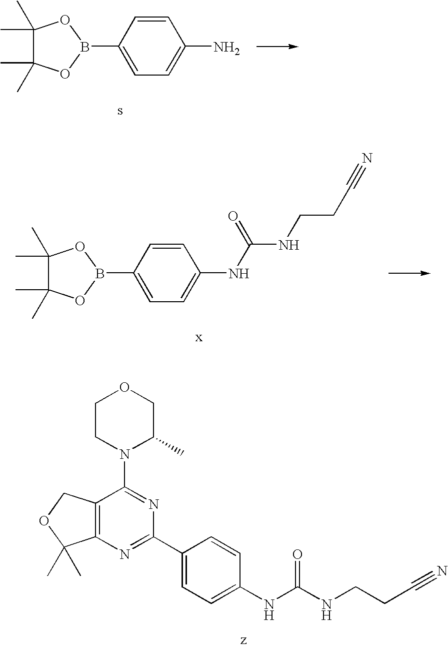

Step 1—Synthesis of 1-(2-cyanoethyl)-3-(4-(4,4,5,5-tetramethyl-1,3,2-dioxaborolan-2-yl)phenyl)urea (x). The compound was made by the procedure for Example 8 Step 1 substituting 3-aminopropanenitrile for 3-oxetanamine.HCl: LC-MS: m/z=+316 (M+H)⁺.

Step 2—Synthesis of (S)-1-(2-cyanoethyl)-3-(4-(7,7-dimethyl-4-(3-methylmorpholino)-5,7-dihydrofuro[3,4-d]pyrimidin-2-yl)phenyl)urea (z). Compound z was made by the procedure for Example 8 Step 2 substituting 1-(2-cyanoethyl)-3-(4-(4,4,5,5-tetramethyl-1,3,2-dioxaborolan-2-yl)phenyl)urea for 1-(oxetan-3-yl)-3-(4-(4,4,5,5-tetramethyl-1,3,2-dioxaborolan-2-yl)phenyl)urea and purification by reverse-phase HPLC: LC-MS: m/z=+437 (M+H)⁺.

### Example 11

### Preparation of 1-((R)-2,3-dihydroxypropyl)-3-(4-(7,7-dimethyl-4-(S)-3-methylmorpholino)-5,7-dihydro furo [3,4-d]pyrimidin-2-yl)phenyl)urea (ae)

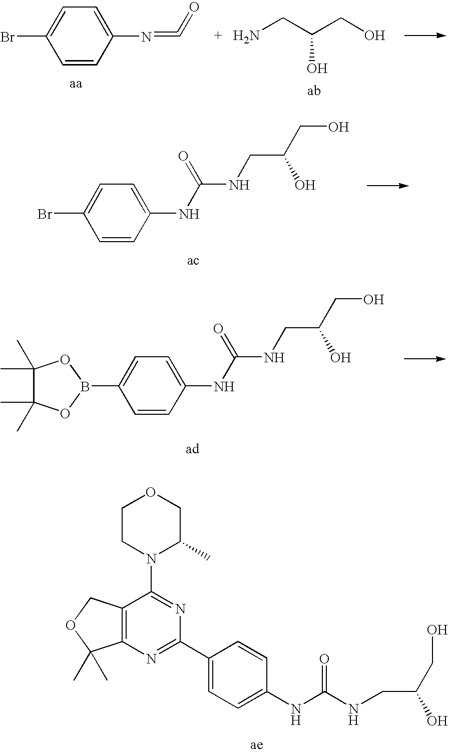

Step 1—Synthesis of (R)-1-(4-bromophenyl)-3-(2,3-dihydroxypropyl)urea (ac). A solution of 1-bromo-4-isocyanatobenzene (aa, 430 mg, 2.17 mmol) and 1,2-dichloroethane (2 mL) was added dropwise to a suspension of (R)-3-aminopropane-1,2-diol (ab, 268 mg, 2.94 mmol) in a mixture of DMF:pyridine:1,2-dichloroethane (1:1:2, 4 mL). The mixture solidified upon completion of addition. Ethyl acetate (30 mL) was added, and stirred for 20 min. The solid was collected on paper, rinsed with ethyl acetate, and dried under vacuum to afford 492 mg (78%) of (R)-1-(4-bromophenyl)-3-(2,3-dihydroxypropyl)urea (ac) as a colorless solid: ¹H NMR (400 MHz, DMSO) δ 8.72 (s, 1H), 7.43-7.25 (m, 4H), 6.15 (t, J=5.6 Hz, 1H), 4.82 (d, J=4.9 Hz, 1H), 4.56 (t, J=5.6 Hz, 1H), 3.55-3.46 (m, 1H), 3.37-3.33 (m, 1H), 3.00-2.94 (m, 1H); LC-MS: m/z=+290 (M+H)⁺.

Step 2—Synthesis of (R)-1-(2,3-dihydroxypropyl)-3-(4-(4,4,5,5-tetramethyl-1,3,2-dioxaborolan-2-yl)phenyl)urea (ad). A mixture of the product from Step 1 (490 mg, 1.7 mmol), bispinocolato diborane (650 mg, 2.6 mmol), potassium acetate (500 mg, 5.1 mmol) and DMSO (4 mL) was heated in a sealed vial at 120° C. for 2 h. The dark solution was poured into saturated NH₄Cl (100 mL) and ethyl acetate (100 mL). Celite was added and the mixture stirred for 20 min., then filtered through more Celite, rinsing with ethyl acetate. The clear phases were separated, and the aq. Extracted with ethyl acetate (3×10 mL). The combined org. phases were washed with brine (1×20 mL), dried (Na₂SO₄), filtered and adsorbed onto Celite. The residue was chromatographed ISCO 12 g column 0-20% IPA in ethyl acetate to afford 195 mg (32%) of compound (ad) as a colorless solid: ¹H NMR (400 MHz, DMSO) δ 8.73 (s, 1H), 7.52 (d, J=8.2 Hz, 2H), 7.37 (d, J=8.2 Hz, 2H), 6.19 (t, J=5.4 Hz, 1H), 4.82 (d, J=5.0 Hz, 1H), 4.56 (t, J=5.6 Hz, 1H), 3.55-3.45 (m, 1H), 3.41-3.30 (m, 2H), 3.07-2.87 (m, 1H), 1.27 (s, 12H); LC-MS: m/z=+337 (M+H)⁺.

Step 3—Synthesis of 14(R)-2,3-dihydroxypropyl)-3-(4-(7,7-dimethyl-4-(S)-3-methylmorpholino)-5,7-dihydrofuro[3,4-d]pyrimidin-2-yl)phenyl)urea (ae). Compound (ae) was made by the procedure described in Example 8 Step 2, substituting (R)-1-(2,3-dihydroxypropyl)-3-(4-(4,4,5,5-tetramethyl-1,3,2-dioxaborolan-2-yl)phenyl)urea for (4,4,5,5-tetramethyl-1,3,2-dioxaborolan-2-yl)phenyl)urea and purification by reverse-phase HPLC: LC-MS: m/z=+458 (M+H)⁺.

### Example 12

### Preparation of 1,3-diethyl(1-(4-(4-morpholino-6,8-dihydro-5H-pyrano[3,4-d]pyrimidin-2-yl))phenylcarbamoyl)urea (ai)

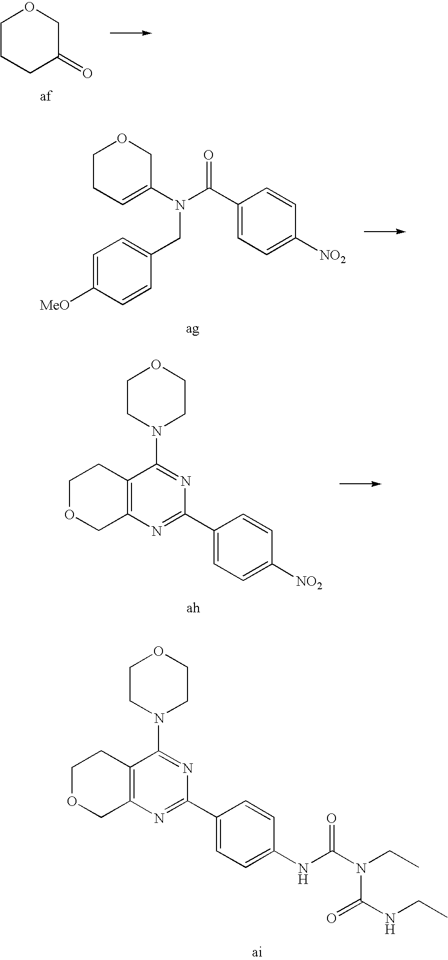

Step 1—Synthesis of ag: To a mixture of dihydro-2H-pyran-3(4H)-one (af, 1.0 mL, 11 mmol) and 4-methoxybenzylamine (1.4 mL, 11 mmol) in CH₂Cl₂ (22 mL) was added titanium(IV) ethoxide (11 mL, 54 mmol). The mixture was heated to 42° C. and stirred for 12 h. The reaction mixture was then cooled to 0° C. and Et₃N (2.8 mL, 20.1 mmol) was added. Simultaneously, a separate vessel was charged with 4-nitrobenzoic acid (1.99 g, 11.9 mmol) and CH₂Cl₂ (5 mL), and 1-Chloro-N,N,2-trimethyl-1-propenylamine (1.72 mL, 13.0 mmol) was added dropwise to this suspension at 0° C. After stirring for 30 min at 0° C. and 10 min at room temperature the clear solution was added via cannula to the above reaction mixture. After stirring for 1 h at room temperature, water (30 mL) was added and the resulting turbid solution is filtered through celite. The filter cake was washed with CH₂Cl₂ (2×), the filtrate is separated, and the aqueous phase is extracted with CH₂Cl₂ (2×). The combined organic extract was dried (Na₂SO₄), filtered and concentrated. The resulting residue was purified by flash column chromatography (40% EA/Hex) to give N-(5,6-dihydro-2H-pyran-3-yl)-N-(4-methoxybenzyl)-4-nitrobenzamide (ag) (1.0 g, 25%): ¹H NMR (400 MHz, CDCl₃) δ 8.21 (d, J=8.7, 2H), 7.65 (d, J=8.7, 2H), 7.29 (dd, J=8.8, 2.5, 2H), 6.87 (d, J=8.6, 2H), 5.46 (s, 1H), 4.76 (s, 2H), 3.81 (s, 3H), 3.76 (s, 2H), 3.47 (s, 2H), 2.01 (s, 2H); LC-MS: m/z=+369 (M+H)⁺.

Step 2—Synthesis of ah: Trifluoromethanesulfonic anhydride (0.090 mL, 0.54 mmol) was added dropwise over 1 min to a stirred mixture of N-(5,6-dihydro-2H-pyran-3-yl)-N-(4-methoxybenzyl)-4-nitrobenzamide (ag) (180 mg, 0.49 mmol), 4-morpholinecarbonitrile (0.054 mL, 0.54 mmol), and 2-chloropyridine (0.055 mL, 0.59 mmol) dissolved in CH₂Cl₂ (4.5 mL) at −78° C. After 5 min, the reaction mixture was warmed to 0° C. for 5 min and then to room temperature for 10 min. The mixture was then quenched with 1 N NaOH (2 mL), separated, and the aqueous phase extracted with CH₂Cl₂ (2×). The combined organic extract was dried (Na₂SO₄), filtered and concentrated. The resulting solid was washed with Et₂O and heptane, and filtered to provide 4-morpholino-2-(4-nitrophenyl)-6,8-dihydro-5H-pyrano[3,4-d]pyrimidine (ah) (120 mg, 72%): ¹H NMR (400 MHz, DMSO) δ 8.53 (d, J=9.0, 2H), 8.33 (d, J=9.0, 2H), 4.68 (s, 2H), 3.84 (t, J=5.1, 2H), 3.78-3.70 (m, 4H), 3.62-3.56 (m, 4H), 2.75 (t, J=5.1, 2H); LC-MS: m/z=+343 (M+H)⁺.

Step 3—Synthesis of ai: To 4-morpholino-2-(4-nitrophenyl)-6,8-dihydro-5H-pyrano[3,4-d]pyrimidine (ah) (0.112 g, 0.327 mmol) and ethanol (4 mL) was added stannous chloride, dihydrate (372 mg, 1.64 mmol) and the mixture was heated to 77° C. and stirred for 2 h. After concentration of the reaction mixture, acetone (5 mL) and 1 N NaOH (5 mL) were added. After separation and extraction of the aqueous phase with acetone, the combined organic extract was dried (Na₂SO₄), filtered and concentrated. To the resulting crude aniline dissolved in DMF (1.5 mL) was added ethyl isocyanate (0.141 mL, 1.79 mmol) and the mixture was heated to 75° C. and stirred for 12 h. After cooling to room temperature, the resulting mixture was purified by reverse-phase HPLC to give the pure desired product ai: ¹H NMR (400 MHz, DMSO) δ 11.61 (s, 1H), 8.23 (d, J=8.7, 2H), 7.77 (t, J=5.3, 1H), 7.57 (d, J=8.7, 2H), 4.62 (s, 2H), 3.81 (dd, J=13.1, 6.2, 4H), 3.76-3.72 (m, 4H), 3.54-3.49 (m, 4H), 3.19 (dt, J=10.9, 6.2, 2H), 2.70 (t, J=4.8, 2H), 1.11 (dt, J=12.6, 6.2, 6H); LC-MS: m/z=+455 (M+H)⁺.

### Example 13

### Preparation of (S)-1-ethyl-3-(4-(4-(3-methylmorpholino)-6,8-dihydro-5H-pyrano[3,4-d]pyrimidin-2-yl)phenyl)urea (al) and (S)-1,3-diethyl(1-(4-(4-3-methylmorpholino-6,8-dihydro-5H-pyrano[3,4-d]pyrimidin-2-yl))phenylcarbamoyl)urea (am)

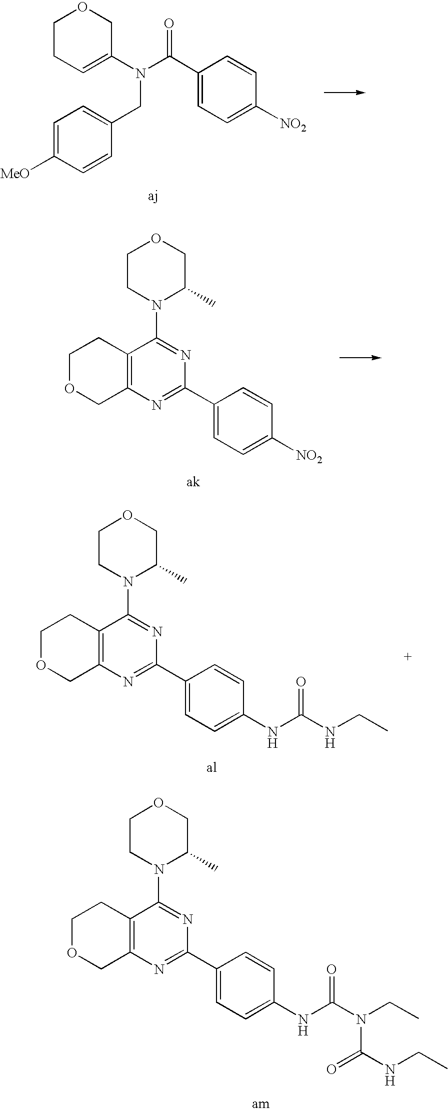

Step 1—Synthesis of ak: Trifluoromethanesulfonic anhydride (0.084 mL, 0.50 mmol) was added dropwise over 1 min to a stirred mixture of N-(5,6-dihydro-2H-pyran-3-yl)-N-(4-methoxybenzyl)-4-nitrobenzamide (b) (167 mg, 0.45 mmol), (S)-3-methylmorpholine-4-carbonitrile (0.063 mL, 0.50 mmol), 2-chloropyridine (0.052 mL, 0.54 mmol), and 2,6-dichloropyridine (13 mg, 0.09 mmol) dissolved in CH₂Cl₂ (4.0 mL) at −78° C. After 5 min, the reaction mixture was warmed to 0° C. for 5 min and then to room temperature for 20 min. The mixture was then quenched with 1 N NaOH (2 mL), separated, and the aqueous phase extracted with CH₂Cl₂ (2×). The combined organic extract was dried (Na₂SO₄), filtered and concentrated. The resulting residue was purified by flash column chromatography (15% EA/CH₂Cl₂) to give (S)-4-(3-methylmorpholino)-2-(4-nitrophenyl)-6,8-dihydro-5H-pyrano[3,4-d]pyrimidine (ak) (130 mg, 81%): ¹H NMR (400 MHz, CDCl₃) δ 8.53 (d, J=9.0, 2H), 8.28 (d, J=9.0, 2H), 4.85-4.71 (m, 2H), 4.19 (d, J=6.6, 1H), 4.03-3.95 (m, 2H), 3.87-3.82 (m, 2H), 3.77-3.69 (m, 3H), 3.61-3.56 (m, 1H), 2.77-2.71 (m, 2H), 1.38 (d, J=6.7, 3H); LC-MS: m/z=+357 (M+H)⁺.

Step 2—Synthesis of (S)-1-ethyl-3-(4-(4-(3-methylmorpholino)-6,8-dihydro-5H-pyrano[3,4-d]pyrimidin-2-yl)phenyl)urea (al) and (S)-1,3-diethyl(1-(4-(4-3-methylmorpholino-6,8-dihydro-5H-pyrano[3,4-d]pyrimidin-2-yl))phenylcarbamoyl)urea (am): To (S)-4-(3-methylmorpholino)-2-(4-nitrophenyl)-6,8-dihydro-5H-pyrano[3,4-d]pyrimidine (e) (0.126 g, 0.354 mmol) and ethanol (4.5 mL) was added stannous chloride, dihydrate (402 mg, 1.77 mmol) and the mixture was heated to 77° C. and stirred for 2 h. After concentration, the reaction mixture was partitioned between 1 N NaOH (5 mL) and CH₂Cl₂ (5 mL) and separated. The organic extract was washed with 1 N NaOH (5 mL), and after separation, the combined aqueous phase was extracted with CH₂Cl₂ (5 mL). The combined organic extract was then dried (Na₂SO₄), filtered and concentrated. To the resulting crude aniline dissolved in DMF (2.5 mL) was added ethyl isocyanate (0.042 mL, 0.531 mmol) and the mixture was heated to 75° C. and stirred for 2 h at which time another 1.5 equiv of ethyl isocyanate was added. After another 2 h at 75° C., the mixture was cooled to room temperature and purified by reverse-phase HPLC to give the pure desired products al and am: ¹H NMR (500 MHz, DMSO) δ 8.77 (s, 1H), 8.15 (d, J=8.7, 2H), 7.47 (d, J=8.7, 2H), 6.28 (t, J=5.5, 1H), 4.69-4.50 (m, 2H), 4.18 (d, J=6.5, 1H), 3.91-3.82 (m, 2H), 3.77 (dt, J=11.1, 5.4, 1H), 3.71-3.57 (m, 4H), 3.45-3.38 (m, 1H), 3.15-3.05 (m, 2H), 2.73-2.61 (m, 2H), 1.26 (d, J=6.6, 3H), 1.05 (t, J=7.2, 3H); LC-MS: m/z=+398 (M+H)⁺; and (g): ¹H NMR (500 MHz, DMSO) δ 8.32 (d, J=8.4, 2H), 7.68 (t, J=5.6, 2H), 7.27 (d, J=8.5, 2H), 4.71-4.57 (m, 2H), 4.23 (d, J=7.3, 1H), 3.92-3.86 (m, 2H), 3.82-3.65 (m, 4H), 3.60 (dd, J=11.3, 9.4, 1H), 3.48-3.42 (m, 1H), 3.12-3.05 (m, 4H), 2.72 (s, 2H), 1.28 (d, J=6.7, 3H), 1.01 (t, J=7.1, 6H); LC-MS: m/z=+469 (M+H)⁺.

### Example 14

### Preparation of 1-ethyl-3-(4-(4′-morpholino-5′,6′-dihydrospiro[cyclopropane-1,7′-pyrano[2,3-d]pyrimidine]-2′-yl)phenyl)urea (ax)

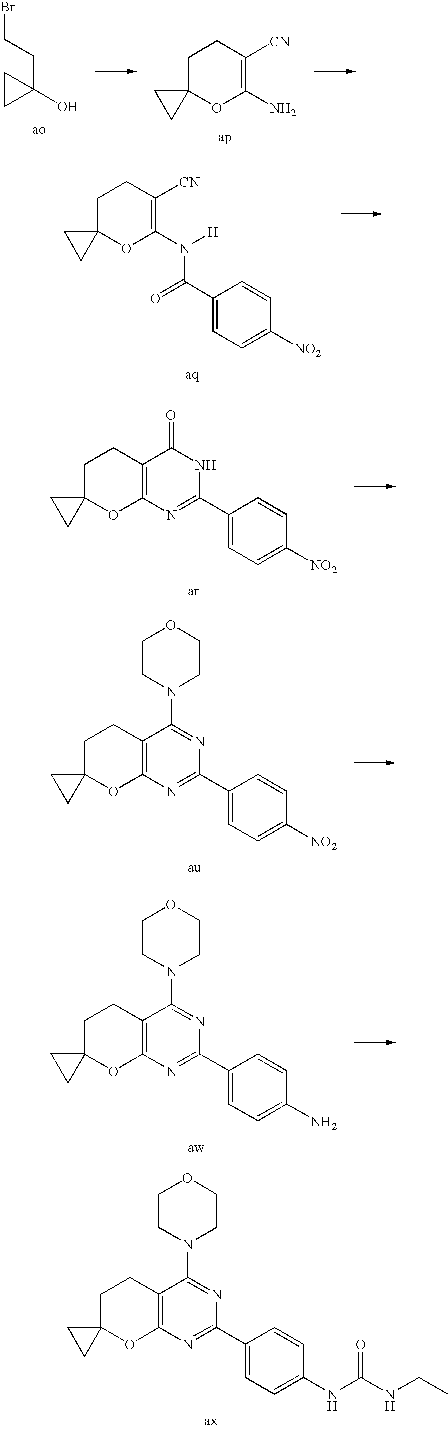

Step 1—Synthesis of ao: 1-(2-bromoethyl)cyclopropanol (11) was prepared according to the procedure outlined in Eur. J. Org. Chem. 2003, 551-561.

Step 2—Synthesis of 5-amino-4-oxaspiro[2.5]oct-5-ene-6-carbonitrile (ap): Ethanol (30 mL, 0.5 mol) was cooled to 0° C., and then sodium metal (1.526 g, 0.06638 mol) was added and stirred until dissolved. Malononitrile (4.20 mL, 0.0667 mol) was then added in 5 portions over 5 minutes to give a milky white suspension. This was then warmed to 40° C., and 1-(2-bromoethyl)cyclopropanol (8.48 g, 0.0514 mol) was dissolved in 5 ml EtOH, with a 2 ml rinse, and all was added dropwise over 15 min. The reaction was stirred 2 h at 40° C., then the NaBr was filtered off, and the resulting solution was concentrated to an orange-ish oil and poured into ice water. NaCl was added to salt out the product, which came out of solution as a thick oil, which was filtered off. The filtrate also showed some of the oil present, and was extracted with EtOAc (3×100 ml). The solids were dissolved the organic extracts and the resulting dark orange solution was dried with MgSO₄, filtered and concentrated onto silica gel. This was then subjected to column chromatography using a 120 g column, with a gradient of 0% to 40% ethyl acetate in heptane. The product containing fractions were combined and evaporated under reduced pressure to give 5-amino-4-oxaspiro[2.5]oct-5-ene-6-carbonitrile (2.80 g, 36%) as a light yellow solid. ¹H NMR (500 MHz, CDCl₃) δ 4.35 (br s, 2H), 2.34 (t, J=6.3 Hz, 2H), 1.77 (t, J=6.3 Hz, 2H), 1.02-0.92 (m, 2H), 0.65-0.54 (m, 2H).

Step 3—Synthesis of N-(6-cyano-4-oxaspiro[2.5]oct-5-en-5-yl)-4-nitrobenzamide (aq): 5-amino-4-oxaspiro[2.5]oct-5-ene-6-carbonitrile (2.744 g, 0.01827 mol) was weighed into a flask, then dissolved in methylene chloride (50 mL, 0.8 mol). Triethylamine (7.9 mL, 0.057 mol) was added, then followed by p-nitrobenzoyl chloride (8.526 g, 0.04595 mol) in a single portion. The solution immediately became orange-yellow. The reaction was stirred at RT overnight, becoming dark brown. The reaction was filtered to remove TEA-HCl, washing with 1:1 hexane/CH₂Cl₂. The filtrate was concentrated and dissolved in tetrahydrofuran (50 mL, 0.6 mol), and 3.00 M of Sodium hydroxide in water (15 mL) was added and heated to reflux for 1 h. The reaction was then cooled and diluted with water and EtOAc. The aqueous phase was extracted with EtOAc (3×100 ml), the combined organics were washed with 1N HCl (1×100 ml), dried with MgSO₄, filtered and concentrated onto silica gel. This material was then subjected to column chromatography using a 40 g column, with a gradient of 0% to 60% ethyl acetate in hexanes. The product containing fractions were combined and evaporated under reduced pressure to give N-(6-cyano-4-oxaspiro[2.5]oct-5-en-5-yl)-4-nitrobenzamide. ¹H NMR (400 MHz, DMSO) δ 10.88 (s, 1H), 8.36 (d, J=8.8 Hz, 2H), 8.09 (d, J=8.8 Hz, 2H), 2.47 (t, J=6.3 Hz, 2H), 1.87 (t, J=6.3 Hz, 2H), 0.96 (t, J=6.2 Hz, 2H), 0.74 (t, J=6.4 Hz, 2H).

Step 4—Synthesis of 2′-(4-nitrophenyl)-5′,6′-dihydrospiro[cyclopropane-1,7′-pyrano[2,3-d]pyrimidin]-4′(3′H)-one (ar): N-(6-cyano-4-oxaspiro[2.5]oct-5-en-5-yl)-4-nitrobenzamide (4.30 g, 0.0144 mol) and benzoic acid (1.904 g, 0.01559 mol) were weighed into a reaction vial equipped with a stirbar. Ethyl orthoformate (50 mL, 0.30 mol) was added, the vial was sealed and flushed with N₂, then heated to 145° C. overnight. In the morning the reaction was cooled and the volatiles were removed under reduced pressure. The resulting solid material was suspended in hot CH₂Cl₂, cooled to 4° C., filtered and washed with cold CH₂Cl₂ to give 2′-(4-nitrophenyl)-5′,6′-dihydrospiro[cyclopropane-1,7′-pyrano[2,3-d]pyrimidin]-4′(3′H)-one. ¹H NMR (400 MHz, DMSO) δ 12.71 (br s, 1H), 8.32 (s, 4H), 2.57 (t, J=6.2 Hz, 2H), 1.90 (t, J=6.3 Hz, 2H), 1.03-0.95 (m, 2H), 0.76-0.68 (m, 2H).

Step 5a—Synthesis of 4′-morpholino-2′-(4-nitrophenyl)-5′,6′-dihydrospiro[cyclopropane-1,7′-pyrano[2,3-d]pyrimidine] (au): 2′-(4-nitrophenyl)-5′,6′-dihydrospiro[cyclopropane-1,7′-pyrano[2,3-d]pyrimidin]-4′(3′H)-one (2.96 g, 0.00989 mol) was suspended in phosphoryl chloride (30 mL, 0.3 mol) and heated to 100° C. under a nitrogen atmosphere for 6 h. The reaction was cooled, then the volatiles were removed under reduced pressure. The residual slurry was poured into 200 ml ice, stirring until all the ice has melted. The tan solids that formed were filtered off and washed with 100 ml water. The resulting 4′-chloro-2′-(4-nitrophenyl)-5′,6′-dihydrospiro[cyclopropane-1,7′-pyrano[2,3-d]pyrimidine] could be carried on without further purification. ¹H NMR (400 MHz, DMSO) δ 8.47 (d, J=8.9 Hz, 2H), 8.34 (d, J=8.9 Hz, 2H), 2.91 (t, J=6.4 Hz, 2H), 2.06 (t, J=6.4 Hz, 2H), 1.12-1.05 (m, 2H), 0.86-0.78 (m, 2H).

Step 5b—4′-chloro-2′-(4-nitrophenyl)-5′,6′-dihydrospiro[cyclopropane-1,7′-pyrano[2,3-d]pyrimidine] (0.785 g, 0.00247 mol) was weighed into a 25 ml roundbottom flask equipped with a stirbar. N,N-dimethylformamide (10 mL, 0.1 mol) and N,N-diisopropylethylamine (0.650 mL, 0.00373 mol) were added, followed by morpholine (0.26 mL, 0.0030 mol). The reaction was heated to 80° C. for 4 h. The reaction was cooled, which produced a precipitate. This mixture was poured into 200 ml water, filtered and washed with 100 ml water. This gave 4′-morpholino-2′-(4-nitrophenyl)-5′,6′-dihydrospiro[cyclopropane-1,7′-pyrano[2,3-d]pyrimidine] as a light yellow powder. ¹H NMR (400 MHz, DMSO) δ 8.48 (d, J=8.8 Hz, 2H), 8.30 (d, J=8.9 Hz, 2H), 3.81-3.72 (m, 4H), 3.57-3.47 (m, 4H), 2.77 (t, J=5.9 Hz, 2H), 1.89 (t, J=5.9 Hz, 2H), 1.07-0.99 (m, 2H), 0.79-0.73 (m, 2H).

Step 6—Synthesis of 4-(4′-morpholino-5′,6′-dihydrospiro[cyclopropane-1,7′-pyrano[2,3-d]pyrimidine]-2′-yl)aniline (aw): 4′-morpholino-2′-(4-nitrophenyl)-5′,6′-dihydrospiro[cyclopropane-1,7′-pyrano[2,3-d]pyrimidine] (90.9 mg, 0.247 mmol) and tin dichloride (236 mg, 1.23 mmol) were weighed into a reaction vial. Ethanol (3 mL, 0.05 mol) was added, and the reaction was stirred and heated to 100° C. for 2 h. LC/MS shows that reaction is fairly clean, and is complete. The volatiles were removed under reduced pressure, and then diluted with water (25 ml) and basified with 1N NaOH to pH 9-10. The aqueous phase was extracted using gentle shaking to avoid emulsions with 10% MeOH in dichloromethane (3×25 ml), and the combined organics were dried over MgSO₄, filtered and concentrated onto silica gel. This material was then subjected to column chromatography using a 4 g column, with a gradient of 0% to 50% ethyl acetate in hexanes. The product containing fractions were combined and evaporated under reduced pressure to give 4-(4′-morpholino-5′,6′-dihydrospiro[cyclopropane-1,7′-pyrano[2,3-d]pyrimidine]-2′-yl)aniline. ¹H NMR (400 MHz, DMSO) δ 7.94 (d, J=8.6 Hz, 2H), 6.56 (d, J=8.6 Hz, 2H), 5.49 (s, 2H), 3.78-3.69 (m, 4H), 3.43-3.36 (m, 4H), 2.68 (t, J=6.0 Hz, 2H), 1.84 (t, J=5.9 Hz, 2H), 1.02-0.94 (m, 2H), 0.76-0.66 (m, 2H).

Step 7—Synthesis of 1-ethyl-3-(4-(4′-morpholino-5′,6′-dihydrospiro[cyclopropane-1,7′-pyrano[2,3-d]pyrimidine]-2′-yl)phenyl)urea (ax): 4-(4′-morpholino-5′,6′-dihydrospiro[cyclopropane-1,7′-pyrano[2,3-d]pyrimidine]-2′-yl)aniline (64 mg, 1.9 mmol) was dissolved in N,N-dimethylformamide (0.7 mL, 9 mmol). Ethyl isocyanate (25 uL, 3.2 mmol) was added in a single portion, and the reaction warmed to 50° C. overnight. After 18 h, LC/MS indicates that the reaction is only partially complete. An additional 25 uL ethyl isocyanate (0.32 mmol, 1.7 eq) was added and the temperature was increased to 60° C. Stirred overnight. This crude mixture was then purified by reverse phase HPLC: ¹H NMR (400 MHz, DMSO) δ 8.66 (s, 1H), 8.11 (d, J=8.7 Hz, 2H), 7.45 (d, J=8.8 Hz, 2H), 6.20 (t, J=5.5 Hz, 1H), 3.80-3.68 (m, 4H), 3.51-3.38 (m, 4H), 3.16-3.05 (m, 2H), 2.71 (t, J=6.0 Hz, 2H), 1.86 (t, J=5.8 Hz, 2H), 1.06 (t, J=7.2 Hz, 3H), 1.01 (t, J=6.0 Hz, 2H), 0.73 (t, J=6.3 Hz, 2H). LC-MS: m/z=+410.2 (M+H)⁺.

### Example 15

### Preparation of 1-ethyl-3-(4-(4-morpholino-6,7-dihydro-5H-pyrano[2,3-d]pyrimidin-2-yl)phenyl)urea (bb)

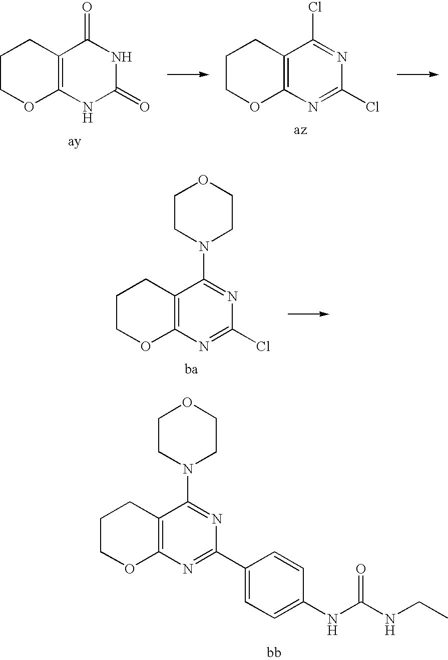

Step 1—Synthesis of 6,7-dihydro-1H-pyrano[2,3-d]pyrimidine-2,4(3H,5H)-dione (ay): Compound ay (6,7-dihydro-1H-pyrano[2,3-d]pyrimidine-2,4(3H,5H)-dione) was prepared according to the procedures outlined in Monatshefte Fur Chemie (2006) 137:1421-1430.

Step 2—Synthesis of compound 2,4-dichloro-6,7-dihydro-5H-pyrano[2,3-d]pyrimidine (az): 6,7-dihydro-1H-pyrano[2,3-d]pyrimidine-2,4(3H,5H)-dione (ay) (849 mg, 5.05 mmol) was added to phosphoryl chloride (1.0E1 mL, 110 mmol) in a 50 ml round bottom flask equipped with a stirbar. The solution was heated to 100° C. and reaction progress was monitored by LC/MS. There was no further formation of product after 4 h. The reaction was cooled, then excess POCl₃ was removed under reduced pressure before adding ice, then solid NaHCO₃ to neutralize and resultant solution was extracted with CH₂Cl₂ (3×20 ml). The combined organics were dried with MgSO₄, filtered and concentrated. The crude material was dissolved in dichloromethane and concentrated onto silica gel. The crude material was purified by column chromatography using a 12 g column, with a gradient of 0% to 40% ethyl acetate in hexanes. The product-containing fractions were combined and evaporated under reduced pressure to give 2,4-dichloro-6,7-dihydro-5H-pyrano[2,3-d]pyrimidine (az) as a white solid. ¹H NMR (400 MHz, CDCl₃) δ 4.43 (t, J=5.3 Hz, 2H), 2.78 (t, J=6.5 Hz, 2H), 2.16-2.05 (m, 2H).

Step 3—Synthesis of 2-chloro-4-morpholino-6,7-dihydro-5H-pyrano[2,3-d]pyrimidine (ba): 2,4-dichloro-6,7-dihydro-5H-pyrano[2,3-d]pyrimidine (az) (215 mg, 0.00105 mol) was dissolved in N,N-Dimethylformamide (1.0 mL, 0.013 mol). N,N-Diisopropylethylamine (275 uL, 0.00158 mol) was added, then morpholine (102 uL, 0.00117 mol) in a single portion. The resulting solution was stirred at room temperature overnight. When the reaction was complete (as monitored by TLC and LC/MS), it was poured into 100 ml H₂O and extracted with EtOAc (3×25 ml). The combined organics were dried with MgSO₄, filtered and concentrated onto silica gel. The crude material was then purified by column chromatography using a 12 g column, with a gradient of 0% to 70% ethyl acetate in hexanes. The product-containing fractions were combined and evaporated under reduced pressure to give 2-chloro-4-morpholino-6,7-dihydro-5H-pyrano[2,3-d]pyrimidine (ba) as a white solid (183 mg, 68%) as well as a higher Rf minor product resulting from 2-substitution. ¹H NMR (400 MHz, CDCl₃) δ 4.36 (t, 2H), 3.79 (d, 4H), 3.47 (d, 4H), 2.57 (t, J=6.2, 2H), 2.01-1.90 (m, J=10.5, 6.1, 2H).

Step 4—Synthesis of 1-ethyl-3-(4-(4-morpholino-6,7-dihydro-5H-pyrano[2,3-d]pyrimidin-2-yl)phenyl)urea (bb): 2-chloro-4-morpholino-6,7-dihydro-5H-pyrano[2,3-d]pyrimidine (27.4 mg, 0.107 mmol), and Tetrakis(triphenylphosphine)palladium(0) (5.6 mg, 0.0048 mmol) were weighed into a microwave vial equipped with a stirbar. The atmosphere was evacuated and replaced with nitrogen 3 times. Acetonitrile (0.5 mL, 10 mmol) and degassed solutions of 1.00 M of Sodium carbonate in Water (0.25 mL) and 1.00 M of Potassium acetate in Water (0.25 mL) were added, the tube was sealed and the mixture microwaved at 100° C. for 30 min. The reaction was diluted with 25 ml water and extracted with EtOAc (3×25 ml). The combined organics were dried with MgSO₄, filtered and concentrated onto silica gel. This material was then subjected to column chromatography using a 12 g column, with a gradient of 0% to 50% ethyl acetate in hexanes. The product containing fractions were combined and evaporated under reduced pressure to give 1-ethyl-3-(4-(4-morpholino-6,7-dihydro-5H-pyrano[2,3-d]pyrimidin-2-yl)phenyl)urea (bb) as a white solid. ¹H NMR (400 MHz, DMSO) δ 8.68-8.58 (m, 1H), 8.12 (d, J=8.7, 2H), 7.45 (d, J=8.7, 2H), 6.16 (t, J=5.6, 1H), 4.37-4.25 (m, 2H), 3.79-3.67 (m, 4H), 3.39 (m, 4H), 3.19-3.03 (m, 2H), 2.59 (t, J=6.0, 2H), 1.92-1.82 (m, 2H), 1.06 (t, J=7.2, 3H). LC-MS: m/z=+384.1 (M+H)⁺.

### Example 16

### Preparation of (S)-1-ethyl-3-(4-(4-(3-methylmorpholino)-6,7-dihydro-5H-pyrano[2,3-d]pyrimidin-2-yl)phenyl)urea (bd)

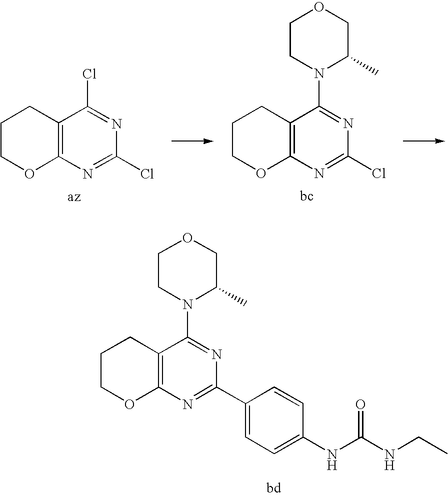

Step 1—Synthesis of (S)-2-chloro-4-(3-methylmorpholino)-6,7-dihydro-5H-pyrano[2,3-d]pyrimidine (bc): 3S-3-methylmorpholine (0.2689 g, 2.658 mmol) was dissolved in N,N-dimethylformamide (1.9 mL, 25 mmol). N,N-diisopropylethylamine (0.50 mL, 2.9 mmol) was added, then 2,4-dichloro-6,7-dihydro-5H-pyrano[2,3-d]pyrimidine (0.44 g, 2.1 mmol) in a single portion. The resulting solution was warmed to 50° C. overnight. The reaction was poured into 200 ml H₂O and the solids filtered off. The crude material was dissolved in CH₂Cl₂ and concentrated onto silica gel. This material was then subjected to column chromatography using a 120 g column, with a gradient of 0% to 50% ethyl acetate in hexanes. The product containing fractions were combined and evaporated under reduced pressure to give (S)-2-chloro-4-(3-methylmorpholino)-6,7-dihydro-5H-pyrano[2,3-d]pyrimidine (bc). The higher Rf product obtained was identified as the 2-regioisomer. ¹H NMR (400 MHz, CDCl₃) δ 4.44-4.26 (m, 2H), 4.04-3.95 (m, 1H), 3.94-3.86 (m, 1H), 3.76 (dd, J=11.3, 2.9, 1H), 3.70-3.60 (m, 2H), 3.56-3.42 (m, 2H), 2.62-2.45 (m, 2H), 2.05-1.85 (m, 2H), 1.32 (d, J=6.8, 3H).

Step 2—Synthesis of (S)-1-ethyl-3-(4-(4-(3-methylmorpholino)-6,7-dihydro-5H-pyrano[2,3-d]pyrimidin-2-yl)phenyl)urea (bd). (S)-2-chloro-4-(3-methylmorpholino)-6,7-dihydro-5H-pyrano[2,3-d]pyrimidine (bc) (47.4 mg, 0.176 mmol), and Tetrakis(triphenylphosphine)palladium(0) (17.0 mg, 0.0147 mmol) were weighed into a microwave vial equipped with a stirbar. The atmosphere was evacuated and replaced with nitrogen 3 times. Acetonitrile (0.80 mL, 15 mmol) and degassed solutions of 1.00 M of Sodium carbonate in Water (0.40 mL) and 1.00 M of Potassium acetate in Water (0.40 mL) were added and the mixture microwaved at 100° C. for 30 min. The reaction was diluted with 25 ml water and extracted with EtOAc (3×25 ml). The combined organics were dried with MgSO₄, filtered and concentrated onto silica gel. This crude material was purified by column chromatography using a 12 g column, with a gradient of 0% to 50% ethyl acetate in hexanes. The product containing fractions were combined and evaporated under reduced pressure to give a white solid. This material showed some impurities and further was purified by reverse phase HPLC to give (S)-1-ethyl-3-(4-(4-(3-methylmorpholino)-6,7-dihydro-5H-pyrano[2,3-d]pyrimidin-2-yl)phenyl)urea (bd). ¹H NMR (500 MHz, DMSO) δ 8.65 (s, 1H), 8.11 (d, J=8.8, 2H), 7.46 (d, J=8.8, 2H), 6.18 (t, J=5.5, 1H), 4.39-4.23 (m, 2H), 4.00 (d, J=6.6, 1H), 3.86 (d, J=11.2, 1H), 3.70 (dd, J=11.2, 2.7, 1H), 3.65-3.57 (m, J=9.7, 2H), 3.53-3.36 (m, J=26.6, 16.5, 8.3, 2H), 3.16-3.07 (m, 2H), 2.58 (t, J=6.1, 2H), 1.97-1.75 (m, 2H), 1.23 (d, J=6.6, 3H), 1.06 (t, J=7.2, 3H). LC-MS: m/z=+398.2 (M+H)⁺.

### Example 17

### Preparation of (S)-1-ethyl-3-(4-(4-(3-methylmorpholino)-7,8-dihydro-5H-pyrano[4,3-d]pyrimidin-2-yl)phenyl)urea (bg)

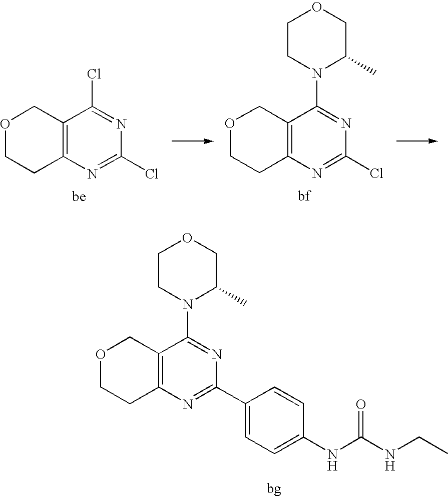

Step 1—Synthesis of (S)-2-chloro-4-(3-methylmorpholino)-7,8-dihydro-5H-pyrano[4,3-d]pyrimidine (bf): (3S-3-methylmorpholine (1.138 g, 11.25 mmol) was dissolved in N,N-dimethylformamide (9.6 mL, 120 mmol). N,N-diisopropylethylamine (2.56 mL, 14.7 mmol) was added, then 2,4-dichloro-7,8-dihydro-5H-pyrano[4,3-d]pyrimidine (be) (2.086 g, 10.17 mmol) in a single portion. The resulting solution was warmed to 50° C. and stirred overnight in a sealed reaction vessel. The reaction was then poured into 100 ml H₂O and extracted with EtOAc (3×25 ml). The combined organics were dried with MgSO₄, filtered and concentrated onto silica gel. This material was then subjected to column chromatography using a 12 g column, with a gradient of 0% to 70% ethyl acetate in hexanes. The product-containing fractions were combined and evaporated under reduced pressure to give (S)-2-chloro-4-(3-methylmorpholino)-7,8-dihydro-5H-pyrano[4,3-d]pyrimidine (bf) as a white solid. The higher Rf minor product was identified as the regioisomeric 4-chloro-2-morpholino-7,8-dihydro-5H-pyrano[4,3-d]pyrimidine. ¹H NMR (400 MHz, CDCl₃) δ 4.54 (q, J=14.0 Hz, 2H), 4.11-3.81 (m, 4H), 3.77-3.46 (m, 5H), 2.99-2.83 (m, 2H), 1.34 (d, J=6.8 Hz, 3H).

Step 2—Synthesis of (S)-1-ethyl-3-(4-(4-(3-methylmorpholino)-7,8-dihydro-5H-pyrano[4,3-d]pyrimidin-2-yl)phenyl)urea (bg): (S)-2-chloro-4-(3-methylmorpholino)-7,8-dihydro-5H-pyrano[4,3-d]pyrimidine (bf) (52.7 mg, 0.195 mmol), and tetrakis(triphenylphosphine)palladium(0) (18.4 mg, 0.0159 mmol) were weighed into a microwave vial equipped with a stirbar. The atmosphere was evacuated and replaced with nitrogen 3 times. Acetonitrile (0.80 mL, 15 mmol) and degassed solutions of 1.00 M of sodium carbonate in water (0.40 mL) and 1.00 M of potassium acetate in water (0.40 mL) were added and the mixture microwaved at 100° C. for 30 min. Some starting material remained, so the reaction was reheated to 110° C. for 25 min. The reaction was diluted ith 25 ml water and extracted with EtOAc (3×25 ml). The combined organics were dried with MgSO₄, filtered and concentrated onto silica gel. This crude material was purified by column chromatography using a 12 g column, with a gradient of 0% to 50% ethyl acetate in hexanes. The product-containing fractions were combined and evaporated under reduced pressure to give a white solid. This material further was purified by reverse phase HPLC to give (S)-1-ethyl-3-(4-(4-(3-methylmorpholino)-7,8-dihydro-5H-pyrano[4,3-d]pyrimidin-2-yl)phenyl)urea (bg). ¹H NMR (500 MHz, DMSO) δ 8.65 (s, 1H), 8.11 (d, J=8.8 Hz, 2H), 7.46 (d, J=8.8 Hz, 2H), 6.18 (t, J=5.5 Hz, 1H), 4.37-4.23 (m, 2H), 4.00 (d, J=6.6 Hz, 1H), 3.86 (d, J=11.2 Hz, 1H), 3.70 (dd, J=11.2, 2.7 Hz, 1H), 3.61 (t, J=9.7 Hz, 2H), 3.49 (d, J=13.6 Hz, 1H), 3.44-3.35 (m, 1H), 3.15-3.06 (m, 2H), 2.58 (t, J=6.1 Hz, 2H), 1.95-1.77 (m, 2H), 1.23 (d, J=6.6 Hz, 3H), 1.06 (t, J=7.2 Hz, 3H). LC-MS: m/z=+398.2 (M+H)⁺.

### Example 18

### Preparation of 1-ethyl-3-(4-(4-morpholino-7,8-dihydro-5H-pyrano[4,3-d]pyrimidin-2-yl)phenyl)urea (bh)

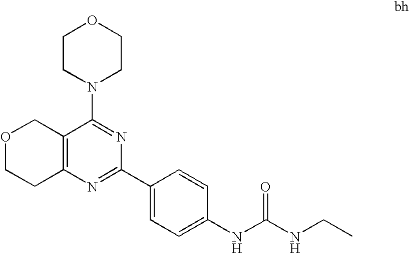

The title compound bh was prepared by the procedure described in Example 17, by substituting 3S-3-methylmorpholine with morpholine: ¹H NMR (500 MHz, DMSO) δ 8.62 (s, 1H), 8.19 (d, J=8.7 Hz, 2H), 7.47 (d, J=8.7 Hz, 2H), 6.14 (t, J=5.6 Hz, 1H), 4.58 (s, 2H), 4.00 (t, J=6.0 Hz, 2H), 3.76-3.68 (m, 4H), 3.42-3.34 (m, 4H), 3.15-3.08 (m, 2H), 2.85 (t, J=6.0 Hz, 2H), 1.06 (t, J=7.2 Hz, 3H). LC-MS: m/z=+384.2 (M+H)⁺.

### Example 19

### Preparation of (S)-1-ethyl-3-(4-(4-(3-ethylmorpholino)-7,8-dihydro-5H-pyrano[4,3-d]pyrimidin-2-yl)phenyl)urea (bi)

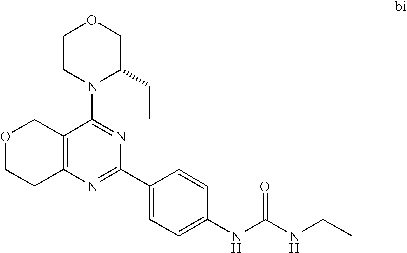

The title compound bi was prepared by the procedure described in Example 17, by substituting 3S-3-methylmorpholine with 3S-3-ethylmorpholine: ¹H NMR (400 MHz, DMSO) δ 8.66 (s, 1H), 8.17 (d, J=8.7 Hz, 2H), 7.47 (d, J=8.8 Hz, 2H), 6.17 (t, J=5.5 Hz, 1H), 4.57 (q, J=14.1 Hz, 2H), 4.11-4.00 (m, 1H), 3.98-3.88 (m, 1H), 3.84 (d, J=9.3 Hz, 1H), 3.80-3.40 (m, 6H), 3.17-3.06 (m, 2H), 2.93-2.77 (m, 2H), 1.86-1.66 (m, 2H), 1.06 (t, J=7.2 Hz, 3H), 0.83 (t, J=7.4 Hz, 3H). LC-MS: m/z=+412.3 (M+H)⁺.

### Example 20

### Preparation of (S)-1-(isoxazol-3-yl)-3-(4-(4-(3-methylmorpholino)-7,8-dihydro-5H-pyrano[4,3-d]pyrimidin-2-yl)phenyl)urea (bl)

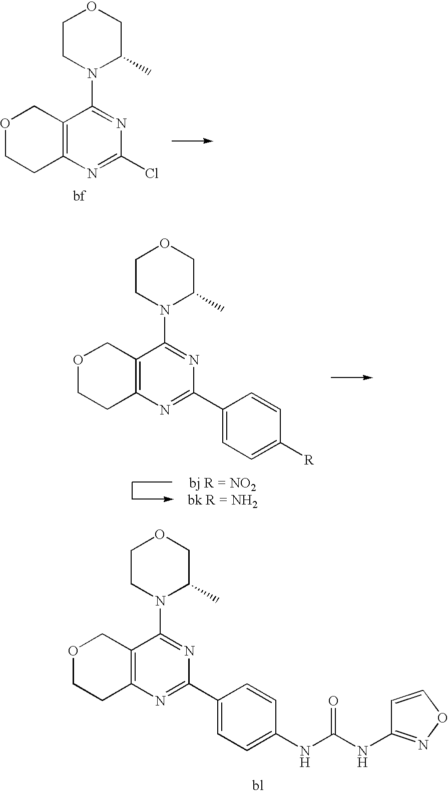

Step 1—Synthesis of (5)-4-(3-methylmorpholino)-2-(4-nitrophenyl)-7,8-dihydro-5H-pyrano[4,3-d]pyrimidine (bj): (S)-2-chloro-4-(3-methylmorpholino)-7,8-dihydro-5H-pyrano[4,3-d]pyrimidine (bf) (287.1 mg, 1.064 mmol), 4-nitrophenylboronic acid pinacol ester (314.1 mg, 1.261 mmol), sodium carbonate (338.4 mg, 3.193 mmol) and tetrakis(triphenylphosphine)palladium(0) (71.5 mg, 0.0619 mmol) were weighed into a microwave vial equipped with a stirbar. The vial was placed under atmospheric nitrogen pressure. Acetonitrile (3.0 mL, 58 mmol) and degassed water (3.0 mL, 170 mmol) were added and the mixture microwaved at 130° C. for 30 min. The reaction was diluted with 25 ml water and extracted with EtOAc (3×25 ml). The combined organics were dried with MgSO₄, filtered and concentrated onto silica gel. This crude material was purified by column chromatography using a 12 g column, with a gradient of 0% to 100% ethyl acetate in hexanes. The product-containing fractions were combined and evaporated under reduced pressure to give (5)-4-(3-methylmorpholino)-2-(4-nitrophenyl)-7,8-dihydro-5H-pyrano[4,3-d]pyrimidine (bj) as a yellow solid. ¹H NMR (400 MHz, CDCl₃) δ 8.57-8.52 (m, 2H), 8.29 (d, J=8.9, 2H), 4.63 (q, J=14.4, 2H), 4.21-3.67 (m, 7H), 3.56-3.49 (m, 2H), 3.08-2.99 (m, 2H), 1.36 (d, J=6.7, 3H).

Step 2—Synthesis of (5)-4-(4-(3-methylmorpholino)-7,8-dihydro-5H-pyrano[4,3-d]pyrimidin-2-yl)aniline (bk): A mixture of (5)-4-(3-methylmorpholino)-2-(4-nitrophenyl)-7,8-dihydro-5H-pyrano[4,3-d]pyrimidine (bj) (292 mg, 0.819 mmol) and stannous chloride, dihydrate (1.0166 g, 4.4654 mmol) in ethanol (15 mL, 260 mmol) was heated to 100° C. for 90 min. The reaction was concentrated in vacuo, diluted with H₂O, then basified with 1 N NaOH to pH=9-10. The aqueous phase was extracted with 10% MeOH/dichloromethane (3×30 mL), and the combined organics were dried over MgSO₄, filtered, and concentrated onto silica gel. This material was then subjected to column chromatography using a 12 g column, with a gradient of 0% to 100% ethyl acetate in hexanes. The product-containing fractions were combined and evaporated under reduced pressure to give (S)-4-(4-(3-methylmorpholino)-7,8-dihydro-5H-pyrano[4,3-d]pyrimidin-2-yl)aniline (bk). ¹H NMR (400 MHz, CDCl₃) δ 8.20 (d, J=8.6, 2H), 6.72 (d, J=8.6, 2H), 4.60 (dd, J=34.3, 14.1, 2H), 4.18-3.59 (m, 9H), 3.52-3.37 (m, 2H), 3.10-2.84 (m, 2H), 1.30 (d, J=6.8, 3H).

Step 3—Synthesis of (S)-1-(isoxazol-3-yl)-3-(4-(4-(3-methylmorpholino)-7,8-dihydro-5H-pyrano[4,3-d]pyrimidin-2-yl)phenyl)urea (bl): To a solution of (S)-4-(4-(3-methylmorpholino)-7,8-dihydro-5H-pyrano[4,3-d]pyrimidin-2-yl)aniline (bk) (86 mg, 0.26 mmol) in 1,2-dichloroethane (5.0 mL, 63 mmol) was added triethylamine (85 uL, 0.61 mmol). The solution was cooled to 0° C. and triphosgene (31.7 mg, 0.107 mmol) was added to the mixture in a single portion. A light colored precipitate formed rapidly. After 5 min at 0° C., the reaction was heated to 70° C. for 40 min. The reaction was then cooled to RT and 3-aminoisoxazole (1.00E2 uL, 1.35 mmol) was added in a single portion and stirred overnight at RT. The volatiles were removed under reduced pressure and the residue was purified by reverse phase HPLC to give (S)-1-(isoxazol-3-yl)-3-(4-(4-(3-methylmorpholino)-7,8-dihydro-5H-pyrano[4,3-d]pyrimidin-2-yl)phenyl)urea (bl). ¹H NMR (400 MHz, DMSO) δ 9.67 (s, 1H), 9.08 (s, 1H), 8.76 (d, J=1.7, 1H), 8.26 (d, J=8.8, 2H), 7.56 (d, J=8.8, 2H), 6.88 (d, J=1.7, 1H), 4.65-4.52 (m, 2H), 4.09-3.83 (m, 4H), 3.75-3.38 (m, 5H), 2.96-2.80 (m, 2H), 1.24 (d, J=6.6, 3H). LC-MS: m/z=+437.2 (M+H)⁺.

### Example 21

### Preparation of (S)-1-(1-methyl-1H-pyrazol-3-yl)-3-(4-(4-(3-methylmorpholino)-7,8-dihydro-5H-pyrano[4,3-d]pyrimidin-2-yl)phenyl)urea (bm)

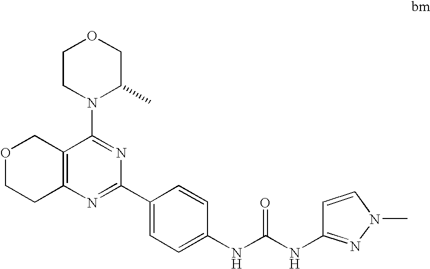

The title compound bm was prepared by the procedure described in Example 20, by substituting 3-aminoisoxazole with 1-methyl-1H-pyrazol-3-amine: ¹H NMR (400 MHz, DMSO) δ 9.17 (s, 1H), 8.97 (s, 1H), 8.24 (d, J=8.7 Hz, 2H), 7.60-7.50 (m, 3H), 6.24 (d, J=1.8 Hz, 1H), 4.58 (q, J=14.3 Hz, 2H), 4.08-3.81 (m, 4H), 3.74 (s, 3H), 3.73-3.37 (m, 5H), 2.91-2.81 (m, 2H), 1.24 (d, J=6.6 Hz, 3H). LC-MS: m/z=+450.2 (M+H)⁺.

### Example 22

### Preparation of (S)-1-(1-methyl-1H-pyrazol-4-yl)-3-(4-(4-(3-methylmorpholino)-7,8-dihydro-5H-pyrano[4,3-d]pyrimidin-2-yl)phenyl)urea (bn)

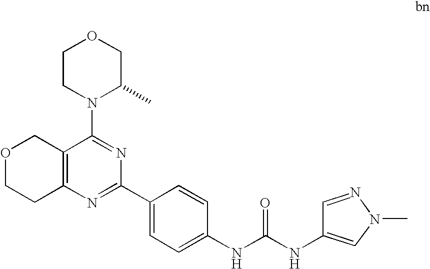

The title compound bn was prepared by the procedure described in Example 20, by substituting 3-aminoisoxazole with 1-methyl-1H-pyrazol-4-amine: ¹H NMR (400 MHz, DMSO) δ 8.85 (s, 1H), 8.42 (s, 1H), 8.22 (d, J=8.8 Hz, 2H), 7.77 (s, 1H), 7.53 (d, J=8.8 Hz, 2H), 7.38 (s, 1H), 4.58 (q, J=14.2 Hz, 2H), 4.07-3.82 (m, 4H), 3.78 (s, 3H), 3.74-3.37 (m, 5H), 2.94-2.77 (m, 2H), 1.24 (d, J=6.6 Hz, 3H). LC-MS: m/z=+450.2 (M+H)⁺.

### Example 23

### Preparation of (S)-1-(4-(4-(3-methylmorpholino)-7,8-dihydro-5H-pyrano[4,3-d]pyrimidin-2-yl)phenyl)-3-(2,2,2-trifluoroethyl)urea (bo)

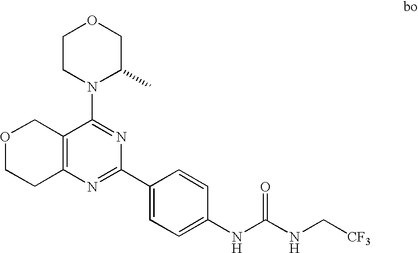

The title compound bo was prepared by the procedure described in Example 20, by substituting 3-aminoisoxazole with 2,2,2-trifluoroethanamine: ¹H NMR (400 MHz, DMSO) 1H NMR (400 MHz, DMSO) δ 9.00 (s, 1H), 8.21 (d, J=8.7 Hz, 2H), 7.51 (d, J=8.8 Hz, 2H), 6.82 (t, J=6.5 Hz, 1H), 4.64-4.51 (m, 2H), 4.07-3.82 (m, 6H), 3.73-3.37 (m, 5H), 2.93-2.79 (m, 2H), 1.23 (d, J=6.6 Hz, 3H). LC-MS: m/z=+452.2 (M+H)⁺.

### Example 24

### Preparation of (S)-1-(2-hydroxyethyl)-3-(4-(4-(3-methylmorpholino)-7,8-dihydro-5H-pyrano[4,3-d]pyrimidin-2-yl)phenyl)urea (bp)

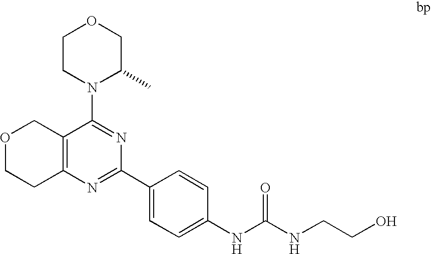

The title compound by was prepared by the procedure described in Example 20, by substituting 3-aminoisoxazole with ethanolamine: ¹H NMR (400 MHz, DMSO) ¹H NMR (400 MHz, DMSO) δ 8.81 (s, 1H), 8.18 (d, J=8.8 Hz, 2H), 7.47 (d, J=8.8 Hz, 2H), 6.28 (t, J=5.6 Hz, 1H), 4.82-4.71 (m, 1H), 4.64-4.50 (m, 2H), 4.08-3.82 (m, 4H), 3.74-3.37 (m, 7H), 3.17 (q, J=5.6 Hz, 2H), 2.93-2.77 (m, 2H), 1.23 (d, J=6.6 Hz, 3H). LC-MS: m/z=+414.2 (M+H)+.

### Example 25

### Preparation of (S)-1-(4-(4-(3-methylmorpholino)-7,8-dihydro-5H-pyrano[4,3-d]pyrimidin-2-yl)phenyl)-3-(oxetan-3-yl)urea (z)

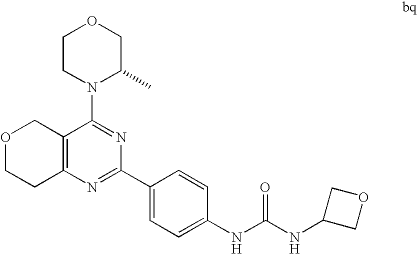

The title compound bq was prepared by the procedure described in Example 20, by substituting 3-aminoisoxazole with oxetan-3-amine: 1H NMR (400 MHz, DMSO) δ 8.79 (s, 1H), 8.19 (d, J=8.8 Hz, 2H), 7.48 (d, J=8.8 Hz, 2H), 6.97 (d, J=6.6 Hz, 1H), 4.83-4.68 (m, 3H), 4.63-4.51 (m, 2H), 4.44 (t, J=5.9 Hz, 2H), 4.07-3.82 (m, 4H), 3.73-3.37 (m, J=68.1, 29.7, 11.1, 2.7 Hz, 5H), 2.93-2.78 (m, 2H), 1.23 (d, J=6.6 Hz, 3H). LC-MS: m/z=+426.2 (M+H)+.

### Example 26

### Preparation of (S)-1-cyclobutyl-3-(4-(4-(3-methylmorpholino)-7,8-dihydro-5H-pyrano[4,3-d]pyrimidin-2-yl)phenyl)urea (br)

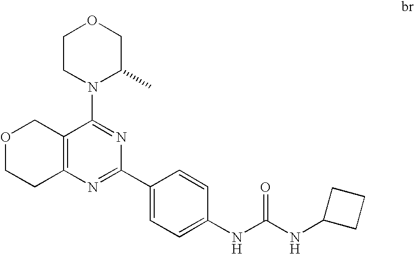

The title compound br was prepared by the procedure described in Example 20, by substituting 3-aminoisoxazole with cyclobutylamine: ¹H NMR (400 MHz, DMSO) δ 8.57 (s, 1H), 8.18 (d, J=8.8 Hz, 2H), 7.46 (d, J=8.8 Hz, 2H), 6.48 (d, J=8.0 Hz, 1H), 4.65-4.50 (m, 2H), 4.20-4.07 (m, 1H), 4.07-3.82 (m, 4H), 3.73-3.37 (m, 5H), 2.93-2.77 (m, 2H), 2.25-2.13 (m, 2H), 1.93-1.78 (m, 2H), 1.69-1.52 (m, 2H), 1.23 (d, J=6.6 Hz, 3H). LC-MS: m/z=+424.2 (M+H)+.

### Example 27

### Preparation of (S)-1-(5-methyl-1,3,4-oxadiazol-2-yl)-3-(4-(4-(3-methylmorpholino)-7,8-dihydro-5H-pyrano[4,3-d]pyrimidin-2-yl)phenyl)urea (bs)

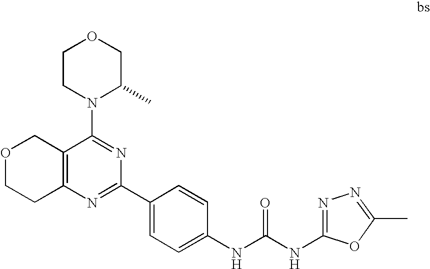

The title compound bs was prepared by the procedure described in Example 20, by substituting 3-aminoisoxazole with 5-methyl-1,3,4-oxadiazol-2-amine: ¹H NMR (400 MHz, DMSO) δ 9.73 (s, 1H), 8.23 (d, J=8.8 Hz, 2H), 7.63 (d, J=8.8 Hz, 2H), 4.66-4.50 (m, 2H), 4.15-3.82 (m, 4H), 3.75-3.41 (m, 5H), 3.17 (d, J=3.1 Hz, 1H), 2.94-2.78 (m, 2H), 2.38 (s, 3H), 1.24 (d, J=6.6 Hz, 3H). LC-MS: m/z=+452.2 (M+H)⁺.

### Example 28

### Preparation of (S)-2-(4-(4-(3-methylmorpholino)-7,8-dihydro-5H-pyrano[4,3-d]pyrimidin-2-yl)phenylamino)pyrimidin-4(3H)-one (bt)

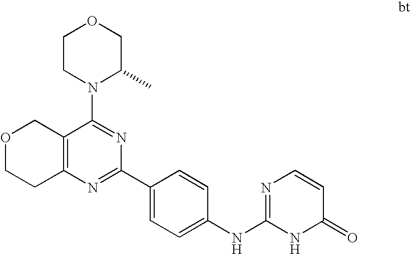

The title compound bt was prepared by the procedure described in Example 30, by substituting (S)-4-(4-(3-methylmorpholino)-6,7-dihydro-5H-pyrano[2,3-d]pyrimidin-2-yl)aniline (by) with (S)-4-(4-(3-methylmorpholino)-7,8-dihydro-5H-pyrano[4,3-d]pyrimidin-2-yl)aniline (bw): ¹H NMR (400 MHz, DMSO) δ 10.93 (br s, 1H), 9.20 (br s, 1H), 8.26 (d, J=8.6 Hz, 2H), 7.85-7.63 (m, 3H), 5.84 (br s, 1H), 4.58 (q, J=14.4 Hz, 2H), 4.07-3.82 (m, 4H), 3.70 (d, J=9.0 Hz, 1H), 3.64-3.35 (m, 4H), 2.93-2.82 (m, 2H), 1.25 (d, J=6.7 Hz, 3H). LC-MS: m/z=+421.1 (M+H)⁺.

### Example 29

### Preparation of (S)-6-(4-(4-(3-methylmorpholino)-7,8-dihydro-5H-pyrano[4,3-d]pyrimidin-2-yl)phenylamino)pyridin-2(1H)-one (bu)

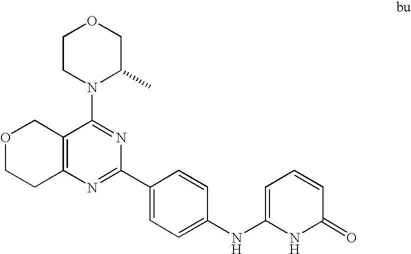

The title compound bu was prepared by the procedure described in Example 30, by substituting (S)-4-(4-(3-methylmorpholino)-6,7-dihydro-5H-pyrano[2,3-d]pyrimidin-2-yl)aniline (by) with (S)-4-(4-(3-methylmorpholino)-7,8-dihydro-5H-pyrano[4,3-d]pyrimidin-2-yl)aniline (bw) and by substituting 4-(benzyloxy)-2-chloropyrimidine with 2-(benzyloxy)-6-chloropyridine. Removal of the benzyl group in step 2 required 18 h reaction time: ¹H NMR (500 MHz, DMSO) δ 10.20 (br s, 1H), 9.10 (br s, 1H), 8.21 (d, J=8.8 Hz, 2H), 7.77 (br s, 2H), 7.42 (t, J=7.8 Hz, 1H), 6.31 (br s, 1H), 6.00 (d, J=7.9 Hz, 1H), 4.58 (q, J=14.3 Hz, 2H), 4.09-3.93 (m, 2H), 3.92-3.83 (m, 2H), 3.71 (dd, J=11.3, 2.6 Hz, 1H), 3.65-3.55 (m, 2H), 3.47 (d, J=13.3 Hz, 1H), 3.43-3.36 (m, 1H), 2.92-2.80 (m, 2H), 1.25 (d, J=6.7 Hz, 3H). LC-MS: m/z=+420.2 (M+H)⁺.

### Example 30

### Preparation of (S)-1-(1-methyl-1H-pyrazol-3-yl)-3-(4-(4-(3-methylmorpholino)-6,7-dihydro-5H-pyrano[2,3-d]pyrimidin-2-yl)phenyl)urea (bx)

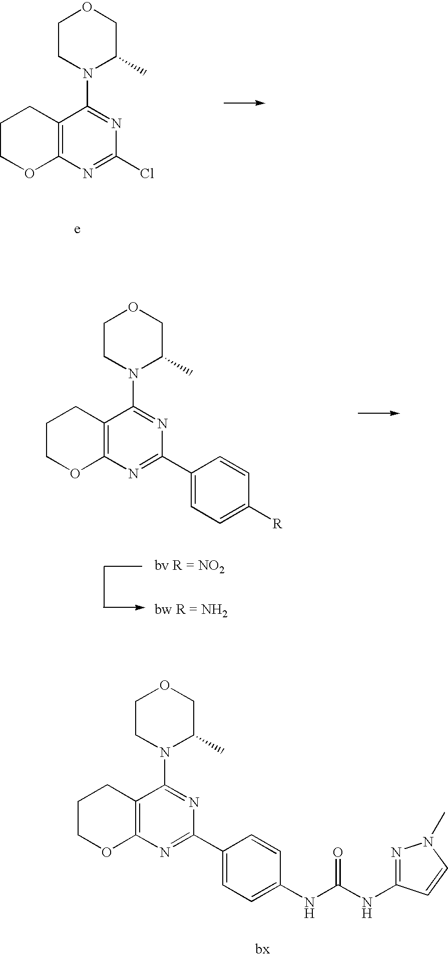

Step 1—Synthesis of (S)-4-(3-methylmorpholino)-2-(4-nitrophenyl)-6,7-dihydro-5H-pyrano[2,3-d]pyrimidine (by): (S)-2-chloro-4-(3-methylmorpholino)-6,7-dihydro-5H-pyrano[2,3-d]pyrimidine (e) (142.4 mg, 0.5279 mmol), and tetrakis(triphenylphosphine)palladium(0) (53.4 mg, 0.0462 mmol) were weighed into a microwave vial equipped with a stirbar. The atmosphere was evacuated and replaced with nitrogen 3 times. Acetonitrile (1.6 mL, 31 mmol) and degassed solutions of 1.00 M of Sodium carbonate in Water (0.80 mL) and 1.00 M of Potassium acetate in Water (0.80 mL) were added and the mixture was microwaved at 130° C. for 60 min. The reaction was diluted with 25 ml water and extracted with EtOAc (3×25 ml). The combined organics were dried with MgSO₄, filtered and concentrated onto silica gel. This material was then subjected to column chromatography using a 12 g column, with a gradient of 0% to 60% ethyl acetate in hexanes. The product containing fractions were combined and evaporated under reduced pressure to give (S)-4-(3-methylmorpholino)-2-(4-nitrophenyl)-6,7-dihydro-5H-pyrano[2,3-d]pyrimidine (by) as a yellow solid. ¹H NMR (500 MHz, CDCl₃) δ 8.54 (d, J=8.6, 2H), 8.27 (d, J=8.6, 2H), 4.43 (m, 2H), 4.10-3.40 (m, 10H), 2.74-2.48 (m, 3H), 2.01 (m, 3H), 1.36 (d, J=6.1, 3H).

Step 2—Synthesis of (S)-4-(4-(3-methylmorpholino)-6,7-dihydro-5H-pyrano[2,3-d]pyrimidin-2-yl)aniline (bw): A mixture of (S)-4-(3-methylmorpholino)-2-(4-nitrophenyl)-6,7-dihydro-5H-pyrano[2,3-d]pyrimidine (by) (104 mg, 0.292 mmol) and stannous chloride, dihydrate (0.373 g, 1.64 mmol) in ethanol (5.0 mL, 86 mmol) was heated to 100° C. for 90 min. The reaction was concentrated in vacuo, diluted with H₂O, then basified with 1N NaOH to pH=9-10. The aqueous phase was extracted with 10% MeOH/dichloromethane (3×30 mL), and the combined organics were dried over MgSO₄, filtered, and concentrated onto silica gel. This material was then subjected to column chromatography using a 4 g column, with a gradient of 0% to 70% ethyl acetate in hexanes. The product containing fractions were combined and evaporated under reduced pressure to give (S)-4-(4-(3-methylmorpholino)-6,7-dihydro-5H-pyrano[2,3-d]pyrimidin-2-yl)aniline (bw).

Step 3—Synthesis of (S)-1-(1-methyl-1H-pyrazol-3-yl)-3-(4-(4-(3-methylmorpholino)-6,7-dihydro-5H-pyrano[2,3-d]pyrimidin-2-yl)phenyl)urea (bx): (S)-4-(4-(3-methylmorpholino)-6,7-dihydro-5H-pyrano[2,3-d]pyrimidin-2-yl)aniline (50 mg, 0.153 mmol) and triethylamine (49 μl, 0.35 mmol, 2.3 eq) were dissolved in dichloroethane (2 mL) and cooled to 0° C. in a reaction vial. Triphosgene (15.9 mg, 0.053 mmol, 0.35 eq) was added in a single portion and stirred for 5 minutes at 0° C. The reaction was then warmed to 70° C. for 1 h, cooled to room temperature, and 1-methyl-3-aminopyrazole (5 eq) was added in a single portion. The reaction was stirred overnight at room temperature. The volatiles were then removed under reduced pressure and the material was purified by reverse phase HPLC to give (S)-1-(1-methyl-1H-pyrazol-3-yl)-3-(4-(4-(3-methylmorpholino)-6,7-dihydro-5H-pyrano[2,3-d]pyrimidin-2-yl)phenyl)urea (bx) (14.9 mg, 22%). ¹H NMR (400 MHz, DMSO) δ 9.20 (s, 1H), 8.99 (s, 1H), 8.17 (d, J=8.7, 2H), 7.59-7.48 (m, 3H), 6.23 (d, J=2.0, 1H), 4.39-4.21 (m, 2H), 4.02 (d, J=6.7, 1H), 3.86 (d, J=11.1, 1H), 3.78-3.36 (m, 8H), 2.58 (dd, J=15.8, 9.5, 2H), 1.89 (m, 1H), 1.24 (d, J=6.6, 3H). LC-MS: m/z=+450.2 (M+H)⁺.

### Example 31

### Preparation of (S)-1-(1-methyl-1H-pyrazol-4-yl)-3-(4-(4-(3-methylmorpholino)-6,7-dihydro-5H-pyrano[2,3-d]pyrimidin-2-yl)phenyl)urea (by)

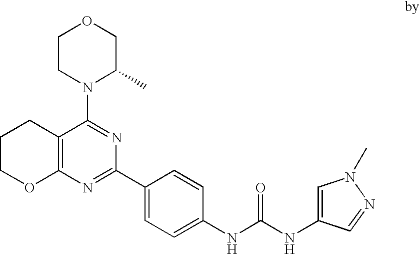

The title compound was prepared by the procedure described in Example 21, by substituting 1-methyl-3-aminopyrazole with 1-methyl-4-aminopyrazole: ¹H NMR (400 MHz, DMSO) δ 8.90 (s, 1H), 8.51 (s, 1H), 8.15 (d, J=8.8, 2H), 7.75 (s, 1H), 7.51 (d, J=8.8, 2H), 7.37 (s, 1H), 4.40-4.22 (m, 1H), 4.14-3.95 (m, 1H), 3.86 (d, J=11.1, OH), 3.78 (s, 3H), 3.71 (dd, J=11.2, 2.6, 1H), 3.67-3.37 (m, 4H), 3.17 (d, J=3.4, 1H), 2.59 (t, J=6.0, 2H), 1.98-1.74 (m, 2H), 1.24 (d, J=6.6, 3H). LC-MS: m/z=+450.2 (M+H)⁺.

### Example 32

### Preparation of (S)-1-(4-(4-(3-methylmorpholino)-6,7-dihydro-5H-pyrano[2,3-d]pyrimidin-2-yl)phenyl)-3-(oxetan-3-yl)urea (bz)

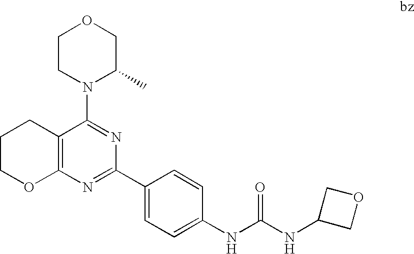

The title compound bz was prepared by the procedure described in Example 21, by substituting 1-methyl-3-aminopyrazole with 3-oxetanamine: ¹H NMR (400 MHz, DMSO) δ 8.78 (s, 1H), 8.12 (d, J=8.8, 2H), 7.46 (d, J=8.8, 2H), 7.00 (d, J=6.6, 1H), 4.83-4.67 (m, 3H), 4.44 (t, J=5.9, 2H), 4.38-4.22 (m, 2H), 4.05 (dd, J=32.8, 5.8, 1H), 3.85 (d, J=11.1, 1H), 3.74-3.35 (m, 5H), 3.17 (d, J=4.2, 1H), 2.57 (dd, J=13.9, 7.7, 2H), 1.96-1.74 (m, 2H), 1.23 (d, J=6.6, 3H). LC-MS: m/z=+426.2 (M+H)⁺.

### Example 33

### Preparation of (S)-1-(2-hydroxyethyl)-3-(4-(4-(3-methylmorpholino)-6,7-dihydro-5H-pyrano[2,3-d]pyrimidin-2-yl)phenyl)urea (ca)

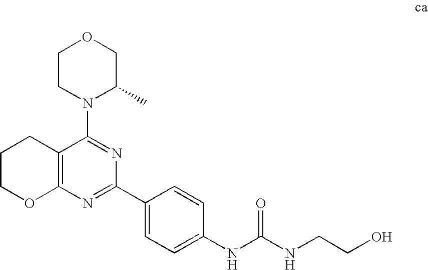

The title compound ca was prepared by the procedure described in Example 21, by substituting 1-methyl-3-aminopyrazole with ethanolamine: ¹H NMR (400 MHz, DMSO) δ 8.80 (s, 1H), 8.11 (d, J=8.7, 2H), 7.45 (d, J=8.8, 2H), 6.29 (t, J=5.6, 1H), 4.75 (s, 1H), 4.38-4.22 (m, 2H), 4.01 (d, J=6.6, 1H), 3.85 (d, J=11.3, 1H), 3.70 (dd, J=11.2, 2.6, 1H), 3.65-3.38 (m, 6H), 3.16 (q, J=5.8, 3H), 2.57 (dd, J=13.8, 7.6, 2H), 1.96-1.76 (m, 2H), 1.23 (d, J=6.6, 3H). LC-MS: m/z=+414.2 (M+H)⁺.

### Example 34

### Preparation of (S)-2-(4-(4-(3-methylmorpholino)-6,7-dihydro-5H-pyrano[2,3-d]pyrimidin-2-yl)phenylamino)pyrimidin-4(3H)-one (cc)

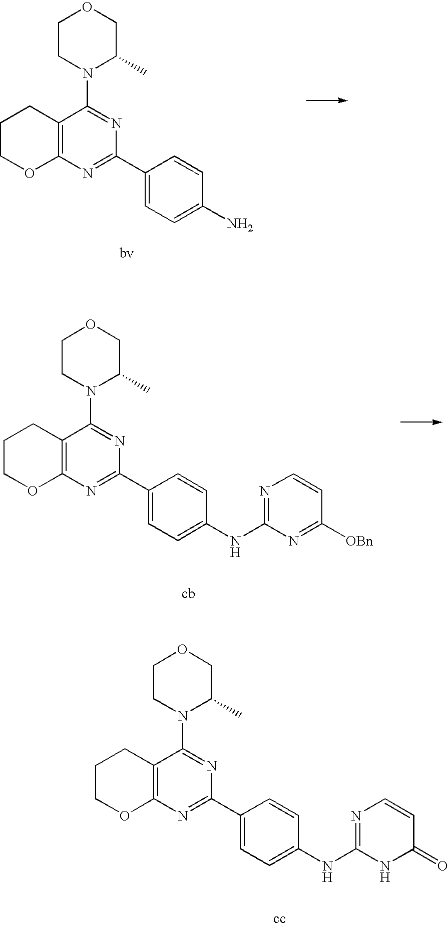

Step 1—Synthesis of (S)-4-(benzyloxy)-N-(4-(4-(3-methylmorpholino)-6,7-dihydro-5H-pyrano[2,3-d]pyrimidin-2-yl)phenyl)pyrimidin-2-amine (cb): (S)-4-(4-(3-methylmorpholino)-6,7-dihydro-5H-pyrano[2,3-d]pyrimidin-2-yl)aniline (ca) (68.6 mg1.00 equiv, 210.17 μmoles) was weighed into a microwave vial with a stirbar. 4-(benzyloxy)-2-chloropyrimidine (57.4 mg, 1.24 equiv, 260.13 μmoles), sodium t-butoxide (31.4 mg, 326.73 mmoles), bis(dibenzylideneacetone)palladium (8.6 mg14.96 μmoles) and 2-dicyclohexylphosphino-2′-(N,N-dimethylamino)biphenyl (7.3 mg18.55 μmoles) were added, then the reaction vial was evacuated and purged with N₂ 3 times. Toluene (2 mL) was added, and the reaction was heated in a CEM microwave at 120° C. for 40 minutes with PowerMax off. The reaction was then filtered through Celite, washing with CH₂Cl₂. The crude material was concentrated under reduced pressure onto silica gel, and was then purified using flash chromatography on a 4 g column using a gradient of 0% to 100% EtOAc in heptane. The product containing fractions were concentrated to give (S)-4-(benzyloxy)-N-(4-(4-(3-methylmorpholino)-6,7-dihydro-5H-pyrano[2,3-d]pyrimidin-2-yl)phenyl)pyrimidin-2-amine (cb) (77 mg0.72 equiv150.80 μmoles71.75% yield) as an oil. ¹H NMR (400 MHz, CDCl₃) δ 8.40-8.33 (m, 2H), 8.18 (d, J=5.7, 1H), 7.66 (d, J=8.8, 2H), 7.49-7.30 (m, 5H), 6.28 (d, J=5.7, 1H), 5.44 (s, 2H), 4.48-4.29 (m, 2H), 4.09-3.45 (m, 6H), 2.72-2.49 (m, 2H), 2.03-1.91 (m, 2H), 1.72-1.50 (m, 2H), 1.34 (d, J=6.7, 3H).

Step 2—Synthesis of (S)-2-(4-(4-(3-methylmorpholino)-6,7-dihydro-5H-pyrano[2,3-d]pyrimidin-2-yl)phenylamino)pyrimidin-4(3H)-one (cc): (S)-4-(benzyloxy)-N-(4-(4-(3-methylmorpholino)-6,7-dihydro-5H-pyrano[2,3-d]pyrimidin-2-yl)phenyl)pyrimidin-2-amine (cb) (77 mg1.00 equiv150.80 μmoles) was weighed into a 25 ml roundbottom flask equipped with a stirbar. Chloroform (3 mL37.39 mmoles) was added, followed by methanesulfonic Acid (1 mL15.25 mmoles) in a single portion. After 15 min, the reaction was diluted with dichloromethane and poured into 50 ml saturated NaHCO₃ and extracted with dichloromethane (4×20 ml). The combined organics were dried with MgSO₄, filtered and concentrated to an amber oil. This was purified by reverse phase HPLC to give (S)-2-(4-(4-(3-methylmorpholino)-6,7-dihydro-5H-pyrano[2,3-d]pyrimidin-2-yl)phenylamino)pyrimidin-4(3H)-one (cc). ¹H NMR (400 MHz, DMSO) δ 8.18 (d, J=8.8, 2H), 7.83-7.75 (m, 3H), 6.57 (s, 1H), 5.84 (d, J=6.3, 1H), 4.38-4.24 (m, 2H), 4.07-3.98 (m, 2H), 3.86 (d, J=10.9, 1H), 3.75-3.39 (m, 7H), 2.58 (t, J=11.5, 2H), 1.88 (s, 2H), 1.25 (d, J=6.6, 3H). LC-MS: m/z=+421.2 (M+H)⁺.

### Example 35

### Preparation of (S)-6-(4-(4-(3-methylmorpholino)-6,7-dihydro-5H-pyrano[2,3-d]pyrimidin-2-yl)phenylamino)pyridin-2(1H)-one (cd)

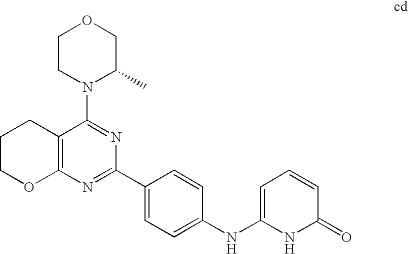

Synthesis of (S)-6-(4-(4-(3-methylmorpholino)-6,7-dihydro-5H-pyrano[2,3-d]pyrimidin-2-yl)phenylamino)pyridin-2(1H)-one (cd): The title compound cd was prepared by the procedure described in Example 34, by substituting 4-(benzyloxy)-2-chloropyrimidine with 2-(benzyloxy)-6-chloropyridine. Removal of the benzyl group in step 2 required 18 h: ¹H NMR (400 MHz, DMSO) δ 10.20 (s, 1H), 9.08 (s, 1H), 8.14 (d, J=8.8, 2H), 7.76 (s, 2H), 7.42 (t, J=7.9, 1H), 6.31 (s, 1H), 6.00 (d, J=7.5, 1H), 4.31 (ddd, J=15.5, 11.3, 7.7, 2H), 4.07-3.95 (m, 1H), 3.86 (d, J=11.2, 1H), 3.76-3.37 (m, 5H), 2.58 (t, J=5.8, 2H), 1.89 (s, 2H), 1.25 (d, J=6.6, 3H). LC-MS: m/z=+420.2 (M+H)⁺.

### Example 36

### Preparation of (S)-4-(3-methylmorpholino)-2-(4-(methylsulfonyl)phenyl)-7,8-dihydro-5H-pyrano[4,3-d]pyrimidine (ce)

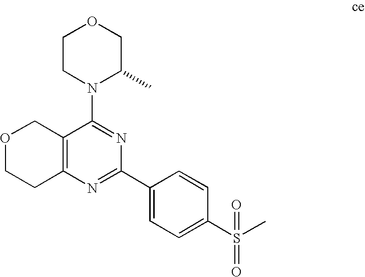

The title compound ce was prepared by the procedure described in Example 6, step 1 by substituting 4-nitrophenylboronic acid pinacol ester with 4-(methylsulfonyl)phenylboronic acid. The product was purified by reverse phase HPLC: ¹H NMR (400 MHz, DMSO) δ 8.53 (d, J=8.6 Hz, 2H), 8.03 (d, J=8.6 Hz, 2H), 4.62 (q, J=14.5 Hz, 2H), 4.09-3.94 (m, 3H), 3.89 (d, J=10.9 Hz, 1H), 3.74-3.38 (m, 5H), 3.25 (s, 3H), 2.92 (t, J=6.0 Hz, 2H), 1.27 (d, J=6.7 Hz, 3H). LC-MS: m/z=+390.1 (M+H)⁺.

### Example 37

### Synthesis of ((S)—N-methyl-4-(4-(3-methylmorpholino)-7,8-dihydro-5H-pyrano[4,3-d]pyrimidin-2-yl)benzenesulfonamide (cf)

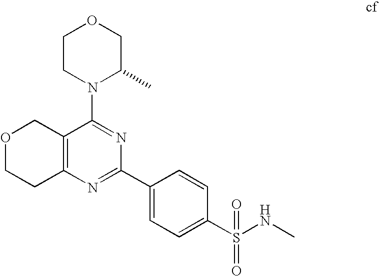

The title compound cf was prepared by the procedure described in Example 6, step 1 by substituting 4-nitrophenylboronic acid pinacol ester with 4-(N-methylsulfamoyl)phenylboronic acid. The product was purified by reverse phase HPLC: ¹H NMR (400 MHz, DMSO) δ 8.49 (d, J=8.4 Hz, 2H), 7.88 (d, J=8.5 Hz, 2H), 7.53 (q, J=4.9 Hz, 1H), 4.62 (q, J=14.5 Hz, 2H), 4.10-3.93 (m, 3H), 3.88 (d, J=11.4 Hz, 1H), 3.74-3.37 (m, 5H), 2.95-2.86 (m, 2H), 2.44 (d, J=4.9 Hz, 3H), 1.26 (d, J=6.7 Hz, 3H). LC-MS: m/z=+405.1 (M+H)+.

### Example 38

### Synthesis of (S)—N-(4-(4-(3-methylmorpholino)-7,8-dihydro-5H-pyrano[4,3-d]pyrimidin-2-yl)phenyl)methanesulfonamide (cg)

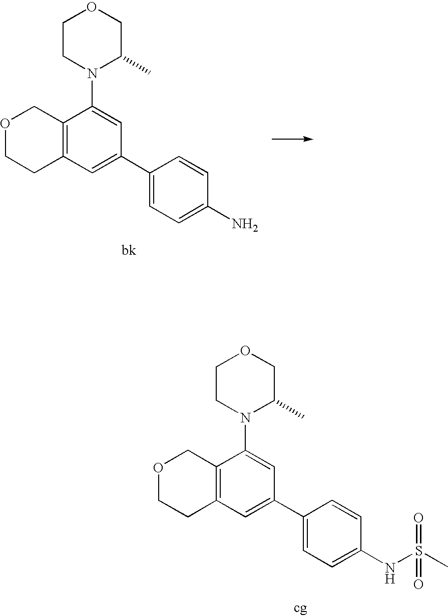

(S)-4-(4-(3-methylmorpholino)-7,8-dihydro-5H-pyrano[4,3-d]pyrimidin-2-yl)aniline (bk) (50.7 mg1.00 equiv, 155.33 μumoles) was weighed into a vial. And to it was added Dichloromethane (2 mL) followed by triethylamine (0.04 mL286.98 μumoles) and methanesulfonyl chloride (0.015 mL193.80 μumoles). The resultant solution was stirred overnight at RT. LC/MS analysis of the reaction mixture indicated that the aniline bk had been consumed. 2 ml 1M NaOH was added in a single portion, the layers were separated and the organic phase was extracted twice more with 2 ml 1M NaOH. The combined aqueous phases were acidified with concentrated HCl, then cooled to 4° C. The product cg crystallized out and was collected by filtration: ¹H NMR (400 MHz, DMSO) δ 10.21 (br s, 1H), 8.24 (d, J=8.6 Hz, 2H), 7.40-7.27 (m, 2H), 4.74-4.53 (m, 2H), 4.06-3.52 (m, 9H), 3.14-3.05 (m, 3H), 3.00-2.86 (m, 2H), 1.39-1.24 (m, 3H). LC-MS: m/z=+405.1 (M+H)+.

### Example 39

### Synthesis of (S)—N-(4-(4-(3-methylmorpholino)-7,8-dihydro-5H-pyrano[4,3-d]pyrimidin-2-yl)phenyl)cyclopropanesulfonamide (ch)

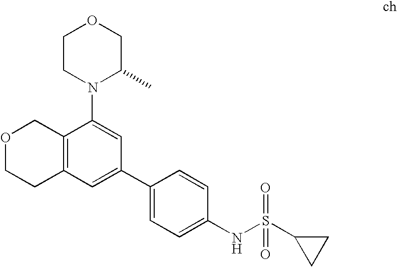

The title compound was prepared by the procedure described in Example 38, by substituting methanesulfonyl chloride with cyclopropylsulfonyl chloride. The product was purified by reverse phase HPLC: ¹H NMR (400 MHz, DMSO) δ 10.22 (br s, 1H), 8.23 (d, J=8.8 Hz, 2H), 7.38 (d, J=8.7 Hz, 2H), 4.76-4.57 (m, 2H), 3.99 (t, J=6.1 Hz, 2H), 3.91 (d, J=7.9 Hz, 1H), 3.68 (s, 2H), 3.62-3.52 (m, 3H), 3.01-2.89 (m, 2H), 2.82-2.69 (m, 1H), 1.33 (d, J=6.5 Hz, 3H), 1.07-0.92 (m, 4H). LC-MS: m/z=+431.1 (M+H)+.

### Example 40

### Preparation of (S)—N-(4-(4-(3-methylmorpholino)-7,8-dihydro-5H-pyrano[4,3-d]pyrimidin-2-yl)phenyl)ethanesulfonamide (ci)

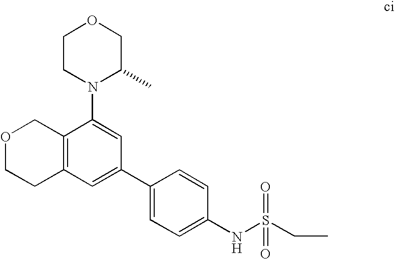

The title compound was prepared by the procedure described in Example 38, by substituting methanesulfonyl chloride with ethanesulfonyl chloride. The product was purified by reverse phase HPLC: ¹H NMR (400 MHz, DMSO) δ 10.24 (br s, 1H), 8.23 (d, J=8.7 Hz, 2H), 7.35 (d, J=8.6 Hz, 2H), 4.66 (dd, J=31.8, 14.6 Hz, 2H), 3.99 (t, J=6.0 Hz, 2H), 3.90 (d, J=8.8 Hz, 1H), 3.68 (s, 2H), 3.55 (dd, J=20.9, 11.6 Hz, 3H), 3.20 (q, J=7.1 Hz, 2H), 3.00-2.87 (m, 2H), 1.32 (d, J=6.1 Hz, 3H), 1.21 (t, J=7.3 Hz, 3H). LC-MS: m/z=+419.1 (M+H)+.

### Example 41

### Preparation of 1-ethyl-3-(4-(4′-morpholino-5′,6′-dihydro spiro[cyclopropane-1,7′-pyrano[2,3-d]pyrimidine]-2′-yl)phenyl)urea (cp)

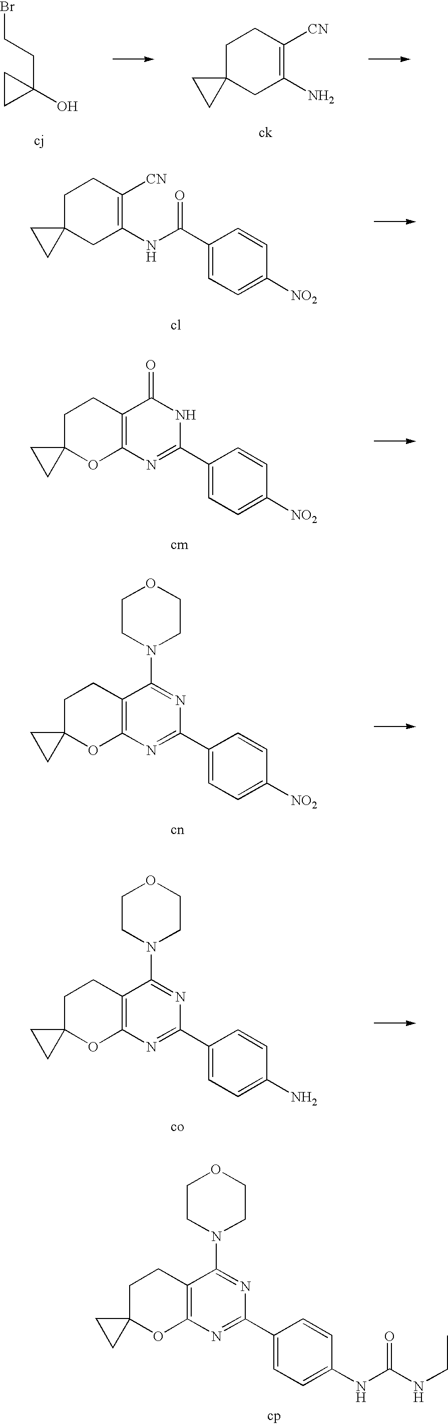

Step 1—Synthesis of cj: 1-(2-bromoethyl)cyclopropanol (cj) was prepared according to the procedure outlined in Eur. J. Org. Chem. 2003, 551-561.

Step 2—Synthesis of 5-amino-4-oxaspiro[2.5]oct-5-ene-6-carbonitrile (ck): Ethanol (30 mL, 0.5 mol) was cooled to 0° C., and then sodium metal (1.526 g, 0.06638 mol) was added and stirred until dissolved. Malononitrile (4.20 mL, 0.0667 mol) was then added in 5 portions over 5 minutes to yield a milky white suspension. This suspension was then warmed to 40° C., and 1-(2-bromoethyl)cyclopropanol (8.48 g, 0.0514 mol) was dissolved in 5 ml EtOH, and added dropwise over 15 min to the reaction solution. The reaction solution was stirred 2 h at 40° C., and then NaBr precipitates were filtered off. The resulting solution was concentrated to an orange-ish oil and poured into ice water. NaCl was added to salt out the product, which came out of solution as a thick oil, which was filtered off. The filtrate also showed some of the oil present, and was extracted with EtOAc (3×100 ml). The solids were dissolved the organic extracts and the resulting dark orange solution was dried with MgSO₄, filtered and concentrated onto silica gel. The crude product was purified by column chromatography using a 120 g column, with a gradient of 0% to 40% ethyl acetate in heptane. The product containing fractions were combined and evaporated under reduced pressure to give 5-amino-4-oxaspiro[2.5]oct-5-ene-6-carbonitrile (2.80 g, 36%) as a light yellow solid. 1H NMR (500 MHz, CDCl₃) δ 4.35 (br s, 2H), 2.34 (t, J=6.3 Hz, 2H), 1.77 (t, J=6.3 Hz, 2H), 1.02-0.92 (m, 2H), 0.65-0.54 (m, 2H).

Step 3—Synthesis of N-(6-cyano-4-oxaspiro[2.5]oct-5-en-5-yl)-4-nitrobenzamide (cl): 5-amino-4-oxaspiro[2.5]oct-5-ene-6-carbonitrile (ck) (2.744 g, 0.01827 mol) was weighed into a flask, and then dissolved in methylene chloride (50 mL, 0.8 mol). To the reaction solution was added triethylamine (7.9 mL, 0.057 mol), followed by p-nitrobenzoyl chloride (8.526 g, 0.04595 mol) in a single portion. The reaction solution immediately became orange-yellow. The reaction mixture was stirred at RT overnight, and turned a dark brown color. The reaction mixture was filtered to remove TEA-HCl, which was washed with 1:1 hexane/CH₂Cl₂. The filtrate was concentrated and dissolved in tetrahydrofuran (50 mL, 0.6 mol), and 3.00 M of Sodium hydroxide in water (15 mL) was added and heated to reflux for 1 h. The reaction mixture was then cooled and diluted with water and EtOAc. The aqueous phase was extracted with EtOAc (3×100 ml), the combined organics were washed with 1N HCl (1×100 ml), dried with MgSO₄, filtered and concentrated onto silica gel. This material was purified by column chromatography using a 40 g column, with a gradient of 0% to 60% ethyl acetate in hexanes. The product containing fractions were combined and evaporated under reduced pressure to give N-(6-cyano-4-oxaspiro[2.5]oct-5-en-5-yl)-4-nitrobenzamide (cl). ¹H NMR (400 MHz, DMSO) δ 10.88 (s, 1H), 8.36 (d, J=8.8 Hz, 2H), 8.09 (d, J=8.8 Hz, 2H), 2.47 (t, J=6.3 Hz, 2H), 1.87 (t, J=6.3 Hz, 2H), 0.96 (t, J=6.2 Hz, 2H), 0.74 (t, J=6.4 Hz, 2H).

Step 4—Synthesis of 2′-(4-nitrophenyl)-5′,6′-dihydrospiro[cyclopropane-1,7′-pyrano[2,3-d]pyrimidin]-4′(3′H)-one (cm): N-(6-cyano-4-oxaspiro[2.5]oct-5-en-5-yl)-4-nitrobenzamide (c1) (4.30 g, 0.0144 mol) and benzoic acid (1.904 g, 0.01559 mol) were weighed into a reaction vial equipped with a stirbar. Ethyl orthoformate (50 mL, 0.30 mol) was added to the reaction mixture and the vial was sealed and flushed with N₂, then heated to 145° C. overnight. The reaction was cooled and the volatiles were removed under reduced pressure. The resulting solid material was suspended in hot CH₂Cl₂, cooled to 4° C., filtered and washed with cold CH₂Cl₂ to give 2′-(4-nitrophenyl)-5′,6′-dihydrospiro[cyclopropane-1,7′-pyrano[2,3-d]pyrimidin]-4′(3′H)-one. ¹H NMR (400 MHz, DMSO) δ 12.71 (br s, 1H), 8.32 (s, 4H), 2.57 (t, J=6.2 Hz, 2H), 1.90 (t, J=6.3 Hz, 2H), 1.03-0.95 (m, 2H), 0.76-0.68 (m, 2H).

Step 5a—Synthesis of 4′-morpholino-2′-(4-nitrophenyl)-5′,6′-dihydrospiro[cyclopropane-1,7′-pyrano[2,3-d]pyrimidine] (cn): 2′-(4-nitrophenyl)-5′,6′-dihydrospiro[cyclopropane-1,7′-pyrano[2,3-d]pyrimidin]-4′(3′H)-one (cm) (2.96 g, 0.00989 mol) was suspended in phosphoryl chloride (30 mL, 0.3 mol) and heated to 100° C. under a nitrogen atmosphere for 6 h. The reaction mixture was cooled, then the volatiles were removed under reduced pressure. The residual slurry was poured into 200 ml ice, stirring until all the ice has melted. The tan solids that formed were filtered off and washed with 100 ml water. The resulting 4′-chloro-2′-(4-nitrophenyl)-5′,6′-dihydrospiro[cyclopropane-1,7′-pyrano[2,3-c]pyrimidine] could be used in subsequent reactions without further purification.

¹H NMR (400 MHz, DMSO) δ 8.47 (d, J=8.9 Hz, 2H), 8.34 (d, J=8.9 Hz, 2H), 2.91 (t, J=6.4 Hz, 2H), 2.06 (t, J=6.4 Hz, 2H), 1.12-1.05 (m, 2H), 0.86-0.78 (m, 2H).

Step 5b—4′-chloro-2′-(4-nitrophenyl)-5′,6′-dihydrospiro[cyclopropane-1,7′-pyrano[2,3-c]pyrimidine] (0.785 g, 0.00247 mol) was weighed into a 25 ml roundbottom flask equipped with a stirbar. N,N-dimethylformamide (10 mL, 0.1 mol) and N,N-diisopropylethylamine (0.650 mL, 0.00373 mol) were added, followed by morpholine (0.26 mL, 0.0030 mol). The reaction mixture was heated to 80° C. for 4 h. The reaction mixture was cooled, resulted in precipitation of the crude product. This mixture was poured into 200 ml water, filtered and washed with 100 ml water to provide 4′-morpholino-2′-(4-nitrophenyl)-5′,6′-dihydrospiro[cyclopropane-1,7′-pyrano[2,3-d]pyrimidine] (cn) as a light yellow powder. ¹H NMR (400 MHz, DMSO) δ 8.48 (d, J=8.8 Hz, 2H), 8.30 (d, J=8.9 Hz, 2H), 3.81-3.72 (m, 4H), 3.57-3.47 (m, 4H), 2.77 (t, J=5.9 Hz, 2H), 1.89 (t, J=5.9 Hz, 2H), 1.07-0.99 (m, 2H), 0.79-0.73 (m, 2H).

Step 6—Synthesis of 4-(4′-morpholino-5′,6′-dihydrospiro[cyclopropane-1,7′-pyrano[2,3-d]pyrimidine]-2′-yl)aniline (co): 4′-morpholino-2′-(4-nitrophenyl)-5′,6′-dihydrospiro[cyclopropane-1,7′-pyrano[2,3-c]pyrimidine] (90.9 mg, 0.247 mmol) and tin dichloride (236 mg, 1.23 mmol) were weighed into a reaction vial. Ethanol (3 mL, 0.05 mol) was added, and the reaction was stirred and heated to 100° C. for 2 h. LC/MS analysis of the crude reaction mixtures showed that reaction is complete. The volatiles were removed under reduced pressure, and then diluted with water (25 ml) and basified with 1N NaOH to pH 9-10. The aqueous phase was extracted using gentle shaking to avoid emulsions with 10% MeOH in dichloromethane (3×25 ml), and the combined organics were dried over MgSO₄, filtered and concentrated onto silica gel. This material was then subjected to column chromatography using a 4 g column, with a gradient of 0% to 50% ethyl acetate in hexanes. The product containing fractions were combined and evaporated under reduced pressure to give 4-(4′-morpholino-5′,6′-dihydrospiro[cyclopropane-1,7′-pyrano[2,3-d]pyrimidine]-2′-yl)aniline (co). ¹H NMR (400 MHz, DMSO) δ 7.94 (d, J=8.6 Hz, 2H), 6.56 (d, J=8.6 Hz, 2H), 5.49 (s, 2H), 3.78-3.69 (m, 4H), 3.43-3.36 (m, 4H), 2.68 (t, J=6.0 Hz, 2H), 1.84 (t, J=5.9 Hz, 2H), 1.02-0.94 (m, 2H), 0.76-0.66 (m, 2H).

Step 7—Synthesis of 1-ethyl-3-(4-(4′-morpholino-5′,6′-dihydrospiro[cyclopropane-1,7′-pyrano[2,3-d]pyrimidine]-2′-yl)phenyl)urea (cp): 4-(4′-morpholino-5′,6′-dihydrospiro[cyclopropane-1,7′-pyrano[2,3-d]pyrimidine]-2′-yl)aniline (co) (64 mg, 1.9 mmol) was dissolved in N,N-dimethylformamide (0.7 mL, 9 mmol). Ethyl isocyanate (25 uL, 3.2 mmol) was added in a single portion, and the reaction warmed to 50° C. overnight. After 18 h, LC/MS analysis of the reaction mixture indicates that the reaction is only partially complete. An additional 25 uL ethyl isocyanate (0.32 mmol, 1.7 eq) was added to the reaction mixture and the temperature was increased to 60° C. and stirred overnight. This crude reaction mixture was then purified by reverse phase HPLC to provide the desired product (cp): ¹H NMR (400 MHz, DMSO) δ 8.66 (s, 1H), 8.11 (d, J=8.7 Hz, 2H), 7.45 (d, J=8.8 Hz, 2H), 6.20 (t, J=5.5 Hz, 1H), 3.80-3.68 (m, 4H), 3.51-3.38 (m, 4H), 3.16-3.05 (m, 2H), 2.71 (t, J=6.0 Hz, 2H), 1.86 (t, J=5.8 Hz, 2H), 1.06 (t, J=7.2 Hz, 3H), 1.01 (t, J=6.0 Hz, 2H), 0.73 (t, J=6.3 Hz, 2H). LC-MS: m/z=+410.2 (M+H)+.

### Example 42

### Preparation of 2-(4-(4′-morpholino-5′,6′-dihydrospiro[cyclopropane-1,7′-pyrano[2,3-d]pyrimidine]-2′-yl)phenylamino)pyrimidin-4(3H)-one (cr)

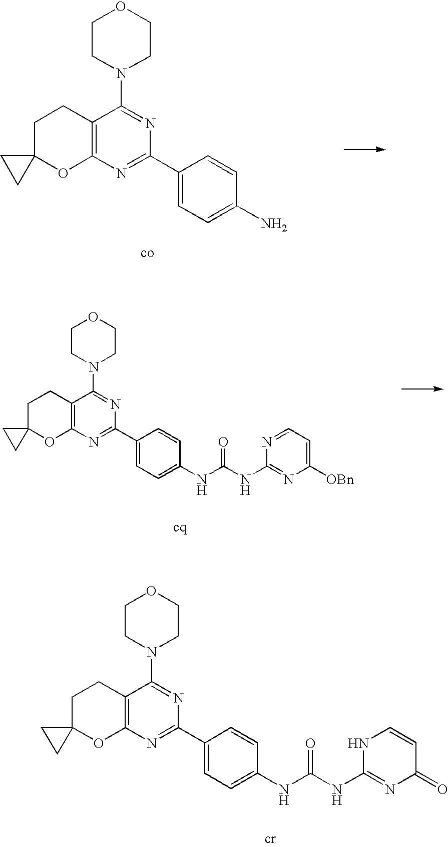

Step 1—Synthesis of 4-(benzyloxy)-N-(4-(4′-morpholino-5′,6′-dihydrospiro[cyclopropane-1,7′-pyrano[2,3-d]pyrimidine]-2′-yl)phenyl)pyrimidin-2-amine (cq): 4-(4′-morpholino-5′,6′-dihydrospiro[cyclopropane-1,7′-pyrano[2,3-d]pyrimidine]-2′-yl)aniline (co) (79.5 mg, 0.235 mmol), 4-(benzyloxy)-2-chloropyrimidine (63.4 mg, 0.287 mmol), bis(dibenzylideneacetone)palladium(0) (8.2 mg, 0.014 mmol), sodium tert-butoxide (35.8 mg, 0.372 mmol), and 2-dicyclohexylphosphino-2′-(N,N-dimethylamino)biphenyl (9.8 mg, 0.025 mmol) were weighed into a microwave vial. The vial was evacuated and purged 3× with N₂, then degassed toluene (2.1 mL, 2.0E1 mmol) was added and the vial sealed. The reaction was microwaved at 120° C. for 20 min. The reaction mixture was filtered through Celite, washing extensively with CH₂Cl₂. This was then concentrated onto silica gel and subjected to column chromatography using a 12 g column, with a gradient of 0% to 100% ethyl acetate in hexanes. The product containing fractions were combined and evaporated under reduced pressure to give 4-(benzyloxy)-N-(4-(4′-morpholino-5′,6′-dihydrospiro[cyclopropane-1,7′-pyrano[2,3-d]pyrimidine]-2′-yl)phenyl)pyrimidin-2-amine: ¹H NMR (400 MHz, DMSO) δ 8.75 (s, 1H), 8.12 (d, J=8.7 Hz, 2H), 7.45 (d, J=8.8 Hz, 2H), 6.98 (d, J=6.5 Hz, 1H), 4.81-4.67 (m, 3H), 4.44 (t, J=5.7 Hz, 2H), 3.79-3.69 (m, 4H), 3.48-3.40 (m, 4H), 2.71 (t, J=5.9 Hz, 2H), 1.86 (t, J=5.9 Hz, 2H), 1.01 (t, J=6.0 Hz, 2H), 0.73 (t, J=6.3 Hz, 2H).

Step 2—Synthesis of 2-(4-(4′-morpholino-5′,6′-dihydrospiro[cyclopropane-1,7′-pyrano[2,3-d]pyrimidine]-2′-yl)phenylamino)pyrimidin-4(3H)-one (cr): To stirred solution of 4-(benzyloxy)-N-(4-(4′-morpholino-5′,6′-dihydrospiro[cyclopropane-1,7′-pyrano[2,3-d]pyrimidine]-2′-yl)phenyl)pyrimidin-2-amine (cq) (88.3 mg, 0.169 mmol) in chloroform (3.00 mL, 37.5 mmol) was added methanesulfonic acid (1.00 mL, 15.4 mmol) in a single portion. The reaction was stirred 1 h at RT. The reaction was then diluted with CH₂Cl₂ and quenched with a saturated aqueous solution of NaHCO₃. The layers were separated and the aqueous phase extracted with CH₂Cl₂ (3×25 ml). The combined organics were dried with MgSO₄, filtered and concentrated. The crude material was purified by reverse phase HPLC to give 2-(4-(4′-morpholino-5′,6′-dihydrospiro[cyclopropane-1,7′-pyrano[2,3-d]pyrimidine]-2′-yl)phenylamino)pyrimidin-4(3H)-one (cr): ¹H NMR (400 MHz, DMSO) δ 10.78 (br s, 1H), 9.02 (br s, 1H), 8.20 (d, J=8.6 Hz, 2H), 7.87-7.57 (m, 3H), 5.86 (br s, 1H), 3.79-3.70 (m, 4H), 3.51-3.42 (m, 4H), 2.76-2.62 (m, 2H), 1.90-1.82 (m, 2H), 1.05-0.98 (m, 2H), 0.77-0.69 (m, 2H). LC-MS: m/z=+433.1 (M+H)+.

### Example 43

### Preparation of 1-(4-(4′-morpholino-5′,6′-dihydrospiro[cyclopropane-1,7′-pyrano[2,3-d]pyrimidine]-2′-yl)phenyl)-3-(oxetan-3-yl)urea (cs)

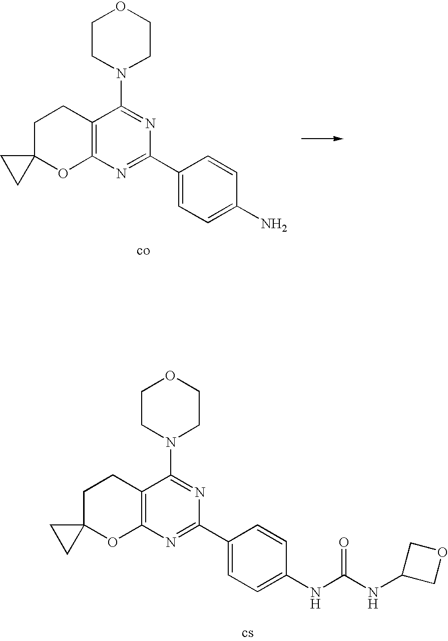

To a solution of 4-(4′-morpholino-5′,6′-dihydrospiro[cyclopropane-1,7′-pyrano[2,3-d]pyrimidine]-2′-yl)aniline (co) (56 mg, 0.16 mmol) in 1,2-dichloroethane (2.0 mL) was added triethylamine (55 uL, 0.39 mmol). The solution was cooled to 0° C. and triphosgene (23.9 mg, 0.0805 mmol) was added to the mixture in a single portion. A light colored precipitate formed rapidly. After 5 min at 0° C., the reaction was heated to 70° C. for 40 min. The reaction was then cooled to RT and 3-oxetanamine (45.0 mg, 0.616 mmol) was added in a single portion and stirred overnight at RT. H₂O (5 ml) was added to the reaction mixture and then the aqueous phase was extracted with CH₂Cl₂ (5×2 ml). The combined organics were concentrated under reduced pressure, then purified by reverse phase HPLC: ¹H NMR (400 MHz, DMSO) δ 8.75 (s, 1H), 8.12 (d, J=8.7 Hz, 2H), 7.45 (d, J=8.8 Hz, 2H), 6.98 (d, J=6.5 Hz, 1H), 4.81-4.67 (m, 3H), 4.44 (t, J=5.7 Hz, 2H), 3.79-3.69 (m, 4H), 3.48-3.40 (m, 4H), 2.71 (t, J=5.9 Hz, 2H), 1.86 (t, J=5.9 Hz, 2H), 1.01 (t, J=6.0 Hz, 2H), 0.73 (t, J=6.3 Hz, 2H). LC-MS: m/z=+438.2 (M+H)+.

### Example 44

### Synthesis of 1-(1-methyl-1H-pyrazol-3-yl)-3-(4-(4′-morpholino-5′,6′-dihydrospiro[cyclopropane-1,7′-pyrano[2,3-d]pyrimidine]-2′-yl)phenyl)urea (ct)

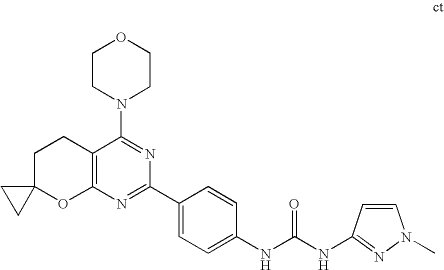

The title compound ct was prepared by the procedure described in Example 43, by substituting 3-oxetanamine with 1-methyl-1H-pyrazol-3-amine: ¹H NMR (400 MHz, DMSO) δ 9.16 (br s, 1H), 8.95 (s, 1H), 8.17 (d, J=8.7 Hz, 2H), 7.56-7.49 (m, 3H), 6.23 (d, J=1.8 Hz, 1H), 3.78-3.70 (m, 7H), 3.49-3.42 (m, 4H), 2.72 (t, J=6.0 Hz, 2H), 1.86 (t, J=6.1 Hz, 2H), 1.05-0.98 (m, 2H), 0.78-0.70 (m, 2H). LC-MS: m/z=+462.2 (M+H)+.

### Example 45

### Preparation of 1-(1-methyl-1H-pyrazol-4-yl)-3-(4-(4′-morpholino-5′,6′-dihydrospiro[cyclopropane-1,7′-pyrano[2,3-d]pyrimidine]-2′-yl)phenyl)urea (cu)

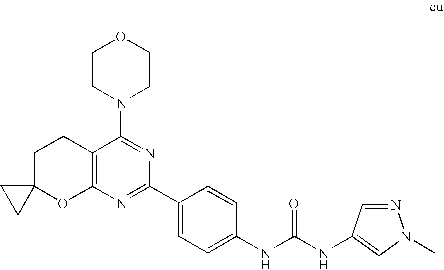

The title compound was prepared by the procedure described in Example 43, by substituting 3-oxetanamine with 1-methyl-1H-pyrazol-4-amine: ¹H NMR (400 MHz, DMSO) δ 8.85 (s, 1H), 8.46 (s, 1H), 8.15 (d, J=8.7 Hz, 2H), 7.75 (s, 1H), 7.51 (d, J=8.8 Hz, 2H), 7.37 (s, 1H), 3.78 (s, 3H), 3.76-3.70 (m, 4H), 3.49-3.41 (m, 4H), 2.72 (t, J=5.9 Hz, 2H), 1.86 (t, J=5.8 Hz, 2H), 1.01 (t, J=5.9 Hz, 2H), 0.74 (t, J=6.2 Hz, 2H). LC-MS: m/z=+462.2 (M+H)+.

### Example 46

### Preparation of 1-(4-(4′-(4-methoxypiperidin-1-yl)-5′,6′-dihydrospiro[cyclopropane-1,7′-pyrano[2,3-d]pyrimidine]-2′-yl)phenyl)-3-(oxetan-3-yl)urea (cv)

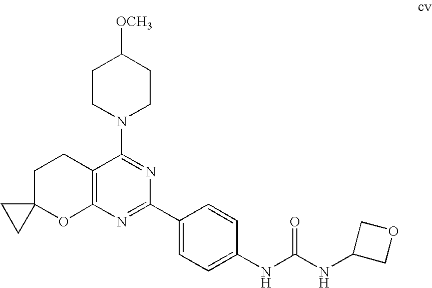

The title compound was prepared by the procedure described in Example 43, by substituting morpholine with 4-methoxypiperidine: ¹H NMR (400 MHz, DMSO) δ 8.72 (s, 1H), 8.11 (d, J=8.7 Hz, 2H), 7.45 (d, J=8.8 Hz, 2H), 6.95 (d, J=6.5 Hz, 1H), 4.82-4.68 (m, 3H), 4.44 (t, J=5.8 Hz, 2H), 3.82-3.67 (m, 2H), 3.51-3.38 (m, 1H), 3.22-3.11 (m, 2H), 2.70 (t, J=5.9 Hz, 2H), 2.03-1.92 (m, 2H), 1.84 (t, J=5.8 Hz, 2H), 1.62-1.48 (m, 2H), 1.05-0.95 (m, J=6.0 Hz, 2H), 0.77-0.68 (m, J=6.2 Hz, 2H). Note: water signal masks OMe singlet. LC-MS: m/z=+466.2 (M+H)+.

### Example 47

### Preparation of 1-(4-(4′-(4-methoxypiperidin-1-yl)-5′,6′-dihydrospiro[cyclopropane-1,7′-pyrano[2,3-d]pyrimidine]-2′-yl)phenyl)-3-(1-methyl-1H-pyrazol-3-yl)urea (cw)

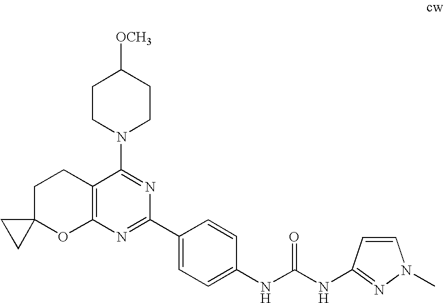

The title compound was prepared by the procedure described in Example 44, by substituting morpholine with 4-methoxypiperidine: ¹H NMR (400 MHz, DMSO) δ 9.16 (br s, 1H), 8.95 (s, 1H), 8.16 (d, J=8.7 Hz, 2H), 7.53 (s, 1H), 7.52 (d, J=8.7 Hz, 2H), 6.23 (d, J=2.1 Hz, 1H), 3.80-3.68 (m, 2H), 3.74 (s, 3H), 3.50-3.39 (m, 1H), 3.17 (t, J=10.2 Hz, 2H), 2.71 (t, J=5.8 Hz, 2H), 2.04-1.92 (m, J=11.6 Hz, 2H), 1.86 (t, J=5.6 Hz, 2H), 1.63-1.50 (m, 1H), 1.04-0.96 (m, 1H), 0.74 (t, 2H). Note: water signal masks OMe singlet. LC/MS: m/z=+490.2 (M+H)+.

### Example 48

### Preparation of 2-(4-(4′-(4-methoxypiperidin-1-yl)-5′,6′-dihydrospiro[cyclopropane-1,7′-pyrano[2,3-d]pyrimidine]-2′-yl)phenylamino)pyrimidin-4(3H)-one (cx)

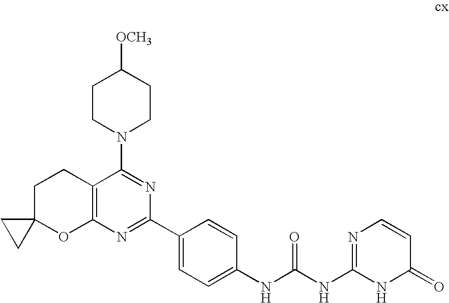

The title compound cx was prepared by the procedure described in Example 42, by substituting morpholine with 4-methoxypiperidine: ¹H NMR (400 MHz, DMSO) δ 9.24 (br s, 1H), 8.18 (d, J=8.7 Hz, 2H), 7.80 (br s, 1H), 7.72 (d, J=8.0 Hz, 2H), 5.86 (br s, 1H), 3.83-3.67 (m, 2H), 3.50-3.39 (m, 2H), 3.18 (t, J=10.2 Hz, 2H), 2.71 (t, J=5.8 Hz, 2H), 2.04-1.93 (m, 2H), 1.85 (t, J=5.7 Hz, 2H), 1.63-1.50 (m, 2H), 1.05-0.98 (m, 2H), 0.77-0.69 (m, 2H). Note: water signal masks OMe singlet. LC/MS: m/z=+461.2 (M+H)+.

### Example 49

### Preparation of (5)-1-ethyl-3-(4-(4′-(3-methylmorpholino)-5′,6′-dihydro spiro[cyclopropane-1,7′-pyrano[2,3-d]pyrimidine]-2′-yl)phenyl)ure a (cz)

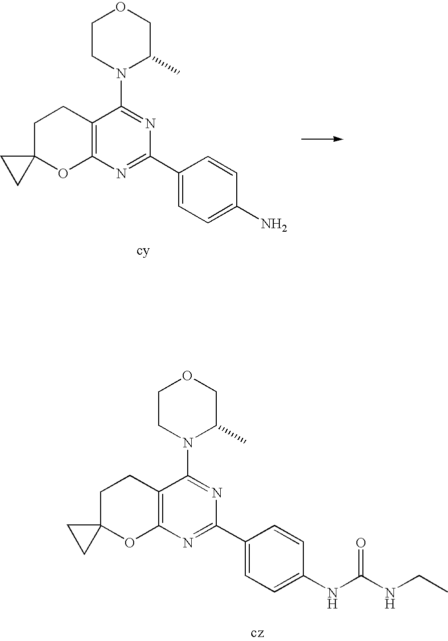

Step 1—Synthesis of (S)-4-(4′-(3-methylmorpholino)-5′,6′-dihydrospiro[cyclopropane-1,7′-pyrano[2,3-d]pyrimidine]-2′-yl)aniline (cy): The title compound was prepared by the procedure described in Example 41, by substituting morpholine with 3S-3-methylmorpholine: ¹H NMR (400 MHz, CDCl₃) δ 8.20 (d, J=8.6 Hz, 2H), 6.67 (d, J=8.6 Hz, 2H), 4.08-3.98 (m, 2H), 3.96-3.81 (m, 3H), 3.81-3.71 (m, 1H), 3.65 (dd, J=11.2, 2.3 Hz, 1H), 3.58-3.45 (m, 2H), 2.78-2.59 (m, 2H), 2.02-1.91 (m, 1H), 1.84-1.73 (m, 1H), 1.33 (d, J=6.7 Hz, 3H), 1.23-1.11 (m, 2H), 0.73-0.56 (m, 2H).

Step 2—Synthesis of (5)-1-ethyl-3-(4-(4′-(3-methylmorpholino)-5′,6′-dihydrospiro[cyclopropane-1,7′-pyrano[2,3-d]pyrimidine]-2′-yl)phenyl)urea: To a solution of (S)-4-(4′-(3-methylmorpholino)-5′,6′-dihydrospiro[cyclopropane-1,7′-pyrano[2,3-d]pyrimidine]-2′-yl)aniline (60.6 mg, 0.172 mmol) in 1,2-dichloroethane (2.0 mL, 25 mmol) was added triethylamine (55.0 uL, 0.395 mmol). The solution was cooled to 0° C. and triphosgene (22.0 mg, 0.0741 mmol) was added to the mixture in a single portion. A light colored precipitate formed rapidly. After 5 min at 0° C., the reaction was heated to 70° C. for 40 min. The reaction was then cooled to RT and 2.00 M of ethylamine in tetrahydrofuran (0.300 mL) was added in a single portion and stirred 3 h at RT. Water (5 ml) was added, then extracted with CH₂Cl₂ (5×2 ml), and the organics were combined. The volatiles were removed under reduced pressure and the resulting crude material was purified by reverse phase HPLC: ¹H NMR (400 MHz, DMSO) δ 8.62 (s, 1H), 8.10 (d, J=8.7 Hz, 2H), 7.45 (d, J=8.7 Hz, 2H), 6.17 (t, J=5.5 Hz, 1H), 4.10-4.01 (m, 1H), 3.86 (d, J=11.1 Hz, 1H), 3.76-3.68 (m, 1H), 3.67-3.37 (m, 4H), 3.17-3.05 (m, 2H), 2.74-2.63 (m, 2H), 1.97-1.74 (m, 2H), 1.26 (d, J=6.6 Hz, 3H), 1.06 (t, J=7.2 Hz, 3H), 1.03-0.92 (m, 2H), 0.80-0.65 (m, 2H). LC/MS: m/z=+424.2 (M+H)+.

### Example 50

### Preparation of (5)-1-(1-methyl-1H-pyrazol-4-yl)-3-(4-(4′-(3-methylmorpholino)-5′,6′-dihydro spiro[cyclopropane-1,7′-pyrano[2,3-d]pyrimidine]-2′-yl)phenyl)urea (da)

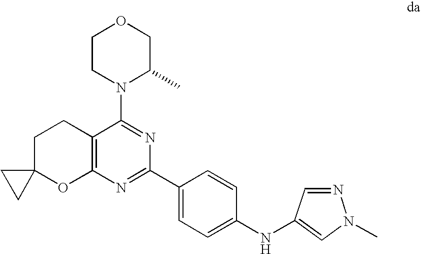

The title compound was prepared by the procedure described in Example 49, by substituting ethylamine with 1-methyl-1H-pyrazol-4-amine: ¹H NMR (400 MHz, DMSO) δ 8.83 (s, 1H), 8.44 (s, 1H), 8.14 (d, J=8.7 Hz, 2H), 7.74 (s, 1H), 7.51 (d, J=8.7 Hz, 2H), 7.37 (s, 1H), 4.13-4.01 (m, 1H), 3.87 (d, J=11.1 Hz, 1H), 3.78 (s, 3H), 3.72 (d, J=8.7 Hz, 1H), 3.68-3.37 (m, 4H), 2.77-2.63 (m, 2H), 1.96-1.75 (m, 2H), 1.26 (d, J=6.6 Hz, 3H), 1.08-0.94 (m, 2H), 0.81-0.64 (m, 2H). LC/MS: m/z=+476.2 (M+H)+.

### Example 51

### Preparation of (5)-1-(4-(4′-(3-methylmorpholino)-5′,6′-dihydrospiro[cyclopropane-1,7′-pyrano[2,3-d]pyrimidine]-2′-yl)phenyl)-3-(oxetan-3-yl)urea (db)

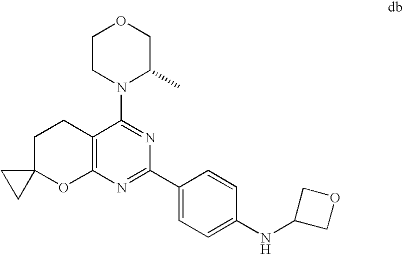

The title compound was prepared by the procedure described in Example 49, by substituting ethylamine with oxetan-3-amine: ¹H NMR (400 MHz, DMSO) δ 8.74 (s, 1H), 8.11 (d, J=8.7 Hz, 2H), 7.45 (d, J=8.7 Hz, 2H), 6.97 (d, J=6.5 Hz, 1H), 4.83-4.67 (m, 3H), 4.44 (t, J=5.8 Hz, 2H), 4.11-4.01 (m, 1H), 3.86 (d, J=11.0 Hz, 1H), 3.75-3.67 (m, 1H), 3.67-3.37 (m, 4H), 2.75-2.63 (m, 2H), 1.97-1.75 (m, 2H), 1.25 (d, J=6.6 Hz, 3H), 1.07-0.94 (m, 2H), 0.80-0.65 (m, 2H). LC/MS: m/z=+452.2 (M+H)+.

### Example 52

### Preparation of (S)-1-(1-methyl-1H-pyrazol-3-yl)-3-(4-(4′-(3-methylmorpholino)-5′,6′-dihydro spiro[cyclopropane-1,7′-pyrano[2,3-d]pyrimidine]-2′-yl)phenyl)urea (dc)

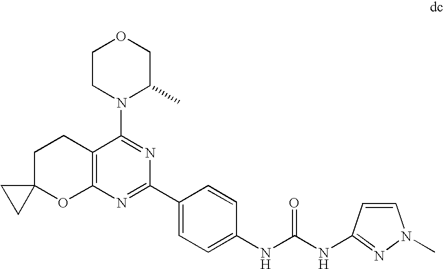

Synthesis of (S)-1-(1-methyl-1H-pyrazol-3-yl)-3-(4-(4′-(3-methylmorpholino)-5′,6′-dihydrospiro[cyclopropane-1,7′-pyrano[2,3-d]pyrimidine]-2′-yl)phenyl)urea (dc): The title compound was prepared by the procedure described in Example 49, by substituting ethylamine with oxetan-3-amine: ¹H NMR (400 MHz, DMSO) δ 9.16 (br s, 1H), 8.95 (s, 1H), 8.16 (d, J=8.7 Hz, 2H), 7.57-7.48 (m, 3H), 6.23 (d, J=1.9 Hz, 1H), 4.12-4.03 (m, 1H), 3.87 (d, J=11.0 Hz, 1H), 3.74 (s, 3H), 3.73-3.67 (m, 1H), 3.68-3.37 (m, 4H), 2.76-2.65 (m, 2H), 1.97-1.76 (m, 2H), 1.27 (d, J=6.6 Hz, 3H), 1.08-0.94 (m, 2H), 0.81-0.66 (m, 2H). LC/MS: m/z=+476.2 (M+H)+.

### Example 53

### Preparation of (S)-1-(4-(4′-(3-methylmorpholino)-5′,6′-dihydrospiro[cyclopropane-1,7′-pyrano[2,3-d]pyrimidine]-2′-yl)phenyl)-3-(4-methyloxazol-2-yl)urea (dd)

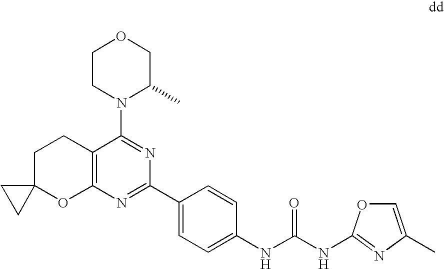

The title compound was prepared by the procedure described in Example 49, by substituting ethylamine with 4-methyloxazol-2-amine: ¹H NMR (400 MHz, DMSO) δ 10.98 (br s, 1H), 10.60 (br s, 1H), 8.19 (d, J=8.6 Hz, 2H), 7.60 (d, J=8.6 Hz, 2H), 7.47 (s, 1H), 4.14-4.02 (m, 1H), 3.87 (d, J=11.2 Hz, 1H), 3.75-3.69 (m, 1H), 3.67-3.37 (m, 3H), 2.79-2.63 (m, 2H), 2.09 (s, 3H), 1.96-1.76 (m, 2H), 1.27 (d, J=6.6 Hz, 3H), 1.09-0.94 (m, 2H), 0.81-0.66 (m, 3H). LC/MS: m/z=+477.2 (M+H)+.

### Example 54

### Preparation of (S)-6-(4-(4′-(3-methylmorpholino)-5′,6′-dihydrospiro[cyclopropane-1,7′-pyrano[2,3-d]pyrimidine]-2′-yl)phenyl amino)pyridin-2(1H)-one (df)

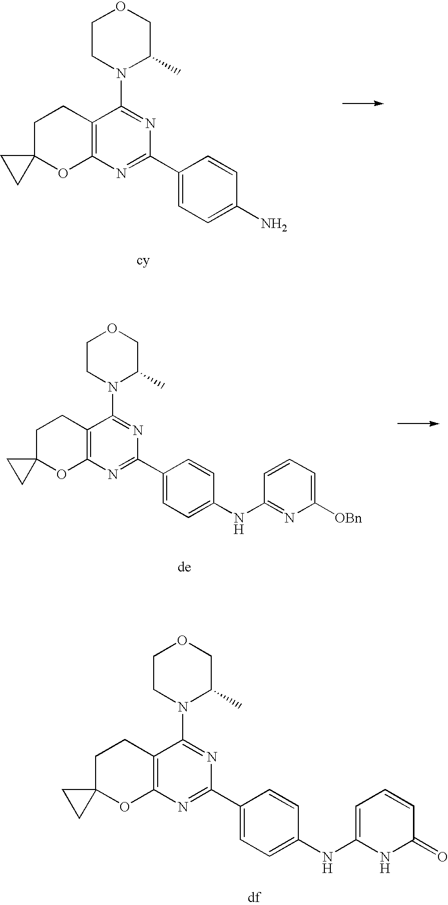

Step 1—Synthesis of (S)-6-(benzyloxy)-N-(4-(4′-(3-methylmorpholino)-5′,6′-dihydrospiro[cyclopropane-1,7′-pyrano[2,3-d]pyrimidine]-2′-yl)phenyl)pyridin-2-amine (de): (S)-4-(4′-(3-methylmorpholino)-5′,6′-dihydrospiro[cyclopropane-1,7′-pyrano[2,3-d]pyrimidine]-2′-yl)aniline (138.7 mg, 0.3936 mmol), 2-bromo-6-benzyloxypyridine (123 mg, 0.465 mmol), bis(dibenzylideneacetone)palladium(0) (16 mg, 0.027 mmol), sodium tert-butoxide (63.8 mg, 0.664 mmol) and 2-dicyclohexylphosphino-2′-(N,N-dimethylamino)biphenyl (62 mg, 0.16 mmol) were weighed into a microwave vial. The vial was evacuated and purged 3× with N₂, and then degassed toluene (3.0 mL) was added, and the vial sealed. The reaction was microwaved at 100° C. for 30 min. The reaction mixture was filtered through Celite, washing extensively with CH₂Cl₂. This was then concentrated onto silica gel and subjected to column chromatography using a 25 g column, with a gradient of 0% to 100% ethyl acetate in hexanes. The product containing fractions were combined and evaporated under reduced pressure to give (S)-6-(benzyloxy)-N-(4-(4′-(3-methylmorpholino)-5′,6′-dihydrospiro[cyclopropane-1,7′-pyrano[2,3-d]pyrimidine]-2′-yl)phenyl)pyridin-2-amine. ¹H NMR (400 MHz, CDCl₃) δ 8.33 (d, J=8.7 Hz, 2H), 7.50-7.28 (m, 8H), 6.50 (s, 1H), 6.45 (d, J=7.8 Hz, 1H), 6.30 (d, J=7.9 Hz, 1H), 5.37 (s, 2H), 4.09-4.02 (m, 1H), 3.94 (d, J=11.2 Hz, 1H), 3.86 (dd, J=11.2, 2.6 Hz, 1H), 3.83-3.73 (m, 1H), 3.68 (dd, J=11.2, 2.2 Hz, 1H), 3.63-3.49 (m, 2H), 2.82-2.63 (m, 2H), 2.03-1.94 (m, 1H), 1.86-1.74 (m, 1H), 1.36 (d, J=6.7 Hz, 3H), 1.31-1.12 (m, 2H), 0.75-0.56 (m, 2H). LC/MS: m/z=+477.2 (M+H)+.

Step 2—Synthesis of (S)-6-(4-(4′-(3-methylmorpholino)-5′,6′-dihydrospiro[cyclopropane-1,7′-pyrano[2,3-d]pyrimidine]-2′-yl)phenylamino)pyridin-2(1H)-one (df): To stirred solution of (S)-6-(benzyloxy)-N-(4-(4′-(3-methylmorpholino)-5′,6′-dihydrospiro[cyclopropane-1,7′-pyrano[2,3-d]pyrimidine]-2′-yl)phenyl)pyridin-2-amine (96 mg, 0.18 mmol) in chloroform (3.00 mL) was added methanesulfonic acid (1.00 mL, 15.4 mmol) in a single portion. The reaction was stirred overnight at RT. The reaction was diluted with CH₂Cl₂, then quenched with a saturated aqueous solution of NaHCO3. The layers were separated and the aqueous phase extracted with CH₂Cl₂ (3×25 ml). The combined organics were dried with MgSO₄, filtered and concentrated. This crude product was purified by reverse phase HPLC: ¹H NMR (400 MHz, DMSO) δ 10.21 (br s, 1H), 9.04 (s, 1H), 8.13 (d, J=8.8 Hz, 2H), 7.81-7.65 (m, 2H), 7.41 (t, J=7.9 Hz, 1H), 6.30 (d, J=6.3 Hz, 1H), 6.00 (d, J=7.8 Hz, 1H), 4.12-4.01 (m, 1H), 3.87 (d, J=11.1 Hz, 1H), 3.77-3.69 (m, 1H), 3.68-3.38 (m, 4H), 2.77-2.62 (m, 2H), 1.96-1.76 (m, 2H), 1.27 (d, J=6.6 Hz, 3H), 1.08-0.94 (m, 2H), 0.81-0.65 (m, 2H). LC/MS: m/z=+446.2 (M+H)+.

### Example 55

### Preparation of (S)-2-(4-(4′-(3-methylmorpholino)-5′,6′-dihydrospiro[cyclopropane-1,7′-pyrano[2,3-d]pyrimidine]-2′-yl)phenylamino)pyrimidin-4(3H)-one (dg)

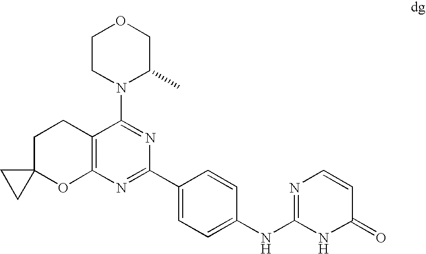

The title compound was prepared by the procedure described in Example 54, by substituting 2-bromo-6-benzyloxypyridine with 2-chloro-6-benzyloxypyrimidine: ¹H NMR (400 MHz, DMSO) δ 10.77 (br s, 1H), 9.05 (br s, 1H), 8.19 (d, J=8.7 Hz, 2H), 7.88-7.62 (m, 3H), 5.86 (br s, 1H), 4.13-4.03 (m, 1H), 3.87 (d, J=11.0 Hz, 1H), 3.72 (d, J=8.8 Hz, 1H), 3.68-3.39 (m, 4H), 2.79-2.64 (m, 2H), 1.96-1.76 (m, 2H), 1.27 (d, J=6.6 Hz, 3H), 1.08-0.95 (m, 2H), 0.80-0.66 (m, 2H). LC/MS: m/z=+447.2 (M+H)+.

### Example 56

### Synthesis of (5)-1-methyl-3-(4-(4′-(3-methylmorpholino)-5′,6′-dihydrospiro[cyclopropane-1,7′-pyrano[2,3-d]pyrimidine]-2′-yl)phenyl)urea (dh)

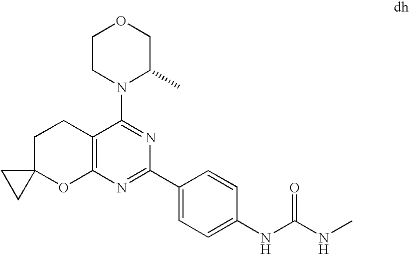

The title compound was prepared by the procedure described in Example 49, by substituting ethylamine with methylamine: ¹H NMR (400 MHz, DMSO) δ 8.71 (s, 1H), 8.10 (d, J=8.7 Hz, 2H), 7.46 (d, J=8.8 Hz, 2H), 6.09 (d, J=4.7 Hz, 1H), 4.10-4.01 (m, 1H), 3.86 (d, J=11.1 Hz, 1H), 3.72 (dd, J=11.3, 2.3 Hz, 1H), 3.68-3.37 (m, 4H), 2.74-2.64 (m, 2H), 2.65 (d, J=4.6 Hz, 3H), 1.96-1.75 (m, 2H), 1.26 (d, J=6.6 Hz, 3H), 1.08-0.94 (m, 2H), 0.81-0.65 (m, 2H). LC/MS: m/z=+410.2 (M+H)+.

### Example 57

### Preparation of (S)-1-(4-(4′-(3-methylmorpholino)-5′,6′-dihydrospiro[cyclopropane-1,7′-pyrano[2,3-d]pyrimidine]-2′-yl)phenyl)-3-(2-(methylsulfonyl)ethyl)urea (di)

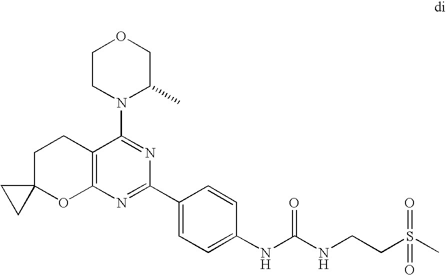

The title compound was prepared by the procedure described in Example 49, by substituting ethylamine with 2-(methylsulfonyl)ethanamine: ¹H NMR (400 MHz, DMSO) δ 8.94 (s, 1H), 8.11 (d, J=8.7 Hz, 1H), 7.47 (d, J=8.8 Hz, 2H), 6.40 (t, J=5.8 Hz, 1H), 4.11-4.00 (m, 1H), 3.86 (d, J=11.2 Hz, 1H), 3.72 (d, J=9.1 Hz, 1H), 3.67-3.37 (m, 6H), 3.34-3.29 (m, 3H), 3.03 (s, 3H), 2.75-2.63 (m, 2H), 1.96-1.75 (m, 2H), 1.26 (d, J=6.6 Hz, 3H), 1.07-0.93 (m, 2H), 0.80-0.65 (m, 2H). LC/MS: m/z=+502.2 (M+H)+.

### Example 58

### Synthesis of (5)-1-methyl-3-(4-(4-(3-methylmorpholino)-7,8-dihydro-5H-pyrano[4,3-d]pyrimidin-2-yl)phenyl)urea (dj)

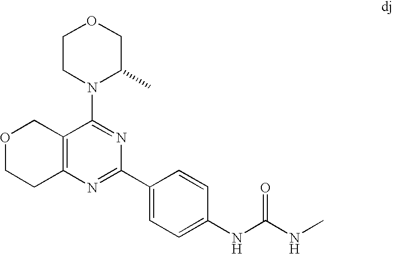

The title compound was prepared by the procedure described in Example 20, by substituting 3-aminoisoxazole with methylamine: ¹H NMR (400 MHz, DMSO) δ 8.71 (s, 1H), 8.18 (d, J=8.7 Hz, 2H), 7.48 (d, J=8.8 Hz, 2H), 6.10-6.03 (m, 1H), 4.64-4.51 (m, 2H), 4.07-3.82 (m, 4H), 3.74-3.65 (m, 1H), 3.65-3.34 (m, 4H), 2.91-2.80 (m, 2H), 2.65 (d, J=4.6 Hz, 3H), 1.23 (d, J=6.6 Hz, 3H). LC/MS: m/z=+384.1 (M+H)+.

### Example 59

### Preparation of (5)-1-(4-(4-(3-methylmorpholino)-7,8-dihydro-5H-pyrano[4,3-d]pyrimidin-2-yl)phenyl)-3-(2-(methylsulfonyl)ethyl)urea (dk)

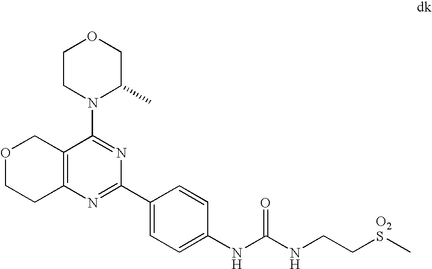

The title compound was prepared by the procedure described in Example 20, by substituting 3-aminoisoxazole with 2-(methylsulfonyl)ethanamine: ¹H NMR (500 MHz, DMSO) δ 8.79 (s, 1H), 8.23-8.15 (m, 3H), 7.50 (d, J=8.7 Hz, 2H), 6.36 (t, J=5.9 Hz, 1H), 4.60 (q, J=14.3 Hz, 2H), 4.10-3.83 (m, 4H), 3.73 (dd, J=11.3, 2.9 Hz, 1H), 3.67-3.53 (m, 4H), 3.52-3.29 (m, 6H), 2.90-2.84 (m, 2H), 1.26 (d, J=6.6 Hz, 3H). LC/MS: m/z=+476.2 (M+H)+.

### Example 60

### 5-(4-((1R,5S)-8-oxa-3-azabicyclo [3.2.1]octan-3-yl)-6,7-dihydro-5H-pyrano[2,3-d]pyrimidin-2-yl)pyridin-2(1H)-one (il)

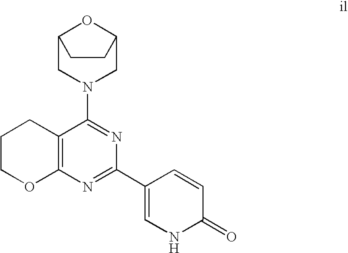

5-(4-((1R,5S)-8-oxa-3-azabicyclo[3.2.1]octan-3-yl)-6,7-dihydro-5H-pyrano[2,3-d]pyrimidin-2-yl)pyridin-2(1H)-one (il) was prepared in a similar manner as described for Example 1 with the exceptions that tetrahydro-2H-pyran-2-one was used in Step 1 instead of dihydro-2H-pyran-3(4H)-one, (1R,5S)-8-oxa-3-azabicyclo[3.2.1]octane was used in Step 5 instead of morpholine and 6-(benzyloxy)pyridin-3-ylboronic acid was used in step 6 instead of 1-ethyl-3-(4-(4,4,5,5-tetramethyl-1,3,2-dioxaborolan-2-yl)phenyl)urea. LC-MS: m/z=341 (M+H). ¹H NMR (400 MHz, DMSO) δ 11.78 (s, 1H), 8.24-8.08 (m, 2H), 6.39 (d, J=9.8 Hz, 1H), 4.36 (s, 2H), 4.32-4.21 (m, 2H), 3.70 (d, J=12.6 Hz, 2H), 3.15 (d, J=11.4 Hz, 2H), 2.56 (dd, J=12.4, 6.3 Hz, 2H), 1.95-1.70 (m, 6H).

### Example 61

### Preparation of 6-(4-((1R,5S)-8-oxa-3-azabicyclo[3.2.1]octan-3-yl)-6,7-dihydro-5H-pyrano[2,3-d]pyrimidin-2-yl)-1H-benzo[d]imidazol-2-amine (dm)

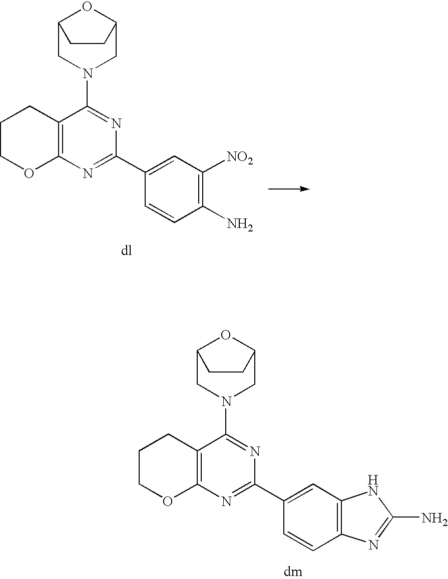

Synthesis of (dm): The title compound was prepared in a similar manner as described for Example 1 with the exceptions that tetrahydro-2H-pyran-2-one was used in Step 1 instead of dihydro-2H-pyran-3(4H)-one, (1R,5S)-8-oxa-3-azabicyclo[3.2.1]octane was used in Step 5 instead of morpholine and 2-nitro-4-(4,4,5,5-tetramethyl-1,3,2-dioxaborolan-2-yl)aniline was used in Step 6 instead of 4-(3-ethylureido)phenylboronic acid pinacol ester to provide (a). LC-MS: m/z=+384 (M+H)+. To crude 4-(4-4-((1R,5S)-8-oxa-3-azabicyclo[3.2.1]octan-3-yl)-6,7-dihydro-5H-pyrano[2,3-d]pyrimidin-2-yl)-2-nitroaniline (a, 0.075 g, 0.020 mmol) dissolved in ethanol (0.571 mL, 9.78 mmol) and water (0.564 mL, 31.3 mmol) was added ammonium chloride (0.042 g, 0.782 mmol) and iron (0.054 g, 0.978 mmol). The reaction mixture was stirred for 30 min at 75° C., cooled to room temperature and then diluted with CH₂Cl₂ and filtered through a pad of silica gel. Saturated aqueous NaHCO₃ solution (5 mL) was then added to the filtrate and after separation, the aqueous layer was extracted with CH₂Cl₂ (2×). The combined organic extract was dried (Na₂SO₄), filtered, concentrated, and the resulting crude aniline was carried on without further purification. To the crude aniline dissolved in methanol (1.13 mL, 28.0 mmol) was added cyanogen bromide (0.090 mL, 3.0 M solution in dichloromethane) at room temperature. After 3 h, the reaction mixture was concentrated to dryness and purified by reverse-phase HPLC to give the pure desired product (dm): ¹H NMR (400 MHz, DMSO) δ 8.08 (s, 1H), 7.98 (d, J=8.0, 1H), 7.18 (d, J=8.0, 1H), 6.91 (br s, 2H), 4.39 (br s, 2H), 4.29 (t, J=4.0 Hz, 2H), 3.73 (br d, J=12.0 Hz, 2H), 3.19 (br d, J=12.0 Hz, 2H), 2.61-2.58 (m, 2H), 1.92-1.82 (m, 6H); LC-MS: m/z=+379 (M+H)⁺.

### Example 62

### Preparation of 1-Ethyl-3-{4-[(1S,9R)-3-((S)-3-methyl-morpholin-4-yl)-12-oxa-4,6-diaza-tricyclo[7.2.1.0-2,7]dodeca-2(7),3,5-trien-5-yl]-phenyl}-urea (do¹) and 1-Ethyl-3-{4-[(1R,9S)-3-((S)-3-methyl-morpholin-4-yl)-12-oxa-4,6-diaza-tricyclo[7.2.1.0%2,78]dodeca-2(7),3,5-trien-5-yl]-phenyl}-urea (do²)

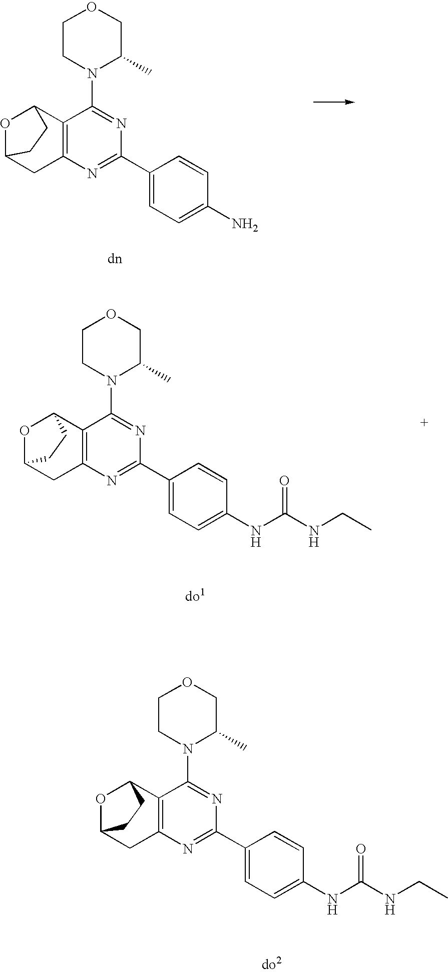

Synthesis of (do¹ and do²): The title compounds were prepared in a similar manner as described for Example 12 with the exceptions that 8-oxabicyclo[3.2.1]octan-3-on-e was used in Step 1 instead of dihydro-2H-pyran-3(4H)-one and (S)-3-methylmorpholine-4-carbonitrile was used instead of 4-morpholinecarbonitrile in Step 2 to provide (dn). LC-MS: m/z=+353 (M+H)⁺. To crude 4-[3-((S)-3-Methyl-morpholin-4-yl)-12-oxa-4,6-diaza-tricyclo [7.2.1.0-2,7]dodeca-2(7),3,5-trien-5-yl]-phenylamine (dn) dissolved in 1,2-dichloroethane (2.99 mL, 37.9 mmol) was added triethylamine (0.122 mL, 0.872 mmol) and triphosgene (0.045 g, 0.152 mmol) at 0° C. After 5 min the reaction mixture was heated to 70° C. for 40 min, cooled to room temperature and ethylamine hydrochloride (0.154 g, 1.90 mmol) was added. After stirring for 12 h at room temperature, water (5 mL) was added and the mixture was extracted with CH₂Cl₂ (3×5 mL). The combined organic extract was dried (Na₂SO₄), filtered, concentrated and purified by chiral super critical fluid chromatography to give the pure desired products (do¹ and do²), the absolute stereochemistry of the isomers has not been assigned: (faster eluting isomer): ¹H NMR (400 MHz, CDCl₃) δ: 8.31 (d, J=8.0 Hz, 2H), 7.35 (d, J=8.0 Hz, 2H), 6.24 (br s, 1H), 5.18 (d, J=8.0 Hz, 1H), 4.82 (t, J=8.0 Hz, 1H), 4.63 (t, J=4.0 Hz, 1H), 4.02-3.92 (m, 3H), 3.73-3.67 (m, 2H), 3.60-3.53 (m, 1H), 3.47-3.44 (m, 1H), 3.37-3.29 (m, 3H), 2.72-2.67 (m, 1H), 2.40-2.27 (m, 2H), 2.13-2.08 (m, 1H), 1.90-1.86 (m, 1H), 1.21-1.16 (m, 6H); LC-MS: m/z=+424 (M+H)⁺; (slower eluting isomer): ¹H NMR (400 MHz, CDCl₃) δ: 8.30 (d, J=8.0 Hz, 2H), 7.35 (d, J=8.0 Hz, 2H), 6.24 (br s, 1H), 5.13 (d, J=8.0 Hz, 1H), 4.82 (t, J=8.0 Hz, 1H), 4.64 (m, 1H), 4.07-4.05 (m, 1H), 3.95-3.91 (m, 1H), 3.86-3.65 (m, 4H), 3.50-3.43 (m, 1H), 3.36-3.29 (m, 3H), 2.68-2.63 (m, 1H), 2.39-2.25 (m, 2H), 2.14-2.10 (m, 1H), 1.87-1.83 (m, 1H), 1.46 (d, J=4.0 Hz, 3H), 1.18 (t, J=4.0 Hz, 3H); LC-MS: m/z=+424 (M+H)⁺.

### Example 63

### Preparation of 1-{4-[(1S,9R)-3-((S)-3-Methyl-morpholin-4-yl)-12-oxa-4,6-diaza-tricyclo[7.2.1.0-2,7]dodeca-2(7),3,5-trien-5-yl]-phenyl}-3-oxetan-3-yl-urea (dp¹) and 1-{4-[(1R,9S)-3-((S)-3-Methyl-morpholin-4-yl)-12-oxa-4,6-diaza-tricyclo[7.2.1.0-2,7]dodeca-2(7),3,5-trien-5-yl]-phenyl}-3-oxetan-3-yl-urea (dp²)

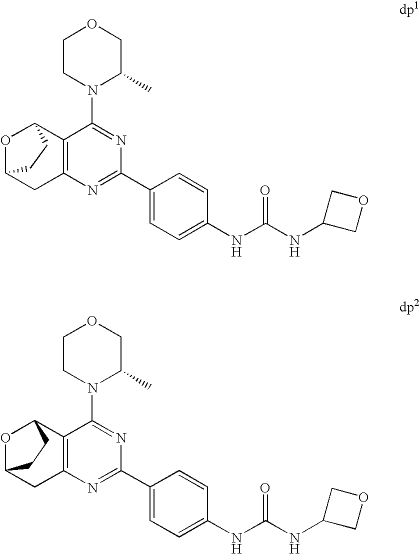

Synthesis of (dp¹ and dp²): The title compounds were prepared in a similar manner as described for Example 62 with the exception that oxetan-3-amine hydrochloride was used instead of ethylamine hydrochloride. The absolute stereochemistry of the two separated diastereomers has yet to be assigned: (faster eluting isomer): ¹H NMR (400 MHz, CDCl₃) δ: 8.33 (d, J=8.0 Hz, 2H), 7.35 (d, J=8.0 Hz, 2H), 6.42 (br s, 1H), 5.24-5.18 (m, 2H), 5.08-4.99 (m, 1H), 4.94 (t, J=8.0 Hz, 2H), 4.83 (t, J=4.0 Hz, 1H), 4.49 (t, J=8.0 Hz, 2H), 4.03-3.92 (m, 3H), 3.73-3.67 (m, 2H), 3.61-3.54 (m, 1H), 3.49-3.44 (m, 1H), 3.37-3.31 (m, 1H), 2.72-2.68 (m, 1H), 2.40-2.28 (m, 2H), 2.13-2.09 (m, 1H), 1.90-1.86 (m, 1H), 1.21 (d, J=8.0 Hz, 3H); LC-MS: m/z=+452 (M+H)⁺; (slower eluting isomer): ¹H NMR (400 MHz, CDCl₃) δ: 8.32 (d, J=8.0 Hz, 2H), 7.35 (d, J=8.0 Hz, 2H), 6.41 (br s, 1H), 5.22 (d, J=8.0 Hz, 1H), 5.13 (d, J=8.0 Hz, 1H), 5.08-4.99 (m, 1H), 4.94 (t, J=8.0 Hz, 2H), 4.82 (t, J=4.0 Hz, 1H), 4.50 (t, J=8.0 Hz, 2H), 4.08-4.03 (m, 1H), 3.95-3.91 (m, 1H), 3.86-3.66 (m, 4H), 3.50-3.44 (m, 1H), 3.37-3.31 (m, 1H), 2.68-2.64 (m, 1H), 2.38-2.28 (m, 2H), 2.15-2.10 (m, 1H), 1.87-1.81 (m, 1H), 1.46 (d, J=8.0 Hz, 3H); LC-MS: m/z=+452 (M+H)⁺.

### Example 64

### Preparation of (S)-6-(4-(3-methylmorpholino)-6,7-dihydro-5H-pyrano[2,3-d]pyrimidin-2-yl)-1H-benzo[d]imidazol-2-amine (dq)

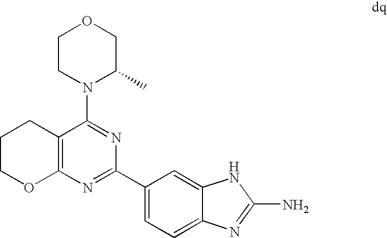

Synthesis of (dq): The title compound was prepared in a similar manner as described for Example 61 with the exception that (S)-3-methylmorpholine was used instead of (1R,5S)-8-oxa-3-azabicyclo[3.2.1]octane: ¹H NMR (400 MHz, CDCl₃) δ 8.28 (s, 1H), 8.16 (d, J=8.4 Hz, 1H), 7.30 (d, J=8.1 Hz, 1H), 4.77 (br s, 1H), 4.51-4.32 (m, 2H), 4.10-3.99 (m, 1H), 3.93 (d, J=10.9 Hz, 1H), 3.84 (d, J=11.1 Hz, 1H), 3.81-3.71 (m, 1H), 3.67 (d, J=11.3 Hz, 1H), 3.63-3.47 (m, 2H), 2.72-2.53 (m, 2H), 2.08-1.94 (m, 4H), 1.35 (d, J=6.6 Hz, 3H); LC-MS: m/z=+367 (M+H)⁺.

### Example 65

### Preparation of (S)-1-(2-hydroxyethyl)-3-(4-(4′-(3-methylmorpholino)-5′,6′-dihydrospiro[cyclopropane-1,7′-pyrano[2,3-d]pyrimidine]-2′-yl)phenyl)urea (dr)

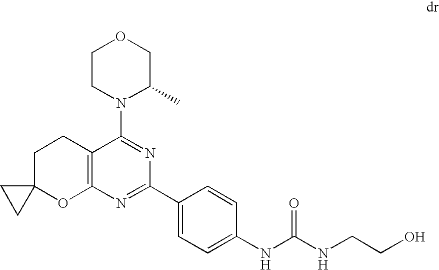

Synthesis of (S)-1-(2-hydroxyethyl)-3-(4-(4′-(3-methylmorpholino)-5′,6′-dihydrospiro[cyclopropane-1,7′-pyrano[2,3-d]pyrimidine]-2′-yl)phenyl)urea (dr): The title compound was prepared by the procedure described in Example 49, by substituting ethylamine with 2-aminoethanol: ¹H NMR (400 MHz, DMSO) δ 8.77 (s, 1H), 8.10 (d, J=8.8 Hz, 2H), 7.45 (d, J=8.8 Hz, 2H), 6.27 (t, J=5.2 Hz, 1H), 4.73 (t, J=5.0 Hz, 1H), 4.11-4.00 (m, 1H), 3.86 (d, J=11.2 Hz, 1H), 3.72 (dd, J=9.1 Hz, 2.6 Hz, 1H), 3.67-3.51 (m, 3H), 3.49-3.41 (m, 3H), 3.17 (q, J=5.6 Hz, 2H), 2.75-2.64 (m, 2H), 1.96-1.76 (m, 2H), 1.26 (d, J=6.6 Hz, 3H), 1.07-0.95 (m, 2H), 0.80-0.66 (m, 2H). LC/MS: m/z=440.2 (M+H)⁺, RT=9.37 min.

### Example 66

### Preparation of (S)-1-(2-cyanoethyl)-3-(4-(4′-(3-methylmorpholino)-5′,6′-dihydrospiro[cyclopropane-1,7′-pyrano[2,3-d]pyrimidine]-2′-yl)phenyl)urea (ds)

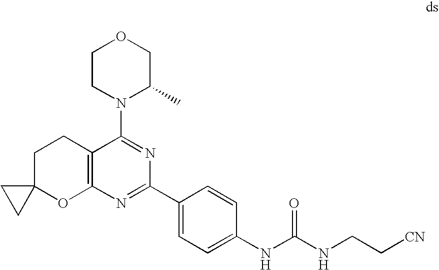

Synthesis of (S)-1-(2-cyanoethyl)-3-(4-(4′-(3-methylmorpholino)-5′,6′-dihydrospiro[cyclopropane-1,7′-pyrano[2,3-d]pyrimidine]-2′-yl)phenyl)urea (ds): The title compound was prepared by the procedure described in Example 49, by substituting ethylamine with 3-aminopropanenitrile: ¹H NMR (400 MHz, DMSO) δ 8.94 (s, 1H), 8.12 (d, J=8.6 Hz, 1H), 7.48 (d, J=8.8 Hz, 2H), 6.61 (t, J=6.0 Hz, 1H), 4.11-4.02 (m, 1H), 3.86 (d, J=11.0 Hz, 1H), 3.72 (dd, J=8.6 Hz,2.7 Hz, 1H), 3.67-3.51 (m, 3H), 3.48-3.30 (m, 4H), 2.73-2.65 (m, 4H), 1.98-1.74 (m, 2H), 1.26 (d, J=6.6 Hz, 3H), 1.07-0.95 (m, 2H), 0.80-0.66 (m, 2H). LC/MS: m/z=449.2 (M+H)⁺, RT=10.58 min.

### Example 67

### Preparation of ((S)-1-methoxy-3-(4-(4′-(3-methylmorpholino)-5′,6′-dihydrospiro[cyclopropane-1,7′-pyrano[2,3-d]pyrimidine]-2′-yl)phenyl)urea (dt)

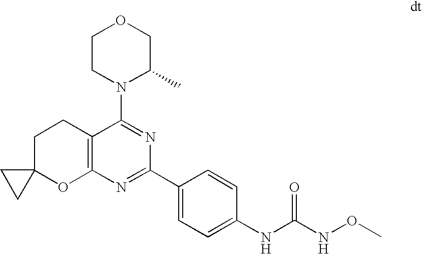

Synthesis of ((S)-1-methoxy-3-(4-(4′-(3-methylmorpholino)-5′,6′-dihydrospiro[cyclopropane-1,7′-pyrano[2,3-d]pyrimidine]-2′-yl)phenyl)urea (dt): The title compound was prepared by the procedure described in Example 49, by substituting ethylamine with O-methylhydroxylamine hydrochloride: ¹H NMR (400 MHz, DMSO) δ 9.58 (s, 1H), 9.01 (s, 1H), 8.14 (d, J=8.9 Hz, 2H), 7.67 (d, J=8.8 Hz, 2H), 4.13-4.03 (m, 1H), 3.87 (d, J=11.1 Hz, 1H), 3.72 (dd, J=11.2 Hz, 2.8 Hz, 1H), 3.68-3.52 (m, 6H), 3.49-3.38 (m, 1H), 2.76-2.64 (m, 2H), 1.97-1.76 (m, 2H), 1.26 (d, J=6.6 Hz, 3H), 1.08-0.95 (m, 2H), 0.80-0.66 (m, 2H). LC/MS: m/z=426.2 (M+H)⁺, RT=10.94 min.

### Example 68

### Preparation of 1-((S)-2,3-dihydroxypropyl)-3-(4-(4′4(S)-3-methylmorpholino)-5′,6′-dihydrospiro-[cyclopropane-1,7′-pyrano[2,3-d]pyrimidine]-2′-yl)phenyl)urea (du)

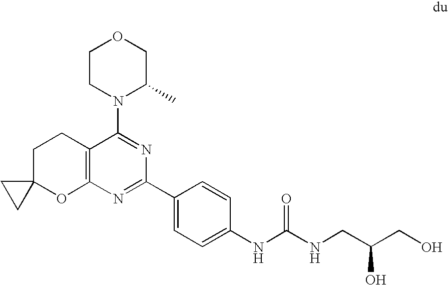

Synthesis of 14(S)-2,3-dihydroxypropyl)-3-(4-(4′4(S)-3-methylmorpholino)-5′,6′-dihydrospiro-[cyclopropane-1,7′-pyrano[2,3-d]pyrimidine]-2′-yl)phenyl)urea (du): The title compound was prepared by the procedure described in Example 49, by substituting ethylamine with (S)-3-aminopropane-1,2-diol: ¹H NMR (400 MHz, DMSO) δ 8.82 (s, 1H), 8.10 (d, J=8.8 Hz, 2H), 7.44 (d, J=8.8 Hz, 2H), 6.23 (t, J=5.4 Hz, 1H), 4.84 (d, J=5.0 Hz, 1H), 4.57 (t, J=5.8 Hz, 1H), 4.11-4.03 (m, 1H), 3.86 (d, J=11.2 Hz, 1H), 3.72 (dd, J=11.2 Hz, 2.5 Hz, 1H), 3.68-3.30 (m, 8H), 3.04-2.94 (m, 1H), 2.75-2.63 (m, 2H), 1.97-1.74 (m, 2H), 1.26 (d, J=6.6 Hz, 3H), 1.07-0.95 (m, 2H), 0.80-0.66 (m, 2H). LC/MS: m/z=470.2 (M+H)⁺, RT=8.98 min.

### Example 69

### Preparation of 1-((R)-2,3-dihydroxypropyl)-3-(4-(4′-((S)-3-methylmorpholino)-5′,6′-dihydrospiro-[cyclopropane-1,7′-pyrano[2,3-d]pyrimidine]-2′-yl)phenyl)urea (dv)

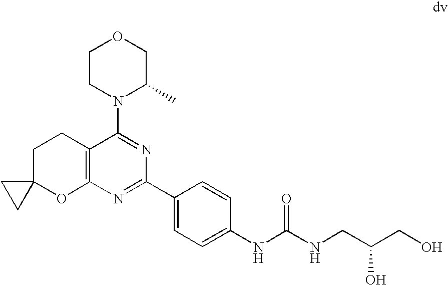

Synthesis of 1-((R)-2,3-dihydroxypropyl)-3-(4-(4′-((S)-3-methylmorpholino)-5′,6′-dihydrospiro-[cyclopropane-1,7′-pyrano[2,3-d]pyrimidine]-2′-yl)phenyl)urea (dv): The title compound was prepared by the procedure described in Example 49, by substituting ethylamine with (R)-3-aminopropane-1,2-diol: ¹H NMR (400 MHz, DMSO) δ 8.81 (s, 1H), 8.11 (d, J=8.8 Hz, 2H), 7.44 (d, J=8.8 Hz, 2H), 6.22 (t, J=5.6 Hz, 1H), 4.83 (d, J=5.0 Hz, 1H), 4.57 (t, J=5.7 Hz, 1H), 4.11-4.02 (m, 1H), 3.86 (d, J=11.2 Hz, 1H), 3.72 (dd, J=11.4 Hz, 2.7 Hz, 1H), 3.67-3.25 (m, 8H), 3.04-2.94 (m, 1H), 2.75-2.63 (m, 2H), 1.96-1.76 (m, 2H), 1.26 (d, J=6.6 Hz, 3H), 1.07-0.96 (m, 2H), 0.80-0.66 (m, 2H). LC/MS: m/z=470.2 (M+H)⁺, RT=9.06 min.

### Example 70

### Preparation of (S)-1-(1-(hydroxymethyl)cyclopropyl)-3-(4-(4′-(3-methylmorpholino)-5′,6′-dihydro spiro[cyclopropane-1,7′-pyrano[2,3-d]pyrimidine]-2′-yl)phenyl)urea (dw)

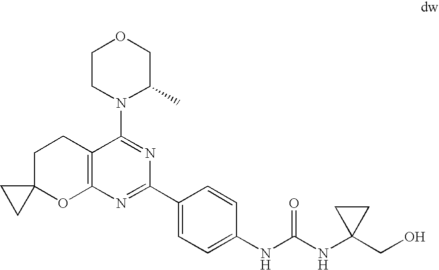

Synthesis of (S)-1-(1-(hydroxymethyl)cyclopropyl)-3-(4-(4′-(3-methylmorpholino)-5′,6′-dihydrospiro[cyclopropane-1,7′-pyrano[2,3-d]pyrimidine]-2′-yl)phenyl)urea (dw): The title compound was prepared by the procedure described in Example 49, by substituting ethylamine with (1-aminocyclopropyl)methanol: ¹H NMR (400 MHz, DMSO) δ 8.63 (s, 1H), 8.11 (d, J=8.7 Hz, 2H), 7.43 (d, J=8.7 Hz, 2H), 6.57 (s, 1H), 4.84 (s, 1H), 4.06 (dd, J=10.4, 5.2 Hz, 1H), 3.86 (d, J=11.0 Hz, 1H), 3.72 (dd, J=11.2, 2.7 Hz, 1H), 3.67-3.50 (m, 3H), 3.48-3.40 (m, 3H), 2.75-2.63 (m, 2H), 1.96-1.86 (m, 1H), 1.86-1.75 (m, 1H), 1.26 (d, J=6.6 Hz, 3H), 1.06-0.95 (m, 2H), 0.80-0.67 (m, 4H), 0.66-0.60 (m, 2H). LC/MS: m/z=466.2 (M+H)⁺, RT=4.53 min.

### Example 71

### Preparation of 1-(4-(4′-((1R,5S)-3-oxa-8-azabicyclo[3.2.1]octan-8-yl)-5′,6′-dihydrospiro[cyclopropane-1,7′-pyrano[2,3-d]pyrimidine]-2′-yl)phenyl)-3-(oxetan-3-yl)urea (dx)

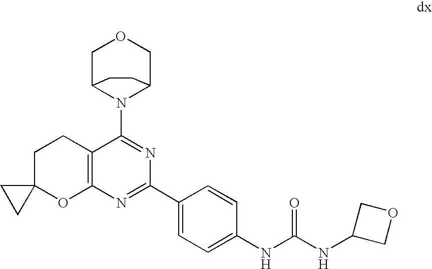

Synthesis of 1-(4-(4′-((1R,5S)-3-oxa-8-azabicyclo[3.2.1]octan-8-yl)-5′,6′-dihydrospiro[cyclopropane-1,7′-pyrano[2,3-d]pyrimidine]-2′-yl)phenyl)-3-(oxetan-3-yl)urea (dx): The title compound was prepared by the procedure described in Example 43, by substituting 4-(4′-morpholino-5′,6′-dihydrospiro[cyclopropane-1,7′-pyrano[2,3-d]pyrimidine]-2′-yl)aniline with 4-(4′-((1R,5S)-3-oxa-8-azabicyclo[3.2.1]octan-8-yl)-5′,6′-dihydrospiro[cyclopropane-1,7′-pyrano[2,3-d]pyrimidine]-2′-yl)aniline, which is prepared by the procedure described for intermediate co (in Example 41, steps 1-5), replacing (1R,5S)-3-oxa-8-azabicyclo[3.2.1]octane for morpholine. ¹H NMR (400 MHz, DMSO) δ 8.74 (s, 1H), 8.10 (d, J=8.8 Hz, 2H), 7.45 (d, J=8.8 Hz, 2H), 6.97 (d, J=6.5 Hz, 1H), 4.83-4.69 (m, 3H), 4.48 (s, 2H), 4.43 (t, J=5.8 Hz, 2H), 3.78 (d, J=10.6 Hz, 2H), 3.62 (d, J=10.2 Hz, 2H), 2.73 (t, J=6.1 Hz, 2H), 1.99-1.83 (m, 6H), 1.00 (t, J=6.1 Hz, 2H), 0.72 (t, J=6.4 Hz, 2H). LC/MS: m/z=464.2 (M+H)⁺, RT=10.60 min.

### Example 72

### Preparation of 1-(4-(4′-((1R,5S)-3-oxa-8-azabicyclo[3.2.1]octan-8-yl)-5′,6′-dihydrospiro[cyclopropane-1,7′-pyrano[2,3-d]pyrimidine]-2′-yl)phenyl)-3-(2-hydroxyethyl)urea (dy)

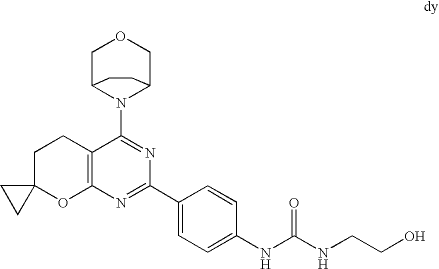

Synthesis of 1-(4-(4′-((1R,5S)-3-oxa-8-azabicyclo[3.2.1]octan-8-yl)-5′,6′-dihydrospiro[cyclopropane-1,7′-pyrano[2,3-d]pyrimidine]-2′-yl)phenyl)-3-(2-hydroxyethyl)urea (dy): The title compound was prepared by the procedure described in Example 71, by substituting oxetan-3-amine hydrochloride with 2-aminoethanol: ¹H NMR (400 MHz, DMSO) δ 8.74 (s, 1H), 8.09 (d, J=8.8 Hz, 2H), 7.44 (d, J=8.8 Hz, 2H), 6.24 (t, J=5.6 Hz, 1H), 4.73 (t, J=5.1 Hz, 1H), 4.47 (s, 2H), 3.78 (d, J=10.7 Hz, 2H), 3.62 (d, J=10.3 Hz, 2H), 3.45 (q, J=5.5 Hz, 2H), 3.16 (q, J=5.6 Hz, 2H), 2.73 (t, J=6.2 Hz, 2H), 1.99-1.82 (m, 6H), 1.00 (t, J=6.1 Hz, 2H), 0.72 (t, J=6.3 Hz, 2H). LC/MS: m/z=452.2 (M+H)⁺, RT=10.08 min.

### Example 73

### Preparation of 1-ethyl-3-(4-(7-(2-hydroxyethyl)-7-methyl-4-morpholino-5,7-dihydrofuro[3,4-d]pyrimidin-2-yl)phenyl)urea (ee) and 1-ethyl-3-(4-(7-methyl-4-morpholino-7-propyl-5,7-dihydrofuro[3,4-d]pyrimidin-2-yl)phenyl)urea (ef)

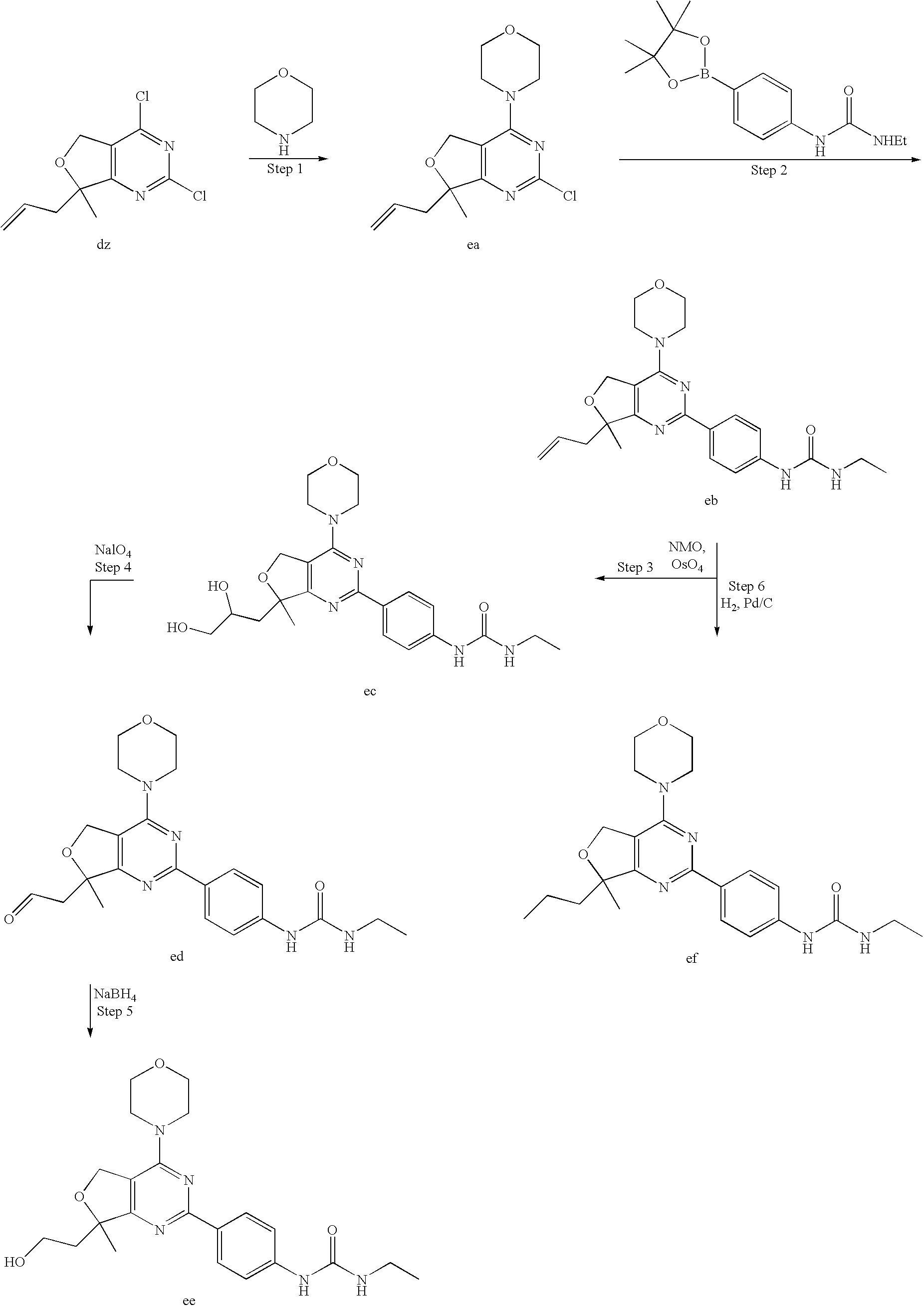

Step 1—Synthesis of 7-allyl-2-chloro-7-methyl-4-morpholino-5,7-dihydrofuro[3,4-d]pyrimidine (ea). To a solution of 7-allyl-2,4-dichloro-7-methyl-5,7-dihydrofuro-[3,4-d]pyrimidine (4.00 g, 16.3 mmol) in DMF (39 mL) and N,N-Diisopropylethylamine (4.27 mL, 24.5 mmol) was added morpholine (1.49 mL, 17.1 mmol) at 0° C., and the reaction was stirred at 0° C. for 90 min. After evaporation, column purification was done with 20% Ethyl Acetate in heptane, and 4.55 g (94% yield) white solid was obtained: ¹H NMR (400 MHz, CDCl₃) δ 5.70 (m, 1H), 5.07 (m, 4H), 3.76 (m, 4H), 3.62 (m, 4H), 2.54 (m, 2H), 1.44 (s, 3H); LC-MS m/z=296 (M+H).

Step 2—Synthesis of 1-(4-(7-allyl-7-methyl-4-morpholino-5,7-dihydrofuro[3,4-d]pyrimidin-2-yl)phenyl)-3-ethylurea (eb). 7-allyl-2-chloro-7-methyl-4-morpholino-5,7-dihydrofuro[3,4-d]pyrimidine (547 mg, 1.85 mmol), tricyclohexylphosphine (64.8 mg, 0.231 mmol), 4-ethylureidophenylboronic acid, pinacol ester (1080 mg, 3.71 mmol) and bis(dibenzylideneacetone)palladium(0) (106 mg, 0.185 mmol) were mixed in acetonitrile (7.80 mL) and 1.27 M of potassium phosphate in water (2.04 mL), and the heterogeneous solution was kept at 90° C. overnight. After evaporation of the solvents, the residue was purified by flash chromatography with 30% ethyl acetate in dichloromethane to afford 414 mg (53% yield) of a white solid: ¹H NMR (400 MHz, CDCl₃) δ 8.36 (d, J=8, 2H), 7.36 (d, J=8, 2H), 6.24 (s, 1H), 5.75 (m, 1H), 5.14 (m, 4H), 4.64 (m, 1H), 3.81 (m, 4H), 3.70 (m, 4H), 3.33 (m, 2H), 2.60 (m, 2H), 1.49 (s, 3H), 1.18 (t, J=7.2, 3H); LC-MS m/z=424 (M+H).

Step 3—Synthesis of 1-(4-(7-(2,3-dihydroxypropyl)-7-methyl-4-morpholino-5,7-dihydrofuro[3,4-d]pyrimidin-2-yl)phenyl)-3-ethylurea (ec). To a solution of 1-(4-(7-allyl-7-methyl-4-morpholino-5,7-dihydrofuro[3,4-d]pyrimidin-2-yl)phenyl)-3-ethylurea (198 mg, 0.468 mmol) in Tetrahydrofuran (4.0 mL) and water (1.3 mL) was added N-methylmorpholine N-oxide (65.7 mg, 0.561 mmol) and 2.5% OsO₄ in tert-butanol (2.5:97.5, osmium tetraoxide:tert-butyl alcohol, 0.40 mL) at 0° C., and the reaction was stirred at room temperature overnight. Sodium sulfite (0.707 g, 5.61 mmol) was added together with water (5 mL), and the mixture was stirred at room temperature for 1 h. Extraction was done with EtOAc. Evaporation of EtOAc gave 213 mg of diol as white solid, which was used without further purification: LC-MS m/z=458 (M+H).

Step 4—Synthesis of 1-ethyl-3-(4-(7-methyl-4-morpholino-7-(2-oxoethyl)-5,7-dihydrofuro[3,4-d]pyrimidin-2-yl)phenyl)urea (ed). To a solution of 1-(4-(7-(2,3-dihydroxypropyl)-7-methyl-4-morpholino-5,7-dihydrofuro[3,4-d]pyrimidin-2-yl)phenyl)-3-ethylurea (213 mg) in THF:H₂O (3:1, 16 mL) was added sodium periodate (150 mg, 0.701 mmol), and the solution was stirred at room temperature for 5 h. A white precipitate was observed. The reaction mixture was diluted with brine, extracted with EtOAc. The combined organic layers were dried over MgSO₄, filtered, concentrated in vacuo, to afford crude aldehyde (257 mg) as gummy solid, which was used without further purification.

Step 5—Synthesis of 1-ethyl-3-(4-(7-(2-hydroxyethyl)-7-methyl-4-morpholino-5,7-dihydrofuro[3,4-d]pyrimidin-2-yl)phenyl)urea (ee). To a solution of 1-ethyl-3-(4-(7-methyl-4-morpholino-7-(2-oxoethyl)-5,7-dihydrofuro[3,4-d]pyrimidin-2-yl)phenyl)urea (257 mg) in THF (13 mL) and MeOH (1 mL) was added sodium tetrahydroborate (71 mg, 1.87 mmol) at 0° C., and the resulting mixture was stirred at room temperature overnight. The reaction was quenched with saturated aqueous NH₄Cl (20 mL) and water (10 mL). The mixture was diluted with EtOAc (200 mL) and the phases separated. The organic phase was dried over MgSO₄, filtered, concentrated under reduced pressure, and yielded 207 mg white powder. Chiral HPLC separation gave two enantiomers: ¹H NMR (400 MHz, DMSO-d₆) δ 8.53 (s, 1H), 8.28 (d, J=8, 2H), 7.37 (d, J=8, 2H), 6.54 (s, 1H), 5.19 (m, 2H), 4.72 (m, 1H), 3.7-3.8 (m, 10H), 3.32 (m, 2H), 2.12 (m, 2H), 1.53 (s, 3H), 1.17 (t, J=7.2, 3H); LC-MS m/z=424 (M+H).

Step 6—Synthesis of 1-ethyl-3-(4-(7-methyl-4-morpholino-7-propyl-5,7-dihydrofuro[3,4-d]pyrimidin-2-yl)phenyl)urea (ef). To a nitrogen-flushed flask containing 1-(4-(7-allyl-7-methyl-4-morpholino-5,7-dihydrofuro[3,4-d]pyrimidin-2-yl)phenyl)-3-ethylurea (51 mg, 0.12 mmol) and palladium on carbon 10% (0.1:0.9, palladium:carbon black, 12.8 mg) was added methanol (2.5 mL), ethyl acetate (9.0 mL) and 1,4-cyclohexadiene (0.57 mL, 6.1 mmol) at room temperature. The reaction was kept at room temperature overnight. After evaporation of the solvents, the residue was purified via chiral HPLC to afford 17 mg of each enantiomer as white powder: ¹H NMR (400 MHz, CDCl₃) δ 8.37 (d, J=8, 2H), 7.36 (d, J=8, 2H), 6.25 (s, 1H), 5.15 (m, 2H), 4.64 (m, 1H), 3.81 (m, 4H), 3.70 (m, 4H), 3.33 (m, 2H), 1.80 (m, 2H), 1.48 (s, 3H), 1.45 (m, 1H), 1.13 (t, J=7.2, 3H), 1.11 (m, 1H), 0.88 (t, J=7.2, 3H); LC-MS m/z=426 (M+H).

### Example 74

### Preparation of Compound ei and ej

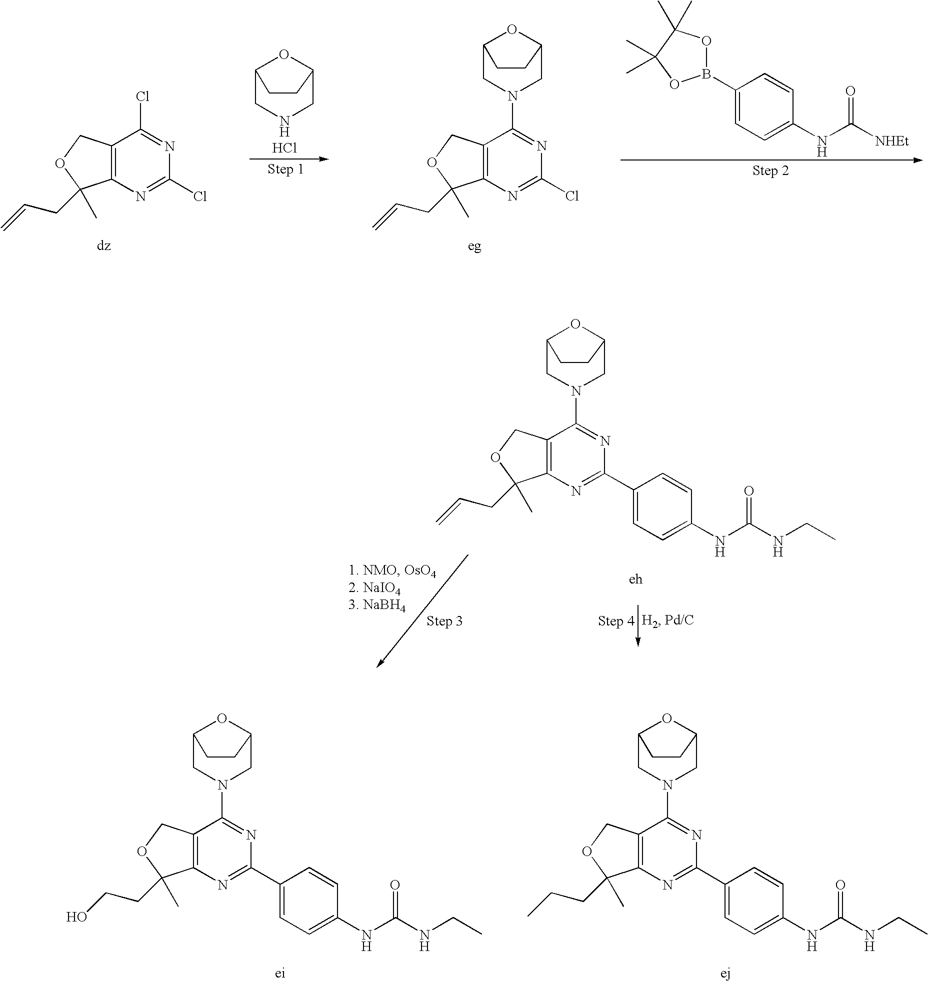

Step 1—Synthesis of 7-allyl-4-((1R,5S)-8-oxa-3-azabicyclo [3.2.1]octan-3-yl)-2-chloro-7-methyl-5,7-dihydrofuro[3,4-d]pyrimidine (eg). The title compound was prepared following the general procedure in Step 1 of Example 73, substituting morpholine for 8-oxa-3-azabicyclo[3.2.1]octane hydrochloride: LC-MS m/z=322 (M+H).

Step 2—Synthesis of 1-(4-(7-allyl-4-((1R,5S)-8-oxa-3-azabicyclo[3.2.1]octan-3-yl)-7-methyl-5,7-dihydrofuro[3,4-d]pyrimidin-2-yl)phenyl)-3-ethylurea (eh). The title compound was prepared following the general procedure in Step 2 of Example 73, substituting 7-allyl-2-chloro-7-methyl-4-morpholino-5,7-dihydrofuro[3,4-d]pyrimidine for 7-allyl-4-((1R,5S)-8-oxa-3-azabicyclo[3.2.1]octan-3-yl)-2-chloro-7-methyl-5,7-dihydrofuro[3,4-d]pyrimidine: LC-MS m/z=450 (M+H).

Step 3—Synthesis of 1-(4-(4-((1R,5S)-8-oxa-3-azabicyclo[3.2.1]octan-3-yl)-7-(2-hydroxyethyl)-7-methyl-5,7-dihydrofuro[3,4-d]pyrimidin-2-yl)phenyl)-3-ethylurea (ei). The title compound was prepared following the general procedure from Step 3 to Step 5 in Example 73, substituting 1-(4-(7-allyl-7-methyl-4-morpholino-5,7-dihydrofuro[3,4-d]pyrimidin-2-yl)phenyl)-3-ethylurea for 1-(4-(7-allyl-4-((1R,5S)-8-oxa-3-azabicyclo[3.2.1]octan-3-yl)-7-methyl-5,7-dihydrofuro[3,4-d]pyrimidin-2-yl)phenyl)-3-ethylurea. The enantiomers were separated by chiral HPLC: ¹H NMR (400 MHz, CDCl₃) δ 8.27 (d, J=8, 2H), 7.36 (d, J=8, 2H), 6.39 (s, 1H), 5.18 (m, 2H), 4.80 (m, 1H), 4.70 (m, 1H), 4.50 (m, 2H), 3.81 (m, 4H), 3.33 (m, 4H), 2.11 (m, 4H), 1.85 (m, 2H), 1.53 (s, 3H), 1.17 (t, J=8, 3H); LC-MS m/z 454 (M+H).

Step 4—Synthesis of 1-(4-(4-((1R,5S)-8-oxa-3-azabicyclo[3.2.1]octan-3-yl)-7-methyl-7-propyl-5,7-dihydrofuro[3,4-d]pyrimidin-2-yl)phenyl)-3-ethylurea (ej). The title compound was prepared following the general procedure in Step 6 of Example 73, substituting 1-(4-(7-allyl-7-methyl-4-morpholino-5,7-dihydrofuro[3,4-d]pyrimidin-2-yl)phenyl)-3-ethylurea for 1-(4-(7-allyl-4-((1R,5S)-8-oxa-3-azabicyclo[3.2.1]octan-3-yl)-7-methyl-5,7-dihydrofuro-[3,4-d]pyrimidin-2-yl)phenyl)-3-ethylurea. The enantiomers were separated by chiral HPLC: ¹H NMR (400 MHz, CDCl₃) δ 8.37 (d, J=8, 2H), 7.35 (d, J=8, 2H), 6.25 (s, 1H), 5.15 (m, 2H), 4.65 (m, 1H), 4.48 (s, 2H), 3.95 (m, 2H), 3.33 (m, 4H), 1.8-2.0 (m, 6H), 1.46 (s, 3H), 1.45 (m, 1H), 1.17 (t, J=7.2, 3H), 1.13 (m, 1H), 0.88 (t, J=7.2, 3H); LC-MS m/z 452 (M+H).

### Example 75

### Preparation of 1-ethyl-3-(4-(7-(3-hydroxypropyl)-7-methyl-4-((S)-3-methylmorpholino)-5,7-dihydro furo [3,4-d]pyrimidin-2-yl)phenyl)urea (el)

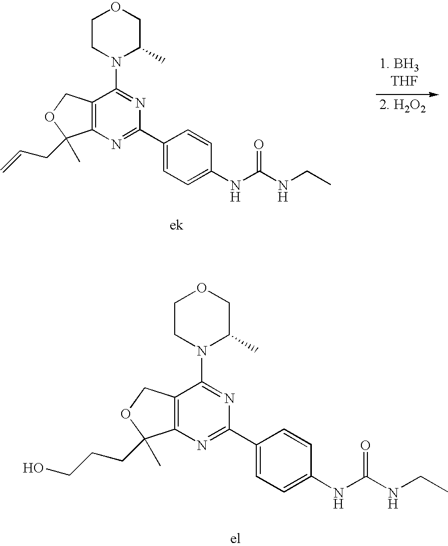

Synthesis of 1-ethyl-3-(4-(7-(3-hydroxypropyl)-7-methyl-4-((S)-3-methylmorpholino)-5,7-dihydrofuro[3,4-d]pyrimidin-2-yl)phenyl)urea (el). To a solution of 1-(4-(7-allyl-7-methyl-4-((S)-3-methylmorpholino)-5,7-dihydrofuro-[3,4-d]pyrimidin-2-yl)phenyl)-3-ethylurea (100 mg, 0.228 mmol) in tetrahydrofuran (2.0 mL, 24.8 mmol) at 0° C. was added 1.0 M of borane-THF complex (0.70 mL) dropwise under N₂. The reaction was allowed to warm slowly to room temperature and stirred overnight. The reaction was cooled at 0° C. then added 1.0 M of borane-THF complex in tetrahydrofuran (0.70 mL). The sample was allowed to warm slowly to room temperature and stirred for 12 hours. The solution cooled at 0° C. then added 1.0 M of borane-THF complex (2.00 mL), and the reaction was allowed to warm slowly to room temperature and stirred overnight. After the hydroboration was completed, 9.79 M of hydrogen peroxide in water (0.467 mL) was added followed by sodium hydroxide (45.7 mg, 1.14 mmol). The reaction was stirred at room temperature for 10 h. The sample was extracted 3 times with EtOAc, dried over MgSO₄, filtered, evaporated, and purified by column with 60% ethyl acetate in dichloromethane as the eluent. The major product (76 mg, 29% yield) was obtained as white solid. The two diastereomers were separated by chiral HPLC: LC-MS m/z 456 (M+H).

### Example 76

### Preparation of 1-ethyl-3-(4-(7-methyl-4-((S)-3-methylmorpholino)-7-(2-morpholinoethyl)-5,7-dihydrofuro[3,4-d]pyrimidin-2-yl)phenyl)urea (eo)

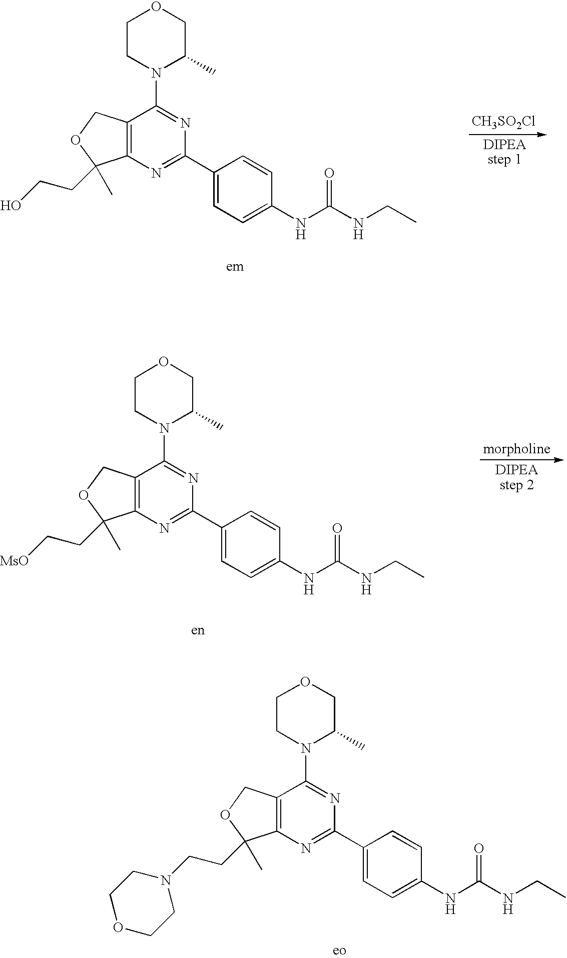

Step 1—Synthesis of 2-(2-(4-(3-ethylureido)phenyl)-7-methyl-4-((S)-3-methylmorpholino)-5,7-dihydrofuro[3,4-d]pyrimidin-7-yl)ethyl methanesulfonate. To a suspension of 1-ethyl-3-(4-(7-(2-hydroxyethyl)-7-methyl-4-(S)-3-methylmorpholino)-5,7-dihydrofuro[3,4-d]pyrimidin-2-yl)phenyl)urea (194 mg, 0.439 mmol) in Methylene chloride (1.0 mL) and chloroform (2.1 mL) was added N,N-diisopropylethylamine (0.230 mL, 1.32 mmol), followed by methanesulfonyl chloride (0.0850 mL, 1.10 mmol) at 0° C. The reaction was slowly warmed up to room temperature, and stirred overnight. Saturated aqueous NaHCO₃ was added into the reaction, followed by dilution with EtOAc after 20 min. The organic layer was washed with NaHCO₃, water and brine to pH˜9. Evaporation of EtOAc gave sticky oil, which turned into a white foam under vacuum. The crude was used without further purification: LC-MS m/z 520 (M+H).

Step 2—Synthesis of 1-ethyl-3-(4-(7-methyl-4-((S)-3-methylmorpholino)-7-(2-morpholinoethyl)-5,7-dihydrofuro[3,4-d]pyrimidin-2-yl)phenyl)urea. To a solution of 2-(2-(4-(3-ethylureido)phenyl)-7-methyl-4-((S)-3-methylmorpholino)-5,7-dihydrofuro[3,4-d]pyrimidin-7-yl)ethyl methanesulfonate (71 mg, 0.14 mmol) in DMF (1.0 mL, 13 mmol) and N,N-diisopropylethylamine (0.0595 mL, 0.342 mmol) was added morpholine (0.020 mL, 0.23 mmol), and the reaction was stirred at 60° C. for 1 day. The product was purified by reverse-phase HPLC, and the two diastereomers were separated by chiral HPLC: ¹H NMR (400 MHz, CDCl₃) of one diastereomer δ 8.37 (d, J=8, 2H), 7.36 (d, J=8, 2H), 6.25 (s, 1H), 5.18 (d, J=8.8, 1H), 5.11 (d, J=8.8, 1H), 4.65 (m, 1H), 4.21 (m, 1H), 4.04 (m, 2H), 3.79 (m, 2H), 3.59 (m, 5H), 3.43 (m, 1H), 3.32 (m, 2H), 2.0-2.7 (m, 6H), 2.15 (m, 1H), 2.00 (m, 1H), 1.48 (s, 3H), 1.36 (d, J=7.2, 3H), 1.18 (t, J=7.2, 3H); ¹H NMR (400 MHz, CDCl₃) of the other diastereomer δ 8.37 (d, J=8, 2H), 7.36 (d, J=8, 2H), 6.24 (s, 1H), 5.15 (m, 2H), 4.63 (m, 1H), 4.22 (m, 1H), 4.04 (m, 2H), 3.79 (m, 2H), 3.58 (m, 5H), 3.43 (m, 1H), 3.32 (m, 2H), 2.0-2.7 (m, 6H), 2.15 (m, 1H), 2.00 (m, 1H), 1.48 (s, 3H), 1.36 (d, J=7.2, 3H), 1.18 (t, J=7.2, 3H); LC-MS m/z 511 (M+H).

### Example 77

### Preparation of 1-(4-(7-(2-(dimethylamino)ethyl)-7-methyl-4-((S)-3-methylmorpholino)-5,7-dihydrofuro[3,4-d]pyrimidin-2-yl)phenyl)-3-ethylurea (ep)

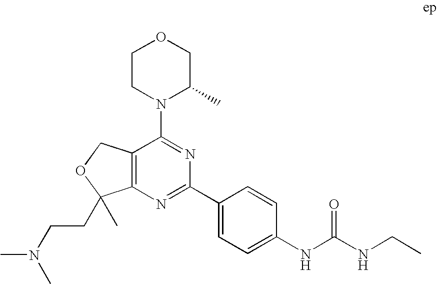

Synthesis of 1-(4-(7-(2-(dimethylamino)ethyl)-7-methyl-4-((S)-3-methylmorpholino)-5,7-dihydrofuro[3,4-d]pyrimidin-2-yl)phenyl)-3-ethylurea (ep). The title compound was prepared following the general procedure in Step 2 of Example 76, substituting morpholine with 2.0 M of dimethylamine in tetrahydrofuran. The diastereomers were separated by chiral HPLC: LC-MS m/z 469 (M+H).

### Example 78

### Preparation of 1-ethyl-3-(4-(7-(2-(ethyl(methyl)amino)ethyl)-7-methyl-4-((S)-3-methylmorpholino)-5,7-dihydrofuro[3,4-d]pyrimidin-2-yl)phenyl)urea (eq)

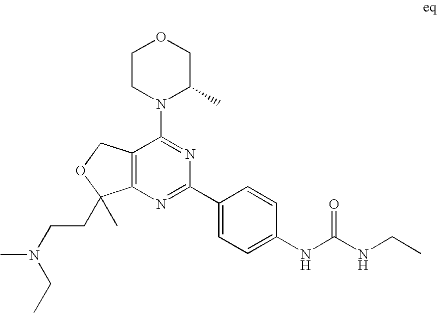

Synthesis of 1-ethyl-3-(4-(7-(2-(ethyl(methyl)amino)ethyl)-7-methyl-4-((S)-3-methylmorpholino)-5,7-dihydrofuro[3,4-d]pyrimidin-2-yl)phenyl)urea (eq). The title compound was prepared following the general procedure in Step 2 of Example 76, substituting morpholine with N-methylethylamine. The diastereomers were separated by chiral HPLC: LC-MS m/z 483 (M+H).

### Example 79

### Preparation of 1-(4-(7-(2-(az etidin-1-yl)ethyl)-7-methyl-4-((S)-3-methylmorp holino)-5,7-dihydro furo [3,4-d]pyrimidin-2-yl)phenyl)-3-ethylurea (er)

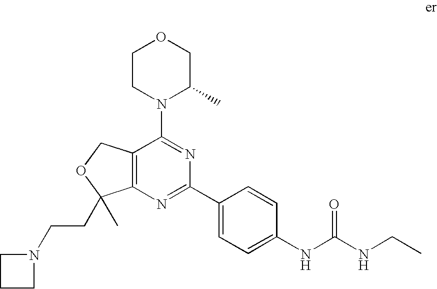

Synthesis of 1-(4-(7-(2-(azetidin-1-yl)ethyl)-7-methyl-4-((S)-3-methylmorpholino)-5,7-dihydrofuro[3,4-d]pyrimidin-2-yl)phenyl)-3-ethylurea. The title compound was prepared following the general procedure in Step 2 of Example 76, substituting morpholine with azetidine. The diastereomers were separated by chiral HPLC: LC-MS m/z 481 (M+H).

### Example 80

### Preparation of 1-(4-(7-(2-(1H-imidazol-1-yl)ethyl)-7-methyl-4-((S)-3-methylmorpholino)-5,7-dihydrofuro[3,4-d]pyrimidin-2-yl)phenyl)-3-ethylurea (es)

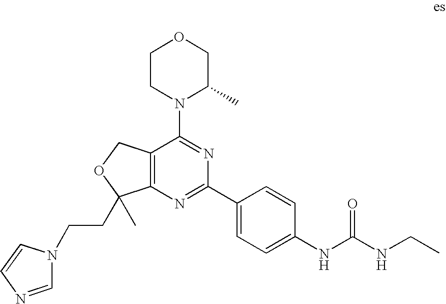

Synthesis of 1-(4-(7-(2-(1H-imidazol-1-yl)ethyl)-7-methyl-4-((S)-3-methylmorpholino)-5,7-dihydrofuro[3,4-d]pyrimidin-2-yl)phenyl)-3-ethylurea (es). The title compound was prepared following the general procedure in Step 2 of Example 76, substituting morpholine with imidazole. The diastereomers were separated by chiral HPLC: LC-MS m/z 492 (M+H).

### Example 81

### Preparation of 1 1-ethyl-3-(4-(7-methyl-7-(2-(2-methyl-1H-imidazol-1-yl)ethyl)-4-((S)-3-methylmorpholino)-5,7-dihydro furo [3,4-d]pyrimidin-2-yl)phenyl)urea (et)

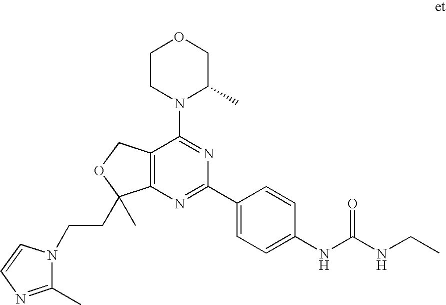

Synthesis of 1 1-ethyl-3-(4-(7-methyl-7-(2-(2-methyl-1H-imidazol-1-yl)ethyl)-4-((S)-3-methylmorpholino)-5,7-dihydrofuro[3,4-d]pyrimidin-2-yl)phenyl)urea (et). The title compound was prepared following the general procedure in Step 2 of Example 76, substituting morpholine with 2-methylimidazole. The diastereomers were separated by chiral HPLC: LC-MS m/z 506 (M+H).

### Example 82

### Preparation of 1-(4-(7-(2-(1H-pyrazol-1-yl)ethyl)-7-methyl-4-((S)-3-methylmorpholino)-5,7-dihydrofuro[3,4-d]pyrimidin-2-yl)phenyl)-3-ethylurea (eu)

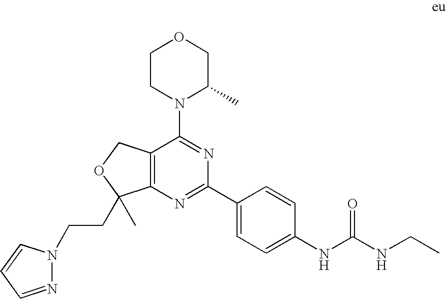

Synthesis of 1-(4-(7-(2-(1H-pyrazol-1-yl)ethyl)-7-methyl-4-((S)-3-methylmorpholino)-5,7-dihydrofuro[3,4-d]pyrimidin-2-yl)phenyl)-3-ethylurea (eu). The title compound was prepared following the general procedure in Step 2 of Example 76, substituting morpholine with pyrazole. The diastereomers were separated by chiral HPLC: LC-MS m/z 492 (M+H).

### Example 83

### Preparation of 1-ethyl-3-(4-(7-methyl-4-((S)-3-methylmorp ho lino)-7-propyl-5,7-dihydro furo [3,4-d]pyrimidin-2-yl)phenyl)urea (ew)

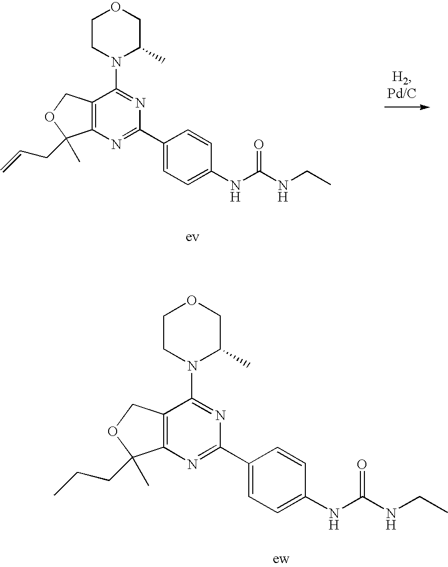

Synthesis of 1-ethyl-3-(4-(7-methyl-4-((S)-3-methylmorpholino)-7-propyl-5,7-dihydrofuro[3,4-d]pyrimidin-2-yl)phenyl)urea (ew). The title compound was prepared following the general procedure in Step 6 of Example 73, substituting 1-(4-(7-allyl-7-methyl-4-morpholino-5,7-dihydrofuro[3,4-d]pyrimidin-2-yl)phenyl)-3-ethylurea with 1-(4-(7-allyl-7-methyl-4-((S)-3-methylmorpholino)-5,7-dihydrofuro[3,4-d]pyrimidin-2-yl)phenyl)-3-ethylurea. The diastereomers were separated by chiral HPLC: LC-MS m/z 440 (M+H).

### Example 84

### Preparation of 1-ethyl-3-(4-(7-(hydroxymethyl)-7-methyl-4-((S)-3-methylmorpholino)-5,7-dihydrofuro[3,4-d]pyrimidin-2-yl)phenyl)urea (fb)

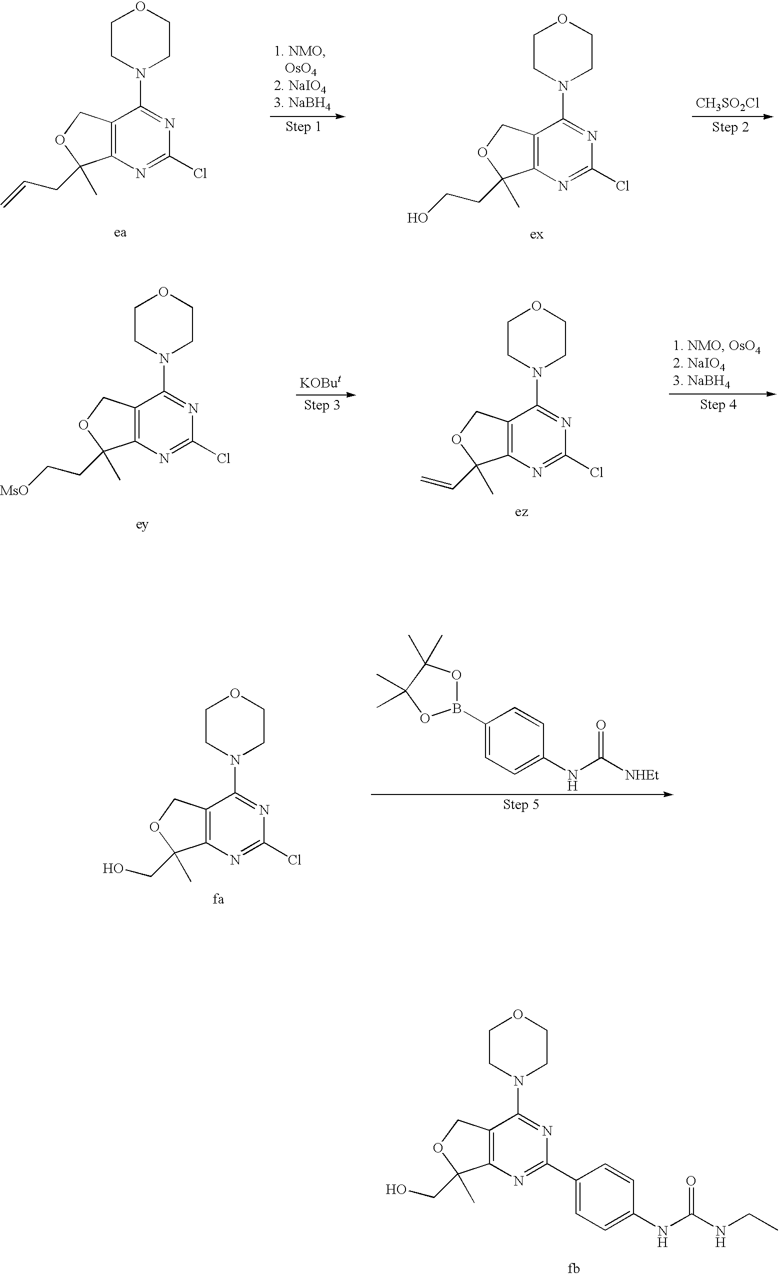

Step 1—Synthesis of 2-(2-chloro-7-methyl-4-morpholino-5,7-dihydrofuro[3,4-d]pyrimidin-7-yl)ethanol (ex). The title compound was prepared following the general procedure from Step 3 to Step 5 in Example 73, substituting 1-(4-(7-allyl-7-methyl-4-morpholino-5,7-dihydrofuro[3,4-d]pyrimidin-2-yl)phenyl)-3-ethylurea for 7-allyl-2-chloro-7-methyl-4-morpholino-5,7-dihydrofuro[3,4-d]pyrimidine: LC-MS m/z 300 (M+H).

Step 2—Synthesis of 2-(2-chloro-7-methyl-4-morpholino-5,7-dihydrofuro[3,4-d]pyrimidin-7-yl)ethyl methanesulfonate (ey). The title compound was prepared following the general procedure in Step 1 of Example 76, substituting 1-ethyl-3-(4-(7-(2-hydroxyethyl)-7-methyl-4-(S)-3-methylmorpholino)-5,7-dihydrofuro[3,4-d]pyrimidin-2-yl)phenyl)urea with 2-(2-chloro-7-methyl-4-morpholino-5,7-dihydrofuro[3,4-d]pyrimidin-7-yl)ethanol. The diastereomers were separated by chiral HPLC: LC-MS m/z 378 (M+H).

Step 3—Synthesis of 2-chloro-7-methyl-4-morpholino-7-vinyl-5,7-dihydrofuro[3,4-d]pyrimidine (ez). To a solution of 2-(2-chloro-7-methyl-4-morpholino-5,7-dihydrofuro[3,4-d]pyrimidin-7-yl)ethyl methanesulfonate (320 mg, 0.85 mmol) in tetrahydrofuran (8.5 mL) was added potassium tert-butoxide (190 mg, 1.7 mmol) at 0° C., and the reaction was stirred at room temperature overnight. The yellow suspension was quenched with water and brine, and diluted with Et₂O-EtOAc (v/v, 3:1, 200 mL). After work-up, the crude yellow oil was used without further purification: LC-MS m/z 282 (M+H).

Step 4—Synthesis of (2-chloro-7-methyl-4-morpholino-5,7-dihydrofuro[3,4-d]pyrimidin-7-yl)methanol (fa). The title compound was prepared following the general procedure in Step 1 of Example 84, substituting 7-allyl-2-chloro-7-methyl-4-morpholino-5,7-dihydrofuro[3,4-d]pyrimidine with 2-chloro-7-methyl-4-morpholino-7-vinyl-5,7-dihydrofuro[3,4-d]pyrimidine: LC-MS m/z 286 (M+H).

Step 5—Synthesis of 1-ethyl-3-(4-(7-(hydroxymethyl)-7-methyl-4-((S)-3-methylmorpholino)-5,7-dihydrofuro[3,4-d]pyrimidin-2-yl)phenyl)urea (fb). The title compound was prepared following the general procedure in Step 2 of Example 73, substituting 7-allyl-2-chloro-7-methyl-4-morpholino-5,7-dihydrofuro[3,4-d]pyrimidine with (2-chloro-7-methyl-4-morpholino-5,7-dihydrofuro[3,4-d]pyrimidin-7-yl)methanol. The enantiomers were separated by chiral HPLC: ¹H NMR (400 MHz, CDCl₃) δ 8.34 (d, J=8, 2H), 7.36 (d, J=8, 2H), 6.31 (s, 1H), 5.22 (m, 2H), 4.66 (m, 1H), 3.7-3.9 (m, 10H), 3.33 (m, 2H), 2.45 (m, 1H), 1.49 (s, 3H), 1.18 (t, J=7.2, 3H); LC-MS m/z 414 (M+H).

### Example 85

### Preparation of 1-(4-((S)-7-allyl-7-methyl-4-((S)-3-methylmorpholino)-5,7-dihydrofuro[3,4-d]pyrimidin-2-yl)phenyl)-3-ethylurea (ek¹) and 1-(4-((R)-7-allyl-7-methyl-4-((S)-3-methylmorpholino)-5,7-dihydrofuro[3,4-d]pyrimidin-2-yl)phenyl)-3-ethylurea (ek²)

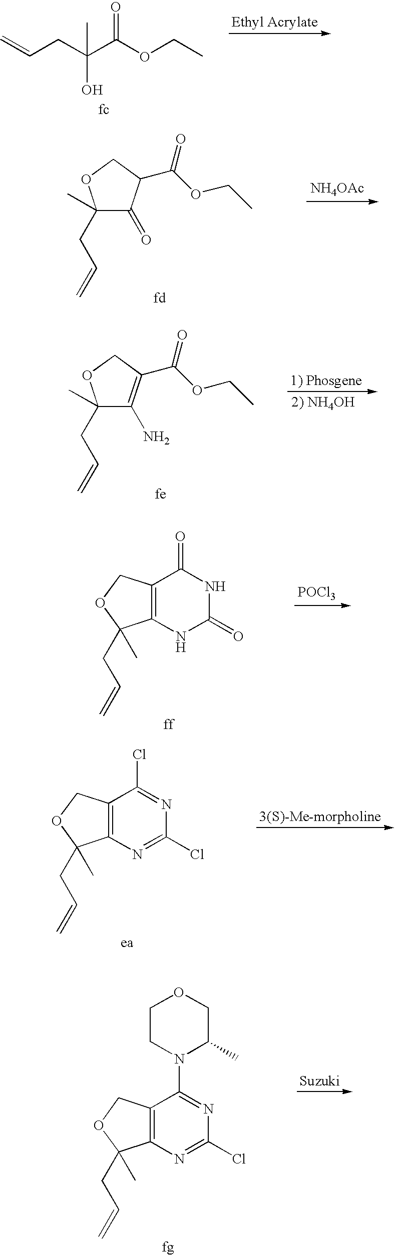

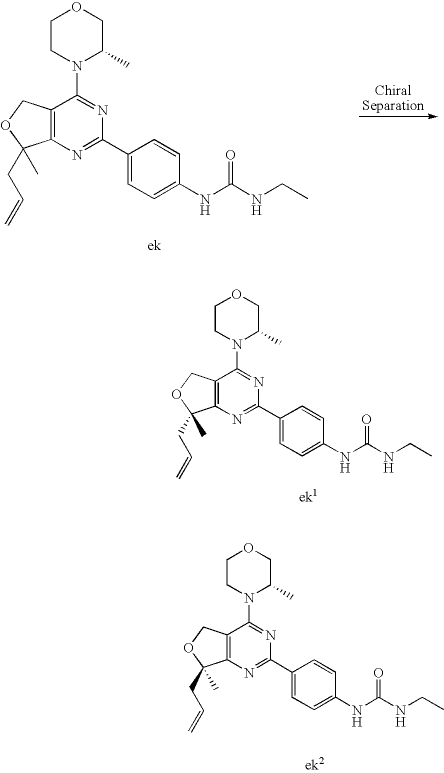

Step 1—Synthesis of ethyl 5-allyl-5-methyl-4-oxotetrahydrofuran-3-carboxylate (fd). To an ice-bath cooled suspension of NaH (1.9 g, 49 mmol) in THF under N₂ was added ethyl 2-hydroxy-2-methylpent-4-enoate [see, Ojima, J. C. S Chem. Comm. 1976, 927] (7.0 g, 44 mmol) dropwise. The clear brown solution was allowed to warm to rt, stirred for 30 min. It was concentrated in vacuo to an oil. The oil was cooled in ice-bath under N₂, and a solution of ethyl acrylate (14 mL, 130 mmol) in DMSO (50 mL) was added to the cooled oil via cannula over 2 min. The resulting mixture was stirred in ice-bath for 15 min, then allowed to warm to rt and stirred for 1.5 h. The mixture was poured into cold 3% aqueous H₂SO₄ (700 mL) over 10 min. It was extracted with ether (3×). The combined organics were washed with brine, dried over MgSO₄, filtered, concentrated in vacuo gave clear liquid (fd). It was carried on without further purification.

Step 2—Synthesis of ethyl 5-allyl-4-amino-5-methyl-2,5-dihydrofuran-3-carboxylate (fe). Crude (fd) from Step 1 (9.4 g, 44 mmol), NH₄OAc (34 g, 443 mmol) and EtOH (200 mL) was heated at 85° C. for overnight. EtOH was removed in vacuo. The residue was diluted with EtOAc. The precipitate was filtered, washed with EtOAc. The combined organics were extracted with 10% NaHCO₃, back extracted the aqueous with EtOAc. The combined EtOAc was washed with water, brine, and dried over MgSO₄, filtered, concentrated in vacuo yellow oil. The material was purified by column chromatography (ISCO, 220 g column), 1-15% EtOAc/Heptane to give 4.7 g (51%) of a light yellow oil (fe); LC-MS: m/z=+212 (M+H)+.

Step 3—Synthesis of 7-allyl-7-methyl-5,7-dihydrofuran[3,4-d]pyrimidine-2,4(1H,3H)-dione (ff). A solution of (fe) (13.5 g, 63.9 mmol) from Step 2 and pyridine (20.7 mL, 256 mmol) dichloromethane (230 mL) was cooled in ice-bath. 20% Phosgene/toluene solution (50.7 mL, 95.8 mmol) was added dropwise, ice-bath was removed, stirred for 2 h. Again cooled in ice-bath, NH₄OH (89 mL, 639 mmol) was added dropwise. After 15 min, it was allowed to warm to rt then heated at 50° C. overnight. The phases were separated, the dichloromethane was washed with 1% NH₄OH (2×100 mL). The combined aqueous phases were extracted with dichloromethane (2×). The aqueous was concentrated in vacuo to small volume and solid precipitated. The solids were collected by filtration, washed with small amount of water, dried, high vac to give 5.9 g (ff) as yellow solid; LC-MS: m/z=+209 (M+H)+.

Step 4—Synthesis of 7-allyl-2,4-dichloro-7-methyl-5,7-dihydrofuro[3,4-d]pyrimidine (ea). To a suspension of (ff) (5.9 g, 28 mmol) from Step 3 and dichloromethane (20 mL) was POCl₃ (35 mL, 375 mmol) slowly. The resulting mixture was heated in a glass sealed-tube at 90° C. overnight. After cooled, it was poured into crushed ice (300 mL), basified by adding NaOH pellets (few at a time) in ice-bath. The dark brown basic mixture was extracted with dichloromethane (3×100 mL). The combined dichloromethane extract was dried over MgSO₄, filtered, concentrated in vacuo to give 5.8 g (85%) of (ea) as dark brown solid. It was carried on without further purification; LC-MS: m/z=+246 (M+H)+.

Step 5—Synthesis of 7-allyl-2-chloro-7-methyl-4-((S)-3-methylmorpholino)-5,7-dihydrofuro[3,4-d]pyrimidine (fg). To a solution of (ea) (5.8 g, 24 mmol) from Step 4 and DIPEA (8.3 mL, 47.7 mmol) in DMF (55 mL) was added a solution of (S)-3-methylmorpholine (2.7 g, 26.2 mmol) in DMF (5 mL) dropwise at rt. The resulting dark solution was stirred at rt overnight. It was diluted with water (400 mL), extracted with EtOAc (3×120 mL). The combined organics were washed with brine, dried over MgSO₄, filtered, concentrated in vacuo, high vac to give 7.6 g (100%) of (fg) as dark oil. It was carried on without further purification; LC-MS: m/z=+310 (M+H)+.

Step 6—Synthesis of 1-(7-allyl-7-methyl-4-((S)-3-methylmorpholino)-5,7-dihydrofuro[3,4-d]pyrimidin-2-yl)phenyl)-3-ethylurea (ek). To a mixture of (fg) (720 mg, 2.3 mmol) from Step 5,4-(ethylureido)phenylboronic acid, pinacol ester (810 mg, 2.8 mmol), tetrakis(triphenylphosphine)palladium (0) (270 mg, 0.23 mmol), 1N aq. KOAc (3.5 mL), 1N aq. Na₂CO₃ (4.6 mL), and acetonitrile (6 mL) was capped in a microwave vial, purged with N₂ for few minutes. It was heated in microwave reactor at 120° C. for 20 min. The mixture was diluted with water, extracted with EtOAc (2×). The combined EtOAC was dried over MgSO₄, filtered, concentrated in vacuo. The residue was purified by column chromatography (ISCO, 40 g column), 5-50% EtOAc/Heptane to give 672 mg (66%) of (ek) as yellow solid. The racemic (ek) was subjected to chiral separations to afford isomers (ek¹) and (ek²).

(isomer 1): ¹H NMR (400 MHz, DMSO) δ 8.68 (s, 1H), 8.20 (d, J=8.8 Hz, 2H), 7.48 (d, J=8.8 Hz, 2H), 6.16 (t, J=5.6 Hz, 1H), 5.79-5.67 (m, 1H), 5.17-4.97 (m, 4H), 4.22 (s, 1H), 4.08-3.91 (m, 2H), 3.67 (dt, J=11.6, 7.1 Hz, 2H), 3.50 (td, J=11.8, 2.7 Hz, 1H), 3.28 (s, 1H), 3.16-3.08 (m, 2H), 1.37 (s, 3H), 1.25 (d, J=6.8 Hz, 3H), 1.06 (t, J=7.2 Hz, 3H); LC-MS: m/z=+438 (M+H)+; (isomer 2): ¹H NMR (400 MHz, DMSO) δ 8.68 (s, 1H), 8.20 (d, J=8.8 Hz, 2H), 7.48 (d, J=8.8 Hz, 2H), 6.16 (t, J=5.5 Hz, 1H), 5.77-5.67 (m, 1H), 5.18 (d, J=11.7 Hz, 1H), 5.08-4.96 (m, 3H), 4.22 (s, 1H), 3.94 (dd, J=11.4, 3.1 Hz, 2H), 3.67 (dt, J=11.6, 7.1 Hz, 2H), 3.50 (td, J=11.9, 2.7 Hz, 1H), 3.30 (d, J=13.1 Hz, 2H), 3.17-3.08 (m, 2H), 1.37 (s, 3H), 1.23 (d, J=6.7 Hz, 3H), 1.06 (t, J=7.2 Hz, 3H); LC-MS: m/z=+438 (M+H)+.

### Example 86

### Preparation of 1-ethyl-3-(4-((S)-7-(2-hydroxyethyl)-7-methyl-4-((S)-3-methylmorpholino)-5,7-dihydrofuro[3,4-d]pyrimidin-2-yl)phenyl)urea (em¹) and 1-ethyl-3-(4-((R)-7-(2-hydroxyethyl)-7-methyl-4-((S)-3-methylmorpholino)-5,7-dihydrofuro[3,4-d]pyrimidin-2-yl)phenyl)urea (em²)

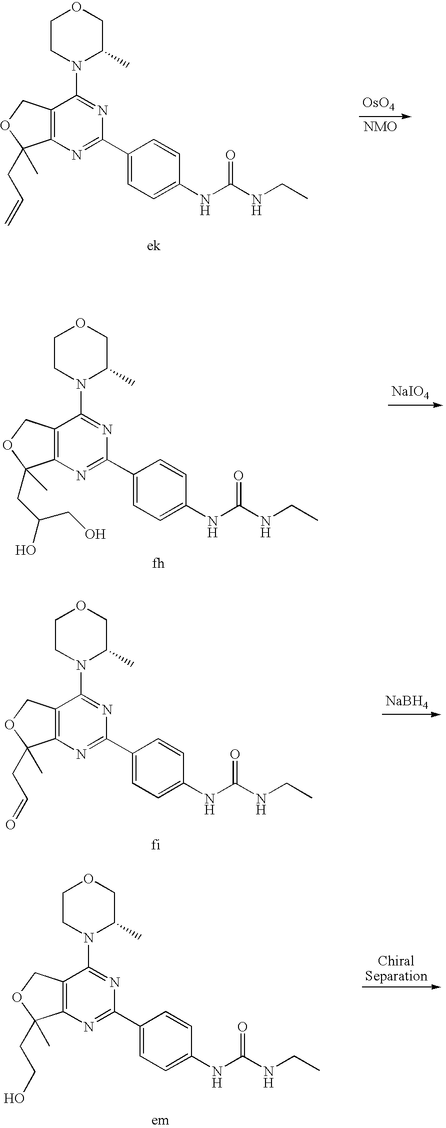

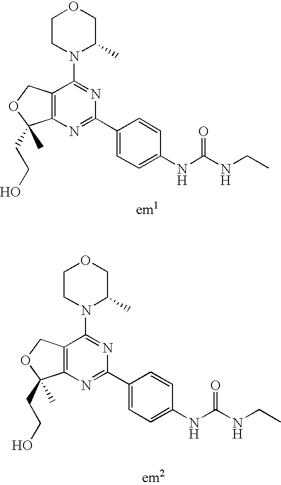

Step 1—Synthesis of 1-ethyl-3-(4-(7-(2-hydroethyl)-7-methyl-4-((S)-3-methylmorpholino)-5,7-dihydrofuro[3,4-d]pyrimidin-2-yl)phenyl)urea (fh). (ek) (100 mg, 0.2 mmol) from Example 85 was dissolved in THF:water (3:1, 6 mL) and cooled in ice-bath. To this solution was added N-methylmorpholine N-oxide (32 mg, 0.27 mmol) and OsO₄ (a couple crystals). The resulting solution was stirred at rt for 48 h. It was quenched by the addition of Na₂SO₃ (340 mg, 2.7 mmol), stirred at rt for 30 min, diluted with water, extracted with EtOAc (2×). The combined EtOAC extract was dried over MgSO₄, filtered, concentrated in vacuo to give crude diol (fh).

Step 2—The crude diol (fh) was dissolved in THF:water (3:1, 6 mL) and NaIO₄ (73 mg, 0.34 mmol) was added in one portion, stirred at rt for 1.5 h. The reaction mixture was diluted with brine, extracted with EtOAc (2×). The combined EtOAC extract was dried over MgSO₄, filtered, concentrated in vacuo to give crude aldehyde (fi).

Step 3—Aldehyde (fi) was dissolved in THF (2 mL) with few drops of MeOH. It was cooled in ice-bath and NaBH₄ was added. The resulting mixture was stirred at rt for 30 min. The reaction was quenched with sat aq NH₄Cl and partitioned with EtOAc (2×). The combined EtOAC extract was dried over MgSO₄, filtered, concentrated in vacuo, and purified by HPLC to give 60 mg (60%) of (em) as white solid. Compound (em) was subjected to chiral separations to afford isomers (em¹) and (em²): (isomer 1): ¹H NMR (400 MHz, DMSO) δ 8.65 (s, 1H), 8.20 (d, J=8.8 Hz, 2H), 7.48 (d, J=8.8 Hz, 2H), 6.14 (t, J=5.7 Hz, 1H), 5.11 (q, J=11.7 Hz, 2H), 4.33 (t, J=5.3 Hz, 1H), 4.24 (s, 1H), 3.94 (d, J=8.0 Hz, 2H), 3.68 (dd, J=26.9, 9.9 Hz, 2H), 3.58-3.47 (m, 2H), 3.32 (s, 1H), 3.11 (dd, J=13.5, 6.4 Hz, 2H), 1.95 (d, J=9.2 Hz, 2H), 1.37 (s, 3H), 1.25 (d, J=6.7 Hz, 3H), 1.06 (t, J=7.1 Hz, 3H), LC-MS: m/z=+442 (M+H)+; (isomer 2): ¹H NMR (400 MHz, DMSO) δ 8.65 (s, 1H), 8.20 (d, J=8.8 Hz, 2H), 7.48 (d, J=8.8 Hz, 2H), 6.14 (t, J=5.6 Hz, 1H), 5.16 (d, J=11.7 Hz, 1H), 5.06 (d, J=11.7 Hz, 1H), 4.33 (t, J=5.3 Hz, 1H), 4.24 (s, 1H), 3.94 (d, J=8.3 Hz, 2H), 3.68 (dt, J=11.7, 7.2 Hz, 2H), 3.53 (ddd, J=23.7, 15.1, 8.4 Hz, 2H), 3.33 (s, 1H), 3.17-3.07 (m, 2H), 2.01-1.91 (m, 2H), 1.37 (s, 3H), 1.25 (d, J=6.7 Hz, 3H), 1.06 (t, J=7.2 Hz, 3H); LC-MS: m/z=+442 (M+H)+.

### Example 87

### Preparation of 3-ethyl-1-(4-((S)-7-(2-hydroxyethyl)-7-methyl-4-((S)-3-methylmorpholino)-5,7-dihydrofuro[3,4-d]pyrimidin-2-yl)phenyl)-1-methylurea (fj¹) and 3-ethyl-1-(4-((R)-7-(2-hydroxyethyl)-7-methyl-4-((S)-3-methylmorpholino)-5,7-dihydrofuro[3,4-d]pyrimidin-2-yl)phenyl)-1-methylurea (fj²)

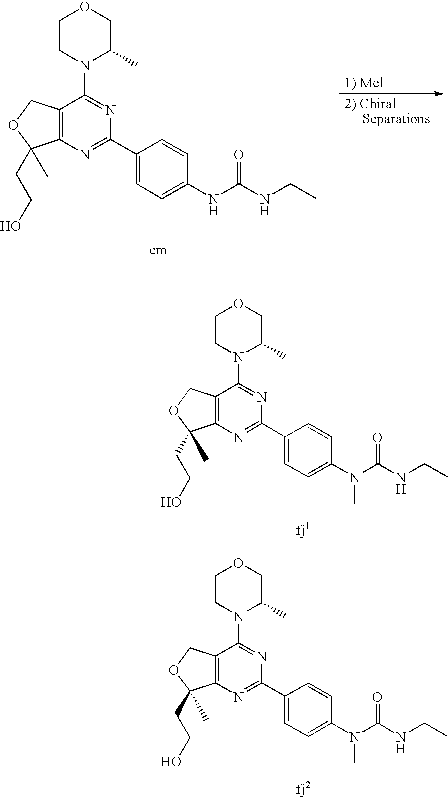

To a solution of (em) (130 mg, 0.29 mmol) and NaOMe (52.5 mg, 0.97 mmol) in THF (4 mL) was added MeI (0.020 mL, 0.32 mmol) at rt. After 6 h at rt, it was quenched by the addition of AcOH (a few drops). The mixture was concentrated in vacuo. The residue was purified by reverse-phase HPLC followed by chiral HPLC to afford products (fj¹) and (fj²). A combined yield of 64 mg (48%); (isomer 1) LC-MS: m/z=+456 (M+H)+; (isomer 2) LC-MS: m/z=+456 (M+H)+.

### Example 88

### Preparation of 1-(4-(7-(cyclopropylmethyl)-7-methyl-4-((S)-3-methylmorp holino)-5,7-dihydro furo [3,4-d]pyrimidin-2-yl)phenyl)-3-ethylurea (fk)

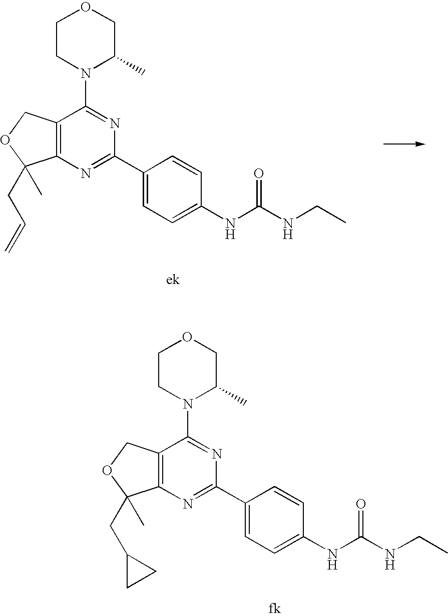

Synthesis of 1-(4-(7-(cyclopropylmethyl)-7-methyl-4-((S)-3-methylmorpholino)-5,7-dihydrofuro[3,4-d]pyrimidin-2-yl)phenyl)-3-ethylurea (fk). A diethyl zinc/toluene (15 wt %) solution (0.4 mL, 0.4 mmol) was carefully added to anhy. dichloromethane (1 mL) under N₂. The solution was cooled in an ice-bath and a solution of TFA (0.040 mL, 0.4 mmol) in dichloromethane (0.3 mL) was added dropwise, stirred for 20 min. A solution of diiodomethane (0.040 mL, 0.4 mmol) in dichloromethane (0.3 mL) was added. After 20 min, a solution of (G) (100 mg, 0.2 mmol) in dichloromethane (1.4 mL) was added. After a few min, ice-bath was removed, stirred at rt for 1.5 h. The reaction mixture was quenched with 0.1 N HCl (5 mL), extracted with EtOAc, dried over MgSO₄, filtered, concentrated in vacuo. The residue was purified by reverse-phase HPLC to give 19 mg (20%) of (fk) as off-white solid; LC-MS: m/z=+452 (M+H)+.

Separation of compound fk to fk¹ and fk²:

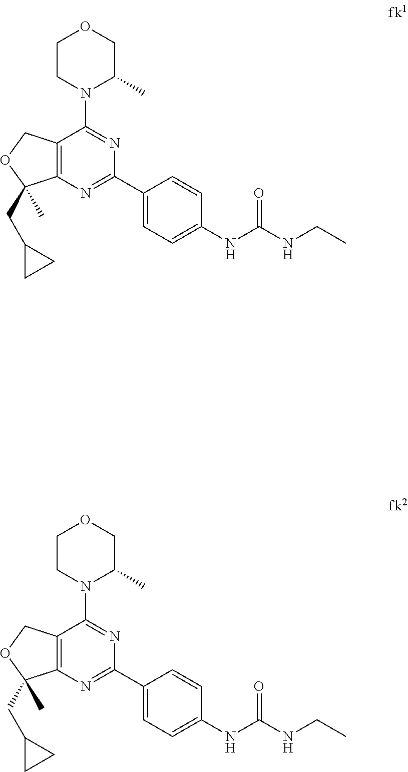

Chiral HPLC separation of (fk) to afford 1-(4-((R)-7-(cyclopropylmethyl)-7-methyl-4-((S)-3-methylmorpholino)-5,7-dihydrofuro [3,4-d.]pyrimidin-2-yl)phenyl)-3-ethylure a (fk¹) and 1-(4-((S)-7-(cyclopropylmethyl)-7-methyl-4-((S)-3-methylmorpholino)-5,7-dihydrofuro[3,4-d]pyrimidin-2-yl)phenyl)-3-ethylurea (fk²); (isomer 1): ¹H NMR (500 MHz, DMSO) δ 8.68 (s, 1H), 8.20 (d, J=8.8 Hz, 2H), 7.47 (d, J=8.8 Hz, 2H), 6.17 (t, J=5.5 Hz, 1H), 5.20 (d, J=11.6 Hz, 1H), 5.13 (d, J=11.7 Hz, 1H), 4.27-4.17 (m, 1H), 4.09-3.97 (m, 1H), 3.94 (d, J=10.9 Hz, 1H), 3.72 (d, J=11.3 Hz, 1H), 3.69-3.64 (m, 1H), 3.51 (dd, J=11.9, 8.9 Hz, 1H), 3.30-3.26 (m, 1H), 3.15-3.08 (m, 2H), 1.66 (ddd, J=26.0, 14.2, 6.8 Hz, 2H), 1.41 (s, 2H), 1.25 (d, J=6.8 Hz, 2H), 1.06 (t, J=7.2 Hz, 2H), 0.67 (s, 1H), 0.37 (dt, J=9.1, 6.5 Hz, 1H), 0.23-0.16 (m, 1H), 0.06 (dd, J=9.1, 4.2 Hz, 1H), −0.13 (dt, J=9.0, 4.3 Hz, 1H); LC-MS: m/z=+452 (M+H)+; (isomer 2): ¹H NMR (500 MHz, DMSO) δ 8.67 (s, 1H), 8.20 (d, J=8.8 Hz, 2H), 7.48 (d, J=8.8 Hz, 2H), 6.16 (t, J=5.6 Hz, 1H), 5.21 (d, J=11.6 Hz, 1H), 5.13 (d, J=11.6 Hz, 1H), 4.25-4.16 (m, 1H), 4.01 (s, 1H), 3.97-3.92 (m, 1H), 3.68 (dt, J=11.5, 7.2 Hz, 2H), 3.50 (dt, J=12.0, 6.1 Hz, 1H), 3.35-3.32 (m, 1H), 3.15-3.09 (m, 2H), 1.67 (ddd, J=21.1, 14.3, 7.0 Hz, 2H), 1.40 (s, 3H), 1.23 (d, J=6.8 Hz, 3H), 1.06 (t, J=7.2 Hz, 3H), 0.66 (s, 1H), 0.36 (td, J=9.2, 5.3 Hz, 1H), 0.18 (dt, J=13.2, 7.2 Hz, 1H), 0.06 (td, J=9.3, 5.0 Hz, 1H), −0.17 (td, J=9.2, 4.9 Hz, 1H); LC-MS: m/z=+452 (M+H)+.

### Example 89

### Preparation of 1-ethyl-3-(4-((R)-7-(2-methoxyethyl)-7-methyl-4-((S)-3-methylmorpholino)-5,7-dihydrofuro[3,4-d]pyrimidin-2-yl)phenyl)urea (fn¹) and 1-ethyl-3-(4-((S)-7-(2-methoxyethyl)-7-methyl-4-((S)-3-methylmorpholino)-5,7-dihydrofuro[3,4-d]pyrimidin-2-yl)phenyl)urea (fn²)

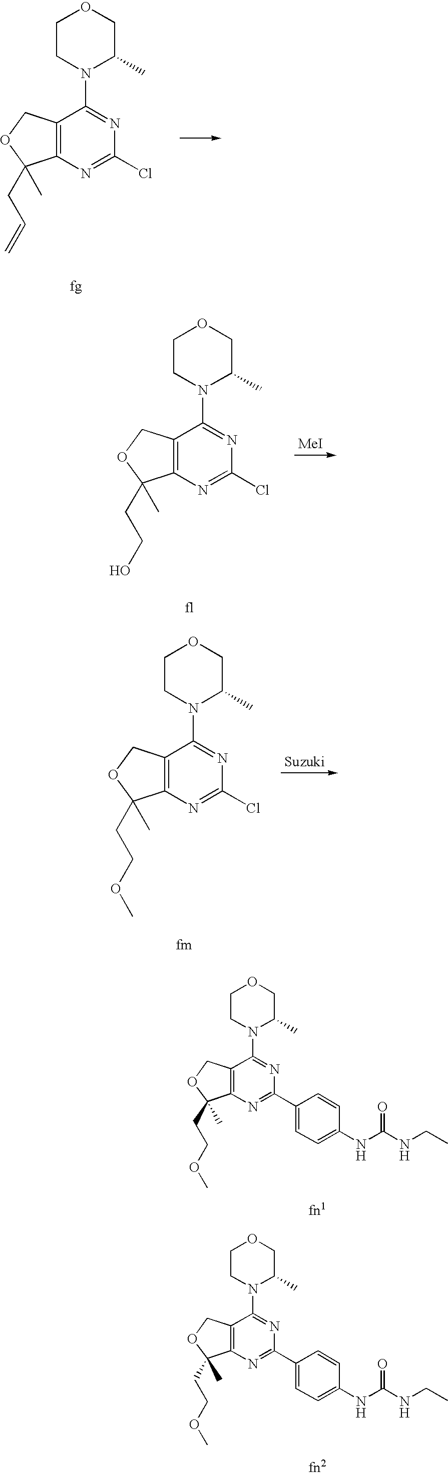

Step 1—Synthesis of 2-(2-chloro-7-methyl-4-((S)-3-methylmorpholino)-5,7-dihydrofuro[3,4-d]pyrimidin-7-yl)ethanol (fl). The title compound was prepared by the general procedure of Example 86, Steps 1-3; LC-MS: m/z=+314 (M+H)+.

Step 2—Synthesis of 2-chloro-7-(2-methoxyethyl)-7-methyl-4-((S)-3-methylmorpholino)-5,7-dihydrofuro[3,4-d]pyrimidine (fm). To a solution of (fl) (370 mg, 1.2 mmol) from Step 1 in THF (8 mL) was added MeI (0.22 mL, 3.5 mmol), followed by NaH (52 mg, 1.3 mmol). The resulting mixture was stirred at rt for 5 h. It was diluted with sat NH₄Cl, extracted with EtOAc (2×). The combined EtOAc extract was dried over MgSO₄, filtered, concentrated in vacuo. The residue was purified by column chromatography (ISCO, 25 g column), 0-20% EtOAc/dichloromethane to give 200 mg (52%) of (fm) as a yellow gum.

LC-MS: m/z=+328 (M+H)+.

Step 3—The title compound was prepared by the procedure of Example 85, Step 6, followed by chiral separations to afford isomers 1-ethyl-3-(4-((R)-7-(2-methoxyethyl)-7-methyl-4-((S)-3-methylmorpholino)-5,7-dihydrofuro[3,4-d]pyrimidin-2-yl)phenyl)urea and 1-ethyl-3-(4-((S)-7-(2-methoxyethyl)-7-methyl-4-((S)-3-methylmorpholino)-5,7-dihydrofuro[3,4-d]pyrimidin-2-yl)phenyl)urea; (isomer 1): ¹H NMR (500 MHz, DMSO) δ 8.63 (s, 1H), 8.20 (d, J=8.7 Hz, 2H), 7.48 (d, J=8.8 Hz, 2H), 6.14 (t, J=5.4 Hz, 1H), 5.17 (d, J=11.7 Hz, 1H), 5.07 (d, J=11.7 Hz, 1H), 4.22 (s, 1H), 4.05-3.91 (m, 2H), 3.68 (dt, J=11.5, 7.1 Hz, 2H), 3.50 (td, J=11.7, 2.6 Hz, 1H), 3.42 (td, J=9.0, 5.6 Hz, 1H), 3.33 (d, J=13.2 Hz, 1H), 3.25-3.20 (m, 1H), 3.16-3.09 (m, 5H), 2.08-1.95 (m, 2H), 1.38 (s, 3H), 1.25 (d, J=6.8 Hz, 3H), 1.06 (t, J=7.2 Hz, 3H); LC-MS: m/z=+456 (M+H)+; (isomer 2): ¹H NMR (500 MHz, DMSO) δ 8.63 (s, 1H), 8.20 (d, J=8.7 Hz, 2H), 7.48 (d, J=8.7 Hz, 2H), 6.14 (t, J=5.5 Hz, 1H), 5.12 (dd, J=25.7, 11.7 Hz, 2H), 4.24 (s, 1H), 4.05-3.91 (m, 2H), 3.74-3.63 (m, 2H), 3.51 (t, J=10.4 Hz, 1H), 3.43 (td, J=9.1, 5.6 Hz, 1H), 3.34 (s, 1H), 3.22 (td, J=9.0, 5.8 Hz, 1H), 3.16-3.09 (m, 5H), 2.08-1.95 (m, 2H), 1.38 (s, 3H), 1.25 (d, J=6.8 Hz, 3H), 1.06 (t, J=7.2 Hz, 3H); LC-MS: m/z=+456 (M+H)+.

### Example 90

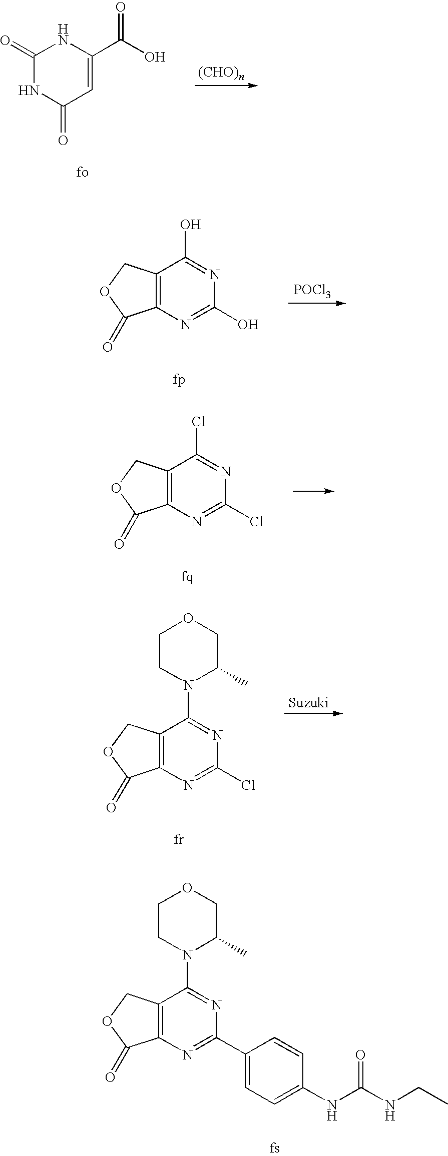

Step 1—Synthesis of 2,4-dihydroxyfuro[3,4-d]pyrimidin-7(5H)-one (fp). To a mixture of orotic acid (fo) (2.0 g, 13 mmol) and paraformaldehyde (1.5 g, 51 mmol) in conc. HCl (20 mL) was heated at 90° C. for 18 h. It was cooled, concentrated in vacuo. To the residue, water was added then concentrated again to dryness. Water (15 mL) was added to the white solid, heated in oil-bath at 70° C. for 30 min. It was allowed to stand at rt overnight. The white solid was collected by filtration, washed with small amount of water, dried, high vac to afford 550 mg (26%) of (fp) as white solid; LC-MS: m/z=+169 (M+H)+.

Step 2—Synthesis of 2,4-dichlorofuro[3,4-d]pyrimidin-7(5H)-one (fq). To a suspension of (fp) (550 mg, 3.3 mml) in dichloromethane (2.5 mL) was added POCl₃ (4.5 mL, 48 mmol), followed by dropwise addition of triethylamine (TEA) (0.91 mL, 6.5 mmol). The resulting was heated at 90° C. for overnight. Solvent was removed in vacuo. The residue was poured into ice, extracted with dichloromethane (3×). The combined dichloromethane extract was dried over MgSO₄, filtered, concentrated in vacuo to give 510 mg (76%) of (fq) as a brown solid. It was carried on without further purification; LC-MS: m/z=+205 (M+H)+.

Step 3—Synthesis of (S)-2-chloro-4-(3-methylmorpholino)furo[3,4-d]pyrimidin-7(5H)-one (fr). A solution of (fq) (250 mg, 1.2 mmol) in dichloromethane (3 mL) was cooled in ice-bath. (S)-3-methylmorpholine (0.14 g, 1.3 mmol) was added followed by DIPEA (0.23 mL, 1.3 mmol). The resulting dark red solution was stirred at rt for 2 h. It was diluted with 1 N HCl, and the phases separated. The aqueous layer was extracted with dichloromethane (2×). The combined dichloromethane extract was dried over MgSO₄, filtered, concentrated in vacuo to give 280 mg (85%) of (fr) as a yellow solid; LC-MS: m/z=+270 (M+H)+.

Step 4—Synthesis of (S)-1-ethyl-3-(4-(4-(3-methylmorpholino)-7-oxo-5,7-dihydrofuro[3,4-d]pyrimidin-2-yl)phenyl)urea (fs). The title compound was prepared by the general procedure of Example 85, Step 6; ¹H NMR (400 MHz, DMSO) δ 8.70 (s, 1H), 8.25 (d, J=8.7 Hz, 2H), 7.52 (d, J=8.8 Hz, 2H), 6.18 (t, J=5.5 Hz, 1H), 5.62 (dd, J=35.5, 15.0 Hz, 2H), 4.60-4.06 (m, 1H), 3.98 (d, J=9.2 Hz, 1H), 3.73 (dd, J=29.8, 10.4 Hz, 2H), 3.55 (t, J=11.8 Hz, 1H), 3.40 (d, J=28.5 Hz, 1H), 3.17-3.08 (m, 2H), 1.32 (d, J=6.7 Hz, 3H), 1.06 (t, J=7.2 Hz, 3H); LC-MS: m/z=+398 (M+H)+.

### Example 91

### Preparation of 1-ethyl-3-(4-((S)-7-methyl-4-((S)-3-methylmorpholino)-7-(2-phenoxyethyl)-5,7-dihydro furo [3,4-d]pyrimidin-2-yl)phenyl)urea (ft¹) and 1-ethyl-3-(4-((R)-7-methyl-4-((S)-3-methylmorpholino)-7-(2-phenoxyethyl)-5,7-dihydrofuro [3,4-d]pyrimidin-2-yl)phenyl)urea (ft²)

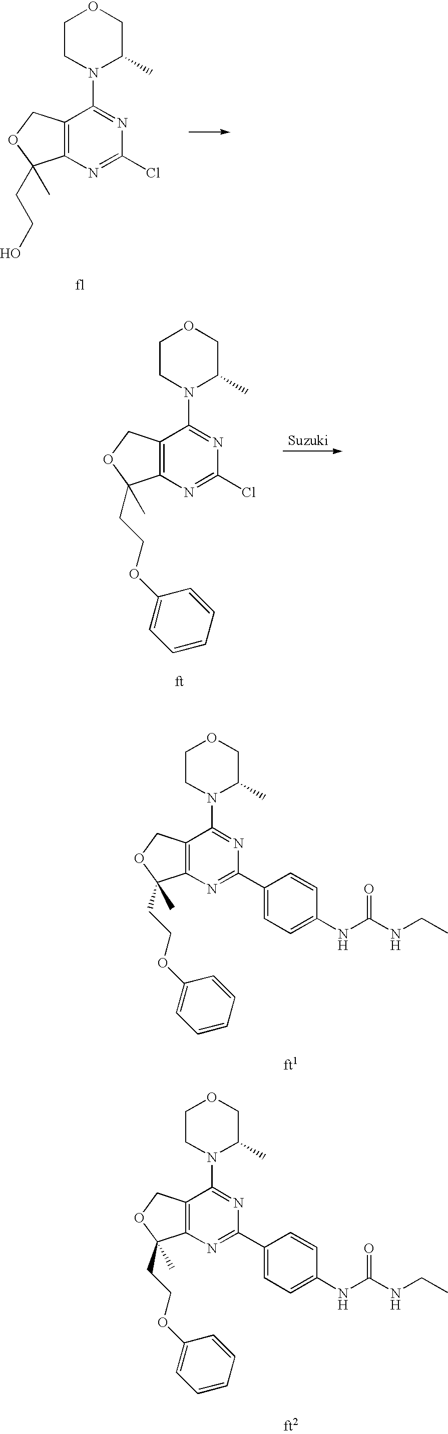

Step 1—To a solution of (fl) (300 mg, 1 mmol), phenol (90 mg, 1 mmol), and triphenylphosphine (200 mg, 1 mmol) in THF (3 mL) was cooled in ice-bath under N₂. Diethyl azodicarboxylate (0.16 mL, 1 mmol) was added dropwise. The resulting yellow solution was stirred at rt overnight. Addition of phenol (45 mg), Ph₃P (100 mg), and DEAD (0.08 mL) were added respectively, and the resultant solution was stirred for 4 h. The reaction mixture was concentrated onto Celite, purified by ISCO, 24 g column, 1-30% EtOAc/Heptane to give 240 mg (60%) of (ft) as white solid; LC-MS: m/z=+390 (M+H)+.

Step 2—The title compounds were prepared by the procedure of Example 85, Step 6, followed by chiral HPLC separation to afford isomers (ft¹) and (ft²).

(isomer 1) LC-MS: m/z=+518 (M+H)⁺; (isomer 2) LC-MS: m/z=+518 (M+H)+.

### Example 92

### Preparation of 1-ethyl-3-(4-((S)-7-methyl-4-((S)-3-methylmorpholino)-7-(2-(pyridin-4-yloxy)ethyl)-5,7-dihydro furo [3,4-d]pyrimidin-2-yl)phenyl)urea (fu¹) and 1-ethyl-3-(4-((R)-7-methyl-4-((S)-3-methylmorp ho lino)-7-(2-(pyridin-4-yloxy)ethyl)-5,7-dihydro furo [3,4-d]pyrimidin-2-yl)phenyl)urea (fu²)

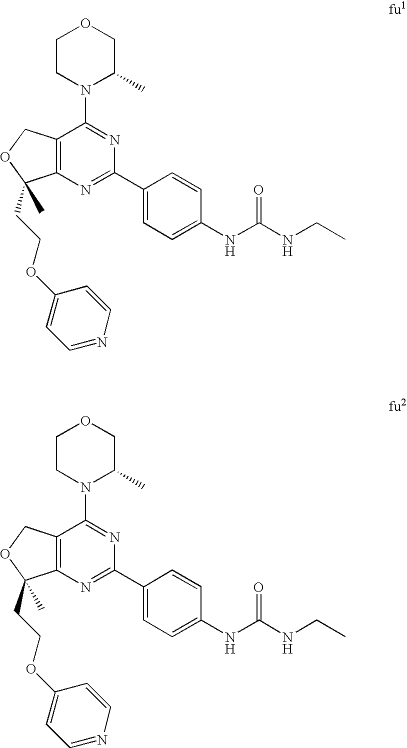

The title compounds were prepared by the general procedures of Example 91 substituting phenol with 4-pyridinol to afford (fu¹) and (fu²); (isomer 1) LC-MS: m/z=+519 (M+H)+; (isomer 2) LC-MS: m/z=+519 (M+H)+.

### Example 93

### Preparation of 1-(4-((R)-7-(2-cyano ethyl)-7-methyl-4-((S)-3-methylmorpholino)-5,7-dihydro furo [3,4-d]pyrimidin-2-yl)phenyl)-3-ethylurea (fx¹) and 1-(4-((S)-7-(2-cyano ethyl)-7-methyl-4-((S)-3-methylmorp ho lino)-5,7-dihydro furo [3,4-d]pyrimidin-2-yl)phenyl)-3-ethylurea (fx²)

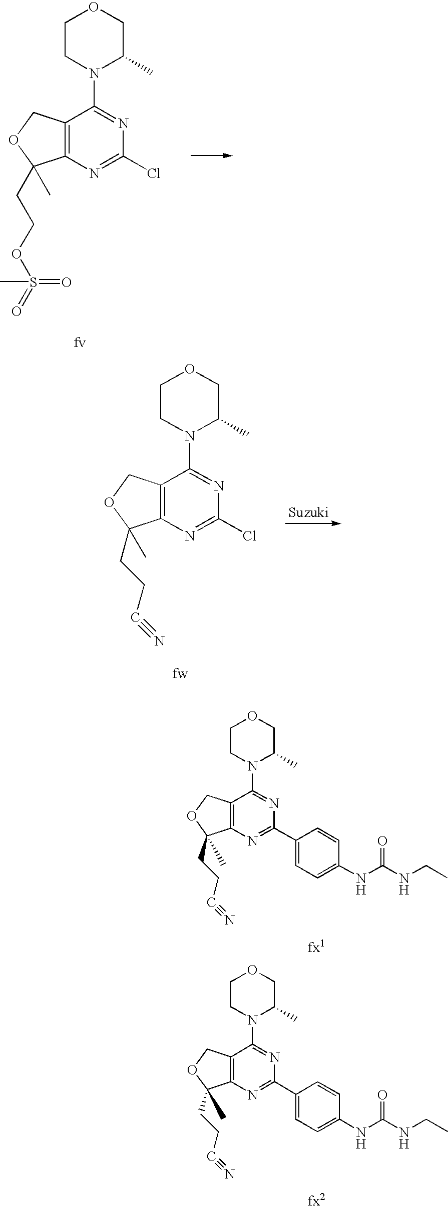

Step 1—Synthesis of 2-(2-chloro-7-methyl-4-((S)-3-methylmorpholino)-5,7-dihydrofuro[3,4-d]pyrimidin-7-yl)ethylmethanesulfonate (fv). To a cool (0° C.) solution of (fl) (700 mg, 2 mmol) from Example 91, Step 1 and DIPEA (0.78 mL, 4.5 mmol) and dichloromethane (15 mL) was added methanesulfonyl chloride (0.43 mL, 5.6 mmol) dropwise. It was stirred at rt for 2 h. It was diluted with dichloromethane, washed with sat NaHCO₃, dried over MgSO₄, filtered, concentrated in vacuo to give 1.1 g (100%) of (fv) as brown gum. It was carried on without further purification; LC-MS: m/z=+392 (M+H)+.

Step 2—Synthesis of 3-(2-chloro-7-methyl-4-((S)-3-methylmorpholino)-5,7-dihydrofuro[3,4-d]pyrimidin-7-yl)propanenitrile (fw). To a solution of (fv) (200 mg, 0.5 mmol) from Step 1 in DMSO (2 mL) was added NaCN (75 mg, 1.5 mmol) in one portion, heated at 45° C. for 2.5 h, then at 50° C. overnight. Reaction proceeded slowly, heated at 70° C. overnight. It was diluted with water, extracted with EtOAc (2×), dried over MgSO₄, filtered, purified by ISCO, 12 g column, 2-20% EtOAc/dichloromethane to give 120 mg (70%) of (fw) as brown oil; LC-MS: m/z=+323 (M+H)+.

Step 3—The title compound was prepared by the procedure of Example 85, Step 6, followed by chiral HPLC separation to afford isomers (fx¹) and (fx²).

(isomer 1) ¹H NMR (400 MHz, DMSO) δ 8.65 (s, 1H), 8.21 (d, J=8.8 Hz, 2H), 7.48 (d, J=8.8 Hz, 2H), 6.14 (t, J=5.6 Hz, 1H), 5.17 (dd, J=30.3, 11.7 Hz, 2H), 4.21 (s, 1H), 4.05 (s, 1H), 3.94 (d, J=11.4 Hz, 1H), 3.74-3.62 (m, 2H), 3.50 (t, J=10.4 Hz, 1H), 3.32 (d, J=14.7 Hz, 1H), 3.16-3.08 (m, 2H), 2.45-2.39 (m, 1H), 2.33 (td, J=14.7, 7.3 Hz, 1H), 2.17-2.00 (m, 2H), 1.39 (s, 3H), 1.26 (d, J=6.7 Hz, 3H), 1.06 (t, J=7.2 Hz, 3H); LC-MS: m/z=+451 (M+H)+; (isomer 2) ¹H NMR (400 MHz, DMSO) δ 8.65 (s, 1H), 8.21 (d, J=8.7 Hz, 2H), 7.48 (d, J=8.8 Hz, 2H), 6.14 (t, J=5.6 Hz, 1H), 5.17 (dd, J=30.0, 11.7 Hz, 2H), 4.27 (s, 1H), 3.94 (d, J=8.4 Hz, 2H), 3.68 (dt, J=11.5, 7.1 Hz, 2H), 3.50 (dd, J=11.6, 9.3 Hz, 1H), 3.38-3.30 (m, 1H), 3.16-3.08 (m, 2H), 2.46-2.41 (m, 1H), 2.37-2.25 (m, 1H), 2.17-2.00 (m, 2H), 1.39 (s, 3H), 1.26 (d, J=6.7 Hz, 3H), 1.06 (t, J=7.2 Hz, 3H); LC-MS: m/z=+451 (M+H)+.

### Example 94

### Preparation of 1-ethyl-3-(4-((S)-7-(hydroxymethyl)-7-methyl-4-((S)-3-methylmorpholino)-5,7-dihydrofuro[3,4-d]pyrimidin-2-yl)phenyl)urea (ga¹) and 1-ethyl-3-(4-((R)-7-(hydroxymethyl)-7-methyl-4-((S)-3-methylmorpholino)-5,7-dihydrofuro[3,4-d]pyrimidin-2-yl)phenyl)urea (ga²)

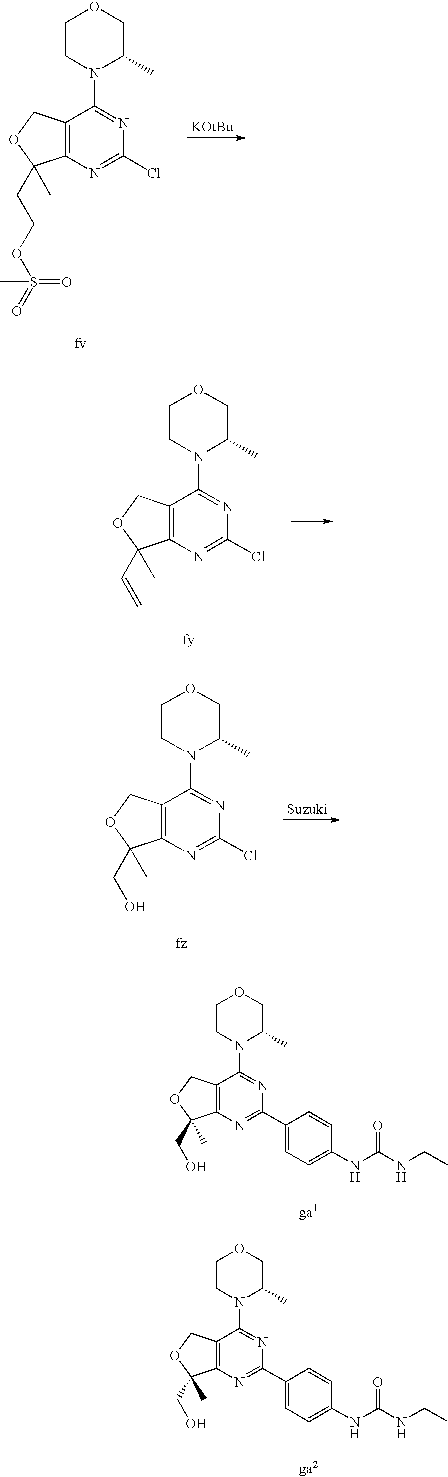

Step 1—Synthesis of 2-chloro-7-methyl-4-((S)-3-methylmorpholino)-7-vinyl-5,7-dihydrofuro[3,4-d]pyrimidine (fy). A solution of (fv) (240 mg, 0.6 mmol) from Example 93, Step 1 in THF (4 mL) was cooled in ice-bath, and KOtBu (140 mg, 1.2 mmol) was added. After 5.5 h, additional KOtBu (70 mg) was added to the reaction mixture. After 45 min, it was quenched with sat NH₄Cl, extracted with EtOAc, dried over MgSO₄, filtered, concentrated in vacuo to give 140 mg (77%) of (fy). It was carried on without further purification; LC-MS: m/z=+296 (M+H)+.

Step 2—Synthesis of (2-chloro-7-methyl-4((S)-3-methylmorpholino)-5,7-dihydrofuro[3,4-d]pyrimidin-7-yl)methanol (fz). The title compound was prepared by the general procedure of Example 86, Step 1-3; LC-MS: m/z=+300 (M+H)+.

Step 3—The title compounds were prepared by the procedure of Example 85, Step 6, followed by chiral HPLC separation to afford isomers (ga¹) and (ga²); (isomer 1) ¹H NMR (400 MHz, DMSO) δ 8.71 (s, 1H), 8.20 (d, J=8.2 Hz, 2H), 7.48 (d, J=8.2 Hz, 2H), 6.20 (t, J=5.3 Hz, 1H), 5.21-5.12 (m, 2H), 4.73 (t, J=5.8 Hz, 1H), 4.25 (s, 1H), 3.94 (d, J=10.9 Hz, 2H), 3.73 (d, J=11.3 Hz, 1H), 3.63 (dd, J=21.5, 9.6 Hz, 2H), 3.51 (dd, J=19.6, 8.0 Hz, 2H), 3.17-3.07 (m, 2H), 1.30 (s, 3H), 1.24 (d, J=6.5 Hz, 3H), 1.06 (t, J=7.1 Hz, 3H); LC-MS: m/z=+428 (M+H)+; (isomer 2) ¹H NMR (400 MHz, DMSO) δ 8.66 (s, 1H), 8.20 (d, J=8.3 Hz, 2H), 7.48 (d, J=8.3 Hz, 2H), 6.15 (t, J=5.5 Hz, 1H), 5.20 (d, J=11.2 Hz, 1H), 5.11 (d, J=11.6 Hz, 1H), 4.74 (t, J=5.9 Hz, 1H), 4.22 (s, 1H), 3.94 (d, J=11.8 Hz, 2H), 3.73 (d, J=11.7 Hz, 1H), 3.63 (dd, J=19.1, 8.1 Hz, 2H), 3.52 (dd, J=20.0, 8.6 Hz, 2H), 3.13 (dd, J=13.4, 6.7 Hz, 2H), 1.30 (s, 3H), 1.26 (d, J=6.6 Hz, 3H), 1.06 (t, J=7.2 Hz, 3H); LC-MS: m/z=+428 (M+H)+.

### Example 95

### Preparation of 2-((S)-2-(2-amino-1H-b enzo[d]imidazol-5-yl)-7-methyl-4-((S)-3-methylmorpholino)-5,7-dihydrofuro [3,4-d]pyrimidin-7-yl)ethanol (gd¹) and 2-((R)-2-(2-amino-1H-b enzo[d]imidazol-5-yl)-7-methyl-4-((S)-3-methylmorp ho lino)-5,7-dihydro furo [3,4-d]pyrimidin-7-yl)ethanol (gd²)

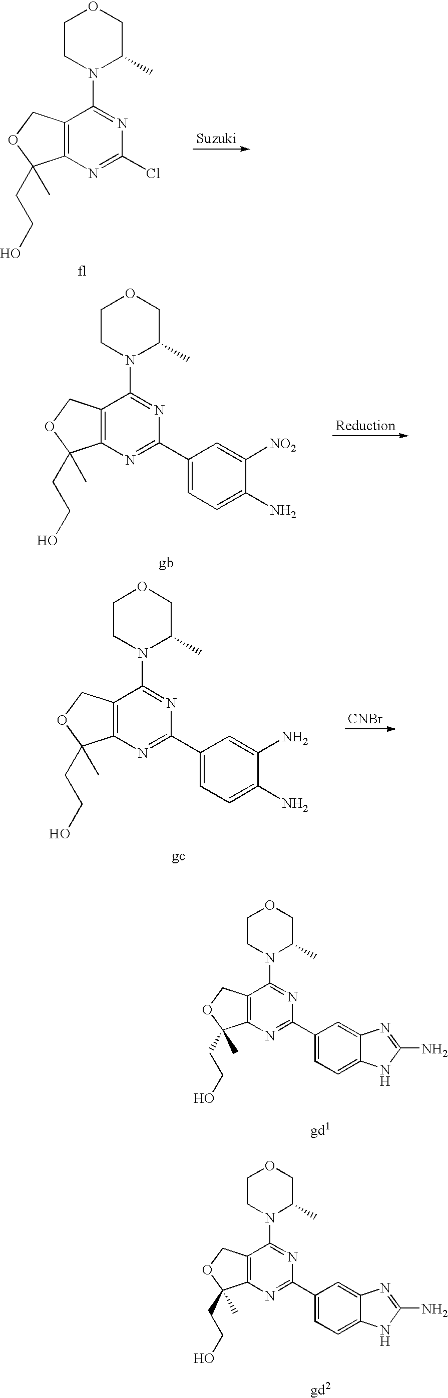

Step 1—Synthesis of 2-(2-(4-amino-3-nitrophenyl)-7-methyl-4-((S)-3-methylmorpholino)-5,7-dihydrofuro[3,4-d]pyrimidin-7-yl)ethanol (gb). The title compound was prepared by the procedure of Example 85, Step 6 substituting 1-ethyl-3-(4-(4,4,5,5-tetramethyl-1,3,2-dioxaborolan-2-yl)phenyl)urea with 2-nitro-4-(4,4,5,5-tetramethyl-1,3,2-dioxaborolan-2-yl)aniline: LC-MS: m/z=+416 (M+H)+.

Step 2—Synthesis of 2-(2-(3,4-diaminophenyl)-7-methyl-4-((S)-3-methylmorpholino)-5,7-dihydrofuro[3,4-d]pyrimidin-7-yl)ethanol (gc). To a mixture of (gb) (50 mg, 0.1 mmol) from Step 1, iron powder (34 mg, 0.6 mmol), and NH₄Cl (26 mg, 0.5 mmol) in EtOH (2 mL) and water (0.5 mL) was heated at 75° C. for 25 min. The mixture was cooled, diluted with dichloromethane, washed with sat NaHCO₃. The dichloromethane extract was dried over MgSO₄, filtered, concentrated in vacuo to give 40 mg (90%) of (gc) as a brown solid. It was carried on without further purification; LC-MS: m/z=+386 (M+H)+.

Step 3—Synthesis of 2-((S)-2-(2-amino-1H-benzo[d]imidazol-5-yl)-7-methyl-4-((S)-3-methylmorpholino)-5,7-dihydrofuro[3,4-d]pyrimidin-7-yl)ethanol(gd¹) and 2-((R)-2-(2-amino-1H-benzo[d]imidazol-5-yl)-7-methyl-4-((S)-3-methylmorpholino)-5,7-dihydrofuro[3,4-d]pyrimidin-7-yl)ethanol (gd²). To a suspension of (gc) (300 mg, 0.8 mmol) from Step 2 in MeOH (10 mL) was added CNBr (3 M solution in dichloromethane, 0.35 mL, 1.0 mmol)at rt, stirred for 2 h. It was concentrated in vacuo, purified by reverse-phase HPLC, followed by chiral HPLC separation to afford isomers (gd¹) and (gd²); (isomer 1) ¹H NMR (400 MHz, DMSO) δ 10.84 (d, J=36.0 Hz, 1H), 8.11 (s, 1H), 8.04-7.91 (m, 1H), 7.11 (d, J=7.7 Hz, 1H), 6.40 (s, 1H), 6.25 (s, 1H), 5.11 (dd, J=22.1, 11.6 Hz, 2H), 4.41 (t, J=5.1 Hz, 1H), 4.25 (s, 1H), 4.03 (s, 2H), 3.70 (dd, J=27.2, 10.1 Hz, 2H), 3.53 (dd, J=21.0, 11.5 Hz, 2H), 3.30 (d, J=4.5 Hz, 2H), 2.03-1.91 (m, 2H), 1.39 (s, 3H), 1.26 (d, J=6.7 Hz, 3H); LC-MS: m/z=+411 (M+H)+; (isomer 2): ¹H NMR (400 MHz, DMSO) δ 10.74 (d, J=32.8 Hz, 1H), 8.12 (s, 1H), 8.00 (s, 1H), 7.12 (d, J=8.3 Hz, 1H), 6.33 (s, 1H), 6.19 (s, 1H), 5.16 (d, J=11.6 Hz, 1H), 5.06 (d, J=11.6 Hz, 1H), 4.35 (t, J=5.3 Hz, 1H), 4.25 (s, 1H), 4.06-3.91 (m, 2H), 3.76-3.65 (m, 2H), 3.60-3.48 (m, 2H), 3.35 (d, J=12.5 Hz, 2H), 1.97 (qt, J=13.7, 7.0 Hz, 2H), 1.39 (s, 3H), 1.26 (d, J=6.7 Hz, 3H); LC-MS: m/z=+411 (M+H)+.

### Example 96

### Preparation of (S)-1-ethyl-3-(4-(4-(3-methylmorpholino)-5,7-dihydro furo [3,4-d]pyrimidin-2-yl)phenyl)urea (gi)

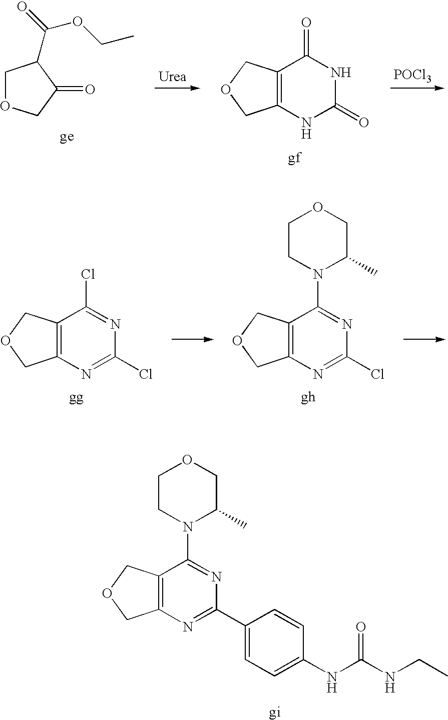

Step 1—Synthesis of ethyl 4-oxotetrahydrofuran-3-carboxylate (ge). NaH (60%, 4.6 g, 116 mmol) was suspended in ether (200 mL) and cooled in ice-bath. To this suspension was added ethyl glycolate (10 mL, 100 mmol) dropwise under N₂. The resulting white slurry was stirred at rt for 45 min. Ether was removed in vacuo to give white solid. It was suspended in DMSO (130 mL), cooled in ice-bath, and ethyl arcylate (13.7 mL, 127 mmol) was added dropwise. The resulting yellow mixture was stirred at rt overnight. The reaction solution was poured into 10% aq. HCl (500 mL) slowly. It was extracted with ether (3×). The combined ether extract was washed with brine, dried over MgSO₄, filtered, concentrated in vacuo to give 14 g (80%) of (ge) as clear yellow liquid. It was carried on without further purification.

Step 2—Synthesis of 5,7-dihydrofuro[3,4-d]pyrimidine-2,4(1H,3H)-dione (gf). To a mixture of (ge) (10 g, 60 mmol) from Step 1 and urea (5.5 g, 92 mmol) in MeOH (45 mL) was added conc. HCl (2.5 mL). The resulting mixture was heated to reflux for 2.5 h. It was cooled, stirred in ice-bath for 15 min. The white precipitate was collected by filtration, washed with water. The solid was suspended in 2 N NaOH (50 mL) and water (15 mL) was added, heated to reflux for 1 h. It was cooled in ice-bath, acidified with conc. HCl. The precipitate was filtered, washed with water, dried to give 5.1 g (50%) of (gf) as white solid: LC-MS: m/z=+155 (M+H)+.

Step 3—Synthesis of 2,4-dichloro-5,7-dihydrofuro[3,4-d]pyrimidine (gg). The title compound was prepared by the procedure of Example 90, Step 2: LC-MS: m/z=+191 (M+H)+.

Step 4—Synthesis of (S)-2-chloro-4-(3-methylmorpholino)-5,7-dihydrofuro[3,4-d]pyrimidine (gh). The title compound was prepared by the procedure of Example 85, Step 5: LC-MS: m/z=+256 (M+H)+.

Step 5—Synthesis of (S)-1-ethyl-3-(4-(4-(3-methylmorpholino)-5,7-dihydrofuro[3,4-d]pyrimidin-2-yl)phenyl)urea (gi). The title compound was prepared by the procedure of Example 85, Step 6: ¹H NMR (400 MHz, DMSO) δ 8.64 (s, 1H), 8.18 (d, J=8.8 Hz, 2H), 7.47 (d, J=8.8 Hz, 2H), 6.16 (t, J=5.6 Hz, 1H), 5.22 (dd, J=29.1, 11.6 Hz, 2H), 4.84 (s, 2H), 4.24 (s, 1H), 4.05-3.91 (m, 2H), 3.74-3.61 (m, 2H), 3.50 (td, J=11.9, 2.8 Hz, 1H), 3.34 (dd, J=13.0, 3.4 Hz, 1H), 3.16-3.08 (m, 2H), 1.25 (d, J=6.8 Hz, 3H), 1.06 (t, J=7.2 Hz, 3H); LC-MS: m/z=+384 (M+H)+.

### Example 97

### Synthesis of 1-ethyl-3-(4-(4-morpholino-5,7-dihydrofuro[3,4-d]pyrimidin-2-yl)phenyl)urea (gj)

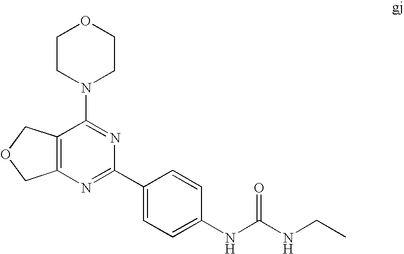

Synthesis of 1-ethyl-3-(4-(4-morpholino-5,7-dihydrofuro[3,4-d]pyrimidin-2-yl)phenyl)urea (gj). The title compound was prepared by the procedures of Example 96 substituting (S)-3-methylmorpholine with morpholine: LC-MS: m/z=+370 (M+H)+.

### Example 98

### Preparation of 1-(4-(4-((1R,5S)-3-oxa-8-azabicyclo[3.2.1]octan-8-yl)-5,7-dihydrofuro[3,4-d]pyrimidin-2-yl)phenyl)-3-ethylurea (gk)

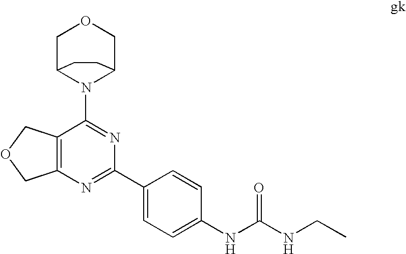

Synthesis of 1-(4-(4-((1R,5S)-3-oxa-8-azabicyclo [3.2.1]octan-8-yl)-5,7-dihydrofuro[3,4-d]pyrimidin-2-yl)phenyl)-3-ethylurea (gk). The title compound was prepared by the procedures of Example 96 substituting (S)-3-methylmorpholine with (1R,5S)-3-oxa-8-azabicyclo[3.2.1]octane: LC-MS: m/z=+396 (M+H)+.

### Example 99

### Preparation of 1-(4-(4-((1R,5S)-8-oxa-3-azabicyclo[3.2.1]octan-3-yl)-5,7-dihydrofuro[3,4-d]pyrimidin-2-yl)phenyl)-3-ethylurea (gl)

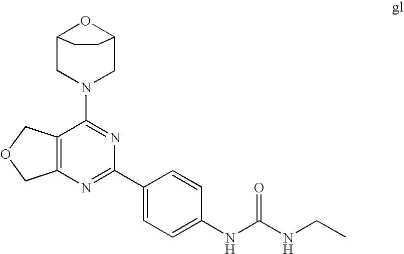

Synthesis of 1-(4-(4-((1R,5S)-8-oxa-3-azabicyclo[3.2.1]octan-3-yl)-5,7-dihydrofuro[3,4-d]pyrimidin-2-yl)phenyl)-3-ethylurea (gl). The title compound was prepared by the procedures of Example 96 substituting (S)-3-methylmorpholine with 8-oxa-3-azabicyclo[3.2.1]octane hydrochloride: LC-MS: m/z=+396 (M+H)+.

### Example 100

### Preparation of 2-(2-(2-aminopyrimidin-5-yl)-7-methyl-4((S)-3-methylmorpholino)-5,7-dihydrofuro[3,4-d]pyrimidin-7-yl)ethanol(gm)

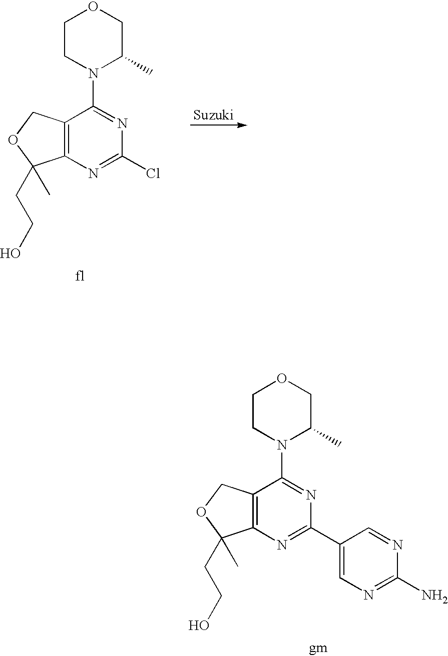

Step 1—The solvents (acetonitrile and water) were degassed overnight by bubbling nitrogen through them. A 2-5 mL microwave tube equipped with a stir bar was charged with (fl) (150 mg, 0.48 mmol), followed by 2-aminopyrimidine-5-boronic acid, pinacol ester (140 mg, 0.62 mmol), bis(triphenylphosphine) palladium (II) chloride (22 mg, 0.032 mmol), sodium carbonate (81 mg, 0.76 mmol), potassium acetate (94 mg, 0.96 mmol). The mixture was dissolved in degassed acetonitrile (3.0 mL)/water (0.9 mL), the microwave vial capped, placed in a Biotage microwave, and microwaved (300 watts, temperature=140° C., time=15 min). The progress of the reaction was checked by LC-MS and (fl) had been completely consumed. Poured contents of the microwave tube into a 125 mL Erlenmeyer flask containing EtOAc (30 mL) and rinsed the tube with additional EtOAc (3×10 mL). Transferred the EtOAc solution from the Erlenmeyer flask to a 125 mL separatory funnel, and washed the EtOAc solution once with water, once with brine. Dried the EtOAc layer (MgSO₄), filtered, concentrated and dried under high vacuum to give 1398 mg of crude product. The diastereomeric mixture of 2-((R,S)-2-(2-aminopyrimidin-5-yl)-7-methyl-4((S)-3-methylmorpholino)-5,7-dihydrofuro[3,4-d]pyrimidin-7-yl)ethanol was purified by R^{p} HPLC followed by a second HPLC chromatography to separate the diastereomers to afford separated compound (diasteromer-1): ¹H NMR (500 MHz, DMSO) δ 9.05 (s, 2H), 7.08 (s, 2H), 5.10 (dd, J=49.4, 11.7 Hz, 2H), 4.29 (t, J=5.3 Hz, 2H), 4.05 (d, J=5.1 Hz, 1H), 3.92 (d, J=8.7 Hz, 1H), 3.67 (dd, J=32.5, 10.1 Hz, 2H), 3.60-3.42 (m, 2H), 1.95 (dd, J=15.0, 7.1 Hz, 2H), 1.36 (s, 3H), 1.24 (d, J=6.7 Hz, 3H). LC-MS: m/z=+373.1 (M+H)+. ret. time=1 min 100% diastereomeric purity (uv 254); and (diastereomer 2)¹H NMR (500 MHz, DMSO) δ 9.05 (s, 2H), 7.08 (s, 2H), 5.11 (dd, J=28.6, 11.7 Hz, 2H), 4.29 (t, J=5.3 Hz, 2H), 4.05 (d, J=5.1 Hz, 1H), 3.93 (d, J=10.6 Hz, 1H), 3.67 (dd, J=31.9, 13 Hz, 2H), 3.58-3.44 (m, 2H), 1.95 (dd, J=14.7, 6.0 Hz, 2H), 1.37 (s, 3H), 1.25 (d, J=6.8 Hz, 3H). LC-MS: m/z=+373.1 (M+H)+. ret. time=1.60 min 100% diastereomeric purity (uv 254)

### Example 101

### Preparation of 5-(7-(2-methoxyethyl)-7-methyl-4-((S)-3-methylmorpholine-5,7-dihydrofuro[3,4-d]pyrimidin-2-yl)pyrimidin-2-amine (go)

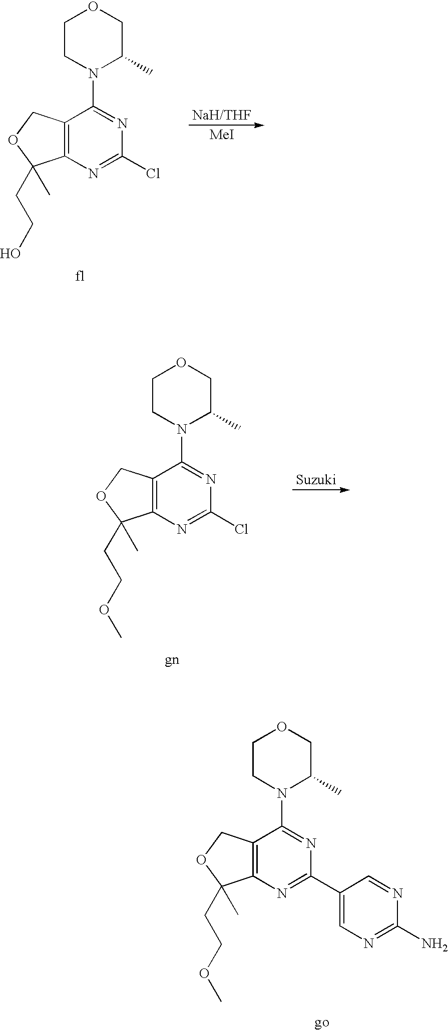

Step 1—To a solution of (fl) (141 mg, 0.449 mmol, in THF (3.5 mL) at 0° C. (ice/water bath), iodomethane was added via syringe (0.084 mL, 1.35 mmol) followed by sodium hydride (37 mg, 60% dispersion in mineral oil, 0.56 mmol). The ice/water bath was removed, the reaction warmed to room temperature and stirred at room temperature for 3 h after which time LC-MS and thin layer chromatography (TLC) (2:3 Ethyl Acetate/dichloromethane) indicated (fl) had been completely consumed. The reaction mixture was diluted with sat'd NH₄Cl, and extracted into EtOAC (40 mL). The layers were separated and the sat'd NH₄Cl extracted again with ethylacetate (20 mL). The ethyl acetate extracts were combined, washed 1× with sat'd NaCl (20 mL), dried (MgSO₄), filtered, and concentrated on a rotary evaporator to dryness. The crude product was purified by silica chromatography (ISCO, 24 gm column, 16×65 mm silica pre column, dichloromethane load onto SiO₂ pre column, 0-80% EtOAc/dichloromethane) to afford 58.3 mg of (gn). ¹H NMR (500 MHz, CDCl₃) δ 5.16-4.96 (m, 2H), 4.17-3.83 (m, 3H), 3.71 (dd, J=28.6, 11.6 Hz, 2H), 3.53 (t, J=11.9 Hz, 1H), 3.47-3.29 (m, 3H), 3.21 (s, 3H), 2.13 (td, J=8.0, 4.0 Hz, 1H), 2.04 (td, J=7.6, 4.1 Hz, 1H), 1.43 (s, 3H), 1.36 (s, 3H), 1.33 (dd, J=6.7, 4.4 Hz, 3H). LC-MS: m/z=+328.1 (M+H)+.

Step 2 The title compound was prepared by the Suzuki procedure with the same stoichiometry and work up as described in Example 100. The reaction was run using 58 mg (0.18 mmol) compound (gn). The diastereomeric mixture (go) was purified and diastereomers separated by chiral HPLC to afford pure diastereomers (diastereomer-1, 11.9 mg): ¹H NMR (400 MHz, DMSO) δ 9.06 (s, 2H), 7.09 (s, 2H), 5.12 (dd, J=41.3, 11.7 Hz, 2H), 4.22 (m 1H), 3.93 (m+d, J=8.4 Hz, 2H), 3.77-3.58 (m, 2H), 3.56-3.34 (m, 2H), 3.28-3.17 (m, 2H), 3.14 (s, 3H), 2.01 (tt J=13.8, 8.0 Hz, 2H), 1.37 (s, 3H), 1.25 (d, J=6.7 Hz, 3H). LC-MS: m/z=+387.2(M+H)+. ret. time=1 min 100% diastereomeric purity (uv 254); and (diastereomer-2), 12.8 mg): ¹H NMR (400 MHz, DMSO) δ 9.05 (s, 2H), 7.09 (s, 2H), 5.11 (q, J=11.8, 2H), 4.21 (m, 1H), 3.92 (m, 1H), 3.67 (m, 2H), 3.56-3.31 (m, 2H), 3.26-3.17 (m, 1H), 3.14 (s, 3H), 2.26-1.83 (m, 2H), 1.36 (s, 3H), 1.24 (d, J=6.8 Hz, 3H). LC-MS: m/z=+387.2(M+H)+. ret. time=1.15 min 100% diastereomeric purity (uv 254)

### Example 102

### Preparation of 5-(7,7-dimethyl-4-morpholino-5,7-dihydrofuro[3,4-d]pyrimidin-2-yl)pyridin-2-amine (gq)

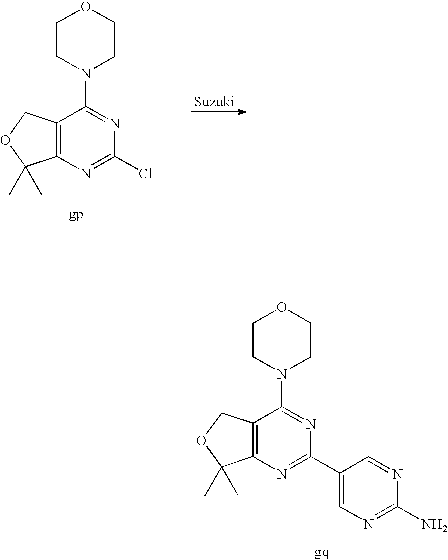

5-(7,7-dimethyl-4-morpholino-5,7-dihydrofuro[3,4-d]pyrimidin-2-yl)pyridin-2-amine was prepared by a Suzuki coupling using the procedure and workup as described for Example 100 except that 2-aminopyridine-5-boronic acid (79 mg, 0.36 mmol) was used instead of 2-aminopyrimidine-5-boronic acid, pinacol ester. 85 mg (0.30 mmol) of gp was consumed to afford 53 mg of crude product. The crude material was purified by RP-HPLC to afford 11.4 mg of the title compound (gq): ¹H NMR (400 MHz, DMSO) δ 8.89 (d, J=2.1 Hz, 1H), 8.24(dd, J=8.7, 2.3 Hz 1H), 6.49 (d, J=8.7 Hz, 1H), 6.35 (s, 2H), 5.11 (s, 2H), 3.87-3.50 (m, 8H), 1.38 (s, 6H). LC-MS: m/z=+328.1 (M+H)+.

### Example 103

### Preparation of (S)-5-(7,7-dimethyl-4-(3-methylmorpholino-5,7-dihydrofuro[3,4-d] 2-amine (gr)

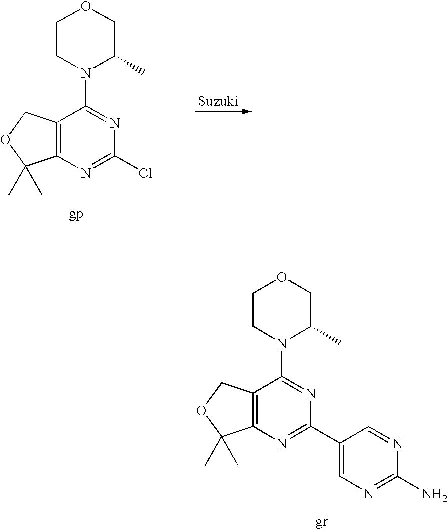

(S)-5-(7,7-dimethyl-4-(3-methylmorpholino-5,7-dihydrofuro[3,4-d]2-amine was prepared by a Suzuki coupling using the procedure and workup as described for Example 100. 309 mg of gp (1.09 mmol) was consumed to yield 32.4 mg of the title compound after RP-HPLC purification: ¹H NMR (400 MHz, DMSO) δ 9.05 (s, 2H), 7.08 (s, 2H), 5.11 (dd, J=30.9, 11.7 Hz, 2H), 4.24 (brs, 1H), 3.92 (dd, J=11.3, 3.0 Hz, 2H), 3.65 (dt J=11.5, 7.1 Hz, 2H), 3.48 (ddd, J=7.3, 2.7, 1.3 Hz, 1H), 3.34 (brs, 1H), 1.38 (s, 6H), 1.25 (d, 6.8 Hz, 3H); LC-MS: m/z=+343.1.1(M+H)+.

### Example 104

### Preparation of 5-(7-methyl-4-((S)-3-methylmorp ho lino)-7-(2-phenoxyethyl)-5,7-dihydro [3,4-d]pyrimidin-2-amine (gs)

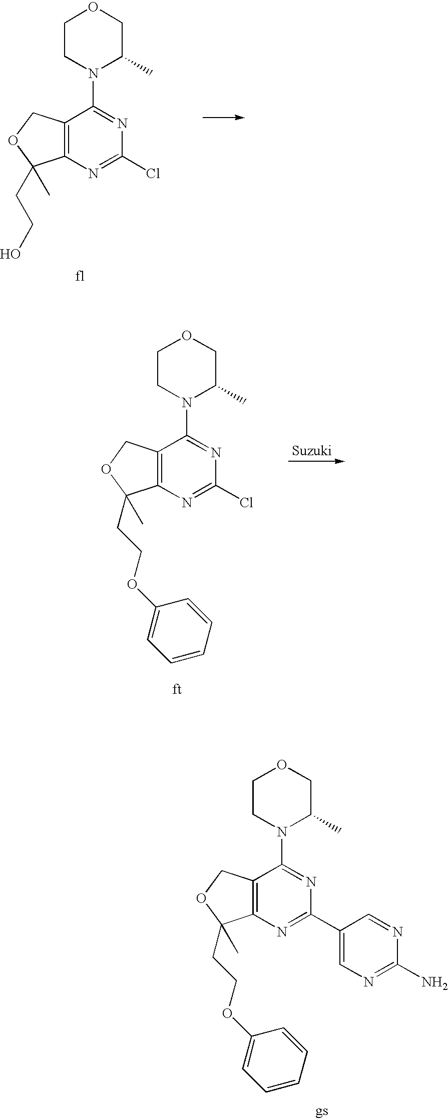

The title compound (gs) was prepared as a mixture of diastereomers by the Suzuki procedure with the same stoichiometrys and work up as described in Example 100. The reaction was run using 93 mg (0.24 mmol) of (ft). The diastereomeric mixture was purified and diastereomers separated by chiral HPLC to afford pure diastereomers; (diastereomer-1, 4.8 mg): ¹H NMR (400 MHz, CDCl₃) δ 9.26 (s, 2H), 7.16 (t, J=8.0 Hz, 2H), 6.85 (t, J=7.3 Hz, 1H), 6.62 (t+brs, J=10.9 Hz, 4H), 5.11 (q, J=11.5 Hz, 2H), 4.27-3.81 (m, 5H), 3.84-3.62 (m, 2H), 3.63-3.48 (m, 1H), 3.35 (td, J=12.9, 3.5 Hz 1H), 2.48 (dt J=14.4, 7.3 Hz, 1H), 2.22 (dt, J=14.4, 5.6 Hz, 1H), 1.52 (s, 3H), 1.34 (d, J=6.8 Hz, 3H). LC-MS: m/z=+449.1(M+H)+. ret. time=1.31 min 100% diastereomeric purity (uv 254), and (diasteromer-2, 3.8 mg): ¹H NMR (400 MHz, CDCl₃) δ 9.19 (s, 2H), 7.15 (dd, J=8.5, 7.5 Hz, 2H), 6.83 (t, J=7.3 Hz, 1H), 6.67 (d, J=7.8 Hz, 2H), 5.36 (brs, 2H), 5.09 (dd, J=41.4, 11.3 Hz, 2H), 4.23-3.84 (m, 5H), 3.85-3.63 (m, 2H), 3.52 (td, J=11.9, 2.8 Hz, 1H), 3.46-3.25 (m, 1H), 2.42 (dd, J=14.3, 7.2 Hz, 1H), 2.33-2.12 (m, 1H), 1.49 (s, 3H), 1.22 (d, J=6.8 Hz, 3H). LC-MS: m/z=+449.1(M+H)+. ret. time=1.53 min 99% diastereomeric purity (UV 254)

### Example 105

### Preparation of 5-(4-((1R,4R)-2-oxa-5-az abicyclo [2.2.1] heptan-5-yl)-7,7-dimethyl-5,7-dihydro furo [3,4-d]pyrimidin-2-yl)pyrimidin-2-amine (gu)

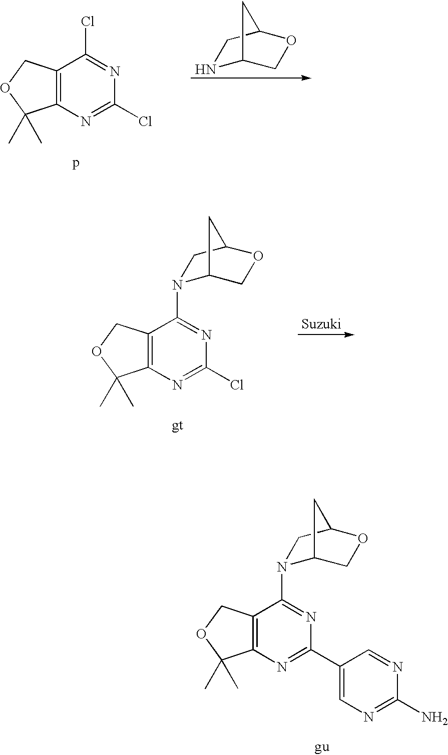

Step 1—A 20 mL vial equipped with a stirring bar and a Teflon cap was charged with (p) (159 mg, 0.726 mmol) and dissolved in anhydrous ethanol/DMF. Gentle heating with a heat gun was needed to effect dissolution. Once dissolved, the solution was cooled to room temperature and with stirring N,N diisopropylethylamine (0.5 mL2.903 mmol) was added via syringe followed by 2-oxo-5-azabicyclo[2.2.1]heptane hydrochloride (127.9 mg, 0.943 mmol, Anthem Pharmaceuticals). The vial was flushed with nitrogen, capped and placed in a pre-heated 45° C. oil bath and heated at 45° C. for 22 h. LC-MS analysis indicated p had been consumed to give one major new UV active product with an M+H+ consistent with (gt). The reaction mixture was transferred to a round bottom flask, the vial rinsed with additional ethanol, and concentrated to dryness on a rotary evaporator. The residue was dissolved in ethyl acetate (30 mL) and transferred to a separatory funnel, rinsing the round bottom with additional ethyl acetate. The ethyl acetate solution was washed 1× with 10% citric acid, 1× with water, and 1× with brine. The combined aqueous extracts were back extracted with ethyl acetate. The combined ethyl acetate extracts were dried (MgSO₄), filtered, concentrated on a rotary evaporater, then dried under high vacuum to afford 224 mg of a crude product as a white foam. LC-MS and NMR indicated the crude product (gt) was of high purity and could be used directly in the next step: ¹H NMR (400 MHz, DMSO) δ 5.19 (d, J=11.9 Hz, 1H), 5.01 (brs, 1H), 4.66 (s, 1H), 3.74 (s, 2H), 3.59 (m, 2H), 2.71 (dd, J=41.3, 15.4 Hz, 1H), 1.86 (s, 2H), 1.33 (s, 6H); LC-MS: m/z=+282.2(M+H)+.

Step 2 The title compound (gu) was prepared by the Suzuki procedure with the same stoichiometry and work up as described in Example 100. The reaction was run using 75 mg (0.27 mmol) (gt) and afforded 63.5 mg of (gu) as a white solid after RP-HPLC purification and lyophilization: ¹H NMR (400 MHz, DMSO) δ 9.06 (s, 2H), 7.09 (s, 2H), 5.21 (d, J=11.9 Hz, 1H), 5.03 (d, J=11.8 Hz, 1H), 4.67 (s, 1H), 4.05-3.75 (m, 2H), 3.65-3.54 (dd, J=18, 9.9 Hz, 2H), 1.89 (br s, 2H), 1.38 (s, 6H). LC-MS: m/z=+341.1(M+H)+.

### Example 106

### Preparation of Synthesis of 5-(4-((1R,5S)-3-oxa-8-azabicyclo[2.2.1]octan-8-yl)-7,7-dimethyl-5,7-dihydrofuro[3,4-d]pyrimidin-2-yl)pyrimidin-2-amine (gw)

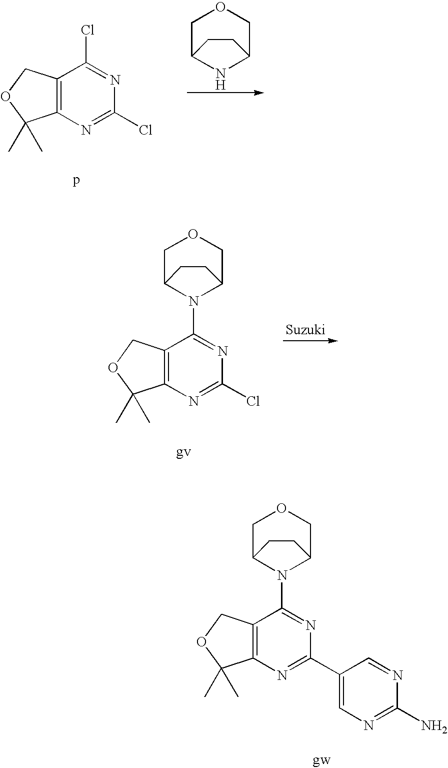

Step 1—Compound (gv) was prepared and worked up as described for compound (gt) in Example 105 except that 3-oxa-8-azabicyclo[3.2.1]octane hydrochloride was used instead of 2-oxo-5-azabicyclo[2.2.1] heptane hydrochloride. After work up 159 mg (0.726 mmol) of compound (p) yielded 229 mg of crude (gv) as an off white solid. LC-MS and NMR indicated the crude product was of high purity and could be used directly in the next step. (gv): ¹H NMR (400 MHz, DMSO) δ 5.00 (s, 2H), 3.59 (dd, J=12.0, 4.0 Hz, 4H), 3.32 (s, 1H), 2.70 (dd, J=40.3, 16.2 Hz, 1H), 1.95 (s, 4H), 1.34 (s, 6H); LC-MS: m/z=+296.3(M+H)+.

Step 2—The title compound was prepared by the Suzuki procedure with the same stoichiometry and work up as described in Example 100. The reaction was run using 91 mg (0.31 mmol) (gv) and afforded 14.9 mg of (gw) as a white solid after RPHPLC purification and lyophilization: (gw): ¹H NMR (400 MHz, DMSO) δ 9.05 (s, 2H), 7.10 (s, 2H), 5.02 (s, 2H), 4.47 (br s, 2H), 3.62 (dd, J=26.7, 10.9 Hz, 4H), 1.99 (m, 4H), 1.39 (s, 6H). LC-MS: m/z=+355.1(M+H)+.

### Example 107

### Preparation of 5-(4-((1R,5S)-8-oxa-3-azabicyclo [3.2.1]octan-8-yl)-7,7-dimethyl-5,7-dihydro furo [3,4-d]pyrimidin-2-yl)pyrimidin-2-amine (gy)

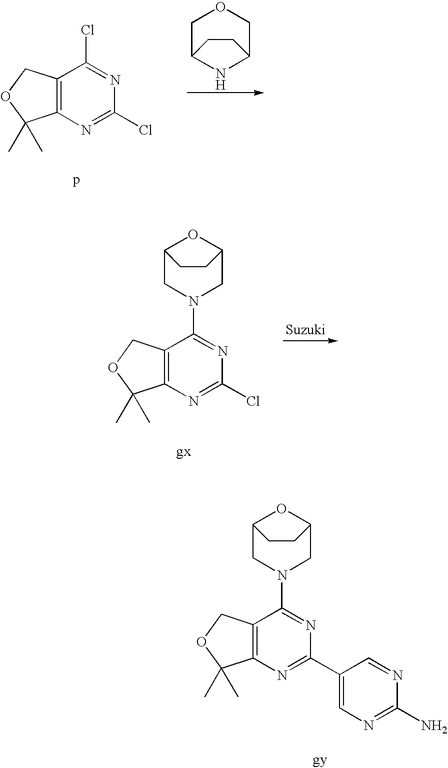

Step 1—A 20 mL vial equipped with a stirring bar and a Teflon cap was charged with 8-oxa-3azabicyclo[3.2.1]octane hydrochloride (101 mg, 0.673 mmol) followed by p (112 mg, 0.511 mmol). The solids were dissolved in anhydrous ethanol/DMF and heated gently with a heating gun to effect dissolution. After cooling to room temperature, N,N diisopropylethylamine (0.5 mL, 2.56 mmol) was added via syringe, the vial flushed with nitrogen, capeed and placed in a pre heated 45° C. heating block. After heating for 65 h at 45° an aliquot was removed and the progress of the reaction determined by LC-MS. Compound (p) was completely consumed and the reaction worked up as described in Example 105 to afford 156 mg of crude compound (gx) as an off white solid. LC-MS and NMR indicated the crude product was of high purity and could be used directly in the next step: (gx)¹H NMR (400 MHz, DMSO) δ 5.10 (s, 2H), 4.38 (br d, J=1.7 Hz, 2H), 3.73 (br s, 1H), 3.32 (s, 1H), 3.20 (d, J=12.2 Hz, 2H), 2.70 (dd, J=41.2, 15.4 Hz, 1H), 2.01-1.51 (m, 4H), 1.32 (s, 6H). LC-MS: m/z=+296.3(M+H)+.

Step 2—The title compound (gy) was prepared by the Suzuki procedure with the same stoichiometry and work up as described in Example 100. The reaction was run using 67 mg (0.23 mmol) compound (gx) and afforded 32.1 mg of compound (gy) as a white solid after RP-HPLC purification and lyophilization: (gy) ¹H NMR (400 MHz, DMSO) δ 9.05 (s, 2H), 7.11 (s, 2H), 5.12 (s, 2H), 4.41 (br s, 2H), 3.90 (br s, 2H), 3.20 (d, J=12.3 Hz, 2H) 1.79(m, 4H), 1.38 (s, 6H). LC-MS: m/z=+355.1(M+H)+.

### Example 108

### Preparation of 1-(4-(7,7-dimethyl-4-morpholino-5-oxo-5,7-dihydrofuro[3,4-d]pyrimidin-2-yl)phenyl)-3-ethylurea (hb)

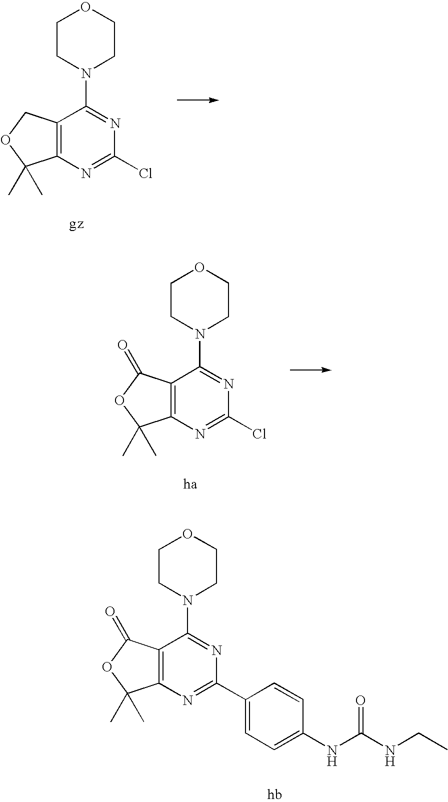

Step 1—Synthesis of (S)-2-chloro-7,7-dimethyl-4-(3-methylmorpholino)furo[3,4-d]pyrimidin-5(7H)-one (ha). A mixture of ether 2-chloro-7,7-dimethyl-4-morpholino-5,7-dihydrofuro[3,4-d]pyrimidine (300 mg, 1.06 mmol), iodosylbenzene (700 mg, 3.17 mmol), potassium bromide (126 mg, 1.06 mmol), Montmorillionite K10 (500 mg), water (4 mL) and acetonitrile (4 ml) was heated at 80° C. in a sealed vial for 6 h). The mixture was diluted with ethyl acetate (10 ml) and filtered through Celite with more ethyl acetate. Saturated NaHCO₃ (10 mL) was added and the phases separated. The organic phase was adsorbed onto Celite and chromatographed, ISCO 12 g column 0-40% ethyl aceate/heptane to afford 211 mg (70%) of (ha) as a colorless solid: ¹H NMR (400 MHz, CDCl₃) δ 4.44 (s, 1H), 4.02 (s, 1H), 3.86-3.75 (m, 2H), 1.62 (s, 3H); ¹³C NMR (101 MHz, CDCl₃ δ 187.03, 166.56, 164.06, 159.00, 98.68, 84.28, 67.18, 49.73, 46.29; HRMS (ES+) m/z 284.0860 (284.0802 cald for C₁₂H₁₅ClN₃O₃M+H).

Step 2—Synthesis of 1-(4-(7,7-dimethyl-4-morpholino-5-oxo-5,7-dihydrofuro[3,4-d]pyrimidin-2-yl)phenyl)-3-ethylurea (hb). A mixture of lactone (ha) (100 mg, 0.34 mmol), [4-ethylureido)phenyl]boronic acid, picacol ester (107 mg, 0.37 mmol), tetrakis(triphenylphosphine)palladium(0) (40 mg, 0.035 mmol), 1.27 M K₃PO₄ (0.45 mL, 0.57 mmol), and acetonitrile (2 mL) was heated at 110° C. in a microwave reactor for 30 min. The mixture was partitioned between saturated NH₄Cl (10 mL) and ethyl acetate (10 mL). The phases were separated and the aq. extracted with ethyl acetate (2×5 mL). The combined organic phases were dried (Na₂SO₄), filtered, adsorbed onto Celite and chromatographed ISCO 12 g column 0-75% ethyl acetate in heptane to afford 116 mg of hb as a colorless solid. A portion of this material was further purified by reverse-phase HPLC: ¹H NMR (500 MHz, DMSO) δ 8.84 (s, 1H), 8.31 (d, J=8.8 Hz, 2H), 7.54 (d, J=8.8 Hz, 2H), 6.22 (t, J=5.5 Hz, 1H), 4.34-3.94 (m, 4H), 3.82-3.66 (m, 4H), 3.20-2.97 (m, 2H), 1.58 (s, 6H), 1.06 (t, J=7.2 Hz, 3H); ¹³C NMR (126 MHz, DMSO) δ 184.21, 166.74, 165.27, 158.38, 154.60, 144.19, 129.72, 128.49, 116.77, 96.33, 83.45, 66.05, 33.87, 24.94, 15.29; HRMS (ES+) m/z 412.2076 (412.1985 cald for C₂₁H₂₆N₅O₄M+H).

### Example 109

### Preparation of (S)-1-(4-(7,7-dimethyl-4-(3-methylmorpholino)-5-oxo-5,7-dihydro furo [3,4-d]pyrimidin-2-yl)phenyl)-3-ethylurea (hd)

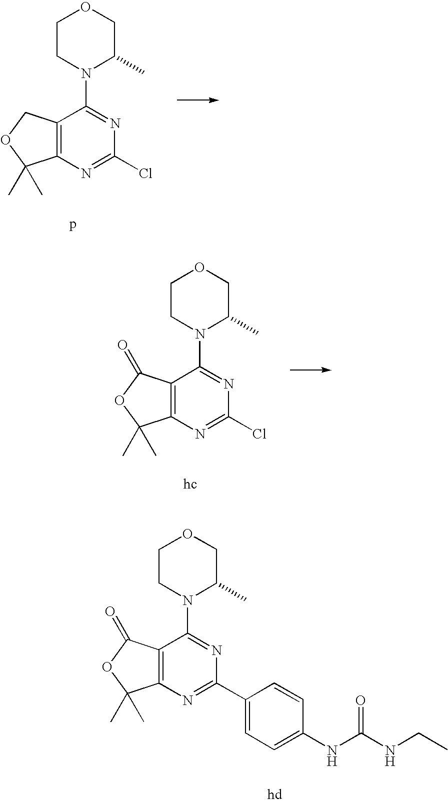

Step 1—Synthesis of (S)-2-chloro-7,7-dimethyl-4-(3-methylmorpholino)furo[3,4-d]pyrimidin-5(7H)-one (hc). Made by the general procedure of Example 108 using (S)-2-chloro-7,7-dimethyl-4-(3-methylmorpholino)-5,7-dihydrofuro[3,4-d]pyrimidine instead of 2-chloro-7,7-dimethyl-4-morpholino-5,7-dihydrofuro[3,4-d]pyrimidine 80% yield: ¹H NMR (400 MHz, CDCl₃ broadend by rotamers) δ 5.49 (s, 1H), 5.23 (s, 1H), 5.00 (s, 1H), 4.72 (s, 1H), 4.03 (d, J=8.1 Hz, 1H), 3.79 (d, J=11.8 Hz, 1H), 3.72 (dd, J=11.8, 2.8 Hz, 1H), 3.49 (d, J=101.6 Hz, 2H), 1.61 (t, J=6.3 Hz, 6H), 1.44 (s, 3H); HRMS (ES+) m/z 298.0958 (298.0958 cald for C₁₃H₁₇ClN₃O₃M+H).

Step 2—Synthesis of (S)-1-(4-(7,7-dimethyl-4-(3-methylmorpholino)-5-oxo-5,7-dihydrofuro[3,4-d]pyrimidin-2-yl)phenyl)-3-ethylurea (hd). Compound (hd) was made by the general procedure of Example 108, step 2: ¹H NMR (500 MHz, DMSO) δ 8.79 (s, 1H), 8.30 (d, J=8.8 Hz, 2H), 7.53 (d, J=8.8 Hz, 2H), 6.20 (t, J=5.5 Hz, 1H), 5.21 (s, 1H), 4.94 (s, 1H), 3.99 (dd, J=12.0, 5.3 Hz, 1H), 3.76 (d, J=11.5 Hz, 1H), 3.67 (dd, J=11.6, 2.9 Hz, 1H), 3.60-3.50 (m, 1H), 3.46 (s, 1H), 3.18-3.02 (m, 2H), 1.58 (s, 3H), 1.58 (s, 3H), 1.35 (d, J=6.8 Hz, 3H), 1.07 (t, J=7.2 Hz, 3H); ¹³C NMR (126 MHz, DMSO) δ 184.31, 166.66, 165.29, 158.31, 154.64, 144.17, 129.68, 128.63, 116.85, 96.37, 83.30, 70.12, 66.26, 33.89, 25.01, 24.97, 15.27; HRMS (ES+) m/z 426.2121 (426.2141 cald for C₂₂H₂₈N₅O₄M+H).

### Example 110

### Preparation of 5-((S)-7-(2-methoxyethyl)-7-methyl-4-((S)-3-methylmorpholino)-5,7-dihydrofuro[3,4-d]pyrimidin-2-yl)benzo[d]oxazol-2-amine (he¹) and 5-((R)-7-(2-methoxyethyl)-7-methyl-4-((S)-3-methylmorpholino)-5,7-dihydrofuro[3,4-d]pyrimidin-2-yl)benzo[d]oxazol-2-amine (he²)

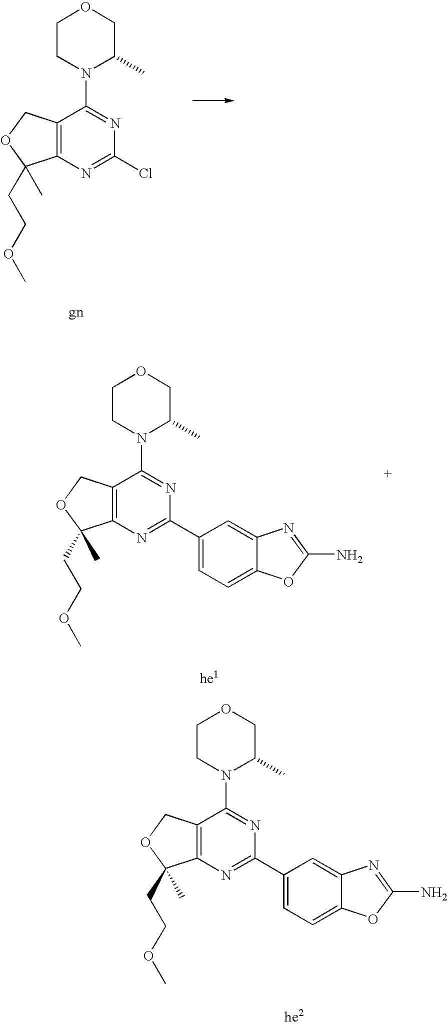

A mixture of tetrakis(triphenylphosphine)pallaium(0) (0.0453 g, 0.0000392 mol), sodium carbonate (0.0781 g, 0.000737 mol), and potassium acetate (0.0947 g, 0.000965 mol) were combined and purged with nitrogen. To the mixture was added 2-chloro-7-(2-methoxyethyl)-7-methyl-4-((S)-3-methylmorpholino)-5,7-dihydrofuro[3,4-d]pyrimidine (gn) (0.152 g, 0.000464 mol;) and 5-(4,4,5,5-tetramethyl-1,3,2-dioxaborolan-2-yl)benzo[d]oxazol-2-amine (0.150 g, 0.000577 mol) in dry acetonitrile (3.40 mL, 0.0651 mol;) and deoxygenated water (2.00 mL, 0.111 mol). The mixture was heated at 90° C. and stirred for 2 days. The mixture was partitioned between water (50 mL) and 10% methanol in dichloromethane (50 mL). The phases were separated and the aq. extracted with 10% methanol in dichloromethane (2×50 mL). The combined organic phases were dried (MgSO₄), filtered, and chromatographed ISCO (12 g, 10-50% ethyl acetate in heptane followed by 5% methanol in dichloromethane) to give 5-(7-(2-methoxyethyl)-7-methyl-4-((S)-3-methylmorpholino)-5,7-dihydrofuro[3,4-d]pyrimidin-2-yl)benzo[d]oxazol-2-amine as a mixture of two diastereomers. The diastereomers were separated by super critical fluid chromatograph to give 18.3 mg and 19.2 mg of each diastereomer (he¹) and (he²). Diastereomer 1 LC-MS: m/z=426 (M+H). ¹H NMR (400 MHz, DMSO) δ 8.14 (d, J=1.5, 1H), 8.06 (dd, J=8.4, 1.6, 1H), 7.45 (s, 2H), 7.38 (d, J=8.4, 1H), 5.19 (d, J=11.8, 1H), 5.09 (d, J=11.8, 1H), 4.26 (s, 1H), 4.07-3.90 (m, 2H), 3.77-3.63 (m, 2H), 3.56-3.47 (m, 1H), 3.47-3.39 (m, 1H), 3.39-3.20 (m, 2H), 3.14 (s, 3H), 2.14-1.95 (m, 2H), 1.39 (s, 3H), 1.26 (d, J=6.8, 3H). Diastereomer 2 LC-MS: m/z=426 (M+H). ¹H NMR (400 MHz, DMSO) δ 8.14 (d, J=1.4, 1H), 8.06 (dd, J=8.3, 1.6, 1H), 7.45 (s, 2H), 7.38 (d, J=8.4, 1H), 5.20-5.07 (m, 2H), 4.26 (s, 1H), 4.09-3.90 (m, 2H), 3.77-3.63 (m, 2H), 3.57-3.48 (m, 1H), 3.47-3.40 (m, 1H), 3.39-3.20 (m, 2H), 3.14 (s, 3H), 2.11-1.95 (m, 2H), 1.40 (s, 3H), 1.27 (d, J=6.7, 3H).

### Example 111

### Preparation of 5-((S)-7-(2-methoxyethyl)-7-methyl-4-((S)-3-methylmorpholino)-5,7-dihydrofuro[3,4-d]pyrimidin-2-yl)pyridin-2-amine (hf¹) and 5-((R)-7-(2-methoxyethyl)-7-methyl-4-((S)-3-methylmorpholino)-5,7-dihydrofuro[3,4-d]pyrimidin-2-yl)pyridin-2-amine (hf²)

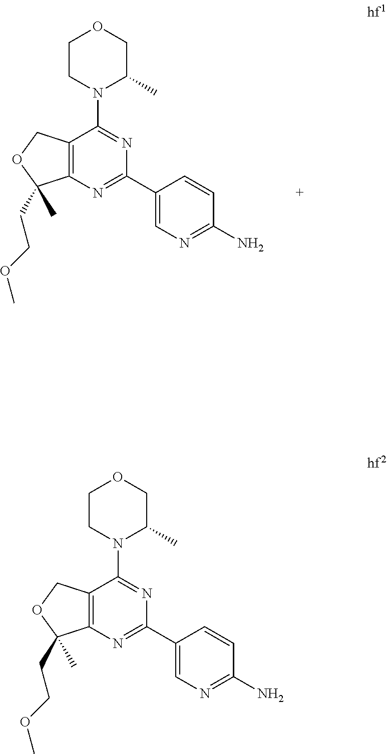

5-((S)-7-(2-methoxyethyl)-7-methyl-4-((S)-3-methylmorpholino)-5,7-dihydrofuro[3,4-d]pyrimidin-2-yl)pyridin-2-amine (hf¹ and 5-((R)-7-(2-methoxyethyl)-7-methyl-4-((S)-3-methylmorpholino)-5,7-dihydrofuro[3,4-d]pyrimidin-2-yl)pyridin-2-amine (hf²) was prepared in a similar manner as described for Example 110 with the exception that 5-(4,4,5,5-tetramethyl-1,3,2-dioxaborolan-2-yl)pyridin-2-amine was used instead of 5-(4,4,5,5-tetramethyl-1,3,2-dioxaborolan-2-yl)benzo[d]oxazol-2-amine. Diastereomer 1 LC-MS: m/z=386 (M+H). ¹H NMR (400 MHz, DMSO) δ 8.88 (d, J=2.1, 1H), 8.22 (dd, J=8.7, 2.3, 1H), 6.49 (d, J=8.7, 1H), 6.35 (s, 2H), 5.15 (d, J=11.6, 1H), 5.05 (d, J=11.6, 1H), 4.21 (s, 1H), 4.04-3.88 (m, 2H), 3.74-3.61 (m, 2H), 3.54-3.45 (m, 1H), 3.45-3.37 (m, 1H), 3.35-3.16 (m, 2H), 3.13 (s, 3H), 2.09-1.91 (m, 2H), 1.36 (s, 3H), 1.24 (d, J=6.7, 3H). Diastereomer 2 LC-MS: m/z=386 (M+H). ¹H NMR (400 MHz, DMSO) δ 8.88 (d, J=2.1, 1H), 8.22 (dd, J=8.7, 2.3, 1H), 6.49 (d, J=8.7, 1H), 6.35 (s, 2H), 5.17-5.04 (m, 2H), 4.21 (s, 1H), 4.03-3.88 (m, 2H), 3.74-3.60 (m, 2H), 3.54-3.46 (m, 1H), 3.46-3.38 (m, 1H), 3.35-3.16 (m, 2H), 3.14 (s, 3H), 2.09-1.91 (m, 2H), 1.36 (s, 3H), 1.24 (d, J=6.7, 3H).

### Example 112

### Preparation of 6-((S)-7-(2-methoxyethyl)-7-methyl-4-((S)-3-methylmorpholino)-5,7-dihydrofuro[3,4-d]pyrimidin-2-yl)benzo[d]oxazol-2-amine (hg¹) and 6-((R)-7-(2-methoxyethyl)-7-methyl-4-((S)-3-methylmorpholino)-5,7-dihydrofuro[3,4-d]pyrimidin-2-yl)benzo[d]oxazol-2-amine (hg2)

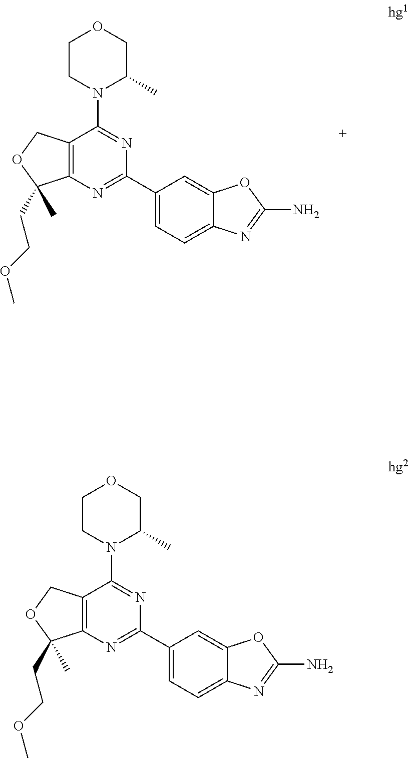

6-((S)-7-(2-methoxyethyl)-7-methyl-4-((S)-3-methylmorpholino)-5,7-dihydrofuro[3,4-d]pyrimidin-2-yl)benzo[d]oxazol-2-amine (hg¹) and 6-((R)-7-(2-methoxyethyl)-7-methyl-4-((S)-3-methylmorpholino)-5,7-dihydrofuro[3,4-d]pyrimidin-2-yl)benzo[d]oxazol-2-amine (hg²) were prepared in a similar manner as described for Example 110 with the exceptions that 6-(4,4,5,5-tetramethyl-1,3,2-dioxaborolan-2-yl)benzo[d]oxazol-2-amine was used instead of 5-(4,4,5,5-tetramethyl-1,3,2-dioxaborolan-2-yl)benzo[d]oxazol-2-amine. Diastereomer 1 LC-MS: m/z=426 (M+H) ¹H NMR (400 MHz, DMSO) δ 8.23-8.18 (m, 2H), 7.57 (s, 2H), 7.24 (d, J=8.6, 1H), 5.18 (d, J=11.8, 1H), 5.08 (d, J=11.8, 1H), 4.26 (s, 1H), 4.08-3.90 (m, 2H), 3.77-3.63 (m, 2H), 3.56-3.47 (m, 1H), 3.47-3.39 (m, 1H), 3.38-3.20 (m, 2H), 3.14 (s, 3H), 2.12-1.95 (m, 2H), 1.39 (s, 3H), 1.26 (d, J=6.7, 3H). Diastereomer 2 LC-MS: m/z=426 (M+H). ¹H NMR (400 MHz, DMSO) δ 8.23-8.17 (m, 2H), 7.57 (s, 2H), 7.28-7.21 (m, 1H), 5.18-5.07 (m, 2H), 4.26 (s, 1H), 4.11-3.89 (m, 2H), 3.76-3.62 (m, 2H), 3.56-3.48 (m, 1H), 3.47-3.40 (m, 1H), 3.38-3.19 (m, 38H), 3.14 (s, 3H), 2.11-1.95 (m, 2H), 1.39 (s, 3H), 1.27 (d, J=6.7, 3H).

### Example 113

### Preparation of 6-((S)-7-(2-methoxyethyl)-7-methyl-4-((S)-3-methylmorpholino)-5,7-dihydrofuro[3,4-d]pyrimidin-2-yl)benzo[d]isoxazol-3-amine (hi¹) and 6-((R)-7-(2-methoxyethyl)-7-methyl-4-((S)-3-methylmorpholino)-5,7-dihydrofuro[3,4-d]pyrimidin-2-yl)benzo[d]isoxazol-3-amine (hi²)

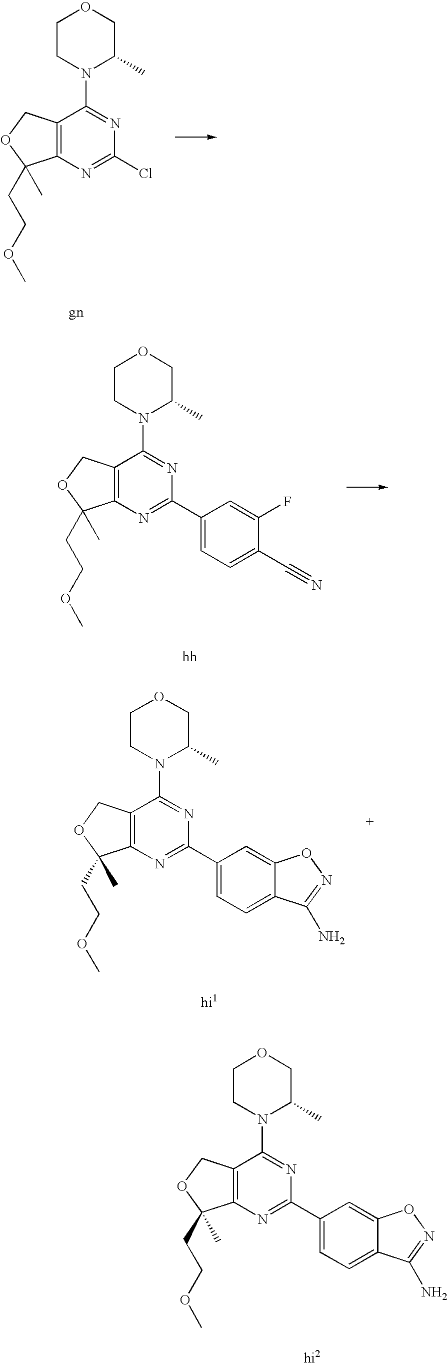

Step 1—Synthesis of 2-fluoro-4-(7-(2-methoxyethyl)-7-methyl-4-((S)-3-methylmorpholino)-5,7-dihydrofuro[3,4-d]pyrimidin-2-yl)benzonitrile (hh): 2-fluoro-4-(7-(2-methoxyethyl)-7-methyl-4-((S)-3-methylmorpholino)-5,7-dihydrofuro[3,4-d]pyrimidin-2-yl)benzonitrile was prepared in a similar manner as described for Example 110 with the exceptions that 4-cyano-3-fluorophenylboronic acid was used instead of 5-(4,4,5,5-tetramethyl-1,3,2-dioxaborolan-2-yl)benzo[d]oxazol-2-amine. LC-MS: m/z=426 (M+H) ¹H NMR (400 MHz, CDCl₃) δ 8.32 (dd, J=8.1, 1.3, 1H), 8.27-8.23 (m, 1H), 7.68 (dd, J=8.1, 6.5, 1H), 5.24-5.08 (m, 2H), 4.21 (s, 1H), 4.11-3.96 (m, 2H), 3.86-3.73 (m, 2H), 3.63 (td, J=11.9, 2.8, 1H), 3.53-3.33 (m, 3H), 3.24 (d, J=1.6, 3H), 2.27-2.06 (m, 2H), 1.50 (d, J=1.7, 3H), 1.38 (dd, J=6.8, 2.9, 3H).

Step 2—6-((S)-7-(2-methoxyethyl)-7-methyl-4-((S)-3-methylmorpholino)-5,7-dihydrofuro[3,4-d]pyrimidin-2-yl)benzo[d]isoxazol-3-amine (hi¹) and 6-((R)-7-(2-methoxyethyl)-7-methyl-4-((S)-3-methylmorpholino)-5,7-dihydrofuro[3,4-d]pyrimidin-2-yl)benzo[d]isoxazol-3-amine (hi²): To a mixture of acetohydroxamic acid (0.175 g, 0.00233 mol) in dry N,N-dimethylformamide (5.8 mL, 0.075 mol) was added potassium tert-butoxide (0.315 g, 0.00281 mol). The mixture was stirred for 30 minutes. A solution of 2-fluoro-4-(7-(2-methoxyethyl)-7-methyl-4-((S)-3-methylmorpholino)-5,7-dihydrofuro[3,4-d]pyrimidin-2-yl)benzonitrile in dry N,N-dimethylformamide (3.00 mL, 0.0387 mol) was added by canula to the mixture. The mixture was stirred overnight. The mixture was partitioned between water (50 mL) and 10% methanol in dichloromethane (50 mL). The phases were separated and the aq. extracted with 10% methanol in dichloromethane (2×50 mL). The combined organic phases were dried (MgSO₄), filtered, and chromatographed ISCO (12 g, 0-100% ethyl acetate in heptane). The diastereomers were separated by super critical fluid chromatography to give 107.4 mg and 102.8 mg of each diastereomer. Diastereomer 1 LC-MS: m/z=426 (M+H). ¹H NMR (400 MHz, DMSO) δ 8.31 (s, 1H), 8.27 (dd, J=8.3, 1.0, 1H), 7.90 (d, J=8.3, 1H), 6.45 (s, 2H), 5.26-5.07 (m, 2H), 4.28 (s, 1H), 4.14-3.91 (m, 2H), 3.77-3.64 (m, 2H), 3.57-3.48 (m, 1H), 3.47-3.22 (m, 3H), 3.14 (s, 3H), 2.15-1.96 (m, 2H), 1.41 (s, 3H), 1.28 (d, J=6.7, 3H). Diastereomer 2 LC-MS: m/z=426 (M+H). ¹H NMR (400 MHz, DMSO) δ 8.31 (s, 1H), 8.27 (d, J=8.3, 1H), 7.90 (d, J=8.3, 1H), 6.45 (s, 2H), 5.24-5.08 (m, 2H), 4.29 (s, 1H), 4.15-3.92 (m, 2H), 3.79-3.63 (m, 2H), 3.57-3.20 (m, 4H), 3.14 (s, 3H), 2.14-1.95 (m, 2H), 1.41 (s, 3H), 1.28 (d, J=6.7, 3H).

### Example 114

### Preparation of 5-(7,7-dimethyl-4-morpholino-5,7-dihydrofuro[3,4-d]pyrimidin-2-yl)benzo[d]oxazol-2-amine (hj)

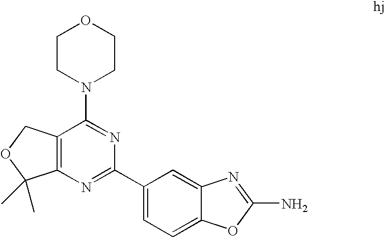

5-(7,7-dimethyl-4-morpholino-5,7-dihydrofuro[3,4-d]pyrimidin-2-yl)benzo[d]oxazol-2-amine was prepared in a similar manner as described for Example 110 with the exceptions that 2-chloro-7,7-dimethyl-4-morpholino-5,7-dihydrofuro[3,4-d]pyrimidine was used instead of 2-chloro-7-(2-methoxyethyl)-7-methyl-4-((S)-3-methylmorpholino)-5,7-dihydrofuro[3,4-d]pyrimidine: LC-MS: m/z=368 (M+H). ¹H NMR (400 MHz, DMSO) δ 8.15 (d, J=1.4, 1H), 8.07 (dd, J=8.4, 1.6, 1H), 7.48 (s, 2H), 7.37 (d, J=8.4, 1H), 5.14 (s, 2H), 3.74-3.69 (m, 4H), 3.68-3.63 (m, 4H), 1.41 (s, 6H).

### Example 115

### Preparation of 6-(7,7-dimethyl-4-morpholino-5,7-dihydrofuro[3,4-d]pyrimidin-2-yl)benzo[d]oxazol-2-amine (hk)

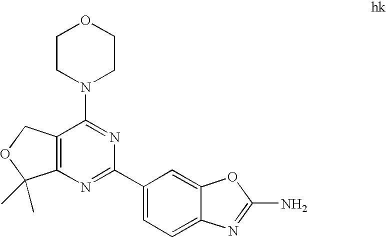

5-(7,7-dimethyl-4-morpholino-5,7-dihydrofuro[3,4-d]pyrimidin-2-yl)benzo[d]oxazol-2-amine was prepared in a similar manner as described for Example 110 with the exceptions that 2-chloro-7,7-dimethyl-4-morpholino-5,7-dihydrofuro[3,4-d]pyrimidine was used instead of 2-chloro-7-(2-methoxyethyl)-7-methyl-4-((S)-3-methylmorpholino)-5,7-dihydrofuro[3,4-d]pyrimidine and 6-(4,4,5,5-tetramethyl-1,3,2-dioxaborolan-2-yl)benzo[d]oxazol-2-amine was used instead of 5-(4,4,5,5-tetramethyl-1,3,2-dioxaborolan-2-yl)benzo[d]oxazol-2-amine. LC-MS: m/z=368 (M+H). ¹H NMR (400 MHz, DMSO) δ 8.21 (s, 2H), 7.60 (s, 2H), 7.24 (d, J=7.7, 1H), 5.14 (s, 2H), 3.79-3.58 (m, 8H), 1.41 (s, 6H).

### Example 116

### Preparation of 6-(7,7-dimethyl-4-morp ho lino-5,7-dihydro furo [3,4-d]pyrimidin-2-yl)benzo[d] isoxazol-3-amine (hl)

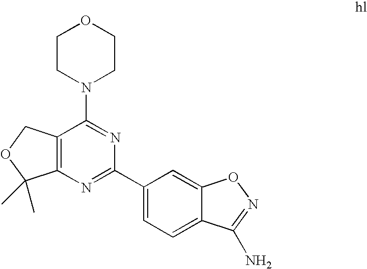

6-(7,7-dimethyl-4-morpholino-5,7-dihydrofuro[3,4-d]pyrimidin-2-yl)benzo[d]isoxazol-3-amine was prepared in a similar manner as described for Example 113 with the exception that 2-chloro-7,7-dimethyl-4-morpholino-5,7-dihydrofuro[3,4-d]pyrimidine was used instead of 2-chloro-7-(2-methoxyethyl)-7-methyl-4-((S)-3-methylmorpholino)-5,7-dihydrofuro[3,4-d]pyrimidine in Step 1. LC-MS: m/z=368 (M+H). ¹H NMR (400 MHz, DMSO) δ 8.32 (s, 1H), 8.28 (d, J=8.3, 1H), 7.90 (d, J=8.3, 1H), 6.47 (s, 2H), 5.17 (s, 2H), 3.78-3.61 (m, 8H), 1.43 (s, 6H).

### Example 117

### Preparation of 5-(7,7-dimethyl-4-morpholino-5,7-dihydrofuro[3,4-d]pyrimidin-2-yl)benzo[d]isoxazol-3-amine (hm)

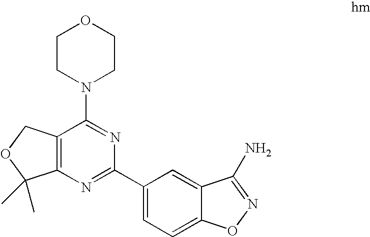

5-(7,7-dimethyl-4-morpholino-5,7-dihydrofuro[3,4-d]pyrimidin-2-yl)benzo[d]isoxazol-3-amine was prepared in a similar manner as described for Example 113 with the exception that 2-chloro-7,7-dimethyl-4-morpholino-5,7-dihydrofuro[3,4-d]pyrimidine was used instead of 2-chloro-7-(2-methoxyethyl)-7-methyl-4-((S)-3-methylmorpholino)-5,7-dihydrofuro[3,4-d]pyrimidine and 2-fluoro-5-(4,4,5,5-tetramethyl-1,3,2-dioxaborolan-2-yl)benzonitrile was used instead of 4-cyano-3-fluorophenylboronic acid in Step 1. LC-MS: m/z=368 (M+H). ¹H NMR (400 MHz, DMSO) δ 8.86 (s, 1H), 8.55 (d, J=8.8, 1H), 7.51 (d, J=8.8, 1H), 6.59 (s, 2H), 5.16 (s, 2H), 3.79-3.64 (m, 8H), 1.43 (s, 6H).

### Example 118

### Preparation of 6-(7,7-dimethyl-4-morp ho lino-5,7-dihydro furo [3,4-d]pyrimidin-2-yl)-1H-benzo[d]imidazol-2-amine (hp)

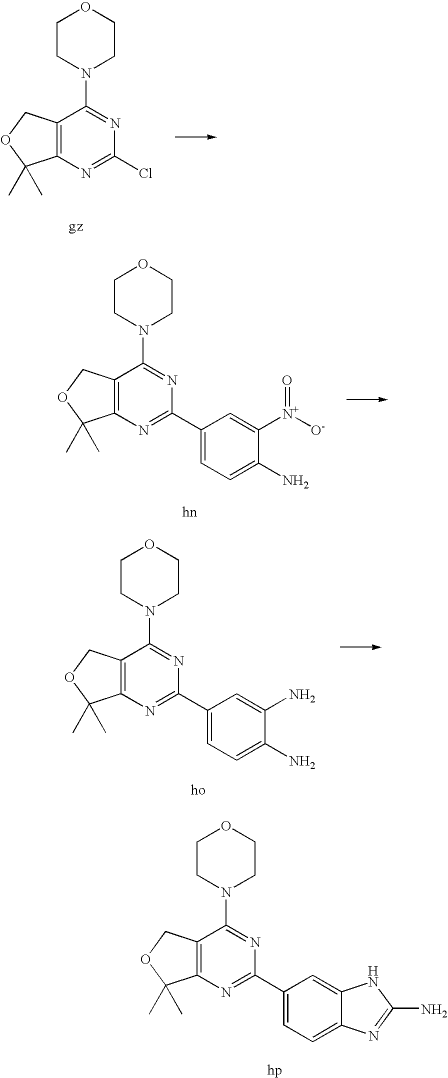

Step 1—Synthesis of 4-(7,7-dimethyl-4-morpholino-5,7-dihydrofuro[3,4-d]pyrimidin-2-yl)-2-nitroaniline (hn): 4-(7,7-dimethyl-4-morpholino-5,7-dihydrofuro[3,4-d]pyrimidin-2-yl)-2-nitroaniline was prepared in a similar manner as described for Example 110 with the exceptions that 2-chloro-7,7-dimethyl-4-morpholino-5,7-dihydrofuro[3,4-d]pyrimidine was used instead of 2-chloro-7-(2-methoxyethyl)-7-methyl-4-((S)-3-methylmorpholino)-5,7-dihydrofuro[3,4-d]pyrimidine and 2-nitro-4-(4,4,5,5-tetramethyl-1,3,2-dioxaborolan-2-yl)aniline was used instead of 5-(4,4,5,5-tetramethyl-1,3,2-dioxaborolan-2-yl)benzo[d]oxazol-2-amine. LC-MS: m/z=372 (M+H) ¹H NMR (400 MHz, CDCl₃) δ 9.18 (d, J=1.8, 1H), 8.45 (dd, J=8.7, 1.9, 1H), 6.85 (d, J=8.7, 1H), 6.24 (s, 2H), 5.15 (s, 2H), 3.85-3.79 (m, 4H), 3.73-3.68 (m, 4H), 1.51 (s, 6H).

Step 2—Synthesis of 4-(7,7-dimethyl-4-morpholino-5,7-dihydrofuro[3,4-d]pyrimidin-2-yl)benzene-1,2-diamine (ho): 4-(7,7-dimethyl-4-morpholino-5,7-dihydrofuro[3,4-d]pyrimidin-2-yl)-2-nitroaniline (0.229 g, 0.617 mmol), iron (0.172 g, 3.08 mmol), and ammonium chloride (0.132 g, 2.47 mmol) were combined and purged with nitrogen. To the mixture was added dry ethanol (1.80 mL, 30.8 mmol) and deoxygenated water (1.78 mL, 98.6 mmol). The mixture was heated at 75° C. and stirred for 3 hours. The mixture filtered through celite. The filtrate was partitioned between saturated bicarbonate solution (50 mL) and 10% methanol in dichloromethane (50 mL). The phases were separated and the aq. extracted with 10% methanol in dichloromethane (2×50 mL). The combined organic phases were dried (MgSO₄), filtered, and concentrated to give 0.229 g 4-(7,7-dimethyl-4-morpholino-5,7-dihydrofuro[3,4-d]pyrimidin-2-yl)benzene-1,2-diamine. LC-MS: m/z=342 (M+H) ¹H NMR (400 MHz, CDCl₃) δ 7.86-7.81 (m, 2H), 6.74 (d, J=8.0, 1H), 5.13 (s, 2H), 3.83-3.78 (m, 4H), 3.72-3.67 (m, 4H), 3.63 (s, 2H), 3.41 (s, 2H), 1.50 (s, 6H).

Step 3—Synthesis of 6-(7,7-dimethyl-4-morpholino-5,7-dihydrofuro[3,4-d]pyrimidin-2-yl)-1H-benzo[d]imidazol-2-amine (hp): To a solution of 4-(7,7-dimethyl-4-morpholino-5,7-dihydrofuro[3,4-d]pyrimidin-2-yl)benzene-1,2-diamine (0.191 g, 0.559 mmol) in dry methanol (3.00 mL, 74.0 mmol) was added 3.0 M of cyanogen bromide in methylene chloride (0.240 mL) dropwise. The mixture was stirred for 3 hours. The mixture was concentrated and purified by HPLC to give 0.1424 g of 6-(7,7-dimethyl-4-morpholino-5,7-dihydrofuro[3,4-d]pyrimidin-2-yl)-1H-benzo[d]imidazol-2-amine. LC-MS: m/z=367 (M+H). ¹H NMR (400 MHz, DMSO) δ 10.78 (s, 1H), 8.13 (s, 1H), 8.01 (d, J=8.1, 1H), 7.12 (d, J=8.3, 1H), 6.35 (s, 2H), 5.13 (s, 2H), 3.75-3.60 (m, 8H), 1.41 (s, 6H).

### Example 119

### Preparation of 5-((S)-7-(2-methoxyethyl)-7-methyl-4-(S)-3-methylmorpholino)-5,7-dihydrofuro[3,4-d]pyrimidin-2-yl)benzo[d]isoxazol-3-amine (hq¹) and 5-((R)-7-(2-methoxyethyl)-7-methyl-4-((S)-3-methylmorpholino)-5,7-dihydrofuro[3,4-d]pyrimidin-2-yl)benzo[d]isoxazol-3-amine (hq²)

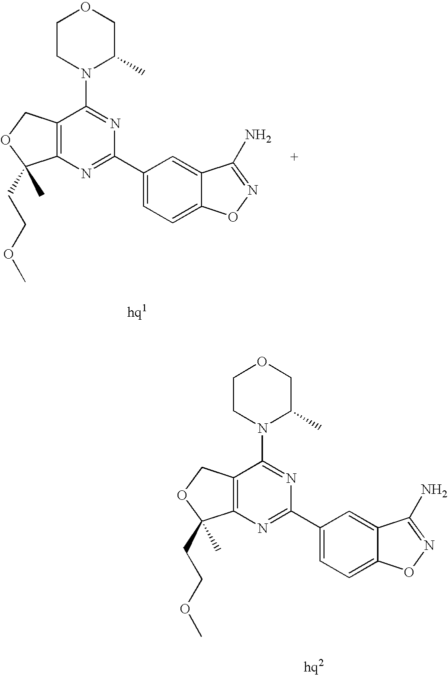

5-((S)-7-(2-methoxyethyl)-7-methyl-4-((S)-3-methylmorpholino)-5,7-dihydrofuro[3,4-d]pyrimidin-2-yl)benzo[d]isoxazol-3-amine (hq¹) and 5-((R)-7-(2-methoxyethyl)-7-methyl-4-((S)-3-methylmorpholino)-5,7-dihydrofuro[3,4-d]pyrimidin-2-yl)benzo[d]isoxazol-3-amine (hq²) were prepared in a similar manner as described for Example 113 with the exception that 3-cyano-4-fluorophenylboronic acid was used instead of in 4-cyano-3-fluorophenylboronic acid Step 1. Diastereomer 1 LC-MS: m/z=426 (M+H). ¹H NMR (400 MHz, DMSO) δ 8.84 (s, 1H), 8.54 (d, J=8.7, 1H), 7.51 (d, J=8.8, 1H), 6.59 (s, 2H), 5.21 (d, J=11.8, 1H), 5.11 (d, J=11.7, 1H), 4.42-3.92 (m, 3H), 3.79-3.64 (m, 2H), 3.58-3.49 (m, 1H), 3.48-3.20 (m, 3H), 3.14 (s, 3H), 2.15-1.97 (m, 2H), 1.41 (s, 3H), 1.28 (d, J=6.6, 3H). Diastereomer 2 LC-MS: m/z=426 (M+H). ¹H NMR (400 MHz, DMSO) δ 8.84 (s, 1H), 8.54 (d, J=8.7, 1H), 7.51 (d, J=8.8, 1H), 6.59 (s, 2H), 5.23-5.09 (m, 2H), 4.46-3.92 (m, 3H), 3.78-3.64 (m, 2H), 3.59-3.49 (m, 1H), 3.48-3.40 (m, 1H), 3.40-3.20 (m, 2H), 3.14 (s, 3H), 2.15-1.97 (m, 2H), 1.42 (s, 3H), 1.28 (d, J=6.6, 3H).

### Example 120

### Preparation of (S)-6-(7,7-dimethyl-4-(3-methylmorpholino)-5,7-dihydrofuro[3,4-d]pyrimidin-2-yl)benzo[d]isoxazol-3-amine (hr)

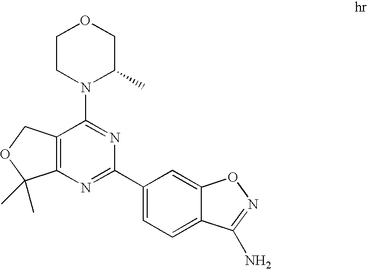

(S)-6-(7,7-dimethyl-4-(3-methylmorpholino)-5,7-dihydrofuro[3,4-d]pyrimidin-2-yl)benzo[d]isoxazol-3-amine was prepared in a similar manner as described for Example 113 with the exception that (S)-2-chloro-7,7-dimethyl-4-(3-methylmorpholino)-5,7-dihydrofuro[3,4-d]pyrimidine was used instead of in 2-chloro-7-(2-methoxyethyl)-7-methyl-44(S)-3-methylmorpholino)-5,7-dihydrofuro[3,4-d]pyrimidine in Step 1. LC-MS: m/z=382 (M+H). ¹H NMR (400 MHz, DMSO) δ 8.31 (s, 1H), 8.27 (d, J=8.4, 1H), 7.89 (d, J=8.3, 1H), 6.47 (s, 2H), 5.20 (d, J=12.2, 1H), 5.12 (d, J=11.9, 1H), 4.33 (s, 1H), 4.16-3.92 (m, 2H), 3.78-3.70 (m, 1H), 3.70-3.63 (m, 1H), 3.57-3.47 (m, 1H), 3.42-3.35 (m, 1H), 1.43 (s, 6H), 1.28 (d, J=6.7, 3H).

### Example 121

### Preparation of (S)-5-(7,7-dimethyl-4-(3-methylmorp ho lino)-5,7-dihydro furo [3,4-d]pyrimidin-2-yl)benzo[d]isoxazol-3-amine (hs)

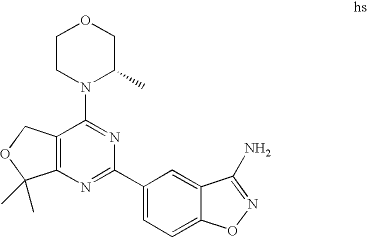

(S)-5-(7,7-dimethyl-4-(3-methylmorpholino)-5,7-dihydrofuro[3,4-d]pyrimidin-2-yl)benzo[d]isoxazol-3-amine was prepared in a similar manner as described for Example 113 with the exception that (S)-2-chloro-7,7-dimethyl-4-(3-methylmorpholino)-5,7-dihydrofuro[3,4-d]pyrimidine was used instead of in 2-chloro-7-(2-methoxyethyl)-7-methyl-4-((S)-3-methylmorpholino)-5,7-dihydrofuro[3,4-d]pyrimidine and 2-fluoro-5-(4,4,5,5-tetramethyl-1,3,2-dioxaborolan-2-yl)benzonitrile was used instead of 4-cyano-3-fluorophenylboronic acid in Step 1. LC-MS: m/z=382 (M+H). ¹H NMR (400 MHz, DMSO) δ 8.84 (s, 1H), 8.54 (d, J=8.8, 1H), 7.51 (d, J=8.9, 1H), 6.59 (s, 2H), 5.20 (d, J=11.6, 1H), 5.12 (d, J=11.8, 1H), 4.44-3.92 (m, 3H), 3.78-3.64 (m, 2H), 3.59-3.48 (m, 1H), 3.42-3.35 (m, 1H), 1.44 (s, 6H), 1.29 (d, J=6.5, 3H).

### Example 122

### Preparation of (S)-6-(7,7-dimethyl-4-(3-methylmorpholino)-5,7-dihydrofuro[3,4-d]pyrimidin-2-yl)-1H-benzo[d]imidazol-2-amine (ht)

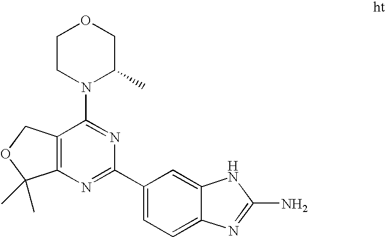

(S)-6-(7,7-dimethyl-4-(3-methylmorpholino)-5,7-dihydrofuro[3,4-d]pyrimidin-2-yl)-1H-benzo[d]imidazol-2-amine was prepared in a similar manner as described for Example 118 with the exception that (S)-2-chloro-7,7-dimethyl-4-(3-methylmorpholino)-5,7-dihydrofuro[3,4-d]pyrimidine was used instead of 2-chloro-7,7-dimethyl-4-morpholino-5,7-dihydrofuro[3,4-d]pyrimidine in Step 1. LC-MS: m/z=381 (M+H). ¹H NMR (400 MHz, DMSO) δ 10.86-10.64 (m, 1H), 8.16-8.08 (m, 1H), 8.06-7.92 (m, 1H), 7.17-7.06 (m, 1H), 6.42-6.15 (m, 2H), 5.16 (d, J=11.7, 1H), 5.08 (d, J=11.7, 1H), 4.28 (s, 1H), 4.11-3.91 (m, 2H), 3.77-3.63 (m, 2H), 3.57-3.46 (m, 1H), 3.41-3.25 (m, 1H), 1.41 (s, 6H), 1.27 (d, J=6.6, 3H).

### Example 123

### Preparation of (S)-5-(7,7-dimethyl-4-(3-methylmorp ho lino)-5,7-dihydro furo [3,4-d]pyrimidin-2-yl)benzo[d]oxazol-2-amine (hu)

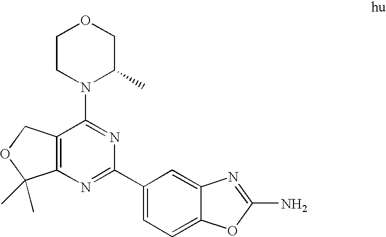

(S)-5-(7,7-dimethyl-4-(3-methylmorpholino)-5,7-dihydrofuro[3,4-d]pyrimidin-2-yl)benzo[d]oxazol-2-amine was prepared in a similar manner as described for Example 110 with the exception that (S)-2-chloro-7,7-dimethyl-4-(3-methylmorpholino)-5,7-dihydrofuro[3,4-d]pyrimidine was used instead of 2-chloro-7-(2-methoxyethyl)-7-methyl-4-((S)-3-methylmorpholino)-5,7-dihydrofuro[3,4-d]pyrimidine. LC-MS: m/z=382 (M+H). ¹H NMR (400 MHz, DMSO) δ 8.15 (s, 1H), 8.06 (d, J=8.3, 1H), 7.48 (s, 2H), 7.38 (d, J=8.4, 1H), 5.17 (d, J=11.6, 1H), 5.10 (d, J=11.7, 1H), 4.30 (s, 1H), 4.11-3.90 (m, 2H), 3.77-3.62 (m, 2H), 3.56-3.46 (m, 1H), 3.40-3.33 (m, 1H), 1.41 (s, 6H), 1.27 (d, J=6.6, 3H).

### Example 124

### Preparation of 6-((S)-7-(2-methoxyethyl)-7-methyl-4-(S)-3-methylmorpholino)-5,7-dihydrofuro[3,4-d]pyrimidin-2-yl)-1H-benzo[d]imidazol-2-amine (hv¹) and (6-((R)-7-(2-methoxyethyl)-7-methyl-4-((S)-3-methylmorpholino)-5,7-dihydrofuro [3,4-d]pyrimidin-2-yl)-1H-benzo[d]imidazol-2-amine (hv²)

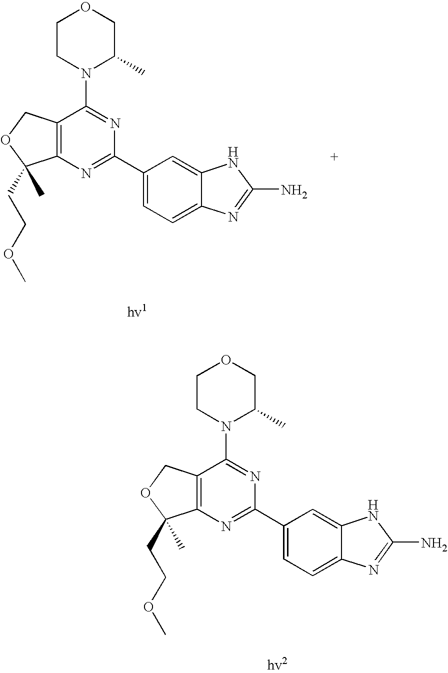

6-((S)-7-(2-methoxyethyl)-7-methyl-4-((S)-3-methylmorpholino)-5,7-dihydrofuro [3,4-d]pyrimidin-2-yl)-1H-b enzo[d]imidazol-2-amine and 6-((R)-7-(2-methoxyethyl)-7-methyl-4-((S)-3-methylmorpholino)-5,7-dihydrofuro [3,4-d]pyrimidin-2-yl)-1H-benzo[d]imidazol-2-amine were prepared in a similar manner as described for Example 9 with the exception that 2-chloro-7-(2-methoxyethyl)-7-methyl-4-((S)-3-methylmorpholino)-5,7-dihydrofuro[3,4-d]pyrimidine was used instead of 2-chloro-7,7-dimethyl-4-morpholino-5,7-dihydrofuro[3,4-d]pyrimidine in Step 1 and the diastereomers were separated in Step 3. Diastereomer 1 LC-MS: m/z=426 (M+H). ¹H NMR (400 MHz, DMSO) δ 10.86-10.65 (m, 1H), 8.12 (s, 1H), 8.06-7.90 (m, 1H), 7.17-7.06 (m, 1H), 6.42-6.14 (m, 2H), 5.19-5.03 (m, 2H), 4.25 (s, 1H), 4.11-3.91 (m, 2H), 3.77-3.62 (m, 2H), 3.57-3.48 (m, 1H), 3.47-3.40 (m, 1H), 3.38-3.27 (m, 1H), 3.26-3.18 (m, 1H), 3.15 (s, 3H), 2.12-1.94 (m, 2H), 1.39 (s, 3H), 1.26 (d, J=6.5, 3H). Diastereomer 2 LC-MS: m/z=426 (M+H). ¹H NMR (400 MHz, DMSO) δ 10.86-10.65 (m, 1H), 8.12 (s, 1H), 8.06-7.90 (m, 1H), 7.17-7.08 (m, 1H), 6.42-6.12 (m, 2H), 5.17 (d, J=11.7, 1H), 5.07 (d, J=11.5, 1H), 4.25 (s, 1H), 4.09-3.91 (m, 2H), 3.78-3.63 (m, 2H), 3.57-3.47 (m, 1H), 3.47-3.39 (m, 1H), 3.38-3.28 (m, 1H), 3.27-3.18 (m, 1H), 3.14 (s, 3H), 2.13-1.94 (m, 2H), 1.39 (s, 3H), 1.26 (d, J=6.6, 3H).

### Example 125

### Preparation of (S)-5-(7,7-dimethyl-4-(3-methylmorp ho lino)-5,7-dihydro furo [3,4-d]pyrimidin-2-yl)-N-methyl-1H-benzo[d]imidazol-2-amine (hw)

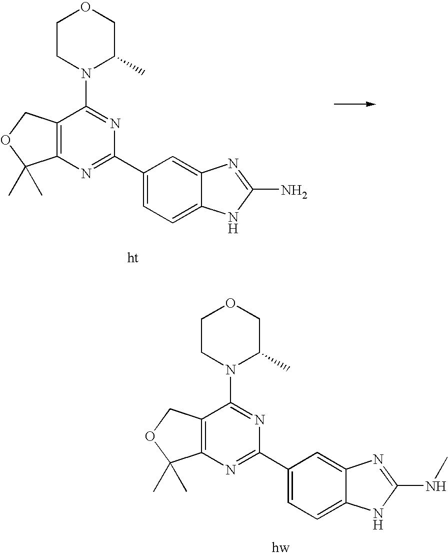

25% Sodium methoxide in methanol (1:3, sodium methoxide:methanol, 0.100 mL) was added to (S)-5-(7,7-dimethyl-4-(3-methylmorpholino)-5,7-dihydrofuro[3,4-d]pyrimidin-2-yl)-1H-benzo[d]imidazol-2-amine (0.0244 g, 0.0000641 mol). The mixture was stirred for 10 minutes. Paraformaldehyde (0.0036 g, 0.00012 mol;) was added to the mixture. The mixture was stirred overnight. Sodium tetrahydroborate (0.0027 g, 0.000071 mol;) to the mixture. The mixture was heated at 65° C. for 2 hours. The mixture was partitioned between water (20 mL) and 10% methanol in dichloromethane (20 mL). The phases were separated and the aq. extracted with 10% methanol in dichloromethane (2×20 mL). The combined organic phases were dried (MgSO₄), filtered, and chromatographed ISCO (4 g, 0-10% methanol in dichloromethane). The residue was purified by HPLC to give 0.0029 g (S)-5-(7,7-dimethyl-4-(3-methylmorpholino)-5,7-dihydrofuro[3,4-d]pyrimidin-2-yl)-N-methyl-1H-benzo[d]imidazol-2-amine. LC-MS: m/z=198 (M+2H). ¹H NMR (500 MHz, DMSO) δ 11.05-10.92 (m, 1H), 8.19-8.12 (m, 1H), 8.06-7.94 (m, 1H), 7.20-7.12 (m, 1H), 6.77 (s, 1H), 5.16 (d, J=11.5, 1H), 5.08 (d, J=11.8, 1H), 3.98-3.92 (m, 1H), 3.76-3.72 (m, 1H), 3.69-3.32 (m, 5H), 2.87 (d, J=4.8, 3H), 1.41 (s, 6H), 1.27 (d, J=6.4, 3H).

### Example 126

### Preparation of (S)-1-ethyl-3-(4-(7-(2-hydroxy-2-methylpropyl)-7-methyl-4-morpholino-5,7-dihydrofuro[3,4-d]pyrimidin-2-yl)phenyl)urea (ib¹) and (R)-1-ethyl-3-(4-(7-(2-hydroxy-2-methylpropyl)-7-methyl-4-morpholino-5,7-dihydrofuro[3,4-d]pyrimidin-2-yl)phenyl)urea (ib²)

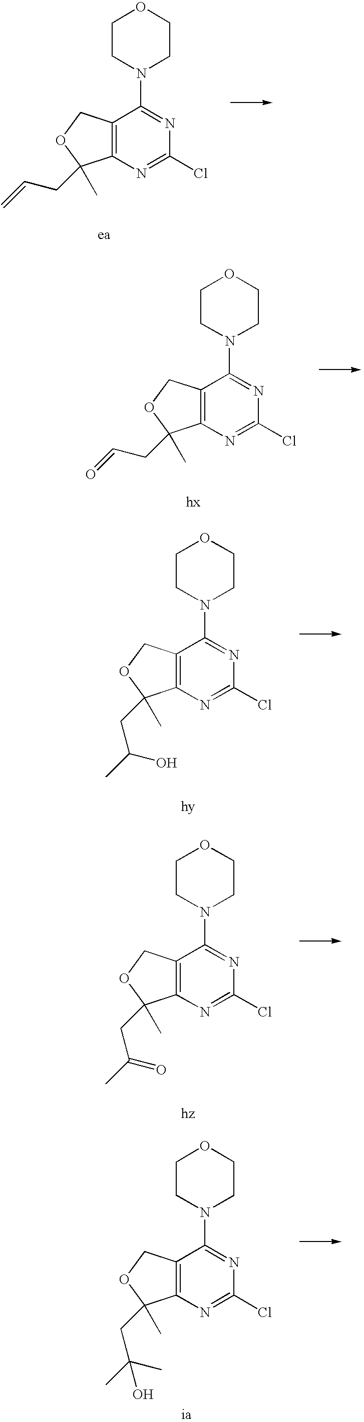

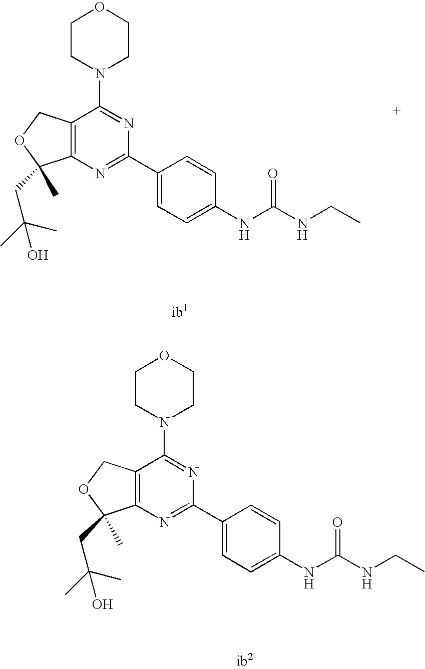

Step 1—Synthesis of 2-(2-chloro-7-methyl-4-morpholino-5,7-dihydrofuro[3,4-d]pyrimidin-7-yl)acetaldehyde: To a solution of 7-allyl-2-chloro-7-methyl-4-morpholino-5,7-dihydrofuro[3,4-d]pyrimidine (2.021 g, 0.006833 mol) in dry acetonitrile (180.0 mL, 3.446 mol) and deoxygenated water (180.0 mL, 9.992 mol) was added 5 drops of 4% osmium tetroxide in water. The mixture was stirred for 10 minutes. Sodium periodate (5.8643 g, 0.027417 mol) was added to the mixture. The mixture was stirred overnight. Added an additional 5 drops of 4% osmium tetroxide in water to the mixture. The mixture was stirred for 3 days. The mixture was partitioned between saturated sodium thiosulfate solution (300 mL) and ethyl acetate. The phases were separated and the aq. extracted with ethyl acetate (2×300 mL). The combined organic phases were dried (MgSO₄), filtered, and chromatographed ISCO (40 g, 0-100% ethyl acetate in heptane) to give 1.308 g of 2-(2-chloro-7-methyl-4-morpholino-5,7-dihydrofuro[3,4-d]pyrimidin-7-yl)acetaldehyde. LC-MS: m/z=298 (M+H) ¹H NMR (500 MHz, CDCl₃) δ 9.66 (s, 1H), 5.17-5.09 (m, 2H), 3.82-3.73 (m, 5H), 3.70-3.59 (m, 4H), 2.94 (d, J=15.5, 1H), 2.87 (dd, J=16.2, 2.8, 1H), 1.52 (s, 3H).

Step 2—Synthesis of 1-(2-chloro-7-methyl-4-morpholino-5,7-dihydrofuro[3,4-d]pyrimidin-7-yl)propan-2-ol: To a solution of 2-(2-chloro-7-methyl-4-morpholino-5,7-dihydrofuro[3,4-d]pyrimidin-7-yl)acetaldehyde (1.308 g, 0.004393 mol) in dry ether (41.0 mL, 0.390 mol) at 0° C. was added 3.0 M of methylmagnesium iodide in ether (4.40 mL) dropwise. The mixture was stirred 0° C. for 1 hour. The mixture was quenched by adding 0.1M HCl (5 mL). The mixture was stirred at −78° C. for 1 hour. The mixture was partitioned between water (50 mL) and ethyl acetate (50 mL). The phases were separated and the aq. extracted with ethyl acetate (2×50 mL). The combined organic phases were washed with sat NaCl solution, dried (MgSO₄), filtered, and chromatographed ISCO (40 g, 0-100% ethyl acetate in heptane) to give 1.286 g 1-(2-chloro-7-methyl-4-morpholino-5,7-dihydrofuro[3,4-d]pyrimidin-7-yl)propan-2-ol. LC-MS: m/z=314 (M+H).

Step 3—Synthesis of 1-(2-chloro-7-methyl-4-morpholino-5,7-dihydrofuro[3,4-d]pyrimidin-7-yl)propan-2-one: To a solution of oxalyl chloride (1.39 mL, 0.0165 mol) in dry methylene chloride (26.6 mL, 0.415 mol) at −78° C. was added dimethyl sulfoxide (2.28 mL, 0.0322 mol) dropwise. The mixture was stirred at −78° C. for 5 minutes before adding 1-(2-chloro-7-methyl-4-morpholino-5,7-dihydrofuro[3,4-d]pyrimidin-7-yl)propan-2-ol (1.192 g, 0.003799 mol) as a solution in dry methylene chloride (26.6 mL, 0.415 mol) dropwise. Triethylamine (9.00 mL, 0.0646 mol) dropwise to the mixture. The mixture was stirred at −78° C. for 1 hour. The mixture was partitioned between phosphate buffer (30 mL) and dichloromethane (50 mL). The phases were separated and the aq. extracted with dichloromethane (2×50 mL). The combined organic phases were dried (MgSO₄), filtered, and chromatographed ISCO (40 g, 0-75% ethyl acetate in heptane) to give 0.815 g 1-(2-chloro-7-methyl-4-morpholino-5,7-dihydrofuro[3,4-d]pyrimidin-7-yl)propan-2-one. LC-MS: m/z=312 (M+H). ¹H NMR (500 MHz, CDCl₃) δ 5.16-5.08 (m, 2H), 3.79-3.74 (m, 4H), 3.66-3.61 (m, 4H), 3.06-2.93 (m, 2H), 2.10 (s, 3H), 1.44 (s, 3H).

Step 4—Synthesis of 1-(2-chloro-7-methyl-4-morpholino-5,7-dihydrofuro[3,4-d]pyrimidin-7-yl)-2-methylpropan-2-ol: 1-(2-chloro-7-methyl-4-morpholino-5,7-dihydrofuro[3,4-d]pyrimidin-7-yl)-2-methylpropan-2-ol was prepared in a similar manner as described for Step 2 with the exception that 1-(2-chloro-7-methyl-4-morpholino-5,7-dihydrofuro[3,4-d]pyrimidin-7-yl)propan-2-one was used instead of 2-(2-chloro-7-methyl-4-morpholino-5,7-dihydrofuro[3,4-d]pyrimidin-7-yl)acetaldehyde. LC-MS: m/z=328 (M+H).

Step 5—Synthesis of (S)-1-ethyl-3-(4-(7-(2-hydroxy-2-methylpropyl)-7-methyl-4-morpholino-5,7-dihydrofuro[3,4-d]pyrimidin-2-yl)phenyl)urea (ib¹) and (R)-1-ethyl-3-(4-(7-(2-hydroxy-2-methylpropyl)-7-methyl-4-morpholino-5,7-dihydrofuro[3,4-d]pyrimidin-2-yl)phenyl)urea (ib²): ibⁱ and ib² were prepared in a similar manner as described for Example 110 with the exception that 1-(2-chloro-7-methyl-4-morpholino-5,7-dihydrofuro[3,4-d]pyrimidin-7-yl)-2-methylpropan-2-ol was used instead of 2-chloro-7-(2-methoxyethyl)-7-methyl-4-((S)-3-methylmorpholino)-5,7-dihydrofuro[3,4-d]pyrimidine and 1-ethyl-3-(4-(4,4,5,5-tetramethyl-1,3,2-dioxaborolan-2-yl)phenyl)urea was used instead of 5-(4,4,5,5-tetramethyl-1,3,2-dioxaborolan-2-yl)benzo[d]oxazol-2-amine. (Enantiomer 1): LC-MS: m/z=456 (M+H). ¹H NMR (400 MHz, DMSO) δ 8.65 (s, 1H), 8.20 (d, J=8.8, 2H), 7.48 (d, J=8.8, 2H), 6.14 (t, J=5.6, 1H), 5.15 (s, 2H), 4.19 (s, 1H), 3.75-3.58 (m, 8H), 3.18-3.06 (m, 2H), 2.06-1.94 (m, 2H), 1.41 (s, 3H), 1.11 (s, 3H), 1.06 (t, J=7.2, 3H), 1.00 (s, 3H). (Enantiomer 2): LC-MS: m/z=456 (M+H). ¹H NMR (400 MHz, DMSO) δ 8.66 (s, 1H), 8.20 (d, J=8.8, 2H), 7.48 (d, J=8.8, 2H), 6.15 (t, J=5.5, 1H), 5.15 (s, 2H), 4.19 (s, 1H), 3.75-3.58 (m, 8H), 3.17-3.06 (m, 2H), 2.06-1.93 (m, 2H), 1.41 (s, 3H), 1.11 (s, 3H), 1.06 (t, J=7.2, 3H), 1.00 (s, 3H).

### Example 127

### Preparation of 1-ethyl-3-(4-((S)-7-(2-hydroxy-2-methylpropyl)-7-methyl-4-(S)-3-methylmorpholino)-5,7-dihydrofuro[3,4-d]pyrimidin-2-yl)phenyl)urea (ie¹) and 1-ethyl-3-(4-4R)-7-(2-hydroxy-2-methylpropyl)-7-methyl-4-((S)-3-methylmorpholino)-5,7-dihydrofuro[3,4-d]pyrimidin-2-yl)phenyl)urea (ie²)

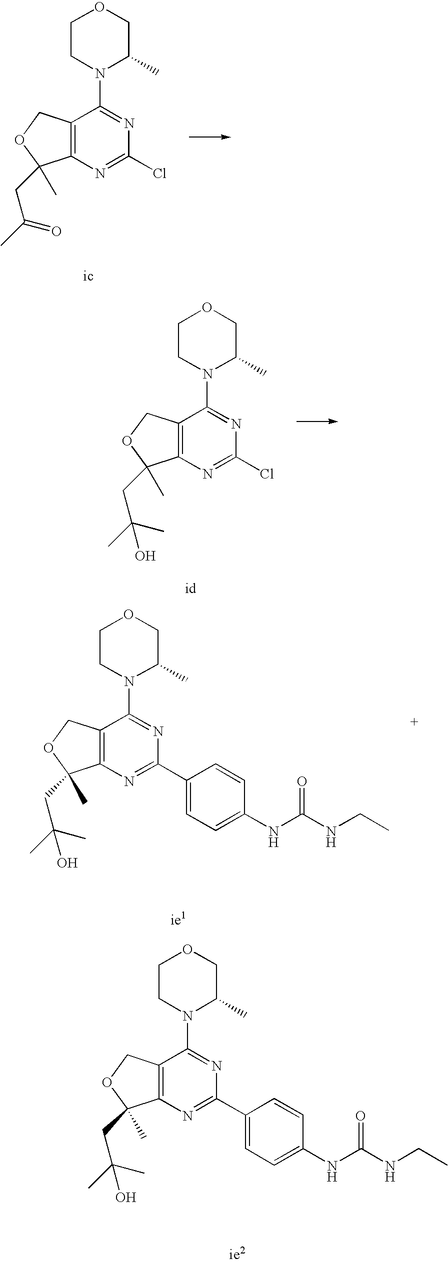

1-ethyl-3-(4-((S)-7-(2-hydroxy-2-methylpropyl)-7-methyl-4-((S)-3-methylmorpholino)-5,7-dihydrofuro[3,4-d]pyrimidin-2-yl)phenyl)urea and 1-ethyl-3-(4-((R)-7-(2-hydroxy-2-methylpropyl)-7-methyl-4-((S)-3-methylmorpholino)-5,7-dihydrofuro[3,4-d]pyrimidin-2-yl)phenyl)urea were prepared in a similar manner as described for Steps 4 and 5 in Example 126 with the exception that 1-(2-chloro-7-methyl-4-((S)-3-methylmorpholino)-5,7-dihydrofuro[3,4-d]pyrimidin-7-yl)-2-methylpropan-2-ol was used instead of 1-(2-chloro-7-methyl-4-morpholino-5,7-dihydrofuro[3,4-d]pyrimidin-7-yl)-2-methylpropan-2-ol. (Diastereomer 1): LC-MS: m/z=157 (M+3H). ¹H NMR (400 MHz, DMSO) δ 8.69 (s, 1H), 8.20 (d, J=8.7, 2H), 7.48 (d, J=8.8, 2H), 6.16 (t, J=5.5, 1H), 5.19 (d, J=11.8, 1H), 5.10 (d, J=11.8, 1H), 4.28-3.90 (m, 4H), 3.76-3.61 (m, 2H), 3.56-3.45 (m, 1H), 3.39-3.24 (m, 1H), 3.17-3.06 (m, 2H), 2.08-1.93 (m, 2H), 1.40 (s, 3H), 1.24 (d, J=6.7, 3H), 1.13-1.02 (m, 6H), 0.98 (s, 3H). (Diastereomer 2): LC-MS: m/z=157 (M+3H). ¹H NMR (400 MHz, DMSO) δ 8.69 (s, 1H), 8.20 (d, J=8.8, 2H), 7.48 (d, J=8.8, 2H), 6.16 (t, J=5.6, 1H), 5.22-5.07 (m, 2H), 4.39-4.13 (m, 2H), 4.11-3.88 (m, 2H), 3.76-3.62 (m, 2H), 3.57-3.46 (m, 1H), 3.40-3.26 (m, 1H), 3.17-3.07 (m, 2H), 2.04-1.93 (m, 2H), 1.41 (s, 3H), 1.24 (d, J=6.7, 3H), 1.11 (s, 3H), 1.06 (t, J=7.2, 3H), 0.99 (s, 3H).

### Example 128

### Preparation of (S)-5-(4-(3-ethylmorpholino)-6,7-dihydro-5H-pyrano[2,3-d]pyrimidin-2-yl)pyrimidin-2-amine (if)

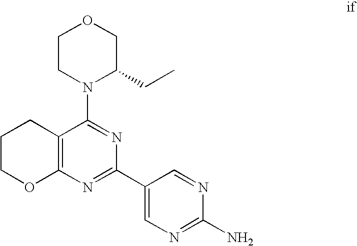

(S)-5-(4-(3-ethylmorpholino)-6,7-dihydro-5H-pyrano[2,3-d]pyrimidin-2-yl)pyrimidin-2-amine (if) was prepared in a similar manner as described for Example 1 with the exceptions that tetrahydro-2H-pyran-2-one was used in Step 1 instead of dihydro-2H-pyran-3(4H)-one, (S)-3-ethylmorpholine was used in Step 5 instead of morpholine and 5-(4,4,5,5-tetramethyl-1,3,2-dioxaborolan-2-yl)pyrimidin-2-amine was used in step 6 instead of 1-ethyl-3-(4-(4,4,5,5-tetramethyl-1,3,2-dioxaborolan-2-yl)phenyl)urea. LC-MS: m/z=343 (M+H). ¹H NMR (400 MHz, DMSO) δ 8.94 (s, 2H), 7.03 (s, 2H), 4.53-4.01 (m, 2H), 3.66 (ddd, J=41.0, 33.7, 11.9 Hz, 6H), 2.63 (dd, J=20.3, 12.6 Hz, 1H), 2.01-1.60 (m, 4H), 0.84 (t, J=7.3 Hz, 3H).

### Example 129

### Preparation of (S)-5-(4-(3-ethylmorpholino)-6,7-dihydro-5H-pyrano[2,3-d]pyrimidin-2-yl)pyridin-2-amine (ig)

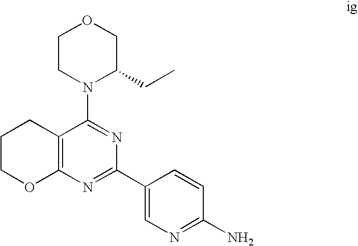

(S)-5-(4-(3-ethylmorpholino)-6,7-dihydro-5H-pyrano[2,3-d]pyrimidin-2-yl)pyridin-2-amine (ig) was prepared in a similar manner as described for Example 1 with the exceptions that tetrahydro-2H-pyran-2-one was used in Step 1 instead of dihydro-2H-pyran-3(4H)-one, (S)-3-ethylmorpholine was used in Step 5 instead of morpholine and 5-(4,4,5,5-tetramethyl-1,3,2-dioxaborolan-2-yl)pyridin-2-amine was used in step 6 instead of 1-ethyl-3-(4-(4,4,5,5-tetramethyl-1,3,2-dioxaborolan-2-yl)phenyl)urea. LC-MS: m/z=342 (M+H). ¹H NMR (400 MHz, DMSO) δ 8.94 (s, 2H), 7.06 (s, 2H), 4.26 (dd, J=27.1, 17.8 Hz, 2H), 3.97-3.45 (m, 6H), 2.72-2.56 (m, 1H), 2.01-1.60 (m, 4H), 0.84 (t, J=7.3 Hz, 3H).

### Example 130

### 5-(4-((1R,5S)-8-oxa-3-azabicyclo [3.2.1]octan-3-yl)-6,7-dihydro-5H-pyrano[2,3-d]pyrimidin-2-yl)pyridin-2-amine (ih)

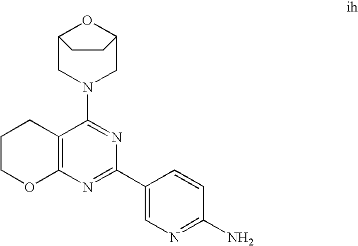

5-(4-((1R,5S)-8-oxa-3-azabicyclo[3.2.1]octan-3-yl)-6,7-dihydro-5H-pyrano[2,3-d]pyrimidin-2-yl)pyridin-2-amine (ih) was prepared in a similar manner as described for Example 1 with the exceptions that tetrahydro-2H-pyran-2-one was used in Step 1 instead of dihydro-2H-pyran-3(4H)-one, (1R,5S)-8-oxa-3-azabicyclo[3.2.1]octane was used in Step 5 instead of morpholine and 5-(4,4,5,5-tetramethyl-1,3,2-dioxaborolan-2-yl)pyridin-2-amine was used in step 6 instead of 1-ethyl-3-(4-(4,4,5,5-tetramethyl-1,3,2-dioxaborolan-2-yl)phenyl)urea. LC-MS: m/z=340 (M+H). ¹H NMR (400 MHz, DMSO) δ 8.77 (d, J=1.9 Hz, 1H), 8.11 (dd, J=8.7, 2.3 Hz, 1H), 6.46 (d, J=8.7 Hz, 1H), 6.30 (s, 2H), 4.41 (s, 2H), 4.37-4.17 (m, 2H), 3.75 (d, J=10.7 Hz, 2H), 3.59 (d, J=10.6 Hz, 2H), 2.60 (t, J=5.9 Hz, 2H), 2.04-1.73 (m, 7H).

### Example 131

### 5-(4-((1R,5S)-8-oxa-3-azabicyclo [3.2.1]octan-3-yl)-6,7-dihydro-5H-pyrano[2,3-d]pyrimidin-2-yl)pyrimidin-2-amine (ii)

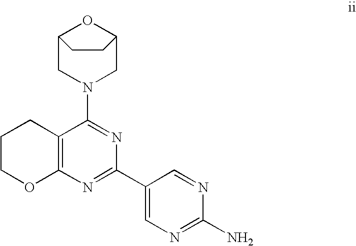

5-(4-((1R,5S)-8-oxa-3-azabicyclo[3.2.1]octan-3-yl)-6,7-dihydro-5H-pyrano[2,3-d]pyrimidin-2-yl)pyrimidin-2-amine (ii) was prepared in a similar manner as described for Example 1 with the exceptions that tetrahydro-2H-pyran-2-one was used in Step 1 instead of dihydro-2H-pyran-3(4H)-one, (1R,5S)-8-oxa-3-azabicyclo[3.2.1]octane was used in Step 5 instead of morpholine and 5-(4,4,5,5-tetramethyl-1,3,2-dioxaborolan-2-yl)pyrimidin-2-amine was used in step 6 instead of 1-ethyl-3-(4-(4,4,5,5-tetramethyl-1,3,2-dioxaborolan-2-yl)phenyl)urea. LC-MS: m/z=341 (M+H). ¹H NMR (400 MHz, DMSO) δ 8.94 (s, 1H), 7.03 (s, 1H), 4.44 (s, 1H), 4.36-4.20 (m, 1H), 3.75 (d, J=10.7 Hz, 1H), 3.59 (d, J=10.5 Hz, 1H), 2.62 (dd, J=19.6, 13.6 Hz, 1H), 1.91 (dd, J=23.1, 7.9 Hz, 3H).

### Example 132

### 5-(4-((1R,5S)-3-oxa-8-azabicyclo[3.2.1]octan-8-yl)-6,7-dihydro-5H-pyrano[2,3-d]pyrimidin-2-yl)pyridin-2-amine (ij)

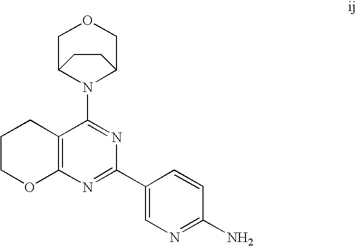

5-(4-((1R,5S)-3-oxa-8-azabicyclo[3.2.1]octan-8-yl)-6,7-dihydro-5H-pyrano[2,3-d]pyrimidin-2-yl)pyridin-2-amine (ij) was prepared in a similar manner as described for Example 1 with the exceptions that tetrahydro-2H-pyran-2-one was used in Step 1 instead of dihydro-2H-pyran-3(4H)-one, (1R,5S)-3-oxa-8-azabicyclo[3.2.1]octane was used in Step 5 instead of morpholine and 5-(4,4,5,5-tetramethyl-1,3,2-dioxaborolan-2-yl)pyridin-2-amine was used in step 6 instead of 1-ethyl-3-(4-(4,4,5,5-tetramethyl-1,3,2-dioxaborolan-2-yl)phenyl)urea. LC-MS: m/z=340 (M+H). ¹H NMR (400 MHz, DMSO) δ 8.78 (d, J=1.8 Hz, 1H), 8.12 (dd, J=8.7, 2.2 Hz, 1H), 6.46 (d, J=8.7 Hz, 1H), 6.30 (s, 2H), 4.37 (s, 2H), 4.32-4.16 (m, 2H), 3.69 (d, J=12.5 Hz, 2H), 3.15 (d, J=12.2 Hz, 3H), 2.56 (dd, J=12.6, 6.4 Hz, 2H), 1.85 (ddd, J=16.6, 10.2, 5.4 Hz, 7H).

### Example 133

### Preparation of 5-(4-((1R,5S)-3-oxa-8-azabicyclo[3.2.1]octan-8-yl)-6,7-dihydro-5H-pyrano[2,3-d]pyrimidin-2-yl)pyrimidin-2-amine (ik)

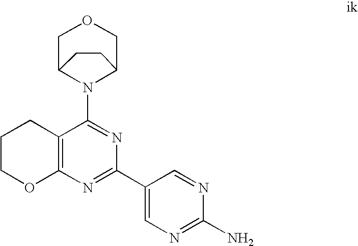

5-(4-((1R,5S)-3-oxa-8-azabicyclo[3.2.1]octan-8-yl)-6,7-dihydro-5H-pyrano[2,3-d]pyrimidin-2-yl)pyrimidin-2-amine (ik) was prepared in a similar manner as described for Example 1 with the exceptions that tetrahydro-2H-pyran-2-one was used in Step 1 instead of dihydro-2H-pyran-3(4H)-one, (1R,5S)-3-oxa-8-azabicyclo[3.2.1]octane was used in Step 5 instead of morpholine and 5-(4,4,5,5-tetramethyl-1,3,2-dioxaborolan-2-yl)pyrimidin-2-amine was used in step 6 instead of 1-ethyl-3-(4-(4,4,5,5-tetramethyl-1,3,2-dioxaborolan-2-yl)phenyl)urea. LC-MS: m/z=341 (M+H). ¹H NMR (400 MHz, DMSO) δ 8.95 (s, 1H), 7.04 (s, 1H), 4.30 (dd, J=22.0, 17.2 Hz, 2H), 3.72 (d, J=12.6 Hz, 1H), 3.16 (d, J=12.2 Hz, 1H), 2.64-2.54 (m, 1H), 1.95-1.71 (m, 3H).

### Example 134

Biological Evaluation of Compounds:

a. In Vitro mTOR Kinase Assay

The kinase activity of mTOR enzyme is assessed by incubating purified recombinant enzyme (mTOR(1360-2549)+GBL, prepared in-house) in a reaction mixture containing ATP, MnCl₂, and a fluorescently labeled mTOR substrate, e.g., GFP-4E-BP1 (Invitrogen, product #PR8808A). The reaction is stopped by an addition of a Terbium-labeled phospho-specific antibody, e.g., Tb-labeled anti-p4E-BP1 T37/T46, (Invitrogen, product #PR8835A), EDTA, and TR-FRET buffer solution (Invitrogen, Product #PR3756B). Product formation is detected by way of time-resolved fluorescence resonance energy transfer (TR-FRET), which occurs when the phosphorylated substrate and labeled antibody are in close proximity due to phospho-specific binding. Enzymatic activity is measured as an increase in TR-FRET signal using a Perkin Elmer Envision plate reader. The assay is performed in a 384-well Proxiplate Plus (Perkin Elmer. Product #6008269) using the following protocol:

Compound activity is tested in 10 point dose curves starting at the highest final concentration of 10 uM. They are serially diluted in 100% DMSO prior to further dilution with assay buffer. The reaction mixture (8 uls) containing 0.25 nM mTOR+GBL enzyme, 400 nM GFP-4E-BP1, 8 uM ATP, 50 mM Hepes pH 7.5, 0.01% Tween 20, 10 mM MnCl₂, 1 mM EGTA, 1 mM DTT, 1% DMSO (+/−compound) is incubated at room temperature for 30 minutes. 8 μL of solution containing 2 nM Tb-anti-p4E-BP1 antibody & 10 mM EDTA diluted TR-FRET buffer is then added and incubated for 30 minutes to stop the reaction. The plate is scanned with the Envision plate reader. Ki values are calculated in Assay Explorer using the Morrison ATP-competitive tight binding equation for Ki apparent determination.

Compounds of the invention (e.g., compounds of Formula I have an activity level (Ki) in the mTOR kinase assay of between about 0.0001 nM and about 5 uM, and in certain embodiments between about 0.0001 nM and about 1 uM, and in certain other embodiments less than between about 0.0001 nM and about 0.5 uM. In the order as each compound appears in Table 1, the compounds listed in Table I have an activity level as follows (in uM): 0.010, 0.002, 0.004, 0.025, 0.001, 0.002, 0.003, 0.004, 0.016, 0.003, 0.013, 0.014, 0.026, 0.001, 0.002, 0.005, 0.001, 0.002, 0.004, 0.001, 0.001, 0.022, 0.001, 0.002, 0.102, 0.343, 0.272, 0.270, 0.001, 4.326, 0.183, 0.002, 1.184, 0.006, 0.001, 0.002, 0.003, 0.003, 0.001, 0.006, 0.002, 0.002, 0.620, 0.884, 0.392, 0.0003, 0.001, 0.001, 0.001, 0.001, 0.001, 0.001, 0.0002, 0.001, 0.005, 0.007, 0.046, 0.003, 0.002, 0.003, 0.001, 0.004, 0.0004, 0.002, 0.003, 0.003, 0.010, 0.002, 0.016, 0.006, 0.002, 0.005, 0.001, 0.004, 0.004, 0.001, 0.010, 0.002, 0.002, 0.001, 0.020, 0.007, 0.003, 0.002, 0.002, 0.002, 2.5 or 1.8, 1.8 or 2.5, 0.027, 0.002, 0.008 or 0.002, 0.002 or 0.008, 0.006 or 0.001, 0.001 or 0.006, 0.002, 0.002, 0.293, 0.015, 0.007, 0.067, 0.057, 0.031, 0.097, 0.032, 0.106, 0.030, 0.076, 0.064 or 0.016, 0.016 or 0.064, 0.393, 0.258, 0.065 or 0.015, 0.015 or 0.065, 0.002, 0.004, 0.017, 0.004, 0.002, 0.221, 0.096, 0.101, 0.030, 0.255, 0.410, 0.003 or 0.0003, 0.0003 or 0.003, 0.047, 0.021, 0.001, 0.001, 0.015, 0.009, 0.002, 0.231, 0.297, 0.227, 0.052, 0.124, 0.269, 0.012, 0.017, 0.567, 0.004, 4.3, 1.5, 0.007 or 0.001, 0.001 or 0.007, 0.0005 or 0.001, 0.001 or 0.0005, 0.0007, 0.0007, 0.024, 0.39, 0.007, 0.001 or 0.0007, 0.0007 or 0.001, 0.088, 0.010, 0.010, 0.043, 0.024, 0.081, 0.122, 0.103, 0.011, 0.010, 0.007, 0.003, 0.002, 0.071, 0.005, 0.002, 0.009, 0.021 or 0.004, 0.004 or 0.021, 0.010, 0.008 and 0.006.

In the above assay data, for separated diasterometric compounds in which the absolute stereochemistry has not been assigned, two alternative assay data points are provided for each compound which corresponds to the assay data points of the separated diastereomers.

b. In Vitro Phospho-AKT Serine 473 Cellular Assay

The assay measures a test compound's inhibition of AKT serine-473 phosphorylation in human prostate adenocarcinoma derived PC-3 (ATCC CRL-1435) cells that have been stimulated with epidermal growth factor (EGF).

The PC-3 cell line is maintained in RPMI1640 media supplemented with 10% FBS, 2 mM Glutamine, and 10 mM HEPES pH 7.4 at 37° C. in a 5% CO₂ humidified incubator.

Cells are seeded in 384-well plates at 7,000 cells/well in 50 μl growth media. After 24 hours, growth media is removed and replaced with RPMI1640 containing no FBS. Cells are treated with 10 concentrations of test compounds or DMSO alone for controls (final DMSO concentration 0.5%) and incubated at 37° C. for 30 minutes. Cells are then stimulated for 10 minutes with 100 ng/ml EGF (final concentration). One column of controls is not stimulated with EGF to observe the signal ratio between stimulated and non-stimulated cells. After 10 minutes, compounds and stimulation media are removed and replaced with 25 μl lysis buffer containing protease inhibitors and phosphatase inhibitors. This buffer contains detergent to bring about cellular disruption. Following complete cellular disruption, 20 μl lysate is transferred to a MesoScale Discovery 384 well 4-spot plate coated with an antibody to AKT (MesoScale Discovery (MSD) product K211CAD-2) which have been previously blocked with 3% bovine serum albumin in Tris buffered saline. Following the transfer of lysate to the MSD plate, AKT in the lysate is captured on the coated antibody by incubation on a shaker at 4° C. for 16 hours. Following the capture step the plate is washed and then incubated for two hours with an antibody to S473 phosphorylated AKT which is conjugated with a Sulfo-Tag. This tag gives a signal when in proximity to the electrode on the base of the MSD plate. Binding the tagged antibody to the captured protein allow detection on a MSD reader.

The EC₅₀ is defined as the concentration at which a given compound achieves 50% decrease of the measured levels of S473 AKT phosphorylation. EC₅₀ values are calculated using MDL Assay Explorer 3.0.1.8 fitting a sigmoidal curve with a variable slope.

The first nine specific compounds described herein have an EC50 activity level of (in uM): 0.085, 0.010, 0.022, 0.237, 0.006, 0.015, 0.108, 0.042 and 0.049.

c. In Vitro Cell Proliferation Assay

Efficacy of Formula I compounds were measured by a cell proliferation assay employing the following protocol:

1. An aliquot of 20 μl of cell culture containing about 10³ cells (PC3 or MDAMB361.1) in medium was deposited in each well of a 384-well, opaque-walled plate.

2. Control wells were prepared containing medium and without cells; Cells were allowed to settle overnight.

3. The compound was added to the experimental wells and incubated for 3 days.

4. The plates were equilibrated to room temperature for approximately 30 minutes.

5. A volume of CellTiter-Glo Reagent equal to the volume of cell culture medium present in each well was added.

6. The contents were mixed for 2 minutes on an orbital shaker to induce cell lysis.

7. The plate was incubated at room temperature for 20 minutes to stabilize the luminescence signal.

8. Luminescence was recorded and reported in graphs as RLU=relative luminescence units.

Alternatively, cells were seeded at optimal density in a 96 well plate and incubated for 4 days in the presence of test compound. Alamar Blue™ was subsequently added to the assay medium, and cells were incubated for 6 h before reading at 544 nm excitation, 590 nm emission. EC₅₀ values were calculated using a sigmoidal dose response curve fit. In the order as each compound appears in Table 1, the compounds listed in Table I have an EC50 value of (in uM, with PC3 cells): 0.194, 0.231, 0.175, 1.05, 0.088, 0.094, 0.189, 0.059, 0.993, 0.541, 1.5, 1.6, 0.313, 0.144, 0.194, 0.341, 0.172, 0.057, 0.325, 0.111, 0.077, 1.2, 0.045, 0.236, na, na, na, na, 0.116, na, na, 0.194, na, 0.342, 0.030, 0.297, 0.18, 0.084, 0.056, 0.392, 0.329, 0.102, na, na, na, 0.067, 0.029, 0.077, 0.172, 0.051, 0.233, 0.040, 0.015, 0.037, 0.595, 0.173, 4.0, 0.162, 0.046, 0.124, 0.108, 0.327, 0.019, 0.099, 0.122, 0.804, 0.853, 1.2, 0.585, 0.475, 0.036, 0.238, 0.013, 2.8, 0.188, 0.015, 1.2, 0.139, 0.479, 0.173, 1.6, 0.445, 0.050, 0.066, 0.045, 0.061, na, na, 0.914, 0.084, 0.188, 0.284, 0.062, 0.322, 0.025, 0.039, 0.045, na, 0.402, 0.274, na, 0.774, na, 0.585, na, 0.743, na, na, 0.238, na, na, na, 0.297, 0.0376, 0.0961, 0.159, 0.092, 0.035, na, na, na, na, 0.671, na, na, 0.209, 0.161, 1.3, 0.884, 0.735, 0.041, 0.454, 0.316, 0.145, na, na, na, 2.5, 0.146, na, 2.1, 1.2, na, 0.203, na, na, 0.453, 0.095, 0.153, 0.051, 0.019, 0.039, 1.3, na, 0.341, 0.066, 0.017, na, 0.231, 0.302, 0.577, 0.621, na, 1.2, na, 0.562, 0.401, 0.596, 0.132, 0.018, na, 0.142, 0.028, 0.374, 0.936, 0.218, 0.331 and 0.154.

“na” means the data is not available.

What is claimed is:

1. A compound of Formula I 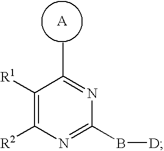
or a pharmaceutically acceptable salt thereof, wherein in Formula I,
A is a 5- to 8-membered heterocyclic ring having from 1 to 3 heteroatoms independently selected from N, O and S as ring vertices, and having from 0 to 2 double bonds; wherein the A ring is further substituted with from 0 to 5 R^{A} substituents selected from the group consisting of —C(O)OR^{a}, —C(O)NR^{a}R^{b}, —NR^{a}R^{b}, —OC(O)R^{c}, —OR^{a}, —SR^{a}, —S(O)₂R^{c}, —S(O)R^{c}, —R^{c}, —(CH₂)₁₋₄—NR^{a}R^{b}, —(CH₂)₁₋₄—NR^{a}C(O)R^{c}, —(CH₂)₁₋₄—OR^{a}, —(CH₂)₁₄—SR^{a}, —(CH₂)₁₋₄—S(O)₂R^{c}, —(CH₂)₁₋₄—S(O)R^{c}, halogen, —NO₂, —CN and —N₃, wherein R^{a} and R^{b} are each independently selected from hydrogen, C₁₋₆ alkyl, C₁₋₆ haloalkyl, C₁₋₆ heteroalkyl, C₂₋₆ alkenyl, C₂₋₆ alkynyl, C₃₋₆ cycloalkyl, phenyl and —(CH₂)₁₋₄(phenyl), and optionally R^{a} and R^{b}, together with the nitrogen atom to which each is attached, are combined to form a 3- to 7-membered heterocyclic ring comprising 1 to 2 heteroatoms selected from N, O and S; R^{c} is selected from C₁₋₆ alkyl, C₁₋₆ haloalkyl, C₁₋₆ heteroalkyl, C₂₋₆ alkenyl, C₂₋₆ alkynyl, C₃₋₆ cycloalkyl, phenyl and —(CH₂)₁₋₄ (phenyl); and any two substituents attached to the same atom in the 5- to 8-membered heterocyclic ring are optionally combined to form a 3- to 5-membered carbocyclic or a 3 to 5-membered heterocyclic ring;
R¹ and R² are combined with the atoms to which they are attached to form a 5- to 8-membered monocyclic or bridged bicyclic heterocyclic ring comprising —O— as one of the ring vertices;
wherein the 5- to 8-membered monocyclic or bridged-bicyclic heterocyclic ring formed by combining R¹ and R² further optionally comprises one additional heteroatom selected from the group consisting of N, O and S, and is substituted with from 0 to 5 R^{R} substituents selected from the group consisting of halogen, —NR^{j}R^{k}, —SR^{j}, —OR^{j}, —C(O)OR^{j}, —C(O)NR^{j}R^{k}, —NHC(O)R^{j}, —OC(O)R^{j}, —R^{m}, —CN, ═O, ═S, ═N—CN, —(CH₂)₁₋₄—CN, —(CH₂)₁₋₄—OR^{j}, —(CH₂)₁₋₄—NR^{j}R^{k}, —C₁₋₄ alkylene-OR^{j}, —C₁₋₄ alkylene-R^{m}, —C₂₋₄ alkenylene-R^{m}, —C₂₋₄-alkynylene-R^{m}, —C₁₋₄ alkylene-C₁₋₉ heteroaryl, C₂₋₄ alkenylene-C₁₋₉ heteroaryl, C₂₋₄ alkynylene-C₁₋₉ heteroaryl, C₁₋₄ alkylene-C₆₋₁₀ aryl, C₂₋₄ alkynylene-C₆₋₁₀ aryl and C₂₋₄ alkynylene-C₆₋₁₀ aryl, wherein R^{j} and R^{k} are each independently selected from hydrogen, C₁₋₆ alkyl, C₁₋₆ haloalkyl, C₁₋₆ heteroalkyl, C₂₋₆ alkenyl, C₂₋₆ alkynyl, C₃₋₇ cycloalkyl, C₂₋₆ heterocycloalkyl, phenyl, pyridyl and —(CH₂)₁₋₄-(Ph), and R^{j} and R^{k}, when attached to the same nitrogen atom, are optionally combined to form a 3- to 6-membered heterocyclic ring comprising 1 to 2 heteroatoms selected from N, O and S; and R^{m} is selected from C₁₋₆ alkyl, C₁₋₆ haloalkyl, C₁₋₆ heteroalkyl, C₂₋₆ alkenyl, C₂₋₆ alkynyl, C₃₋₇ cycloalkyl, C₂₋₆ heterocycloalkyl and —(CH₂)₁₋₄-(Ph), and wherein a C₃₋₇ cycloalkyl, C₂₋₆ heterocycloalkyl, C₁₋₉ heteroaryl or C₆₋₁₀ aryl portion of a R^{R} substituent is substituted with from 0 to 3 substituents selected from the group consisting of F, Cl, Br, I, —NH(C₁₋₄ alkyl), —N(diC₁₋₄ alkyl), O(C₁₋₄ alkyl), C₁₋₆ alkyl, C₁₋₆ heteroalkyl, —C(O)O(C₁₋₄ alkyl), —C(O)NH(C₁₋₄alkyl), —C(O)N(diC₁₋₄ alkyl), —NO₂, —CN; wherein when R¹ and R² are combined to form a monocyclic 5- to 8-membered heterocyclic ring then any two R^{R} substitutents attached to the same atom or adjacent carbon atoms in said 5- to 8-membered heterocyclic ring are optionally combined to form a 3- to 7-membered cycloalkyl ring or a 3- to 7-membered heterocycloalkyl ring comprising 1 to 2 heteroatoms selected from N, O and S as ring vertices;
B is a member selected from the group consisting of phenylene and 5- to 6-membered heteroarylene, and is substituted with from 0 to 4 R^{B} substituents selected from halogen, —CN, —N₃, —NO₂, —C(O)ORⁿ, —C(O)NRⁿR^{o}, —NRⁿC(O)R^{o}, —NRⁿC(O)NRⁿR^{o}, —NRⁿR^{o}, —(CH₂)₁₋₄—C(O)Or, —(CH₂)₁₋₄—C(O)NRⁿR^{o}, —(CH₂)₁₋₄—ORⁿ, —(CH₂)₁₋₄—NRⁿR^{o}, —(CH₂)₁₋₄—SR^{p} and R^{p}; wherein Rⁿ and R^{o} are independently selected from hydrogen and C₁₋₆ alkyl, C₁₋₆ haloalkyl, C₁₋₆ heteroalkyl, C₂₋₆ alkenyl, C₂₋₆ alkynyl, C₃₋₇ cycloalkyl, C₂₋₆ heterocycloalkyl, phenyl and —(CH₂)₁₋₄-(phenyl) or when attached to the same nitrogen atom, Rⁿ and R^{o} are optionally are combined to form a 3- to 6-membered heterocyclic ring comprising 1 to 2 heteroatoms selected from N, O and S; R^{p} is C₁₋₆ alkyl, C₁₋₆ haloalkyl, C₁₋₆ heteroalkyl, C₂₋₆ alkenyl, C₂₋₆ alkynyl, C₃₋₇ cycloalkyl, C₂₋₆ heterocycloalkyl, phenyl and —(CH₂)₁₋₄-(phenyl), wherein any two substituents, not including the D group, located on adjacent atoms of B are optionally combined to form a 5- to 6-membered carbocyclic, heterocyclic, aryl or heteroaryl ring;
D is a member selected from the group consisting of —NR³C(O)NR⁴R⁵, —NR⁴R⁵, —C(O)NR⁴R⁵, —OC(O)OR⁴, —OC(O)NR⁴R⁵, —NR³C(═N—CN)NR⁴R⁵, —NR³C(═N—OR⁴)NR⁴R⁵, —NR³C(═N—NR⁴)NR⁴R⁵, —NR³C(O)R⁴, —NR³C(O)OR⁴, —NR³S(O)₂NR⁴R⁵, —NR³S(O)₂R⁴, —NR³C(═S)NR⁴R⁵ and —S(O)₂R⁴R⁵, wherein R³ is selected from the group consisting of hydrogen, C₁₋₆ alkyl, C₁₋₆ haloalkyl and C₂₋₆ alkenyl; R⁴ and R⁵ are each independently selected from the group consisting of hydrogen, C₁₋₆ alkyl, C₁₋₆ haloalkyl, C₁₋₆ alkylamino-C(═O)—, C₂₋₆ alkenyl, C₂₋₆ alkynyl, C₃₋₁₀ cycloalkyl, C₂₋₉ heterocycloalkyl, C₆₋₁₀ aryl and C₁₋₉ heteroaryl, and R⁴ and R⁵, when attached to the same nitrogen atom, are optionally combined to form a 5- to 7-membered heterocyclic or 5- to 6-membered heteroaryl ring comprising 1 to 3 heteroatoms selected from N, O and S; and wherein R³, R⁴ and R⁵ are further substituted with from 0 to 3 R^{D} substituents independently selected from the group consisting of halogen, —NO₂, —CN, —NR^{q}R^{r}, —OR^{q}, —SR^{q}, —C(O)OR^{q}, —C(O)NR^{q}R^{r}, —NR^{q}C(O)R^{r}, —NR^{q}C(O)OR^{s}, —(CH₂)₁₋₄—NR^{q}R^{r}, —(CH₂)₁₋₄—OR^{q}, —(CH₂)₁₋₄—SR^{q}, —(CH₂)₁₋₄—C(O)OR^{q}, —(CH₂)₁₋₄—C(O)NR^{q}R^{r}, —(CH₂)₁₋₄—NR^{q}C(O)R^{r}, —(CH₂)₁₋₄—NR^{q}C(O)OR^{r}, —(CH₂)₁₋₄—CN, —(CH₂)₁₋₄—NO₂, —S(O)R^{r}, —S(O)₂R^{r}, —(CH₂)₁₋₄R^{s}, ═O, and —R^{s}; wherein R^{q} and R^{r} is selected from hydrogen, C₁₋₆ alkyl, C₁₋₆ haloalkyl, C₂₋₆ alkenyl, C₂₋₆ alkynyl, C₁₋₆ heteroalkyl, C₃₋₇ cycloalkyl, C₂₋₆ heterocycloalkyl, C₆₋₁₀ aryl, C₁₋₉ heteroaryl; and R^{s}, at each occurrence, is independently selected from C₁₋₆ alkyl, C₁₋₆ haloalkyl, C₃₋₇ cycloalkyl, C₂₋₆ heterocycloalkyl, C₆₋₁₀ aryl and C₁₋₉ heteroaryl; and wherein the D group and a substituent located on an adjacent atom of the B ring are optionally combined to form a 5- to 6-membered heterocyclic or heteroaryl ring optionally substituted with 1 to 2 R^{D} substituents.

2. The compound of claim 1, wherein R¹ and R² are combined to form a 5- to 8-membered heterocyclic ring comprising —O— as the only heteroatom in the 5- to 8-membered heterocyclic ring.

3. The compound of claim 1, wherein in Formula I the A ring comprises from 0 to 1 double bond.

4. The compound of claim 1, wherein A is a 5- to 8-membered monocyclic or bicyclic-bridged heterocyclic ring and is further substituted with from 0 to 3 R^{A} substituents selected from the group consisting of —C(O)OR^{a}, —C(O)NR^{a}R^{b}, —NR^{a}R^{b}, —OC(O)R^{c}, —OR^{a}, —SR^{a}, —S(O)₂R^{c}, —S(O)R^{c}, —R^{c}, —(CH₂)₁₋₄—NR^{a}R^{b}, —(CH₂)₁₋₄—OR^{a}, halogen, —NO₂, —CN and —N₃, wherein R^{a} and R^{b} are each independently selected from hydrogen, C₁₋₆ alkyl, C₁₋₆ haloalkyl, C₁₋₆ heteroalkyl and C₃₋₆ cycloalkyl, and optionally R^{a} and R^{b}, together with the nitrogen atom to which each is attached, are combined to form a 3- to 6-membered ring; R^{c} is selected from C₁₋₆ alkyl, C₁₋₆ haloalkyl, C₁₋₆ heteroalkyl, C₂₋₆ alkenyl, C₂₋₆ alkynyl, C₃₋₆ cycloalkyl, phenyl and —(CH₂)₁₋₄ (phenyl); and wherein any two substituents located on the same atom of the A ring are optionally combined to form a 3- to 5-membered cycloalkyl ring;
B is selected from the group consisting of 1,4-phenylene, 2,5-pyridylene and 3,6-pyridylene and is substituted with from 0 to 2 substituents selected from halogen, —CN, —N₃, —NO₂, —C(O)ORⁿ, —C(O)NRⁿR^{o}, —NRⁿC(O)R^{o}, —NRⁿC(O)NRⁿR^{o}, —ORⁿ, —NRⁿR^{o} and R^{p}; wherein Rⁿ and R^{o} are independently selected from hydrogen and C₁₋₆ alkyl, C₁₋₆ haloalkyl, C₁₋₆ heteroalkyl, C₃₋₇ cycloalkyl and C₂₋₆ heterocycloalkyl, or when attached to the same nitrogen atom, Rⁿ and R^{o} are optionally are combined to form a 3- to 6-membered ring; R^{p} is C₁₋₆ alkyl, C₁₋₆ haloalkyl, C₃₋₇ cycloalkyl and C₂₋₆ heterocycloalkyl;
D is a member selected from the group consisting of —NR³C(O)NR⁴R⁵, —NR⁴R⁵, —C(O)NR⁴R⁵, —OC(O)NR⁴R⁵, —NR³C(═N—CN)NR⁴R⁵, —NR³C(O)R⁴, —NR³C(O)OR⁴, —NR³S(O)₂NR⁴R⁵, —NR³S(O)₂R⁴, —NR³C(═S)NR⁴R⁵ and —S(O)₂R⁴R⁵ wherein R³ is selected from the group consisting of hydrogen, C₁₋₆ alkyl, C₁₋₆ haloalkyl and C₂₋₆ alkenyl; R⁴ and R⁵ are each independently selected from the group consisting of hydrogen, C₁₋₆ alkyl, C₁₋₆ haloalkyl, C₂₋₆ alkenyl, C₂₋₆ alkynyl, C₃₋₇ cycloalkyl, C₂₋₆ heterocycloalkyl, C₆₋₁₀ aryl and C₁₋₉ heteroaryl, and R⁴ and R⁵, when attached to the same nitrogen atom, are optionally combined to form a 5- to 7-membered heterocyclic or 5- to 6-membered heteroaryl ring; and wherein R³, R⁴ and R⁵ are further substituted with from 0 to 3 R^{D} substituents independently selected from the group consisting of halogen, —NO₂, —CN, —NR^{q}R^{r}, —OR^{q}, —SR^{q}, —C(O)OR^{q}, —C(O)NR^{q}R^{r}, —NR^{q}C(O)R^{r}, —NR^{q}C(O)OR^{s}, —(CH₂)₁₋₄—NR′V, —(CH₂)₁₋₄—OR^{q}, —(CH₂)₁₋₄—SR^{q}, —(CH₂)₁₋₄—C(O)OR^{q}, —(CH₂)₁₋₄—C(O)NR^{q}R^{r}, —(CH₂)₁₋₄—NR^{q}C(O)R^{r}, —(CH₂)₁₋₄—NR^{q}C(O)OR^{r}, —(CH₂)₁₋₄—CN, —(CH₂)₁₋₄—NO₂, —S(O)R^{r}, —S(O)₂R^{r}, ═O, and —R^{s}; wherein R^{q} and R^{r} is each independently selected from hydrogen, C₁₋₆ alkyl, C₁₋₆ haloalkyl, C₂₋₆ alkenyl, C₂₋₆ alkynyl, C₁₋₆ heteroalkyl, C₃₋₇ cycloalkyl, C₂₋₆ heterocycloalkyl, C₆₋₁₀ aryl, C₁₋₉ heteroaryl; and R^{s}, at each occurrence, is independently selected from C₁₋₄ alkyl, C₁₋₄ haloalkyl, C₃₋₇ cycloalkyl, C₂₋₆ heterocycloalkyl, C₆ aryl and C₁₋₅ heteroaryl; and wherein the D group and a substituent located on an adjacent atom of the B ring are optionally combined to form a 5- to 6-membered heterocyclic or heteroaryl ring.

5. The compound of claim 1, wherein said compound has the Formula II-A:

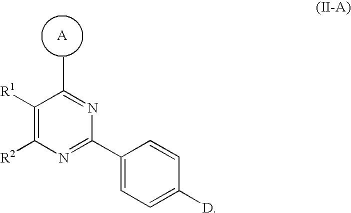

6. The compound of claim 1, wherein A is a 5- to 7-membered monocyclic or bicyclic bridged heterocyclic ring and is further substituted with from 0 to 3 R^{A} substituents selected from the group consisting of —C(O)OR^{a}, —C(O)NR^{a}R^{b}, —NR^{a}R^{b}, —OR^{a}, —SR^{a}, —S(O)₂R^{c}, —S(O)R^{c}, —R^{c}, halogen, —NO₂, —CN and —N₃, wherein R^{a} and R^{b} are each independently selected from hydrogen, C₁₋₄ alkyl, C₁₋₄ haloalkyl, C₁₋₄ heteroalkyl and C₃₋₆ cycloalkyl, and optionally R^{a} and R^{b}, together with the nitrogen atom to which each is attached, are combined to form a 3- to 6-membered ring; R^{c} is selected from C₁₋₄ alkyl, C₁₋₄ haloalkyl, C₁₋₄ heteroalkyl, C₂₋₆ alkenyl, C₂₋₆ alkynyl and C₃₋₆ cycloalkyl.

7. The compound of claim 6, wherein the A ring is a ring selected from the group consisting of morpholin-4-yl, 3,4-dihydro-2H-pyran-4-yl, 3,6-dihydro-2H-pyran-4-yl, tetrahydro-2H-pyran-4-yl, 1,4-oxazepan-4-yl, 2-oxa-5-azabicyclo[2.2.1]heptan-5-yl, piperazin-1-yl and piperidin-1-yl, and is substituted with from 0 to 2 R^{A} substituents selected from the group consisting of —C(O)OR^{a}, —C(O)NR^{a}R^{b}, —NR^{a}R^{b}, —OR^{a}, —SR^{a}, —S(O)₂R^{c}, —S(O)R^{c}, —R^{c}, halogen, —NO₂, —CN and —N₃, wherein R^{a} and R^{b} are each independently selected from hydrogen, C₁₋₄ alkyl, C₁₋₄ haloalkyl, C₁₋₄ heteroalkyl, C₂₋₆ alkenyl and C₃₋₆ cycloalkyl, wherein optionally R^{a} and R^{b}, together with the nitrogen atom to which each is attached, are combined to form a 3- to 6-membered heterocyclic ring, and R^{c} is selected from C₁₋₄ alkyl, C₁₋₄ haloalkyl, C₁₋₄ heteroalkyl, C₂₋₆ alkenyl, C₃₋₆ cycloalkyl.

8. The compound of claim 7, wherein the A ring is selected from the group consisting of morpholin-4-yl, 3-methyl-morpholin-4-yl, 3-ethyl-morpholin-4-yl, 3,4-dihydro-2H-pyran-4-yl, 3,6-dihydro-2H-pyran-4-yl, tetrahydro-2H-pyran-4-yl, 1,4-oxazepan-4-yl, 2-oxa-5-azabicyclo[2.2.1]heptan-5-yl and 4-methoxypiperidin-1-yl.

9. The compound of claim 1, wherein R¹ and R² are combined to form a 5- to 7-membered monocyclic heterocyclic ring, wherein the 5- to 7-membered ring is substituted with from 0 to 5 R^{R} substituents selected from the group consisting of halogen,—R^{m}, —C₁₋₄ alkylene-R^{m}, —C₂₋₄ alkenylene-R^{m}, —C₂₋₄ alkynylene-R^{m}, wherein R^{m} is selected from C₁₋₆ alkyl, C₁₋₆ haloalkyl, C₁₋₆ heteroalkyl, C₂₋₆ alkenyl, C₂₋₆ alkynyl, C₃₋₇ cycloalkyl, C₂₋₆ heterocycloalkyl and —(CH₂)₁₋₄-(Ph), and wherein halogen is selected from F, Cl and Br, wherein any two substituents attached to the same atom or to adjacent atoms in said 5- to 7-membered heterocyclic ring are optionally combined to form a 3- to 6-membered cycloalkyl or 3- to 6-membered heterocycloalkyl ring having 1 to 2 heteroatoms selected from N, O and S as ring vertices.

10. The compound of claim 9, wherein R^{m} is selected from C₁₋₆ alkyl and C₁₋₆ heteroalkyl, and any two R^{m} groups located on the same or adjacent atoms is optionally combined to form a 3- to 6-membered cycloalkyl ring or a 3- to 6-membered heterocycloalkyl ring having 1 to 2 heteroatoms selected from N, O and S as ring vertices.

11. The compound of claim 9, wherein the 5- to 7-membered heterocyclic ring formed by combining R¹ and R² comprises a carbon atom substituted with two R^{R} substituents independently selected from F, Cl, Br and R^{m} as a ring vertex.

12. The compound of claim 1, wherein in a compound of Formula I or Formula II-A, the ring formed by combining R¹ and R², as fused to the pyrimidine ring of Formula I, has a structure selected from the group consisting of ii-A, ii-B, ii-C, ii-D, ii-E, ii-F, ii-G, ii-H, ii-J, ii-K, ii-L, ii-M, ii-N, ii-O, ii-P, ii-Q, ii-R, ii-S, ii-T, ii-U, ii-V, ii-W, ii-X, ii-Y, ii-Z, ii-AA, ii-BB and ii-CC shown below:

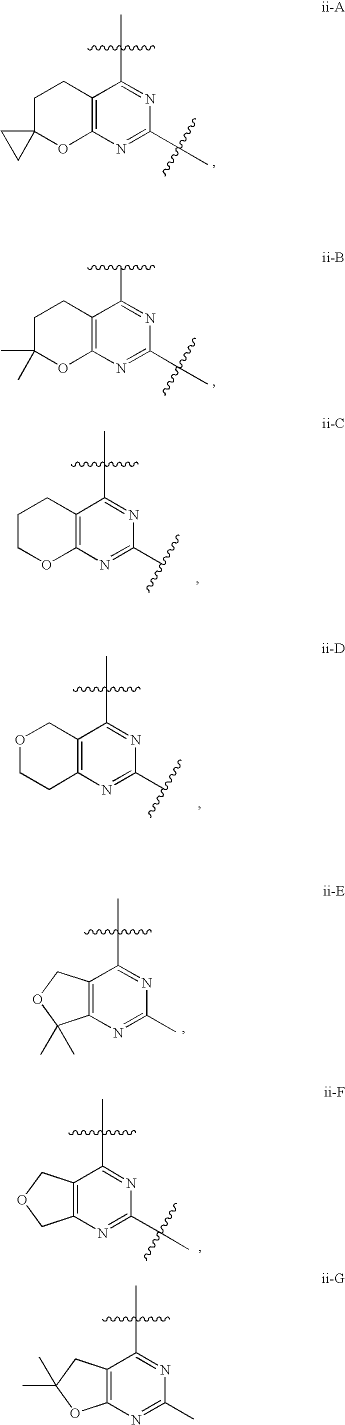

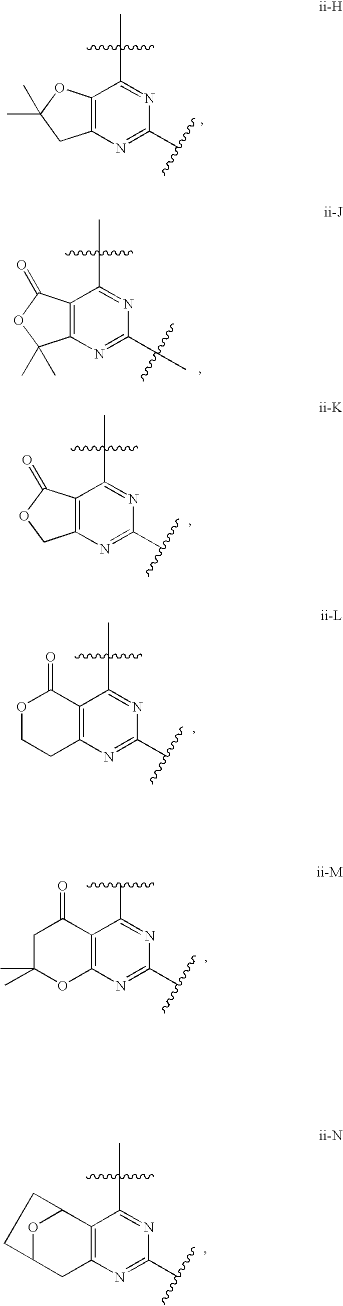

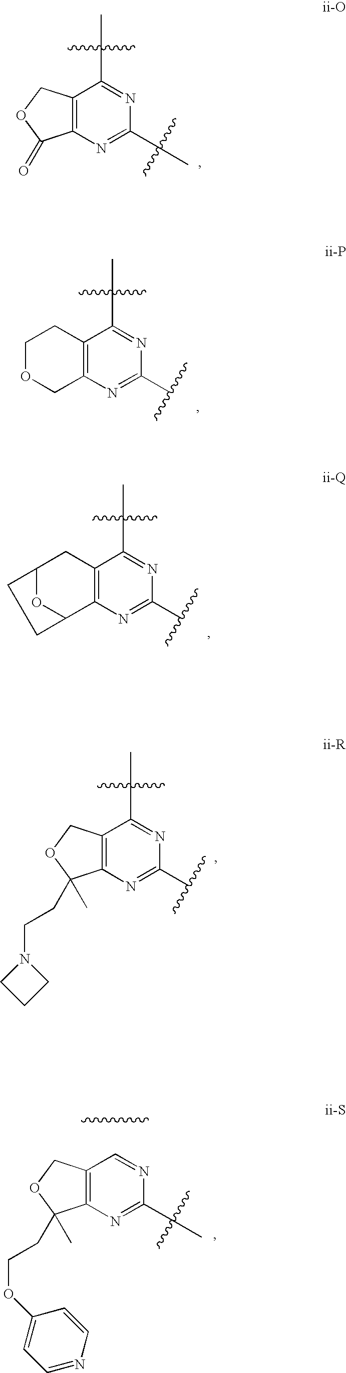

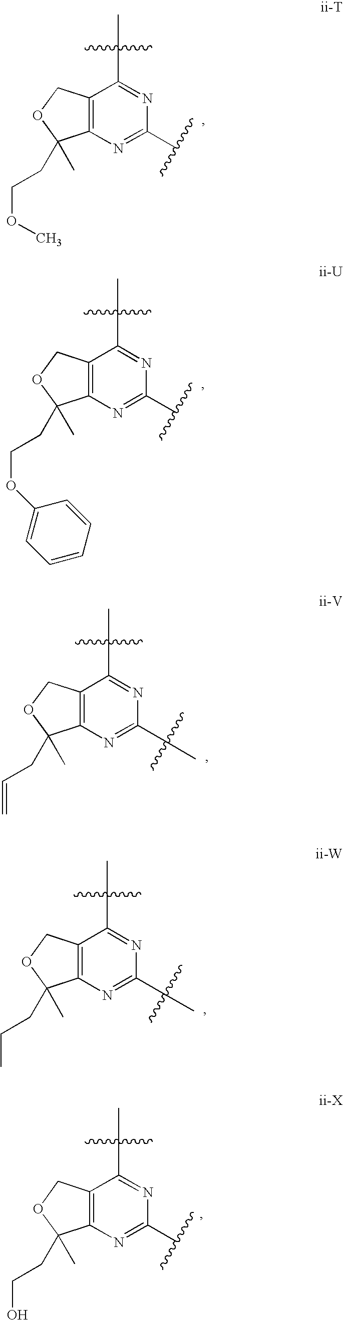

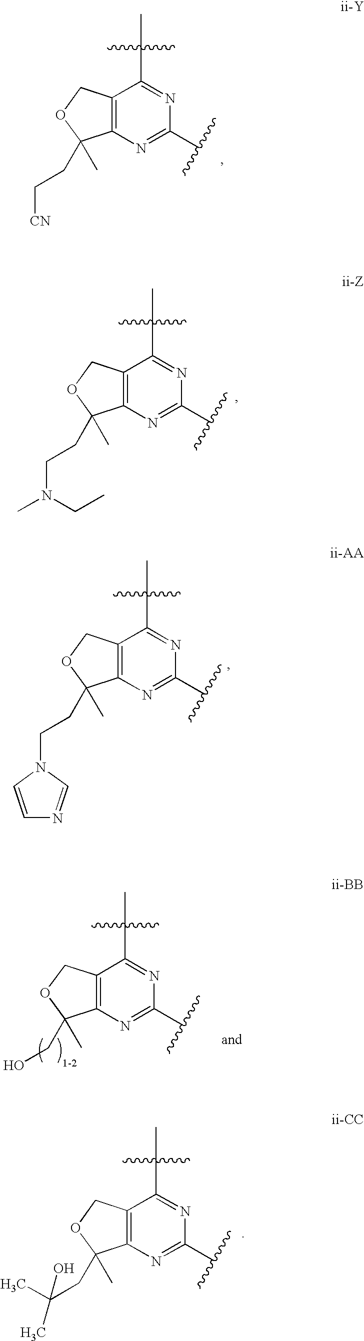

13. The compound of claim 1, wherein D is selected from the group consisting of —NR³C(O)NR⁴R⁵, —NR⁴R⁵, —C(O)NR⁴R⁵, —NR³C(═N—CN)NR⁴R⁵, —NR³C(O)R⁴, —NR³C(O)OR⁴, —NR³S(O)R⁴, —NR³C(═S)NR⁴R⁵ and —S(O)₂NR⁴R⁵.

14. The compound of claim 13, wherein D is selected from —NR³C(O)NR⁴R⁵ and —NR⁴R⁵, wherein R³ is hydrogen; R⁴ and R⁵ are each independently selected from the group consisting of hydrogen, C₁₋₆ alkyl, C₁₋₆ haloalkyl, C₃₋₇ cycloalkyl, C₂₋₆ heterocycloalkyl, C₆₋₁₀ aryl and C₁₋₉ heteroaryl, wherein R⁴ and R⁵ are each independently optionally substituted; and R⁴ and R⁵, when attached to the same nitrogen atom, are optionally combined to form a 5- to 7-membered heterocyclic or 5- to 10-membered heteroaryl ring comprising 1 to 3 heteroatoms selected from N, O and S as ring vertices.

15. The compound of claim 14, wherein D is —NR⁴R⁵, wherein R⁴ is hydrogen or C₁₋₃ alkyl, and R⁵ is selected from phenyl, C₁₋₅ heteroaryl, and C₂₋₆ heterocycloalkyl, wherein R⁵ is substituted with from 0 to 3 R^{D} substituents.

16. The compound of claim 15, R⁵ is selected from the group consisting of: 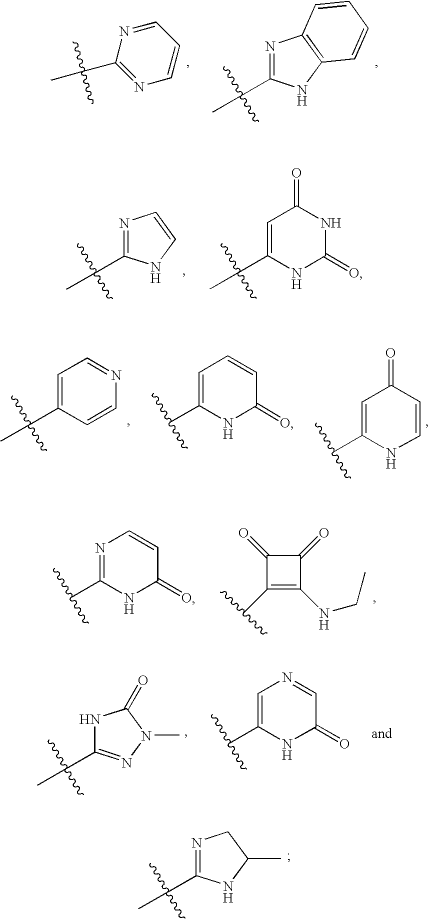
wherein from 0 to 3 hydrogen atoms attached to a carbon or nitrogen atom of R⁵ is optionally independently replaced with a R^{D} substitutents selected from the group consisting of halogen, F, Cl, Br, halogen, —NO₂, —CN, —NR^{q}R^{r}, —OR^{q}, —(CH₂)₁₋₄R^{s}, ═O, and —R^{s}; wherein R^{q} and R^{r} is selected from hydrogen, C₁₋₆ alkyl, C₁₋₆ haloalkyl, C₂₋₆ alkenyl, C₂₋₆ alkynyl, C₁₋₆ heteroalkyl; and R^{s}, at each occurrence, is independently selected from C₁₋₆ alkyl, C₁₋₆ haloalkyl, C₃₋₇ cycloalkyl and C₂₋₆ heterocycloalkyl.

17. The compound of claim 14, wherein D is —NR³C(O)NR⁴R⁵, wherein R³ is hydrogen; R⁴ and R⁵ are each independently selected from the group consisting of hydrogen, C₁₋₆ alkyl, C₁₋₆ heteroalkyl, C₃₋₇ cycloalkyl and C₂₋₆ heterocycloalkyl, wherein R⁴ and R⁵ at each occurrence are each independently optionally substituted.

18. The compound of claim 17, wherein R³ is hydrogen, R⁴ is hydrogen or C₁₋₃ alkyl, R⁵ is selected from the group consisting of methyl, ethyl, propyl, isopropyl, butyl, tert-butyl, isobutyl, cyclopropylmethyl, pentyl, hexyl, oxazolyl, isoxazolyl, pyrazolyl, pyrrolyl, furanyl, thiophenyl, tetrahydrofuranyl, tetrahydropyranyl, oxetanyl, oxadiazolyl, phenyl, pyridinyl, cyclobutyl, cyclopropyl, cyclopentyl, cyclohexyl, wherein the R⁵ group is substituted with from 0 to 3 R^{D} substituents selected from the group consisting of halogen, F, Cl, Br, R^{m}, —NO₂, —CN, —NR^{q}R^{r}, —OR^{q}, —C(O)₂NR^{q}R^{r}, —NR^{q}C(O)R^{r}, —S(O)₂R^{r}, —SR^{q} and phenyl.

19. The compound of claim 18, wherein R⁵ is selected from the group consisting of: 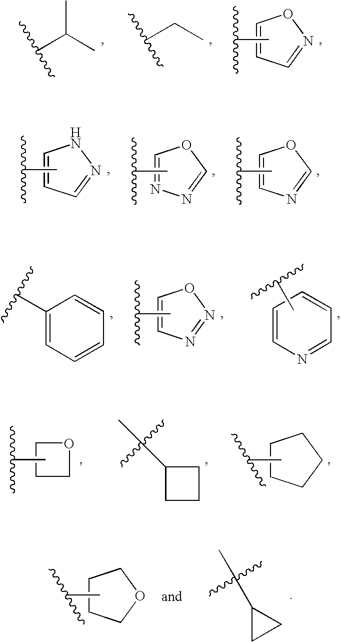
wherein from 0 to 3 hydrogen atoms attached to a carbon or nitrogen atom of R⁵ is optionally independently replaced with a R^{D} substitutent selected from the group consisting of halogen, C₁₋₃ haloalkyl, C₁₋₃ alkyl, —NR^{q}R^{r}, —OR^{q}, —S(O)₂R^{r}, halogen, F, Cl, and Br.

20. The compound of claim 1, wherein D is selected from the group set forth in FIG. 1, FIG. 2 or FIG. 3.

21. The compound of claim 20, wherein D is selected from the group consisting:

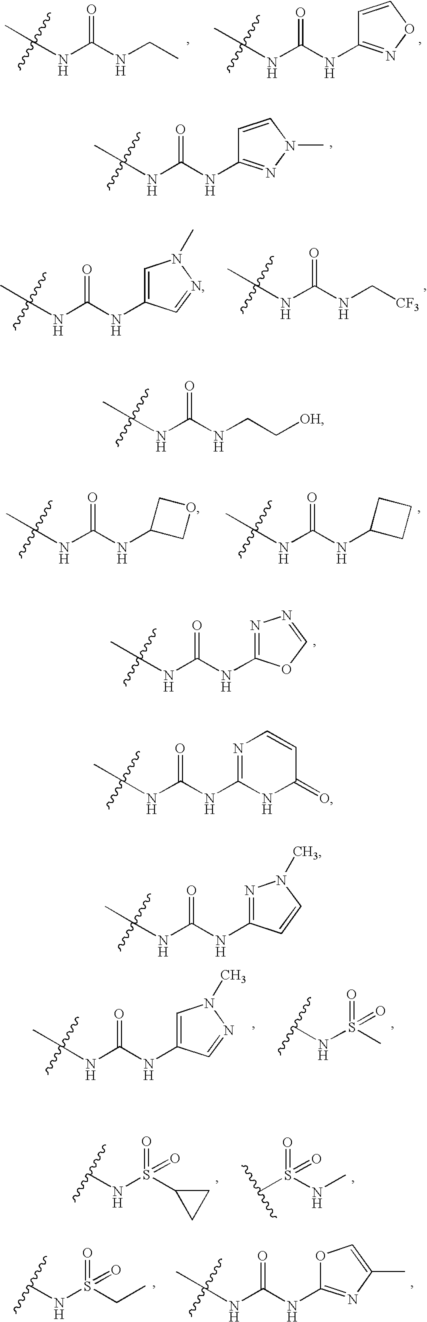

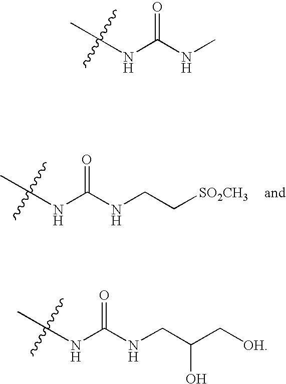

22. The compound of claim 1, wherein said compound is selected from the group consisting of:
1-ethyl-3-(4-(4-morpholino-6,7-dihydro-5H-pyrano[2,3-d]pyrimidin-2-yl)phenyl)urea;
(S)-1-ethyl-3-(4-(4-(3-methylmorpholino)-6,7-dihydro-5H-pyrano[2,3-d]pyrimidin-2-yl)phenyl)urea;
(S)-1-ethyl-3-(4-(4-(3-methylmorpholino)-7,8-dihydro-5H-pyrano[4,3-d]pyrimidin-2-yl)phenyl)urea;
1-ethyl-3-(4-(4-morpholino-7,8-dihydro-5H-pyrano[4,3-d]pyrimidin-2-yl)phenyl)urea;
(S)-1-ethyl-3-(4-(4-(3-ethylmorpholino)-7,8-dihydro-5H-pyrano[4,3-d]pyrimidin-2-yl)phenyl)urea;
(S)-1-(isoxazol-3-yl)-3-(4-(4-(3-methylmorpholino)-7,8-dihydro-5H-pyrano[4,3-d]pyrimidin-2-yl)phenyl)urea;
(S)-1-(1-methyl-1H-pyrazol-3-yl)-3-(4-(4-(3-methylmorpholino)-7,8-dihydro-5H-pyrano[4,3-d]pyrimidin-2-yl)phenyl)urea;
(S)-1-(1-methyl-1H-pyrazol-4-yl)-3-(4-(4-(3-methylmorpholino)-7,8-dihydro-5H-pyrano[4,3-d]pyrimidin-2-yl)phenyl)urea;
(S)-1-(4-(4-(3-methylmorpholino)-7,8-dihydro-5H-pyrano[4,3-d]pyrimidin-2-yl)phenyl)-3-(2,2,2-trifluoroethyl)urea;
(S)-1-(2-hydroxyethyl)-3-(4-(4-(3-methylmorpholino)-7,8-dihydro-5H-pyrano[4,3-d]pyrimidin-2-yl)phenyl)urea;
(S)-1-(4-(4-(3-methylmorpholino)-7,8-dihydro-5H-pyrano[4,3-d]pyrimidin-2-yl)phenyl)-3-(oxetan-3-yl)urea;
(S)-1-cyclobutyl-3-(4-(4-(3-methylmorpholino)-7,8-dihydro-5H-pyrano[4,3-d]pyrimidin-2-yl)phenyl)urea;
(S)-1-(5-methyl-1,3,4-oxadiazol-2-yl)-3-(4-(4-(3-methylmorpholino)-7,8-dihydro-5H-pyrano[4,3-d]pyrimidin-2-yl)phenyl)urea;
(S)-1-ethyl-3-(4-(4-(3-ethylmorpholino)-6,7-dihydro-5H-pyrano[2,3-d]pyrimidin-2-yl)phenyl)urea;
(S)-2-(4-(4-(3-methylmorpholino)-7,8-dihydro-5H-pyrano[4,3-d]pyrimidin-2-yl)phenylamino)pyrimidin-4(3H)-one;
(S)-6-(4-(4-(3-methylmorpholino)-7,8-dihydro-5H-pyrano[4,3-d]pyrimidin-2-yl)phenylamino)pyridin-2(1H)-one;
(S)-1-(1-methyl-1H-pyrazol-3-yl)-3-(4-(4-(3-methylmorpholino)-6,7-dihydro-5H-pyrano[2,3-d]pyrimidin-2-yl)phenyl)urea;
(S)-1-(1-methyl-1H-pyrazol-4-yl)-3-(4-(4-(3-methylmorpholino)-6,7-dihydro-5H-pyrano[2,3-d]pyrimidin-2-yl)phenyl)urea;
(S)-1-(4-(4-(3-methylmorpholino)-6,7-dihydro-5H-pyrano[2,3-d]pyrimidin-2-yl)phenyl)-3-(oxetan-3-yl)urea;
(S)-1-(2-hydroxyethyl)-3-(4-(4-(3-methylmorpholino)-6,7-dihydro-5H-pyrano[2,3-d]pyrimidin-2-yl)phenyl)urea;
(S)-1-(4-(7,7-dimethyl-4-(3-methylmorpholino)-5,7-dihydrofuro [3,4-d]pyrimidin-2-yl)phenyl)-3-ethylurea;
1-(4-(4-((1S,4 S)-2-oxa-5-azabicyclo [2.2.1]heptan-5-yl)-6,7-dihydro-5H-pyrano[2,3-d]pyrimidin-2-yl)phenyl)-3-ethylurea;
(S)-2-(4-(4-(3-methylmorpholino)-6,7-dihydro-5H-pyrano[2,3-d]pyrimidin-2-yl)phenylamino)pyrimidin-4(3H)-one;
(S)-6-(4-(4-(3-methylmorpholino)-6,7-dihydro-5H-pyrano[2,3-d]pyrimidin-2-yl)phenylamino)pyridin-2(1H)-one;
(S)-4-(3-methylmorpholino)-2-(4-(methylsulfonyl)phenyl)-7,8-dihydro-5H-pyrano[4,3-d]pyrimi dine;
(S)—N-methyl-4-(4-(3-methylmorpholino)-7,8-dihydro-5H-pyrano[4,3-d]pyrimidin-2-yl)benzenesulfonamide;
(S)—N-(4-(4-(3-methylmorpholino)-7,8-dihydro-5H-pyrano[4,3-d]pyrimidin-2-yl)phenyl)methanesulfonamide;
(S)—N-(4-(4-(3-methylmorpholino)-7,8-dihydro-5H-pyrano[4,3-d]pyrimidin-2-yl)phenyl)cyclopropanesulfonamide;
(S)-6-(4-(7,7-dimethyl-4-(3-methylmorpholino)-5,7-dihydrofuro [3,4-d]pyrimidin-2-yl)phenylamino)pyridin-2(1H)-one;
1-ethyl-1-((ethylamino)carbonyl)-3-(4-(4-morp holino-6,8-dihydro-5H-pyrano[3,4-d]pyrimidin-2-yl)phenyl)urea;
(S)—N-(4-(4-(3-methylmorpholino)-7,8-dihydro-5H-pyrano[4,3-d]pyrimidin-2-yl)phenyl)ethanesulfonamide;
(S)-1-ethyl-3-(4-(4-(3-methylmorpholino)-6,8-dihydro-5H-pyrano[3,4-d]pyrimidin-2-yl)phenyl)urea;
(S)-1-ethyl-1-((ethylamino)carbonyl)-3-(4-(4-(3-methylmorp holino)-6,8-dihydro-5H-pyrano[3,4-d]pyrimi din-2-yl)phenyl)urea;
1-ethyl-3-(4-(4-morpholino-7,8-dihydro-6H-pyrano[3,2-d]pyrimidin-2-yl)phenyl)urea;
(S)-2-(4-(4-(3-ethylmorpholino)-7,8-dihydro-5H-pyrano[4,3-d]pyrimi din-2-yl)phenylamino)pyrimidin-4(3H)-one;
(S)-6-(4-(4-(3-ethylmorpholino)-7,8-dihydro-5H-pyrano[4,3-d]pyrimi din-2-yl)phenylamino)pyridin-2(1H)-one;
(S)-1-(4-(4-(3-ethylmorpholino)-7,8-dihydro-5H-pyrano[4,3-d]pyrimi din-2-yl)phenyl)-3-(oxetan-3-yl)urea;
1-ethyl-3-(4-(4′-morpholino-5′,6′-dihydrospiro[cyclopropane-1,7′-pyrano[2,3-d]pyrimidine]-2′-yl)phenyl)urea;
2-(4-(4′-morpholino-5′,6′-dihydrospiro[cyclopropane-1,7′-pyrano[2,3-d]pyrimidine]-2′-yl)phenylamino)pyrimidin-4(3H)-one;
1-(4-(4′-morpholino-5′,6′-dihydrospiro[cyclopropane-1,7′-pyrano[2,3-d]pyrimidine]-2′-yl)phenyl)-3-(oxetan-3-yl)urea;
1-(1-methyl-1H-pyrazol-3-yl)-3-(4-(4′-morpholino-5′,6′-dihydrospiro[cyclopropane-1,7′-pyrano[2,3-d]pyrimidine]-2′-yl)phenyl)urea;
1-(1-methyl-1H-pyrazol-4-yl)-3-(4-(4′-morpholino-5′,6′-dihydrospiro[cyclopropane-1,7′-pyrano[2,3-d]pyrimidine]-2′-yl)phenyl)urea;
1-(4-(4′-(4-methoxypiperidin-1-yl)-5′,6′-dihydrospiro[cyclopropane-1,7′-pyrano[2,3-d]pyrimidine]-2′-yl)phenyl)-3-(oxetan-3-yl)urea;
1-(4-(4′-(4-methoxypiperidin-1-yl)-5′,6′-dihydrospiro[cyclopropane-1,7′-pyrano[2,3-d]pyrimidine]-2′-yl)phenyl)-3-(1-methyl-1H-pyrazol-3-yl)urea;
2-(4-(4′-(4-methoxypiperidin-1-yl)-5′,6′-dihydrospiro[cyclopropane-1,7′-pyrano[2,3-d]pyrimidine]-2′-yl)phenylamino)pyrimidin-4(3H)-one;
(S)-1-ethyl-3-(4-(4′-(3-methylmorpholino)-5′,6′-dihydrospiro[cyclopropane-1,7′-pyrano[2,3-d]pyrimidine]-2′-yl)phenyl)urea;
(S)-1-(1-methyl-1H-pyrazol-4-yl)-3-(4-(4′-(3-methylmorpholino)-5′,6′-dihydrospiro[cyclopropane-1,7′-pyrano[2,3-d]pyrimidine]-2′-yl)phenyl)urea;
(S)-1-(4-(4′-(3-methylmorpholino)-5′,6′-dihydrospiro[cyclopropane-1,7′-pyrano[2,3-d]pyrimidine]-2′-yl)phenyl)-3-(oxetan-3-yl)urea;
(S)-1-(1-methyl-1H-pyrazol-3-yl)-3-(4-(4′-(3-methylmorpholino)-5′,6′-dihydrospiro[cyclopropane-1,7′-pyrano[2,3-d]pyrimidine]-2′-yl)phenyl)urea;
(S)-1-(4-(7,7-dimethyl-4-(3-methylmorpholino)-5,7-dihydrofuro [3,4-d]pyrimidin-2-yl)phenyl)-3-(1-methyl-1H-pyrazol-4-yl)urea;
(S)-1-(4-(4′-(3-methylmorpholino)-5′,6′-dihydrospiro[cyclopropane-1,7′-pyrano[2,3-d]pyrimidine]-2′-yl)phenyl)-3-(4-methyloxazol-2-yl)urea;
(S)-6-(4-(4′-(3-methylmorpholino)-5′,6′-dihydrospiro[cyclopropane-1,7′-pyrano[2,3-d]pyrimidine]-2′-yl)phenylamino)pyridin-2(1H)-one;
(S)-2-(4-(4′-(3-methylmorpholino)-5′,6′-dihydrospiro[cyclopropane-1,7′-pyrano[2,3-d]pyrimidine]-2′-yl)phenylamino)pyrimidin-4(3H)-one;
(S)-1-methyl-3-(4-(4′-(3-methylmorpholino)-5′,6′-dihydrospiro[cyclopropane-1,7′-pyrano[2,3-d]pyrimidine]-2′-yl)phenyl)urea;
(S)-1-(4-(4′-(3-methylmorpholino)-5′,6′-dihydrospiro[cyclopropane-1,7′-pyrano[2,3-d]pyrimidine]-2′-yl)phenyl)-3-(2-(methylsulfonyl)ethyl)urea;
(S)-1-methyl-3-(4-(4-(3-methylmorpholino)-7,8-dihydro-5H-pyrano[4,3-d]pyrimidin-2-yl)phenyl)urea;
(S)-1-(4-(4-(3-methylmorpholino)-7,8-dihydro-5H-pyrano[4,3-d]pyrimidin-2-yl)phenyl)-3-(2-(methylsulfonyl)ethyl)urea;
(S)-1-(4-(7,7-dimethyl-4-(3-methylmorpholino)-5,7-dihydrofuro[3,4-d]pyrimidin-2-yl)phenyl)-3-(oxetan-3-yl)urea;
(S)-1-(4-(7,7-dimethyl-4-(3-methylmorpholino)-5,7-dihydrofuro[3,4-d]pyrimidin-2-yl)phenyl)-3-(2-hydroxyethyl)urea;
(S)-1-(2-cyanoethyl)-3-(4-(7,7-dimethyl-4-(3-methylmorpholino)-5,7-dihydrofuro[3,4-d]pyrimidin-2-yl)phenyl)urea;
1-(4-(4-((1R,5S)-3-oxa-8-azabicyclo[3.2.1]octan-8-yl)-6,7-dihydro-5H-pyrano[2,3-d]pyrimidin-2-yl)phenyl)-3-ethylurea;
1-((R)-2,3-dihydroxypropyl)-3-(4-(7,7-dimethyl-4-((S)-3-methylmorpholino)-5,7-dihydrofuro[3,4-d]pyrimidin-2-yl)phenyl)urea;
(S)-1-(2-hydroxyethyl)-3-(4-(4′-(3-methylmorpholino)-5′,6′-dihydrospiro[cyclopropane-1,7′-pyrano[2,3-d]pyrimidine]-2′-yl)phenyl)urea;
(S)-1-(2-cyanoethyl)-3-(4-(4′-(3-methylmorpholino)-5′,6′-dihydrospiro[cyclopropane-1,7′-pyrano[2,3-d]pyrimidine]-2′-yl)phenyl)urea;
1-(4-(7,7-dimethyl-4-morpholino-5-oxo-5,7-dihydrofuro[3,4-d]pyrimidin-2-yl)phenyl)-3-ethylurea;
14(S)-2,3-dihydroxypropyl)-3-(4-(4′4(S)-3-methylmorpholino)-5′,6′-dihydrospiro[cyclopropane-1,7′-pyrano[2,3-d]pyrimidine]-2′-yl)phenyl)urea;
(S)-1-methoxy-3-(4-(4′-(3-methylmorpholino)-5′,6′-dihydrospiro[cyclopropane-1,7′-pyrano[2,3-d]pyrimidine]-2′-yl)phenyl)urea;
1-((R)-2,3-dihydroxypropyl)-3-(4-(4′-((S)-3-methylmorpholino)-5′,6′-dihydrospiro[cyclopropane-1,7′-pyrano[2,3-d]pyrimidine]-2′-yl)phenyl)urea;
1-(4-(7-(benzyloxymethyl)-4-((S)-3-methylmorpholino)-7,8-dihydro-5H-pyrano[4,3-d]pyrimidin-2-yl)phenyl)-3-ethylurea;
1-Ethyl-3-{4-[(1R,9S)-3-((S)-3-methyl-morpholin-4-yl)-12-oxa-4,6-diaza-tricyclo[7.2.1.0-2,7]dodeca-2(7),3,5-trien-5-yl]-phenyl}-urea;
1-Ethyl-3-{4-[(1S,9R)-3-((S)-3-methyl-morpholin-4-yl)-12-oxa-4,6-diaza-tricyclo [7.2.1.0-2,7]dodeca-2(7),3,5-trien-5-yl]-phenyl}-urea;
1-(4-(4-((1R,5S)-3-oxa-8-azabicyclo [3.2.1]octan-8-yl)-6,7-dihydro-5H-pyrano[2,3-d]pyrimidin-2-yl)phenyl)-3-(oxetan-3-yl)urea;
1-ethyl-3-(4-(7-(2-hydroxyethyl)-7-methyl-4-(S)-3-methylmorpholino)-5,7-dihydrofuro [3,4-d]pyrimidin-2-yl)phenyl)urea;
2-(4-(7-(hydroxymethyl)-4-((S)-3-methylmorpholino)-7,8-dihydro-5H-pyrano[4,3-d]pyrimidin-2-yl)phenylamino)pyrimidin-4(3H)-one;
1-ethyl-3-(4-((R)-7-(2-hydroxyethyl)-7-methyl-4-((S)-3-methylmorpholino)-5,7-dihydrofuro [3,4-d]pyrimidin-2-yl)phenyl)urea;
1-ethyl-3-(4-((S)-7-(2-hydroxyethyl)-7-methyl-4-((S)-3-methylmorpholino)-5,7-dihydrofuro [3,4-d]pyrimidin-2-yl)phenyl)urea;
1-{4-[(1R,9S)-3-((S)-3-Methyl-morpholin-4-yl)-12-oxa-4,6-diaza-tricyclo [7.2.1.0-2,7]dodeca-2(7),3,5-trien-5-yl]-phenyl}-3-oxetan-3-yl-urea;
1-{4-[(1S,9R)-3-((S)-3-Methyl-morpholin-4-yl)-12-oxa-4,6-diaza-tricyclo [7.2.1.0-2,7]dodeca-2(7),3,5-trien-5-yl]-phenyl}-3-oxetan-3-yl-urea;
1-(4-(4′-((1R,5S)-3-oxa-8-azabicyclo [3.2.1]octan-8-yl)-5′,6′-dihydrospiro[cyclopropane-1,7′-pyrano[2,3-d]pyrimidine]-2′-yl)phenyl)-3-(oxetan-3-yl)urea;
1-(4-(4′-((1R,5S)-3-oxa-8-azabicyclo [3.2.1]octan-8-yl)-5′,6′-dihydrospiro[cyclopropane-1,7′-pyrano[2,3-d]pyrimidine]-2′-yl)phenyl)-3-(2-hydroxyethyl)urea;
(S)-1-(1-(hydroxymethyl)cyclopropyl)-3-(4-(4′-(3-methylmorpholino)-5′,6′-dihydrospiro[cyclopropane-1,7′-pyrano[2,3-d]pyrimidine]-2′-yl)phenyl)urea;
1-ethyl-3-(4-(7-(hydroxymethyl)-4-((S)-3-methylmorpholino)-7,8-dihydro-5H-pyrano[4,3-d]pyrimidin-2-yl)phenyl)urea;
(S)-1-(4-(7,7-dimethyl-4-(3-methylmorpholino)-7,8-dihydro-5H-pyrano[4,3-d]pyrimidin-2-yl)phenyl)-3-ethylurea;
1-(4-((R)-7-allyl-7-methyl-4-((S)-3-methylmorpholino)-5,7-dihydrofuro [3,4-d]pyrimidin-2-yl)phenyl)-3-ethylurea;
1-(4-((S)-7-allyl-7-methyl-4-(S)-3-methylmorpholino)-5,7-dihydrofuro [3,4-d]pyrimidin-2-yl)phenyl)-3-ethylurea;
1-(4-(7-(cyclopropylmethyl)-7-methyl-4-((S)-3-methylmorpholino)-5,7-dihydrofuro [3,4-d]pyrimidin-2-yl)phenyl)-3-ethylurea;
3-ethyl-1-(4-((S)-7-(2-hydroxyethyl)-7-methyl-4-((S)-3-methylmorp holino)-5,7-dihydro furo [3,4-d]pyrimidin-2-yl)phenyl)-1-methylurea;
3-ethyl-1-(4-((R)-7-(2-hydroxyethyl)-7-methyl-4-((S)-3-methylmorpholino)-5,7-dihydro furo [3,4-d]pyrimidin-2-yl)phenyl)-1-methylurea;
1-ethyl-3-(4-(4-morpholino-7-(pyridin-2-yl)-7,8-dihydro-5H-pyrano[4,3-d]pyrimidin-2-yl)phenyl)ure a;
1-ethyl-3-(4-(7-methyl-4-((S)-3-methylmorpholino)-7-propyl-5,7-dihydrofuro [3,4-d]pyrimidin-2-yl)phenyl)urea;
1-ethyl-3-(4-((S)-7-(2-methoxyethyl)-7-methyl-4-((S)-3-methylmorpholino)-5,7-dihydrofuro [3,4-d]pyrimidin-2-yl)phenyl)urea;
1-ethyl-3-(4-((R)-7-(2-methoxyethyl)-7-methyl-4-((S)-3-methylmorpholino)-5,7-dihydrofuro [3,4-d]pyrimidin-2-yl)phenyl)urea;
1-ethyl-3-(4-((S)-7-(3-hydroxypropyl)-7-methyl-4-((S)-3-methylmorpholino)-5,7-dihydrofuro [3,4-d]pyrimidin-2-yl)phenyl)urea;
1-ethyl-3-(4-((R)-7-(3-hydroxypropyl)-7-methyl-4-((S)-3-methylmorpholino)-5,7-dihydro furo [3,4-d]pyrimidin-2-yl)phenyl)urea;
1-ethyl-3-(4-((7S)-7-(2-hydroxypropyl)-7-methyl-4-((S)-3-methylmorpholino)-5,7-dihydrofuro [3,4-d]pyrimidin-2-yl)phenyl)urea;
1-ethyl-3-(4-((7R)-7-(2-hydroxypropyl)-7-methyl-4-((S)-3-methylmorpholino)-5,7-dihydrofuro [3,4-d]pyrimidin-2-yl)phenyl)urea;
1-ethyl-3-(4-((S)-7-methyl-4-((S)-3-methylmorpholino)-7-(2-morpholinoethyl)-5,7-dihydrofuro [3,4-d]pyrimidin-2-yl)phenyl)urea;
1-ethyl-3-(4-((R)-7-methyl-4-((S)-3-methylmorpholino)-7-(2-morpholinoethyl)-5,7-dihydrofuro [3,4-d]pyrimidin-2-yl)phenyl)urea;
1-ethyl-3-(4-((S)-7-methyl-7-(2-(2-methyl-1H-imidazol-1-yl)ethyl)-4-((S)-3-methylmorpholino)-5,7-dihydrofuro [3,4-d]pyrimidin-2-yl)phenyl)urea;
1-ethyl-3-(4-((R)-7-methyl-7-(2-(2-methyl-1H-imidazol-1-yl)ethyl)-4-((S)-3-methylmorpholino)-5,7-dihydrofuro [3,4-d]pyrimidin-2-yl)phenyl)urea;
1-(4-((R)-7-(2-(azetidin-1-yl)ethyl)-7-methyl-4-((S)-3-methylmorp holino)-5,7-dihydro furo [3,4-d]pyrimidin-2-yl)phenyl)-3-ethylurea;
1-(4-((S)-7-(2-(azetidin-1-yl)ethyl)-7-methyl-4-((S)-3-methylmorpholino)-5,7-dihydrofuro[3,4-d]pyrimidin-2-yl)phenyl)-3-ethylurea;
5-(4-((1R,5S)-3-oxa-8-azabicyclo[3.2.1]octan-8-yl)-6,7-dihydro-5H-pyrano[2,3-d]pyrimidin-2-yl)pyrimidin-2-amine;
5-(4-((1R,5S)-3-oxa-8-azabicyclo[3.2.1]octan-8-yl)-6,7-dihydro-5H-pyrano[2,3-d]pyrimidin-2-yl)pyridin-2-amine;
5-(4-((1R,5S)-8-oxa-3-azabicyclo[3.2.1]octan-3-yl)-6,7-dihydro-5H-pyrano[2,3-d]pyrimidin-2-yl)pyrimidin-2-amine;
5-(4-((1R,5S)-8-oxa-3-azabicyclo[3.2.1]octan-3-yl)-6,7-dihydro-5H-pyrano[2,3-d]pyrimidin-2-yl)pyridin-2-amine;
(S)-1-ethyl-3-(4-(4-(3-methylmorpholino)-7-oxo-5,7-dihydrofuro[3,4-d]pyrimidin-2-yl)phenyl)urea;
1-ethyl-3-(4-((S)-7-methyl-4-((S)-3-methylmorpholino)-7-(2-(pyridin-4-yloxy)ethyl)-5,7-dihydrofuro[3,4-d]pyrimidin-2-yl)phenyl)urea;
1-ethyl-3-(4-((R)-7-methyl-4-((S)-3-methylmorpholino)-7-(2-(pyridin-4-yloxy)ethyl)-5,7-dihydrofuro[3,4-d]pyrimidin-2-yl)phenyl)urea;
5-((S)-7-(2-methoxyethyl)-7-methyl-4-(S)-3-methylmorpholino)-5,7-dihydrofuro[3,4-d]pyrimidin-2-yl)pyrimidin-2-amine;
5-((R)-7-(2-methoxyethyl)-7-methyl-4-((S)-3-methylmorpholino)-5,7-dihydrofuro[3,4-d]pyrimidin-2-yl)pyrimidin-2-amine;
1-ethyl-3-(4-(7-methyl-4-(3-methylmorpholino)-7-(2-phenoxyethyl)-5,7-dihydrofuro[3,4-d]pyrimidin-2-yl)phenyl)urea;
1-ethyl-3-(4-(7-methyl-4-(3-methylmorpholino)-7-(2-phenoxyethyl)-5,7-dihydrofuro[3,4-d]pyrimidin-2-yl)phenyl)urea;
(S)-1-(4-(7-allyl-7-methyl-4-morpholino-5,7-dihydrofuro[3,4-d]pyrimidin-2-yl)phenyl)-3-ethylurea;
(R)-1-(4-(7-allyl-7-methyl-4-morpholino-5,7-dihydrofuro[3,4-d]pyrimidin-2-yl)phenyl)-3-ethylurea;
5-(7,7-dimethyl-4-morpholino-5,7-dihydrofuro[3,4-d]pyrimidin-2-yl)pyridin-2-amine;
(R)-1-ethyl-3-(4-(7-methyl-4-morpholino-7-propyl-5,7-dihydrofuro[3,4-d]pyrimidin-2-yl)phenyl)urea;
(S)-1-ethyl-3-(4-(7-methyl-4-morpholino-7-propyl-5,7-dihydrofuro[3,4-d]pyrimidin-2-yl)phenyl)urea;
5-((S)-7-(2-methoxyethyl)-7-methyl-4-((S)-3-methylmorpholino)-5,7-dihydrofuro[3,4-d]pyrimidin-2-yl)benzo[d]oxazol-2-amine;
5-((R)-7-(2-methoxyethyl)-7-methyl-4-((S)-3-methylmorpholino)-5,7-dihydrofuro[3,4-d]pyrimidin-2-yl)benzo[d]oxazol-2-amine;
5-((S)-7-(2-methoxyethyl)-7-methyl-4-((S)-3-methylmorpholino)-5,7-dihydrofuro[3,4-d]pyrimidin-2-yl)pyridin-2-amine;
5-((R)-7-(2-methoxyethyl)-7-methyl-4-((S)-3-methylmorpholino)-5,7-dihydrofuro[3,4-d]pyrimidin-2-yl)pyridin-2-amine;
6-((S)-7-(2-methoxyethyl)-7-methyl-4-((S)-3-methylmorpholino)-5,7-dihydrofuro[3,4-d]pyrimidin-2-yl)benzo[d]oxazol-2-amine;
6-((R)-7-(2-methoxyethyl)-7-methyl-4-((S)-3-methylmorpholino)-5,7-dihydrofuro[3,4-d]pyrimidin-2-yl)benzo[d]oxazol-2-amine;
(S)-1-ethyl-3-(4-(7-(2-hydroxyethyl)-7-methyl-4-morpholino-5,7-dihydrofuro[3,4-d]pyrimidin-2-yl)phenyl)urea;
(R)-1-ethyl-3-(4-(7-(2-hydroxyethyl)-7-methyl-4-morpholino-5,7-dihydrofuro[3,4-d]pyrimidin-2-yl)phenyl)urea;
1-ethyl-3-(4-((R)-7-(2-(ethyl(methyl)amino)ethyl)-7-methyl-4-((S)-3-methylmorpholino)-5,7-dihydrofuro[3,4-d]pyrimidin-2-yl)phenyl)urea;
1-ethyl-3-(4-((S)-7-(2-(ethyl(methyl)amino)ethyl)-7-methyl-4-((S)-3-methylmorpholino)-5,7-dihydrofuro[3,4-d]pyrimidin-2-yl)phenyl)urea;
1-(4-((R)-7-(2-cyanoethyl)-7-methyl-4-((S)-3-methylmorpholino)-5,7-dihydrofuro[3,4-d]pyrimidin-2-yl)phenyl)-3-ethylurea;
1-(4-((S)-7-(2-cyanoethyl)-7-methyl-4-((S)-3-methylmorpholino)-5,7-dihydrofuro[3,4-d]pyrimidin-2-yl)phenyl)-3-ethylurea;
(S)-5-(7,7-dimethyl-4-(3-methylmorpholino)-5,7-dihydrofuro[3,4-d]pyrimidin-2-yl)pyrimidin-2-amine;
1-(4-((R)-7-(2-(1H-imidazol-1-yl)ethyl)-7-methyl-4-((S)-3-methylmorpholino)-5,7-dihydrofuro[3,4-d]pyrimidin-2-yl)phenyl)-3-ethylurea;
1-(4-((S)-7-(2-(1H-imidazol-1-yl)ethyl)-7-methyl-4-((S)-3-methylmorpholino)-5,7-dihydrofuro[3,4-d]pyrimidin-2-yl)phenyl)-3-ethylurea;
5-((S)-7-methyl-4-((S)-3-methylmorpholino)-7-(2-phenoxyethyl)-5,7-dihydrofuro[3,4-d]pyrimidin-2-yl)pyrimidin-2-amine;
5-((R)-7-methyl-4-((S)-3-methylmorpholino)-7-(2-phenoxyethyl)-5,7-dihydrofuro[3,4-d]pyrimidin-2-yl)pyrimidin-2-amine;
6-((S)-7-(2-methoxyethyl)-7-methyl-4-((S)-3-methylmorpholino)-5,7-dihydrofuro[3,4-d]pyrimidin-2-yl)benzo[d]isoxazol-3-amine;
6-((R)-7-(2-methoxyethyl)-7-methyl-4-((S)-3-methylmorpholino)-5,7-dihydrofuro[3,4-d]pyrimidin-2-yl)benzo[d]isoxazol-3-amine;
5-(7,7-dimethyl-4-morpholino-5,7-dihydrofuro[3,4-d]pyrimidin-2-yl)benzo[d]oxazol-2-amine;
6-(7,7-dimethyl-4-morpholino-5,7-dihydrofuro[3,4-d]pyrimidin-2-yl)benzo[d]oxazol-2-amine;
6-(4-((1R,5S)-8-oxa-3-azabicyclo[3.2.1]octan-3-yl)-6,7-dihydro-5H-pyrano[2,3-d]pyrimidin-2-yl)-1H-benzo[d]imidazol-2-amine;
6-(7,7-dimethyl-4-morpholino-5,7-dihydrofuro[3,4-d]pyrimidin-2-yl)benzo[d]isoxazol-3-amine;
5-(7,7-dimethyl-4-morpholino-5,7-dihydrofuro[3,4-d]pyrimidin-2-yl)benzo[d]isoxazol-3-amine;
6-(7,7-dimethyl-4-morpholino-5,7-dihydrofuro[3,4-d]pyrimidin-2-yl)-1H-benzo[d]imidazol-2-amine;
5-((S)-7-(2-methoxyethyl)-7-methyl-4-((S)-3-methylmorpholino)-5,7-dihydrofuro[3,4-d]pyrimidin-2-yl)benzo[d]isoxazol-3-amine;
5-((R)-7-(2-methoxyethyl)-7-methyl-4-((S)-3-methylmorpholino)-5,7-dihydrofuro[3,4-d]pyrimidin-2-yl)benzo[d]isoxazol-3-amine;
1-ethyl-3-(4-((S)-7-(hydroxymethyl)-7-methyl-4-((S)-3-methylmorpholino)-5,7-dihydrofuro[3,4-d]pyrimidin-2-yl)phenyl)urea;
1-ethyl-3-(4-((R)-7-(hydroxymethyl)-7-methyl-4-((S)-3-methylmorpholino)-5,7-dihydrofuro[3,4-d]pyrimidin-2-yl)phenyl)urea;
1-(4-((R)-7-allyl-4-((1R,5S)-8-oxa-3-azabicyclo[3.2.1]octan-3-yl)-7-methyl-5,7-dihydrofuro[3,4-d]pyrimidin-2-yl)phenyl)-3-ethylurea;
1-(4-((S)-7-allyl-44(1R,5S)-8-oxa-3-azabicyclo[3.2.1]octan-3-yl)-7-methyl-5,7-dihydrofuro[3,4-d]pyrimidin-2-yl)phenyl)-3-ethylurea;
1-(4-((S)-4-((1R,5S)-8-oxa-3-azabicyclo[3.2.1]octan-3-yl)-7-methyl-7-propyl-5,7-dihydrofuro[3,4-d]pyrimidin-2-yl)phenyl)-3-ethylurea;
1-(4-((R)-4-((1R,5S)-8-oxa-3-azabicyclo[3.2.1]octan-3-yl)-7-methyl-7-propyl-5,7-dihydrofuro[3,4-d]pyrimidin-2-yl)phenyl)-3-ethylurea a;
(S)-6-(7,7-dimethyl-4-(3-methylmorpholino)-5,7-dihydrofuro[3,4-d]pyrimidin-2-yl)benzo[d]isoxazol-3-amine;
(S)-5-(7,7-dimethyl-4-(3-methylmorpholino)-5,7-dihydrofuro[3,4-d]pyrimidin-2-yl)benzo[d]isoxazol-3-amine;
(S)-6-(7,7-dimethyl-4-(3-methylmorpholino)-5,7-dihydrofuro[3,4-d]pyrimidin-2-yl)-1H-benzo[d]imidazol-2-amine;
1-(4-((S)-4-((1R,5S)-8-oxa-3-azabicyclo[3.2.1]octan-3-yl)-7-(2-hydroxyethyl)-7-methyl-5,7-dihydrofuro[3,4-d]pyrimidin-2-yl)phenyl)-3-ethylurea;
1-(4-((R)-4-((1R,5S)-8-oxa-3-azabicyclo[3.2.1]octan-3-yl)-7-(2-hydroxyethyl)-7-methyl-5,7-dihydrofuro[3,4-d]pyrimidin-2-yl)phenyl)-3-ethylurea;
5-(4-((1R,4R)-2-oxa-5-azabicyclo[2.2.1]heptan-5-yl)-7,7-dimethyl-5,7-dihydrofuro[3,4-d]pyrimidin-2-yl)pyrimidin-2-amine;
5-(4-((1R,5S)-3-oxa-8-azabicyclo[3.2.1]octan-8-yl)-7,7-dimethyl-5,7-dihydrofuro[3,4-d]pyrimidin-2-yl)pyrimidin-2-amine;
5-(4-((1R,5S)-8-oxa-3-azabicyclo[3.2.1]octan-3-yl)-7,7-dimethyl-5,7-dihydrofuro[3,4-d]pyrimidin-2-yl)pyrimidin-2-amine;
(S)-5-(7,7-dimethyl-4-(3-methylmorpholino)-5,7-dihydrofuro[3,4-d]pyrimidin-2-yl)benzo[d]oxazol-2-amine;
6-(7-(2-methoxyethyl)-7-methyl-4-((S)-3-methylmorpholino)-5,7-dihydrofuro[3,4-d]pyrimidin-2-yl)-1H-benzo[d]imidazol-2-amine;
6-((R)-7-(2-methoxyethyl)-7-methyl-4-((S)-3-methylmorpholino)-5,7-dihydrofuro[3,4-d]pyrimidin-2-yl)-1H-benzo[d]imidazol-2-amine;
(S)-6-(4-(3-methylmorpholino)-6,7-dihydro-5H-pyrano[2,3-d]pyrimidin-2-yl)-1H-benzo[d]imidazol-2-amine;
(S)-5-(4-(3-ethylmorpholino)-6,7-dihydro-5H-pyrano[2,3-d]pyrimidin-2-yl)pyrimidin-2-amine;
(S)-5-(4-(3-ethylmorpholino)-6,7-dihydro-5H-pyrano[2,3-d]pyrimidin-2-yl)pyridin-2-amine;
(S)-5-(7,7-dimethyl-4-(3-methylmorpholino)-5,7-dihydrofuro[3,4-d]pyrimidin-2-yl)-N-methyl-1H-benzo[d]imidazol-2-amine;
2-((S)-2-(2-amino-1H-benzo[d]imidazol-5-yl)-7-methyl-4-((S)-3-methylmorpholino)-5,7-dihydrofuro[3,4-d]pyrimidin-7-yl)ethanol;
1-ethyl-3-(4-((S)-7-(2-hydroxy-2-methylpropyl)-7-methyl-4-(S)-3-methylmorpholino)-5,7-dihydrofuro[3,4-d]pyrimidin-2-yl)phenyl)urea;
1-ethyl-3-(4-((R)-7-(2-hydroxy-2-methylpropyl)-7-methyl-4-((S)-3-methylmorpholino)-5,7-dihydrofuro[3,4-d]pyrimidin-2-yl)phenyl)urea;
2-((R)-2-(2-amino-1H-benzo[d]imidazol-5-yl)-7-methyl-4-((S)-3-methylmorpholino)-5,7-dihydrofuro[3,4-d]pyrimidin-7-yl)ethanol;
(S)-1-ethyl-3-(4-(7-(2-hydroxy-2-methylpropyl)-7-methyl-4-morpholino-5,7-dihydrofuro[3,4-d]pyrimidin-2-yl)phenyl)urea;
(R)-1-ethyl-3-(4-(7-(2-hydroxy-2-methylpropyl)-7-methyl-4-morpholino-5,7-dihydrofuro[3,4-d]pyrimidin-2-yl)phenyl)urea;
(S)-1-ethyl-3-(4-(4-(3-methylmorpholino)-5,7-dihydrofuro[3,4-d]pyrimidin-2-yl)phenyl)urea;
(R)-1-ethyl-3-(4-(7-(hydroxymethyl)-7-methyl-4-morpholino-5,7-dihydrofuro[3,4-d]pyrimidin-2-yl)phenyl)urea;
(S)-1-ethyl-3-(4-(7-(hydroxymethyl)-7-methyl-4-morpholino-5,7-dihydrofuro[3,4-d]pyrimidin-2-yl)phenyl)urea;
1-ethyl-3-(4-(4-morpholino-5,7-dihydrofuro[3,4-d]pyrimidin-2-yl)phenyl)urea;
1-(4-(4-((1R,5S)-3-oxa-8-azabicyclo[3.2.1]octan-8-yl)-5,7-dihydrofuro[3,4-d]pyrimidin-2-yl)phenyl)-3-ethylurea; and
1-(4-(4-(8-oxa-3-azabicyclo[3.2.1]octan-3-yl)-5,7-dihydrofuro[3,4-d]pyrimidin-2-yl)phenyl)-3-ethylurea.

23. A pharmaceutical composition comprising a compound of claim 1 and a pharmaceutically acceptable carrier, diluent or excipient.

24. A method for the treatment of cancer in a mammal comprising administering to a mammal in need thereof a therapeutically acceptable amount of a compound of claim 1, wherein the cancer is selected from breast, ovary, cervix, prostate, testis, genitourinary tract, esophagus, larynx, glioblastoma, neuroblastoma, stomach, skin, keratoacanthoma, lung, epidermoid carcinoma, large cell carcinoma, non-small cell lung carcinoma (NSCLC), small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, hairy cells, buccal cavity and pharynx (oral), lip, tongue, mouth, pharynx, small intestine, colon-rectum, large intestine, rectum, brain and central nervous system, Hodgkin's and leukemia.

25. The method of claim 24, wherein said cancer is selected from breast, NSCLC, small cell carcinoma, liver carcinoma, lymphoid disorders, sarcoma, colon-rectum, rectum, leukemia.

26. The method of claim 24, wherein a compound of claim 1 is administered in combination with another chemotherapeutic agent.

27. The method of claim 24, wherein said mammal is a human.

28. A method of inhibiting the activity of mTOR kinase in a mammal comprising administering to the mammal a therapeutically acceptable amount of a compound of claim 1.

29. A compound of Formula I used for the treatment of a cancer selected from the group consisting of breast, ovary, cervix, prostate, testis, genitourinary tract, esophagus, larynx, glioblastoma, neuroblastoma, stomach, skin, keratoacanthoma, lung, epidermoid carcinoma, large cell carcinoma, non-small cell lung carcinoma (NSCLC), small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, hairy cells, buccal cavity and pharynx (oral), lip, tongue, mouth, pharynx, small intestine, colon-rectum, large intestine, rectum, brain and central nervous system, Hodgkin's and leukemia.

30. The compound of claim 29, wherein said cancer is selected from breast, NSCLC, small cell carcinoma, liver carcinoma, lymphoid disorders, sarcoma, colon-rectum, rectum, leukemia.

31. The use of a compound of Formula I in the manufacture of a medicament for the treatment of a cancer selected from the group consisting of breast, ovary, cervix, prostate, testis, genitourinary tract, esophagus, larynx, glioblastoma, neuroblastoma, stomach, skin, keratoacanthoma, lung, epidermoid carcinoma, large cell carcinoma, non-small cell lung carcinoma (NSCLC), small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, hairy cells, buccal cavity and pharynx (oral), lip, tongue, mouth, pharynx, small intestine, colon-rectum, large intestine, rectum, brain and central nervous system, Hodgkin's and leukemia.
